(19) United States
(12) Patent Application Publication
(10) Pub. No.: US 2011/0236389 A1
(43) Pub. Date: Sep. 29, 2011

(54) Human Angiostatin Interacting and Tumor Metastasis Involving Protein Variants and Uses Thereof

(75) Inventors: Jian Ni, Jiangsu (CN); Yan Peng, Jiangsu (CN); Weili Bao, Jiangsu (CN); Jianhe Chen, Jiangsu (CN); Xia Zhang, Jiangsu (CN); Lili Yu, Jiangsu (CN)

(73) Assignee: HUMAN ANTIBODOMICS DEVELOPMENT (SIP) LIMITED, Suzhou, Jiangsu (CN)

(21) Appl. No.: 12/601,007

(22) PCT Filed: May 22, 2007
(86) PCT No.: PCT/CN2007/001669

§ 371 Date: Nov. 20, 2009

(51) Int. Cl. A61K 39/395; C12N 15/63; C12N 5/10; C07H 21/04; C12N 1/00; C07K 14/47; C07K 19/00; C07K 16/18; A61K 38/17; A61P 3/10; A61P 35/00; C07K 7/08; A61P 3/04; A61P 3/06; A61P 9/12

(52) U.S. Cl. 424/139.1; 435/320.1; 435/325; 435/419; 536/23.5; 435/243; 530/350; 530/387.9; 514/21.2; 530/326; 530/327

(57) ABSTRACT

The invention provides human angiostatin interacting and tumor metastasis involving protein (HAI-TMIP) isoforms, namely HAI-TMIP variants 1 to 7, and human protein complexes comprising HAI-TMIP variant 1, 2, 3, 4, 5, 6 or 7. The invention also provides antibodies that immunospecifically bind to HAI-TMIP variant 1, 2, 3, 4, 5, 6 or 7 or a complex comprising such a variant, and uses of such antibodies. The present invention provides peptides which may be used as immunogens to distinguish between the HAI-TMIP variants. The invention further provides compounds that modulate the expression and/or activity of HAI-TMIP variant 1, 2, 3, 4, 5, 6 or 7, or a complex comprising such a variant, and uses of such compounds in the prevention, treatment and/or management of various disorders, in particular, cancer.

### 1. FIELD OF THE INVENTION

The invention provides human angiostatin interacting and tumor metastasis involving protein (HAI-TMIP) isoforms, namely HAI-TMIP variants 1 to 7, and human protein complexes comprising HAI-TMIP variant 1, 2, 3, 4, 5, 6 or 7. The invention also provides antibodies that immunospecifically bind to human HAI-TMIP variant 1, 2, 3, 4, 5, 6 or 7 or a complex comprising such a variant, and uses of such antibodies. The present invention also provides peptides which may be used as immunogens to produce antibodies that distinguish between HAI-TMIP variants. The invention further provides compounds that modulate the expression and/or activity of human HAI-TMIP variant 1, 2, 3, 4, 5, 6 or 7, or a complex comprising such a variant, and uses of such compounds in the prevention, treatment and/or management of various disorders, in particular, cancer.

### 2. BACKGROUND OF THE INVENTION

### 2.1 Cancer

A neoplasm, or tumor, is a neoplastic mass resulting from abnormal uncontrolled cell growth which can be benign or malignant. Benign tumors generally remain localized. Malignant tumors generally have the potential to invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behaves differently depending upon its origin. Cancerous cells destroy the part of the body in which they originate and then spread to other part(s) of the body where they start new growth and cause more destruction.

More than 1.2 million Americans develop cancer each year. Cancer is the second leading cause of death in the United States and, if current trends continue, cancer is expected to be the leading cause of death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

Currently, cancer therapy may involve surgery, chemotherapy, hormonal therapy and/or radiation treatment to eradicate neoplastic cells in a patient (, e.g., Stockdale, 1998, “Principles of Cancer Patient Management,” in Scientific American: Medicine, vol. 3, Rubenstein and Federman, eds., ch. 12, sect. IV). Recently, cancer therapy may also involve biological therapy or immunotherapy. All of these approaches can pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single compound and, although it can be effective, is often used to prevent or delay recurrence of cancer after other treatments have removed the majority of the cancer cells. Biological therapies/immunotherapies are limited in number and each therapy is generally effective for only a very specific type of cancer.

With respect to chemotherapy, there are a variety of chemotherapeutic compounds available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (, e.g., Gilman et al., 1990, Goodman and Gilman's: The Pharmacological Basis of Therapeutics, 8^{th} Ed. (Pergamom Press, New York)). These compounds, which include alkylating compounds, such as nitrosourea, anti-metabolites, such as methotrexate and hydroxyurea, and other compounds, such as etoposides, campathecins, bleomycin, doxorubicin, daunorubicin, etc., although not necessarily cell cycle specific, kill cells during S phase because of their effect on DNA replication. Other compounds, specifically colchicine and the vinca alkaloids, such as vinblastine and vincristine, interfere with microtubule assembly resulting in mitotic arrest. Chemotherapy protocols generally involve administration of a combination of chemotherapeutic a gents to increase the efficacy of treatment.

Despite the availability of a variety of chemotherapeutic compounds, chemotherapy has many drawbacks (, e.g., Stockdale, 1998, “Principles Of Cancer Patient Management” in Scientific American Medicine, vol. 3, Rubenstein and Federman, eds., ch. 12, sect. X). Almost all chemotherapeutic compounds are toxic, and chemotherapy causes significant, and often dangerous, side effects, including severe nausea, bone marrow depression, immunosuppression, etc. Additionally, even with administration of combinations of chemotherapeutic compounds, many tumor cells are resistant or develop resistance to the chemotherapeutic compounds. In fact, those cells resistant to the particular chemotherapeutic compounds used in the treatment protocol often prove to be resistant to other drugs, even those compounds that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic compounds for the treatment of cancer and new, more effective, therapy combinations for the treatment of cancer.

### 2.2 ATP Synthase

ATP synthase, also called F₁F₀-ATPase, is an enzymatic complex responsible for ATP synthesis in mitochondria, procaryote membranes, and chloroplasts, and belongs to a family of molecular rotary motors (Cross, R. L. (2004) Nature 427, 407-408). In the mitochondria, ATP synthase converts the energy of a proton gradient generated by the respiratory chain into ATP by making a high-energy chemical bond between inorganic phosphate (Pi) and ADP. The enzyme in mammals is composed of 17 subunits, 5 of which make up the easily detached F₁ with the stoichiometry α3β3γδε, the remainder are components of 2 stalk domains and the proton pumping F₀ part of the machinery.

F₁F₀ ATP synthase expression was believed to be localized exclusively to mitochondria (Cross, R. L. (2004) Nature 427, 407-408). However, reports are accumulating that components of ATP synthase exist on the outer face of the plasma membrane where they serve as a receptor for multiple ligands and participate in diverse processes such as regulation of lipid metabolism (Martinez et al., 2003, Nature 421: 75-79), control of proliferation and differentiation in endothelial cells (Moser et al, 1999, Proc. Natl. Acad. Sci. U.S.A. 96: 2811-2816; Moser et al, 2001, Proc. Natl. Acad. Sci. U.S.A. 98: 6656-6661), or immune recognition of tumors (Das et al., 1994, J. Exp. Med. 180, 273-281). ATP synthase has been identified on the plasma membrane of several cell types including hepatocytes where it acts as the HDL receptor through binding of apolipoprotein A-I and regulates lipoprotein internalization (Martinez et al., 2003, Nature 421: 75-79), on endothelial cells where it binds angiostatin, regulates surface ATP levels, and modulates endothelial cell proliferation and differentiation (Moser et al, 1999, Proc. Natl. Acad. Sci. U.S.A. 96: 2811-2816; Moser et al., 2001, Proc. Natl. Acad. Sci. U.S.A. 98:6656-6661), and on the surface of cancer cells (Wang et al, 2006, Mol. Cell. Proteomics. 5:43-52) where it is recognized by the antigen receptor of circulating cytotoxic lymphocytes of the gamma, delta subtype and thus it promotes an innate tumor cell recognition and lysis (Das et al., 1994, J. Exp. Med. 180: 273-281).

### 3. SUMMARY OF THE INVENTION

The present invention is based, in part, on the discovery of human angiostatin interacting and tumor metastasis involving protein (HAI-TMIP) isoforms, in particular, HAI-TMIP variants 1, 2, 3, 4, 5, 6 and 7. The present invention provides isolated nucleic acid molecules comprising the nucleotide sequence of HAI-TMIP variant 3 (SEQ ID NO:3 or 37; FIG. 1), HAI-TMIP variant 4 (SEQ ID NO:4 or 38; FIG. 2), HAI-TMIP variant 5 (SEQ ID NO:5 or 39; FIG. 3), HAI-TMIP variant 6 (SEQ ID NO:6 or 40; FIG. 4), or HAI-TMIP variant 7 (SEQ ID NO:7 or 41; FIG. 5), or derivatives, analogs or fragments thereof. The present invention also provides isolated nucleic acid molecules comprising (a) the nucleotide sequence which encodes a polypeptide comprising the amino acid sequence of HAI-TMIP variant 3, 4, 6 or 7 (SEQ ID NO:8; FIG. 6) or HAI-TMIP variant 5 (SEQ ID NO:9; FIG. 7), or a fragment thereof; (b) a nucleotide sequence comprising nucleic acid residues 123 to 309 of HAI-TMIP variant 4 (SEQ ID NO:4); (c) a nucleotide sequence comprising nucleic acid residues 468 to 557 of HAI-TMIP variant 5 (SEQ ID NO:5); (d) a nucleotide sequence which hybridizes to the complement of the nucleotide sequence of HAI-TMIP variant 3 (SEQ ID NO:3 or 37), HAI-TMIP variant 4 (SEQ ID NO:4 or 38), HAI-TMIP variant 5 (SEQ ID NO:5 or 39), HAI-TMIP variant 6 (SEQ ID NO:6 or 40), or HAI-TMIP variant 7 (SEQ ID NO:7 or 41), or a fragment thereof; (e) a nucleotide sequence which hybridizes to the complement of a nucleotide sequence encoding a polypeptide comprising the amino acid sequence of HAI-TMIP variant 3, 4, 6 or 7 (SEQ ID NO:8) or HAI-TMIP variant 5 (SEQ ID NO:9), or a fragment thereof; or (f) a nucleotide sequence that is at least 75%, preferably at least 80%, at least 85%, at least 90%, or at least 95% identical to the nucleotide sequence of HAI-TMIP variant 3 (SEQ ID NO:3 or 37), HAI-TMIP variant 4 (SEQ ID NO:4 or 38), HAI-TMIP variant 5 (SEQ ID NO:5 or 39), HAI-TMIP variant 6 (SEQ ID NO:6 or 40), or HAI-TMIP variant 7 (SEQ ID NO:7 or 41). The present invention also provides isolated nucleic acid molecules that encode a fusion proteins in which the nucleic acid molecules comprise a nucleotide sequence of HAI-TMIP variant 1, 2, 3, 4, 5, 6 or 7 and a nucleotide sequence encoding a heterologous amino acid sequence. The present invention also provides vectors and host cells comprising a nucleotide sequence described herein as well as host cells comprising such vectors. The present invention further provides methods for producing and purifying HAI-TMIP variant 1, 2, 3, 4, 5, 6 or 7, or an analog, derivative or fragment thereof.

The present invention provides an isolated polypeptide comprising the amino acid sequence of HAI-TMIP variant 1, 2, 3, 4, 6 or 7, or a derivative, fragment or analog thereof. In particular, the present invention provides an isolated polypeptide comprising the amino acid sequence of HA-TMIP variants 3, 4, 6 or 7 (SEQ ID NO:8) or HAI-TMIP variant 5 (SEQ ID NO:9), or a derivative, fragment or analog thereof. The present invention also provides an isolated polypeptide comprising: (a) the amino acid sequence encoded by the nucleotide sequence of HAI-TMIP variant 3 (SEQ ID NO:3 or 37), HAI-TMIP variant 4 (SEQ ID NO:4 or 38), HAI-TMIP variant 5 (SEQ ID NO:5 or 39), HAI-TMIP variant 6 (SEQ ID NO:6 or 40) or HAI-TMIP variant 7 (SEQ ID NO:7 or 41); (b) an amino acid sequence encoded by a nucleotide sequence that hybridizes over its full length to the nucleotide sequence of HAI-TMIP variant 3 (SEQ ID NO:3 or 37), HAI-TMIP variant 4 (SEQ ID NO:4 or 38), HAI-TMIP variant 5 (SEQ ID NO:5 or 39), HAI-TMIP variant 6 (SEQ ID NO:6 or 40) or HAI-TMIP variant 7 (SEQ ID NO:7 or 41); (c) an amino acid sequence that is at least 75%, preferably at least 80%, at least 85%, at least 90%, or at least 95% identical to the amino acid sequence of HAI-TMIP variant 3, 4, 6 or 7 (SEQ ID NO:8); or (d) an amino acid sequence that is at least 60%, preferably at least 65%, at least 70%, at least 75%, at least 80%, at least 85%, at least 90%, or at least 95% identical to the amino acid sequence of HAI-TMIP variant 5 (SEQ ID NO:9). The present invention also provides fusion protein comprising the amino acid sequence of a polypeptide described herein and a heterologous amino acid sequence. The present invention further provides purified complexes comprising a polypeptide described herein, as well as methods for producing and purifying such complexes. In one embodiment, the purified complexes are F1 subcomplexes of ATP synthase. In another embodiment, the purified complexes are F 0F1 ATP synthase holoenzyme complexes. In specific embodiments, such complexes have ATP synthase activity.

The present invention provides peptides which may be used as immunogens to produce antibodies that distinguish HAI-TMIP variants. In particular, the present invention provides the following peptides which may be used as immunogens to produce antibodies that distinguish HAI-TMIP variants:

| | |
|---|---|
| peptide 1 ARNFHASNTHLQKTC | (SEQ ID NO: 25) |
| peptide 2 MSSILEERILGADC | (SEQ ID NO: 26) |
| peptide 3 MQTGIKAVDSLVPC | (SEQ ID NO: 27) |
| peptide 4 CASNTHLQKTGTAE | (SEQ ID NO: 28) |
| peptide 5 CVSQHQALLGTIRA | (SEQ ID NO: 29) |

Peptides 1 and 4 can be used as an immunogen to generate antibodies that bind to HAI-TMIP variants 1 and 2. Peptide 2 can be used as an immunogen to generate antibodies that bind to HAI-TMIP variants 1, 2, 3, 4, 6 and 7 (and not HAI-TMIP variant 5). Peptide 4 can be used to generate antibodies that bind to HAI-TMIP variant 1. Peptides 3 and 5 can be used as an immunogen to generate antibodies that bind to HAI-TMIP variants 1, 2, 3, 4, 5, 6 and 7.

The present invention provides isolated antibodies that immunospecifically bind to an HAI-TMIP variant (e.g., HAI-TMIP variant 1, 2, 3, 4, 5, 6 or 7), or an F₁ ATP synthase subcomplex or F₀F₁ ATP synthase holoenzyme comprising an HAI-TMIP variant (e.g., HAI-TMIP variant 1, 2, 3, 4, 5, 6, or 7). In a specific embodiment, the present invention provides isolated antibodies that immunospecifically bind to a polypeptide encoding a nucleic acid molecule comprising the nucleotide sequence of HAI-TMIP variant 3 (SEQ ID NO:3 or 37; FIG. 1), HAI-TMIP variant 4 (SEQ ID NO:4 or 38; FIG. 2), HAI-TMIP variant 5 (SEQ ID NO:5 or 39; FIG. 3), HAI-TMIP variant 6 (SEQ ID NO:6 or 40; FIG. 4), or HAI-TMIP variant 7 (SEQ ID NO:7 or 41; FIG. 5), or derivatives, analogs or fragments thereof. In another embodiment, the present invention provides isolated antibodies that immunospecifically bind to a polypeptide comprising the amino acid sequence of HAI-TMIP variant 3, 4, 6 or 7 (SEQ ID NO:8) or HAI-TMIP variant 5 (SEQ ID NO:9), or a derivative, fragment or analog thereof. In another embodiment, the present invention provides isolated antibodies that immunospecifically bind to a complex comprising a polypeptide, wherein the polypeptide comprising the amino acid sequence of HAI-TMIP variant 3, 4, 6 or 7 (SEQ ID NO:8) or HAI-TMIP variant 5 (SEQ ID NO:9), or a derivative, fragment or analog thereof. In accordance with these embodiments, and in certain embodiments, such antibodies are conjugated to a diagnostic or therapeutic agent.

The present invention provides methods for identifying compounds that modulate the expression and/or activity of a HAI-TMIP variant (e.g., HAI-TMIP variant 1, 2, 3, 4, 5, 6, or 7), or an analog, derivative or fragment thereof. In particular, the present invention provides methods for identifying compounds that modulate the expression of a HAI-TMIP variant (e.g., HAI-TMIP variant 1, 2, 3, 4, 5, 6 or 7), or an analog, derivative or fragment thereof, the methods comprising: (a) contacting a cell expressing the HAI-TMIP variant, or analog, derivative or fragment with a test compound; (b) determining the amount of the HAI-TMIP variant, analog, derivative or fragment present in (a); and (c) comparing the amount present in (a) to that present in a corresponding control that has not been contacted with the test compound, so that if the amount of the HAI-TMIP variant, analog, derivative or fragment is altered relative to the amount in the control, a compound that modulates the expression of the HAI-TMIP variant, analog, derivative or fragment is identified. In one embodiment, the present invention provides a method for identifying a compound that modulates the expression of a polypeptide encoded by a nucleic acid molecule comprising the nucleotide sequence of SEQ ID NO:3, 4, 5, 6, 7, 37, 38, 39, 40 or 41, the method comprising: (a) contacting a cell expressing the polypeptide with a test compound; (b) determining the amount of the polypeptide present in (a); and (c) comparing the amount in (a) to that present in a corresponding control cell that has not been contacted with the test compound, so that if the amount of the polypeptide is altered relative to the amount in the control, a compound that modulates the expression of the polypeptide is identified. In another embodiment, the present invention provides a method of identifying a compound that modulates the expression of a polypeptide comprising the amino acid sequence of HAI-TMIP variant 3, 4, 5, 6, or 7 (SEQ ID NO:8 or 9), the method comprising: (a) contacting a cell expressing the polypeptide with a test compound; (b) determining the amount of the polypeptide present in (a); and (c) comparing the amount in (a) to that present in a corresponding control cell that has not been contacted with the test compound, so that if the amount of the polypeptide is altered relative to the amount in the control, a compound that modulates the expression of the polypeptide is identified.

The present invention provides methods for identifying compounds that modulate the formation and/or stability of a complex comprising a polypeptide comprising the amino acid sequence of a HAI-TMIP variant (e.g., HAI-TMIP variant 1, 2, 3, 4, 5, 6, or 7 or an analog, derivative or a fragment thereof. In particular, the present invention provides methods for identifying compounds that modulate the formation of a complex comprising a HAI-TMIP variant (e.g., HAI-TMIP 1, 2, 3, 4, 5, 6 or 7), an analog, derivative or a fragment thereof, the methods comprising: (a) contacting a cell expressing the subunits of the complex with a test compound; (b) measuring the amount of the complex formed; and (c) comparing the amount of the complex with the test compound, so that if the amount of complex formed is altered relative to the amount in the control, a compound that modulates the formation of the complex is identified. In one embodiment, the present invention provides a method for identifying a compound that modulates the formation of a complex comprising a polypeptide, wherein the polypeptide comprises the amino acid sequence of HAI-TMIP variant 3, 4, 5, 6, or 7 (SEQ ID NO:8 or 9), the method comprising: (a) contacting a cell expressing the subunits of the complex with a test compound; (b) measuring the amount of the complex formed; and (c) comparing the amount of the complex formed in (a) to that present in a corresponding control cell that has not been contacted with the test compound, so that if the amount of the complex formed is altered relative to the amount in the control, a compound that modulates the formation of the complex is identified.

The present invention provides methods for identifying a compound that modulates the stability of a complex comprising a HAI-TMIP variant (e.g., HAI-TMIP variant, 1, 2, 3, 4, 5, 6 or 7), or an analog, derivative, or fragment thereof, the methods comprising: (a) incubating the complex in the presence of a test compound under conditions conducive to maintaining the complex; and (b) determining the amount of the complex, wherein a difference in the amount of the complex determined in step (b) relative to the amount of the complex in the absence of the compound indicates the compound modulates the stability of the complex. In one embodiment, the present invention provides a method for identifying a compound that modulates the stability of a complex comprising a polypeptide, wherein the polypeptide comprises the amino acid sequence of HAI-TMIP variant 3, 4, 5, 6, or 7 (SEQ ID NO:8 or 9), the method comprising: (a) incubating the complex in the presence of a test compound under conditions conducive to maintaining the complex; and (b) determining the amount of the complex, wherein a difference in the amount of the complex determined in step (b) relative to the amount of the complex determined in the absence of the compound indicates that the compound modulates the stability of the complex.

The present invention provides methods for identifying compounds that modulate the activity of a complex comprising a HAI-TMIP variant (e.g., HAI-TMIP variant 1, 2, 3, 4, 5, 6, or 7), or an analog, derivative or fragment thereof. In particular, the present invention provides methods for identifying compounds that modulate the activity of a complex comprising a HAI-TMIP variant (e.g., HAI-TMIP variant 1, 2, 3, 4, 5, 6, or 7), or an analog, derivative or fragment thereof, the methods comprising: (a) contacting a compound with the complex and a labelled ATP substrate; and (b) measuring the amount of ATP hydrolysis, wherein a difference in the amount of ATP hydrolized measured in step (b) relative to the amount of ATP hydrolyzed in the absence the compound indicates that the compound modulates ATP synthase activity. In a specific embodiment, the present invention provides a method for identifying a compound that modulates the ATP synthase activity of a complex comprising a polypeptide, wherein the polypeptide comprises the amino acid sequence of HAI-TMIP variant 3, 4, 5, 6, or 7, or an analog, derivative or fragment thereof, the method comprising: (a) contacting a member of a library of compounds with the complex and a labelled ATP substrate; and (b) measuring the amount of ATP hydrolysis, wherein a difference in the amount of ATP hydrolized measured in step (b) relative to the amount of ATP hydrolyzed in the absence the compound indicates that the compound modulates ATP synthase activity.

The present invention provides compounds that modulate the expression and/or activity of a HAI-TMIP variant (e.g., HAI-TMIP variant 1, 2, 3, 4, 5, 6, or 7), or an analog, derivative or fragment thereof. Non-limiting examples of such compounds include antisense oligonucleotides, RNA interference (RNAi), aptamers, antibodies, small molecules, and nucleic acid molecules comprising nucleotide sequences encoding a HAI-TMIP variant (e.g., HAI-TMIP variant 1, 2, 3, 4, 5, 6, or 7), or an analog, derivative or fragment thereof. In a specific embodiment, the modulator is RNA(i) and the RNA(i) has the nucleotide sequence of SEQ ID NO:31, 32, 33, 34 or 35. In another embodiment, the modulator is an antibody. The present invention also provides compositions, including pharmaceutical compositions, comprising compounds that modulate the expression and/or activity of a HAI-TMIP variant (e.g., HAI-TMIP variant 1, 2, 3, 4, 5, 6, or 7), or an analog, derivative or fragment thereof.

The present invention also provides compounds that modulate the stabilization and/or formation of a complex comprising a polypeptide comprising the amino acid sequence of a HAI-TMIP variant (e.g., HAI-TMIP variant 1, 2, 3, 4, 5, 6, or 7), or an analog, derivative or fragment thereof. Non-limiting examples of such compounds include antibodies and small molecules. The present invention also provides compositions, including pharmaceutical compositions, comprising compounds that modulate the stabilization and/or formation of a complex comprising a polypeptide comprising the amino acid sequence of a HAI-TMIP variant (e.g., HAI-TMIP variant 1, 2, 3, 4, 5, 6, or 7), or an analog, derivative or fragment. In one embodiment, the present invention provides compositions comprising an inhibitor of a complex comprising a HAI-TMIP variant (e.g., HAI-TMIP variant 1, 2, 3, 4, 5, 6, or 7). In another embodiment, the present invention provides compositions comprising an activator of a complex comprising a HAI-TMIP variant (e.g., HAI-TMIP variant 1, 2, 3, 4, 5, 6, or 7).

The present invention provides methods of modulating angiogenesis, the method comprising administering a compound identified in accordance with the methods described herein. In certain embodiments, the compound increases angiogenesis in an angiogenesis model, described herein or known to one of skill in the art, by at least 15%, preferably at least 50%, at least 75%, at least 90%, or at least 95% relative to a negative control (e.g., PBS). In a specific embodiment, the compound increases angiogenesis in a chick choroallantoic membrane (CAM) model or retina angiogenesis model by at least 25%, preferably at least 50%, at least 75%, at least 90%, or at least 95% relative to a negative control. In other embodiments, the compound decreases angiogenesis in an angiogenesis model, described herein or known to one of skill in the art, by at least 15%, preferably at least 50%, at least 75%, at least 90%, or at least 95% relative to a negative control (e.g., PBS). In a specific embodiment, the compound decreases angiogenesis in a chick choroallantoic membrane (CAM) model or retina angiogenesis model by at least 25%, preferably at least 50%, at least 75%, at least 90%, or at least 95% relative to a negative control.

The present invention provides methods of preventing, treating and/or managing disorders characterized by, associated with or caused by aberrant angiogenesis, the method comprising administering to a subject in need thereof a compound that reduces the expression and/or activity of a HAI-TMIP variant (e.g., HAI-TMIP variant 1, 2, 3, 4, 5, 6, or 7), and/or decreases the formation and/or stability of a complex comprising a HAI-TMIP variant (e.g., HAI-TMIP variant 1, 2, 3, 4, 5, 6, or 7). Non-limiting examples of disorders characterized by, associated with or caused by aberrant angiogenesis include, but are not limited to, cancers, asthma, ischemia, atherosclerosis, scleroderma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retinopathy of prematurity, retrolental fibroplasias, diabetic neovascularization, peptic ulcer, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In a specific embodiment, the disorder is cancer (e.g., breast cancer). In certain embodiments, the disorder is not a carcinoma. In other embodiments, the disorder is not prostate cancer, colon cancer, breast cancer, leukemia (e.g., myelogenous leukemia).

The present invention provides methods of preventing, treating and/or managing a condition in which the promotion of angiogenesis would be beneficial, the methods comprising administering to a subject in need thereof a compound that increases the expression and/or activity of a HAI-TMIP variant (e.g., HAI-TMIP variant 1, 2, 3, 4, 5, 6, or 7), and/or decreases the formation and/or stability of a complex comprising a HAI-TMIP variant (e.g., HAI-TMIP variant 1, 2, 3, 4, 5, 6, or 7). Conditions in which the promotion of angiogenesis is desirable include, but are not limited to, conditions involving wounds, conditions involving occluded vessels, conditions in which organs and/or tissues have insufficient vascularization, and conditions such as spinal cord injuries that may benefit from vascularization. Specific disorders which can be prevented, treated and/or managed in accordance with methods the invention by administering an activator include, but are not limited to, ischemic heart disease, peripheral vascular disease, thromboembolic disease, stroke, vasculititis (Buerger's disease, Wegener's granulomatosis, and Giant Cell Arteritis), and surface ulcers involving the vascular endothelium (e.g., diabetic, haemophiliac, and varicose ulcers). Activators can also be used to promote wound healing, repair vascular damage following myocardial infarction, and to promote angiogenesis following surgical incisions, transplantation, and grafting (e.g., skin grafting and vascular grafting).

The present invention provides methods of inhibiting or reducing the growth of a cancer cell, the methods comprising contacting an inhibitor of HAI-TMIP with the cancer cell. The present invention also provides methods of inducing apoptosis of cancer cells, the methods comprising contacting an antibody with the cancer cells, wherein the antibody immunospecifically binds to or neutralizes HAI-TMIP. In a specific embodiment, the inhibitor inhibits or reduces the expression and/or activity of a HAI-TMIP (e.g., HAI-TMIP variant 1, 2, 3, 4, 5, 6 and/or 7). In another embodiment, the inhibitor reduces or prevents the formation of a complex of the invention. In yet another embodiment, the inhibitor destabilizes a complex of the invention. Non-limiting examples of cancer cells include breast cancer cells, colon cancer cells, lung cancer cells, kidney cancer cells, pancreatic cancer cells, melanoma cells, ovarian cancer cells, and uterine cancer cells. In a specific embodiment, the cancer cells are not prostate cancer cells. The cancer cells (e.g., breast cancer cells) may be drug resistant.

The present invention provides methods of preventing, treating and/or managing cancer, the methods comprising administering to a subject in need thereof a compound that modulates the expression and/or activity of a HAI-TMIP variant (e.g., an HAI-TMIP variant 1, 2, 3, 4, 5, 6, or 7), and/or modulates the formation and/or stability of a complex comprising a HAI-TMIP variant (e.g., HAI-TMIP variant 1, 2, 3, 4, 5, 6, or 7). In certain embodiment, the present invention provides methods of preventing, treating and/or managing cancer, the method comprising administering to a subject in need thereof a compound that decreases the expression and/or activity of HAI-TMIP variant 1, 2, 3, 4, 5, 6, or 7, and/or decreases the formation and/or stability of a complex comprising HAI-TMIP variant 1, 2, 3, 4, 5, 6, or 7. In other embodiments, the present invention provides methods of preventing, treating and/or managing cancer, the method comprising administering to a subject in need thereof a compound that increases the expression and/or activity of HAI-TMIP variant 3, and/or increases the formation and/or stability of a complex comprising HAI-TMIP variant 3.

The present invention provides methods of treating, preventing or managing cancer (e.g., breast cancer), the method comprising administering to subject in need thereof an inhibitor of HAI-TMIP activity. In some embodiments, the inhibitor is a protein that directly interacts with HAI-TMIP or ATP synthase complex. In certain embodiments, the inhibitor is an antibody that immunospecifically binds to a HAI-TMIP (e.g., HAI-TMIP variant 1, 2, 3, 4, 5, 6, or 7) or complex comprising a HAI-TMIP variant (e.g., HAI-TMIP variant 1, 2, 3, 4, 5, 6, or 7). In a specific embodiment, such an antibody is produced using an immunogen having the amino acid of: ARNFHASNTHLQKTC (SEQ ID NO:25); MSSILEERILGADC (SEQ ID NO:26); MQTGIKAVDSLVPC (SEQ ID NO:27); CASNTHLQKTGTAE (SEQ ID NO:28); or CVSQHQALLGTIRA (SEQ ID NO:29). In certain embodiments, the antibody reduces cancer cell proliferation by at least 10%, preferably at least 20%, at least 50%, at least 75%, at least 80%, at least 90%, or at least 95% relative to a negative control. In accordance with this embodiment, the reduction in cancer cell proliferation may be a reduction as determined in an in vitro cell proliferation assay.

In a specific embodiment, the present invention provides methods of preventing, treating and/or managing breast cancer, the methods comprising administering to a subject in need thereof an inhibitor of a HAI-TMIP variant or a complex comprising a HAI-TMIP variant. In one embodiment, the inhibitor is an antibody. In certain embodiments, the inhibitor (e.g., an antibody) inhibits or reduces cancer cell proliferation and/or metastasis.

The present invention provides methods of modulating the elimination of cholesterol by the liver, by modulating the expression and/or activity of a HAI-TMIP variant (e.g., HAI-TMIP variant 1, 2, 3, 4, 5, 6 and/or 7), or the formation and/or activity of an F₀F₁ ATP synthase holoenzyme or an F₁ ATP synthase subcomplex. The present invention also provides methods for preventing, treating and/or managing a disorder characterized by, associated with or caused by elevated levels of cholesterol, the methods comprising administering to a subject in need thereof a compound that increases the expression and/or activity of a HAI-TMIP variant (e.g., HAI-TMIP variant 1, 2, 3, 4, 5, 6, or 7), and/or increases the formation and/or stability of an F₀F₁ ATP synthase holoenzyme or an F₁ ATP synthase subcomplex (e.g., a complex comprising HAI-TMIP variant 1, 2, 3, 4, 5, 6, or 7). Disorders that are characterized by, associated with or caused by elevated levels of cholesterol in the blood include, but are not limited to, cardiovascular disease, type 2 diabetes, coronary heart disease, stroke, pancreatitis, hyperlipidemia, obesity, atherosclerosis and gout. The present invention also provides methods of modulating energy metabolism, the methods comprising administering to a subject in need thereof a compound that modulates the expression and/or activity of a HAI-TMIP variant (e.g., HAI-TMIP variant 1, 2, 3, 4, 5, 6, or 7), and/or modulates the formation and/or stability of an F₀F₁ ATP synthase holoenzyme or an F₁ ATP synthase subcomplex (e.g., a complex comprising HAI-TMIP variant 1, 2, 3, 4, 5, 6, or 7).

The present invention provides methods for detecting or diagnosing cancer in a subject, the methods comprising: (a) determining the expression level of the nucleic acid molecule encoding a HAI-TMIP variant (e.g., a HAI-TMIP variant, such as HAI-TMIP variant 1, 2, 3, 4, 5, 6, or 7) in the subject or a sample from the subject; and (b) comparing the level of expression in (a) to a corresponding control, wherein cancer is detected or diagnosed if there is a difference in the expression level of the nucleic acid molecule in (a) relative to the expression of the nucleic acid molecule in the control. In one embodiment, the present invention provides methods for detecting or diagnosing cancer in a subject, the method comprising: (a) determining the expression level of the nucleic acid molecule encoding HAI-TMIP variant 1, 2, 3, 4, 5, 6, or 7 in the subject or a sample from the subject; and (b) comparing the level of expression in (a) to a corresponding negative control, wherein cancer is detected or diagnosed if there is an increase in the expression level of the nucleic acid molecule in (a) relative to the expression of the nucleic acid molecule in the control. In another embodiment, the present invention provides methods for detecting or diagnosing cancer in a subject, the method comprising: (a) determining the expression level of the nucleic acid molecule encoding HAI-TMIP variant 3 in the subject or a sample from the subject; and (b) comparing the level of expression in (a) to a corresponding negative control, wherein cancer is detected or diagnosed if there is an decrease in the expression level of the nucleic acid molecule in (a) relative to the expression of the nucleic acid molecule in the control. In some embodiments, the cancer is metastic. In other embodiments, the cancer is breast cancer.

The present invention provides methods for detecting or diagnosing cancer in a subject, the methods comprising: (a) determining the expression level of a HAI-TMIP variant (e.g., such as HAI-TMIP variant 1, 2, 3, 4, 5, 6, or 7) in the subject or a sample from the subject; and (b) comparing the level of expression in (a) to a corresponding control, wherein cancer is detected or diagnosed if there is a difference in the expression level of the HAI-TMIP variant in (a) relative to the expression of the HAI-TMIP variant in the control. In one embodiment, the present invention provides methods for detecting or diagnosing cancer in a subject, the method comprising: (a) determining the expression level of HAI-TMIP variant 1, 2, 4, 5, 6, or 7 in the subject or a sample from the subject; and (b) comparing the level of expression in (a) to a corresponding negative control, wherein cancer is detected or diagnosed if there is an increase in the expression level of HAI-TMIP variant 1, 2, 4, 5, 6, or 7 in (a) relative to the expression of the HAI-TMIP variant 1, 2, 4, 5, 6, or 7 in the control. In another embodiment, the present invention provides methods for detecting or diagnosing cancer in a subject, the method comprising: (a) determining the expression level of HAI-TMIP variant 3 in the subject or a sample from the subject; and (b) comparing the level of expression in (a) to a corresponding negative control, wherein cancer is detected or diagnosed if there is an decrease in the expression level of HAI-TMIP variant 3 in (a) relative to the expression of the HAI-TMIP variant 3 in the control. In some embodiments, the cancer is metastatic. In other embodiments, the cancer is breast cancer.

The present invention provides methods for detecting or diagnosing cancer in a subject, the methods comprising: (a) determining the amount of a complex comprising a HAI-TMIP variant (e.g., such as HAI-TMIP variant 1, 2, 3, 4, 5, 6, or 7) in the subject or a sample from the subject; and (b) comparing the level of expression in (a) to a corresponding control, wherein cancer is detected or diagnosed if there is a difference in the complex in (a) relative to the expression of the amount of the complex in the control. In some embodiments, the cancer is metastatic. In other embodiments, the cancer is breast cancer.

The present invention provides methods for detecting or diagnosing cancer (e.g., metastatic cancer) in a subject, the methods comprising: (a) determining the amount of autoantibody that immunospecifically binds to a polypeptide described in Section 5.2 in the subject or a sample from the subject; and (b) comparing the amount of autoantibody in (a) to a corresponding control, wherein cancer is detected or diagnosed if there is an alteration in the amount of autoantibody in (a) relative to the amount of autoantibody in the control. The present invention also provides methods for detecting or diagnosing cancer (e.g., metastic cancer) in a subject, the methods comprising: (a) determining the amount of autoantibody that immunospecifically binds to a complex of the invention in the subject or a sample from the subject; and (b) comparing the amount of autoantibody in (a) to a corresponding control, wherein cancer is detected or diagnosed if there is an alteration in the amount of autoantibody in (a) relative to the amount of autoantibody in the control. In a specific embodiment, cancer is detected if there is an increase in the amount of autoantibody relative to as negative control (e.g., a healthy cancer-free subject or a sample from a healthy, cancer-free subject). Non-limiting examples of cancers that can be detected or diagnosed include breast cancer, lung cancer, liver cancer, prostate cancer, stomach cancer, and colon cancer. Non-limiting examples of cancer that can be detected or diagnosed include breast cancer, lung cancer, liver cancer, prostate cancer, stomach cancer and colon cancer.

The present invention provides kits and articles of manufacture comprising nucleic acid molecules comprising a nucleotide sequences encoding HAI-TMIP variant 3, 4, 5, 6, or 7, or an analog, derivative or fragment thereof. The present invention also provides kits and articles of manufacture comprising isolated polypeptides comprising the amino acid sequence of HAI-TMIP variant (e.g., HAI-TMIP variant 1, 2, 3, 4, 5, 6, or 7, or an analog, derivative or fragment thereof. The present invention also provides kits and articles of manufacture comprising a compound that modulates the expression and/or activity of a HAI-TMIP variant (e.g., HAI-TMIP variant 1, 2, 3, 4, 5, 6 and/or 7), and/or the formation, stabilization and/or activity of an F₀F₁ ATP synthase holoenzyme or an F₁ ATP synthase subcomplex comprising a HAI-TMIP (e.g., HAI-TMIP variant 1, 2, 3, 4, 5, 6 and/or 7). In particular, the present invention provides kits and articles of manufacture comprising an antibody that immunospecifically bind to HAI-TMIP variant (e.g., HAI-TMIP variant 1, 2, 3, 4, 5, 6 and/or 7), or a complex comprising a HAI-TMIP variant (e.g., HAI-TMIP variant 1, 2, 3, 4, 5, 6, or 7).

### 3.1 DEFINITIONS

As used herein, “a” or “an” means at least one, unless clearly indicated otherwise.

As used herein, the terms “about” or “approximately”, unless otherwise indicated, refer to a value that is no more than 10% above or below the value being modified by the term.

As used herein, the term “activator” refers to a compound that: (i) increases the expression of a HAI-TMIP variant (e.g., HAI-TMIP variant 1, 2, 3, 4, 5, 6 and/or 7); (ii) increases the activity of a HAI-TMIP variant (e.g., HAI-TMIP variant 1, 2, 3, 4, 5, 6 and/or 7); (iii) increases the formation of an F₁ ATP synthase subcomplex or an F₀F₁ ATP synthase holoenzyme (e.g., a complex of the invention); and/or (iv) increases the ATP synthase activity of an F₁ ATP synthase subcomplex or an F₀F₁ ATP synthase holoenzyme (e.g., a complex of the invention). Non-limiting examples of such compounds are in Section 5.16. In a specific embodiment, an activator specifically or selectively affects the expression and/or activity of a HAI-TMIP variant (e.g., a HAI-TMIP such as HAI-TMIP variant 1, 2, 3, 4, 5, 6 and/or 7), or the formation and/or activity of a complex of the invention. In certain embodiments, an activator competes with a ligand (e.g., angiostatin and/or lipid-free apoA-I) for binding to a HAI-TMIP variant (e.g., HAI-TMIP such as HAI-TMIP variant 1, 2, 3, 4, 5, 6 and/or 7) an F₁ ATP synthase subcomplex, or an F₀F₁ ATP synthase holoenzyme. In specific embodiments, the activator reduces the binding of a ligand (e.g., angiostatin and/or lipid-free apoA-I) to a HAI-TMIP (e.g., HAI-TMIP variant 1, 2, 3, 4, 5, 6 and/or 7), an F₁ ATP synthase, subcomplex or an F₀F₁ ATP synthase holoenzyme by at least 10% preferably at least 25%, at least 30%, at least 40%, at least 50%, at least 60%, at least 75%, at least 80%, at least 85%, at least 90%, or at least 95% relative to a negative control (e.g., PBS) as measured in a competition assay such as an ELISA. In certain embodiments, the activator prevents or reduces the binding of angiostatin to the F₀F₁ ATP synthase holoenzyme and/or F₁ ATP synthase subcomplex. In specific embodiments, the activator reduces the binding of angiostatin to the F₀F₁ ATP synthase holoenzyme and/or F₁ ATP synthase subcomplex by at least 10%, preferably at least 25%, at least 50%, at least 75%, at least 80%, at least 90%, or at least 95%, relative to a negative control in an assay described herein or known to one of skill in the art.

As used herein, the term “agent” refers to any molecule, compound, and/or substance which exerts a biological effect. An agent may be used, for example, in the prevention, treatment, management and/or diagnosis of a disorder.

As used herein, the terms “antibody” and “antibodies” refer to molecules that contain an antigen binding site, e.g., immunoglobulins. Immunoglobulin molecules can be of any type (e.g., IgG, IgE, IgM, IgD, IgA and IgY), class (e.g., IgG1, IgG2, IgG3, IgG4, IgA1 and IgA2) or subclass. Antibodies include, but are not limited to, monoclonal antibodies, multispecific antibodies, human antibodies, humanized antibodies, camelised antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above.

As used herein, the term “ATP synthase subunit-binding agent” or “HAI-TMIP-binding agent” refers to an agent which binds to a HAI-TMIP variant gene product. In certain embodiments, the binding agent is a proteinaceous agent, for example, a peptide, an antibody, or an antibody fragment such as an F(ab)₂ fragment. In other embodiments, the binding agent is a nucleic acid, for example a probe or primer.

As used herein, the term “cancer” refers to a neoplasm or tumor resulting from abnormal uncontrolled growth of cells. Non-limiting examples of cancers include those described in Section 5.18.2.2, infra. The term “cancer” encompasses a disease involving both pre-malignant and malignant cancer cells. In some embodiments, cancer refers to a localized overgrowth of cells that has not spread to other parts (e.g., organs or tissues) of a subject, e.g., a benign tumor. In other embodiments, cancer refers to a malignant tumor, which has invaded and destroyed neighboring body structures and spread to distant sites. In yet other embodiments, the cancer is associated with a specific cancer antigen.

As used herein, the term “chimeric antibody” refers to an antibody in which the constant region comes from an antibody of one species (typically human) and the variable region comes from an antibody of another species (typically rodent).

As used herein, the term “component protein(s)” and “protein component(s)” in the context of a protein complex refers to a subunit(s) of the F₁ subcomplex of an ATP synthase or an F₀F₁ ATP synthase holoenzyme. In one embodiment, the subunit is HAI-TMIP variant 1, 2, 3, 4, 5, 6 or 7. In a specific embodiment, the subunit is HAI-TMIP variant 3, 4, 5, 6, or 7.

As used herein, the term “decreased” with respect to the amount of a gene product in a subject or a sample from a subject refers to a decrease in the amount of the gene product in a subject or a sample from a subject relative to the amount of the gene product in a control subject(s) or a control sample or predetermined reference range. In a specific embodiment, the amount of a gene product in the subject or in the sample from the subject is decreased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the amount of the gene product in the control subject or control sample or predetermined reference range. In another specific embodiment, the amount of a gene product in a subject or a sample from a subject is decreased by 5-95%, 25%-95%, 50%-95% or 1 to 25 fold, 2 to 25 fold, 2 to 15 fold, 2 to 10 fold, or 2 to 5 fold relative to the amount of the gene product in a control subject(s) or a control sample or predetermined reference range.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis in the presence of tunicamycin, etc. Further, a derivative of a proteinaceous agent may contain one or more non-classical amino acids. A derivative of a proteinaceous agent possesses a similar or identical function as the proteinaceous agent from which it was derived. The term “derivative” in the context of a proteinaceous agent also refers to a proteinaceous agent that possesses a similar or identical function as a second proteinaceous agent (i.e., the proteinaceaous agent from which the derivative was derived) but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, ° at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy. In a specific embodiment, a derivative is a functionally active derivative.

As used herein, the terms “disorder” and “disease” are used interchangeably to refer to a condition in a subject.

As used herein, the phrase “elderly human” refers to a human between 65 years old or older, preferably 70 years old or older.

As used herein, the term “effective amount” in the context of the administration of a therapy generally refers to the amount of a therapy which is sufficient to reduce or ameliorate the severity and/or duration of a disorder or one or more symptoms thereof, ameliorate the symptoms of a disorder, prevent the advancement of a disorder, cause regression of a disorder, prevent the recurrence, development, or onset of a disorder or a symptom thereof, and/or enhance or improve the prophylactic or therapeutic effect(s) of another therapy.

As used herein, the term “fragment” is the context of a fragment of a proteinaceous agent (e.g., a protein or polypeptide) a fragment is composed of 8 or more amino acids, 10 or more amino acids, 15 or more amino acids, 20 or more amino acids, 25 or more amino acids, 50 or more amino acids, 75 or more amino acids, 100 or more amino acids, 150 or more amino acids, 200 or more amino acids, 10 to 150 amino acids, 10 to 200 amino acids, 10 to 250 amino acids, 10 to 300 amino acids, 50 to 100 amino acids, 50 to 150 amino acids, 50 to 200 amino acids, 50 to 250 amino acids or 50 to 300 amino acids of a proteinaceous agent.

As used herein, the term “functionally active derivative” in the context of proteinaceous agent is a derivative of a proteinaceous agent that retains at least one function of the polypeptide or protein from which the derivative is derived. In a specific embodiment, a functionally active derivative retains at least two, three, four, or five functions of the protein or polypeptide from which the derivative is derived. In a specific embodiment, the functionally active derivative retains the ability of the protein from which it is derived to bind to a specific third protein (e.g., an ATP synthase β subunit) or form a specific complex with ATP synthase activity.

As used herein, the term “functionally equivalent” in the context of a HAI-TMIP isoform is a proteinaceous agent that exhibits at least one, preferably at least 2, at least 3 or more activity(ies) of HAI-TMIP variant 1, 2, 3, 4, 5, 6 or 7 as measured in an in vivo and/or in vitro assay. In a specific embodiment, a proteinaceous agent is functionally equivalent to HAI-TMIP variant 1, 2, 3, 4, 5, 6 or 7 if it binds to a human ATP synthase β subunit or forms a specific complex with ATP synthase activity as measured by an assay described herein or known in the art.

As used herein, the term “functionally active fragment” refers to a fragment of a polypeptide or protein that retains at least one, preferably at least two, at least three or more function(s). In a specific embodiment, the functionally active fragment of a HAI-TMIP isoform retains the ability to bind to an ATP synthase β subunit or form a specific complex with an ATP synthase activity as measured by an assay described herein or known in the art.

As used herein, the term “heterologous” in the context of an entity (e.g., a fusion protein) refers to an element that is part of an entity (e.g., a fusion protein) that is composed of one or more other elements, wherein the elements are not normally found or associated together. For example, in the context of a fusion protein, two or more amino acid sequences not normally found or associated together in nature are joined, (by, e.g., conjugation).

The terms “human ATP synthase a subunit,” “human angiostatin interacting and tumor metastasis involving protein” and “HAI-TMIP” are used interchangeably.

As used herein, the phrase “human adult” refers to a human 18 years of age or older.

As used herein, the phrase “human child” refers to a human between 24 months of age and 18 years of age.

As used herein, the phrase “human infant” refers to a human less than 24 months of age, preferably less than 12 months of age, less than 6 months of age, less than 3 months of age, less than 2 months of age, or less than 1 month of age.

As used herein, the term “humanized antibody” and “humanized immunoglobulin” refer to an immunoglobulin comprising a human framework, at least one and preferably all complementarity determining regions (CDRs) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. See, e.g. Queen et al, U.S. Pat. Nos. 5,5301,101; 5,585,089; 5,693,762; and 6,180,370 (each of which is incorporated by reference in its entirety).

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95%, 98%, 99% or 99.5%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1×SSC, 0.2% SDS at about 68° C. In a specific, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides. In a specific embodiment, high stringency conditions comprise hybridization in a buffer consisting of 6×SSC, 50 mM Tris-HCl (pH=7.5), 1 mM EDTA, 0.02% PVP, 0.02% Ficoll, 0.02% BSA and 100 μg/ml denatured salmon sperm DNA, for 48 hours at 65° C., washing in a buffer composed of 2×SSC, 0.01% PVP, 0.01% Ficoll and 0.01% BSA, for 45 minutes at 37° C., and washing in a buffer composed of 0.1×SSC, for 45 minutes at 50° C.

As used herein, the term “immunospecifically binds” “specifically binds” and analogous terms refer to agents, in particular proteinaceous agents (e.g., peptides, polypeptides, proteins, fusion proteins and antibodies) that specifically bind to an antigen and do not specifically bind to other antigens. An agent, e.g., a proteinaceous agent, that immunospecifically binds to an antigen may bind to other antigens with lower affinity as determined by, e.g., immunoassays, BIAcore, or other assays known in the art. Antibodies that immunospecifically bind to an antigen may be cross-reactive with related antigens. In a specific, antibodies that immunospecifically bind to an antigen do not cross-react with other antigens. An antibody binds specifically to an antigen when it binds to the antigen with higher affinity than to any cross-reactive antigen as determined using experimental techniques, such as radioimmunoassays (RIAs) and enzyme-linked immunosorbent assays (ELISAs)., e.g., Paul, ed., 1989, Fundamental Immunology, 2^{nd} ed., Raven Press, New York at pages 332-336 for a discussion regarding antibody specificity.

As used herein, the term “in combination” refers to the use of more than one therapies (e.g., one or more prophylactic and/or therapeutic agents). The use of the term “in combination” does not restrict the order in which therapies are administered to a subject with a disorder. A first therapy (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours; 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disorder or a symptom thereof.

As used herein, the term “increased” with respect to the amount of a gene product in a subject or a sample from a subject refers to an increase in the amount of the gene product in a subject or a sample from a subject relative to the amount of the gene product in a control subject(s) or a control sample or predetermined reference range. In a specific embodiment, the amount of a gene product in the subject or in the sample from the subject is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the amount of the gene product in the control subject(s) or control sample or predetermined reference range. In another specific embodiment, the amount of a gene product in a subject or a sample from a subject is increased by 5-95%, 25%-95%, 50%-95%, or 1 to 25 fold, 2 to 25 fold, 2 to 15 fold, 2 to 10 fold, or 2 to 5 fold relative to the amount of the gene product in a control subject(s) or a control sample or predetermined reference range.

As used herein, the term “inhibitor” refers to a compound that: (i) inhibits or reduces the expression of a HAI-TMIP variant (e.g., HAI-TMIP variant 1, 2, 3, 4, 5, 6 and/or 7); (ii) inhibits or reduces the activity of a HAI-TMIP variant (e.g., HAI-TMIP variant 3, 4, 5, 6 and/or 7); (iii) reduces or prevents the formation of an F₁ ATP synthase subcomplex or an F₀F₁ ATP synthase holoenzyme (e.g., a complex of the invention); (iv) destabilizes an F₁ ATP synthase subcomplex or an F₀F₁ ATP synthase holoenzyme (e.g., a complex of the invention); and/or (v) inhibits or reduces the ATP synthase activity of an F₁ ATP synthase subcomplex or an F₀F₁ ATP synthase holoenzyme (e.g., a complex of the invention). Non-limiting examples of such compounds are in Section 5.15. In a specific embodiment, an inhibitor specifically or selectively affects the expression and/or activity of a HAI-TMIP variant, or the formation and/or activity of a complex of the invention. In another specific embodiment, an inhibitor specifically or selectively affects a HAI-TMIP, preferably HAI-TMIP variant 3, 4, 5, 6 and/or 7. In certain embodiments, an inhibitor competes with a ligand (e.g., angiostatin and/or lipid-free apoA-I) for binding to a HAI-TMIP variant (e.g., HAI-TMIP such as HAI-TMIP variant 1, 2, 3, 4, 5, 6 and/or 7), an F₁ ATP synthase subcomplex or an F₀F₁ ATP synthase holoenzyme. In specific embodiments, the inhibitor reduces the binding of a ligand (e.g., angiostatin and or lipid-free apoA-I) to a HAI-TMIP variant (e.g., such as HAI-TMIP variant 1, 2, 3, 4, 5, 6 and/or 7), an F₁ ATP synthase subcomplex or an FR₁ ATP synthase holoenzyme by at least 10%, preferably at least 25%, at least 30%, at least 40%, at least 50%, at least 60%, at lest 75%, t least 80%, at least 85%, at least 90%, or at least 95% relative to a negative control (e.g., PBS) as measured in a competition assay such as an ELISA. In certain embodiments, the inhibitor increases the binding of angiostatin to the F₀F₁ ATP synthase holoenzyme and/or F₁ ATP synthase subcomplex. In specific embodiments, the inhibitor increases the binding of angiostatins to the F₀F₁ ATP synthase holoenzyme by at least 10%, preferably at least 25%, at least 50%, at least 75%, at least 80%, at least 90%, or at least 95% relative to a negative control in an assay described herein or known to one of skill in the art.

As used herein, the terms “manage,” “managing,” and “management” in the context of the administration of therapy to a subject refer to the beneficial effects that a subject derives from a therapy (e.g., a prophylactic or therapeutic agent), while not resulting in a cure of the disorder. In certain embodiments, a subject is administered one or more therapies (e.g., one or more prophylactic or therapeutic agents) to “manage” the disorder so as to prevent the progression or worsening of the condition.

As used herein, the terms “modulator” and “modulators” refers to an agent that: (1) modulates the expression of a HAI-TMIP variant (e.g., such as HAI-TMIP variant 1, 2, 3, 4, 5, 6 or 7); (ii) modulates the activity of a HAI-TMIP variant (e.g., such as HAI-TMIP variant 1, 2, 3, 4, 5, 6 or 7); (iii) modulates the formation of an F₁ ATP synthase subcomplex or an F₀F₁ ATP synthase holoenzyme, (iv) modulates the stability of an F₁ ATP synthase subcomplex or an F₀F₁ ATP synthase holoenzyme; and/or (v) modulates the ATP synthase activity of an F₁ ATP synthase subcomplex or an F₀F₁ ATP synthase holoenzyme modulators include, but are not limited to the activators and inhibitors described herein. In certain embodiments, a modulator competes with a ligand (e.g., angiostatin and/or apoA-I) for binding to a HAI-TMIP variant (e.g., HAI-TMIP variant 1, 2, 3, 4, 5, 6 and/or 7), an F₁ ATP synthase subcomplex or an F₀F₁ ATP synthase holoenzyme as measured in a competition assay such as an ELISA.

As used herein, the terms “peptide, polypeptide, and protein” are used to refer to amino acid sequences of various approximate lengths. For example, a peptide refers to a chain of two or more amino acids joined by peptide bonds, generally of less than about 50 amino acid residues, while a polypeptide refers to a longer chain of amino acids. It is appreciated that the terms “peptide” and “polypeptide” are not meant to refer to a precise length of a chain of amino acid residues and that in certain contexts, the two terms may be used interchangeably.

Percent Identity: To determine the percent identity of amino acid sequences or nucleic acid sequences encoding, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e.,% identity=number of identical overlapping positions/total number of positions×100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score-100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the terms “prevent”, “preventing” and “prevention” in the context of the administration of a therapy to a subject refer to the prevention or inhibition of the recurrence, onset, and/or development of a disorder or a symptom thereof in a subject, or an improved or enhanced prophylactic effect resulting from the administration of a therapy (e.g., a prophylactic or therapeutic agent) or a combination of therapies (e.g., a combination of prophylactic or therapeutic agents). In some embodiments, such terms refer to one or more of the following: (1) a reduction or stabilization in the cancer cell population; (2) an increase in the length of remission; (3) a decrease in the recurrence rate of cancer; (4) an increase in the time to recurrence of cancer; and/or (5) an increase in the survival of the patient.

As used herein, the term “population” refers to a group of individuals from which samples are taken for statistical measurement. Preferably, the individuals are human. The size of the population will vary depending on what is needed to obtain a statistically significant measurement. In specific embodiments, the size of the population is 5, 10, 25, 50, 100, 150, 250, 500, 750, 1000, 5 to 50, 5 to 100, 50 to 150, 50 to 250, 10 to 500, 250 to 750, 500 to 1000, or more than 1000 individuals.

As used herein, the term “predetermined reference range” refers to a reference range for a particular biological entity, e.g., the expression and/or the activity of a gene product, for a subject or a population of subjects. Each laboratory may establish its own reference range for each particular assay, or a standard reference range for each assay may be made available and used locally, regionally, nationally, or worldwide.

As used herein, the term “prophylactic agent” refers to any molecule, compound, and/or substance for use in the prevention of a disorder.

As used herein, the phrase “prophylactically effective amount” refers to the amount of a therapy (e.g., prophylactic agent) which is sufficient to result in the prevention or inhibition of the development, recurrence and/or onset of a disorder or a symptom thereof, and/or to enhance or improve the prophylactic effect(s) of another therapy (e.g., another prophylactic agent). Examples of prophylactically effective amounts of therapies are provided infra.

As used herein, the term “protocol” refers to a regimen for dosing and timing of the administration of one or more agents and/or compositions for the prevention, treatment, and/or management of a disorder or a symptom thereof.

As used herein, the terms “purified” and “isolated” in the context of a compound or agent (including, e.g., proteinaceous agents such as antibodies) that is chemically synthesized refers to a compound or agent that is substantially free of chemical precursors or other chemicals when chemically synthesized. In a specific embodiment, the compound or agent is 60%, 65%, 75%, 80%, 85%, 90%, 95%, or 99% free (by dry weight) of other, different compounds or agents.

As used herein, the terms “purified” and “isolated” when used in the context of a compound or agent (including proteinaceous agents such as antibodies) that can be obtained from a natural source, e.g., cells, refers to a compound or agent which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials.

An “isolated” nucleic acid sequence or nucleotide sequence is one which is separated from other nucleic acid molecules which are present in a natural source of the nucleic acid sequence or nucleotide sequence. Moreover, an “isolated”, nucleic acid sequence or nucleotide sequence, such as a cDNA molecule, can be substantially free of other cellular material or culture medium when produced by recombinant techniques, or substantially free of chemical precursors when chemically synthesized. In certain embodiments, an “isolated” nucleic acid sequence or nucleotide sequence is a nucleic acid sequence or nucleotide sequence that is recombinantly expressed in a heterologous cell.

As used herein, the term “small molecules” and analogous terms include, but are not limited to, peptides, peptidomimetics, amino acids, amino acid analogs, polynucleotides, polynucleotide analogs, nucleotides, nucleotide analogs, other organic and inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, organic or inorganic compounds having a molecular weight less than about 100 grams per mole, and salts, esters, and other pharmaceutically acceptable forms of such compounds. Salts, esters, and other pharmaceutically acceptable forms of such compounds are also encompassed.

As used herein, the terms “stabilize” and “stabilization” in the context of a complex of the invention, cancer cells, or disease status, refer to a less than a 10% change in the amount of complex, the amount of cancer cells, or disease status.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the term “therapeutic agent” refers to any molecule, compound, and/or substance for use in the treatment and/or management a disorder.

As used herein, the term “therapeutically effective amount” refers to that amount of a therapy (e.g., a therapeutic agent) sufficient to ameliorate a disorder or a symptom thereof, prevent advancement or progression of a disorder, reduce the duration of a disorder or a symptom thereof, reduce the severity of a disorder or a symptom thereof, cause regression of a disorder, and/or to enhance or improve the therapeutic effect(s) of another therapy.

As used herein, the terms “therapies” and “therapy” can refer to any protocol(s), method(s), composition(s), and/or agent(s) that can be used in the prevention, treatment and/or management of a disorder or one or more symptoms thereof. In certain embodiments, the terms “therapy” and “therapies” refer to chemotherapy, radiation therapy, surgery, hormonal therapy, biological therapy, immunotherapy and/or other therapies useful in the prevention, management and/or treatment of a disorder or one or more symptoms thereof.

As used herein, the terms “treat”, “treatment”, and “treating” in the context of the administration of a therapy to a subject refer to the reduction in the progression, severity and/or duration of a disorder and/or a symptom thereof, amelioration of a symptom of a disorder resulting from the administration of one or more therapies including but not limited to, the administration of one or more prophylactic or therapeutic agents. In specific embodiments, such terms refer to one, two, three or more of the following results: (1) the reduction of growth and/or formation of a tumor; (2) a reduction of the growth, formation and/or number of cancerous cells; (3) eradication, removal or control of primary, regional or metastatic cancer (e.g., the minimization or delay of the spread of cancer); (4) a reduction in mortality; (5) an increase in survival rate of a patient population; (6) an increase in the number of patients in remission; (7) a decrease in hospitalization rate; (8) a decrease in hospitalization lengths; and/or (8) stabilization of the disorder.

Concentrations, amounts, cell counts, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. BRIEF DESCRIPTION OF THE FIGURES

FIG. 1 depicts the nucleotide sequence of HAI-TMIP variant 3. The ATG codon beings at nucleotide position 134 and the stop codon begins at nucleotide position 1643. The ATG codon and stop codon are boxed, and the non-coding region is in italics.

FIG. 2 depicts the nucleotide sequence of HAI-TMIP variant 4. The ATG codon begins at nucleotide position 321 and the stop codon begins at nucleotide position 1830. The ATG codon and stop codon are boxed, and the non-coding region is in italics.

FIG. 3 depicts the nucleotide sequence of HAI-TMIP variant 5. The ATG codon begins at nucleotide position 638 and the stop codon begins at nucleotide position 1733. The ATG codon and stop codon are boxed, and the non-coding region is in italics.

FIG. 4 depicts the nucleotide sequence of HAI-TMIP variant 6. The ATG codon beings at nucleotide position 55 and the stop codon begins at nucleotide position 1564. The ATG codon and stop codon are boxed, and the non-coding region is in italics.

FIG. 5 depicts the nucleotide sequence of HAI-TMIP variant 7. The ATG codon begins at nucleotide position 162 and the stop codon begins at nucleotide position 1671. The ATG codon and stop codon are boxed, and the non-coding region is in italics.

FIG. 6 depicts the amino acid sequence of HAI-TMIP variants 3, 4, 6 and 7.

FIG. 7 depicts the amino acid sequence for HAI-TMIP variant 5.

FIG. 8 shows the exon and intron structures of HAI-TMIP variants 1 to 7.

FIGS. 9A-9G depict an alignment of the nucleotide sequences of HAI-TMIP variants 1 to 7. The nucleotides that are shared by the variants are shaded.

FIG. 10 is an alignment of the amino acid sequence of HAI-TMIP variants 1, 3 and 5. The seven gene splicing variants encode three different protein variants. The amino acid sequence for HAI-TMIP variants 1 and 2 are identical. The amino acid sequence for HAI-TMIP variants 3, 4, 6 and 7 are identical. The amino acid residues that are shared by the variants are shaded.

FIG. 11. Synthesis of polypeptides of HAI-TMIP to be used as antigens for producing antibodies to distinguish variants 1, 3 and 5.

FIG. 12. Detection of autoantibodies to HAI-TMIP in serums of different kinds of cancers.

FIGS. 13A and 13B. A) Differential display map of HAI-TMIP between MHCC97-L and MHCC97-H. B) MALDI-TOF/MS tryptic peptide mass fingerprint.

FIGS. 14A-14F. Binding of antibody directed against the HAI-TMIP on the surface of breast cancer cells by flow cytometry. MDA-MB-231 and MCF-10F cells were analysed by fluorescence-assisted flow cytometry. Histogram plots are shown for HAI-TMIP at intact cells, which selected as cells excluding propidium iodide.

A) and D). MDA-MB-231 and MCF-10F cells incubated with secondary antibody only.

B) and E). MDA-MB-231 and MCF-10F cells incubated with an isotypic control mouse IgG_{2b}.

C) and F). MDA-MB-231 and MCF-10F cells incubated with an antibody against HAI-TMIP.

FIG. 15. Inhibition of breast cancer cells proliferation by anti-HAI-TMIP antibody. MDA-MB-231 and MCF-10F were plated at a density of 5,000 cells/well in media depleted of fetal calf serum (FCS) overnight to allow the cells to become quiescent. In some experiments antibody directed against HAI-TMIP was added at concentrations of 5 μg/mL or 10 μg/mL. CCK-8 solution was added after 24 hours, and the absorbance of formazan was quantitated on a Thermomax plate reader at a wavelength of 490 nm according to the manufacturer's specifications. The absorbance values used to calculate the percent proliferation of the cells.

FIG. 16. The anti-HAI-TMIP antibody (otherwise referred to as anti-ATP synthase a subunit antibody) attenuates the migration of MDA-MB-231 cells on fibronectin and laminin. The migration of MDA-MB-231 cells was measured using the transwell inserts coated with FN (10 μg/mL) or LN 1 (10 μg/mL). The migration medium was DMEM containing 0.1% heat-inactivated BSA in top wells and containing 1% fetal calf serum in bottom wells. In each individual experiment, cells that migrated through the filters were counted from at least three randomly selected fields. Results were obtained from at least three individual experiments and represented as the member of migration cells. The bar graph represent means±S.D. **: p<0.001 on both FN and on LN between the anti-HAI-TMIP antibody and mIgG2b. Notably, no difference was seen in MCF-10F cells on both FN and on LN1 (data not shown).

FIG. 17 is a graph showing the cytotoxic effect of 1 μg/ml adriamycin (ADM) in combination with 0 μg/ml (first column), 1 μg/ml (second column), 2 μg/ml (third column), or 4 μg/ml (fourth column) of anti-HAI-TMIP antibody, or with 4 μg/ml of isotype mouse anti-IgG2b (fifth column) as control on MDA-MB-435 cells. The cells were incubated for 48 hours with antibody and ADM.

FIG. 18 is a graph showing the cytotoxic effect of 6 μg/ml adriamycin (ADM) alone (first column) or in combination with 1 μg/ml (second column), 2 μg/ml (third column), or 4 μg/ml (fourth column) of anti-HAI-TMIP antibody on MCF-7 cells. The fifth column in the graph shows the cytotoxic effect of 6 μg/ml of ADM and 4 μg/ml of anti-IgG2b antibody on MCF-7 cells. The cells were incubated for 48 hours with ADM or ADM and antibody. Each value represents the mean and standard deviation from triplicate determination.

FIG. 19 is a graph showing the cytotoxic effect of 10 μg/ml adriamycin (ADM) alone or in combination with 2 μg/ml of anti-HAI-TMIP antibody on MCF-7/ADR cells. The cells were incubated for 24, 36, 48 and 72 hours with ADM or ADM and antibody.

FIG. 20 shows the PI and annexin V staining by MDA-MB-435 cells treated with 4 μg/ml of adriamycin (ADM) alone or 4 μg/ml of ADM and 1 μg/ml of anti-HAI-TMIP antibody. The cells were incubated for 48 hours with ADM or ADM and antibody.

FIG. 21 shows the PI and annexin V staining by MCF7 cells treated with 4 μg/ml of adriamycin (ADM) alone or in combination with 1 μg/ml or 4 μg/ml of anti-HAI-TMIP antibody. The cells were incubated for 48 hours with ADM or ADM and antibody.

### 5. DETAILED DESCRIPTION OF THE INVENTION

### 5.1 Nucleotide Sequence of HAI-TMIPs

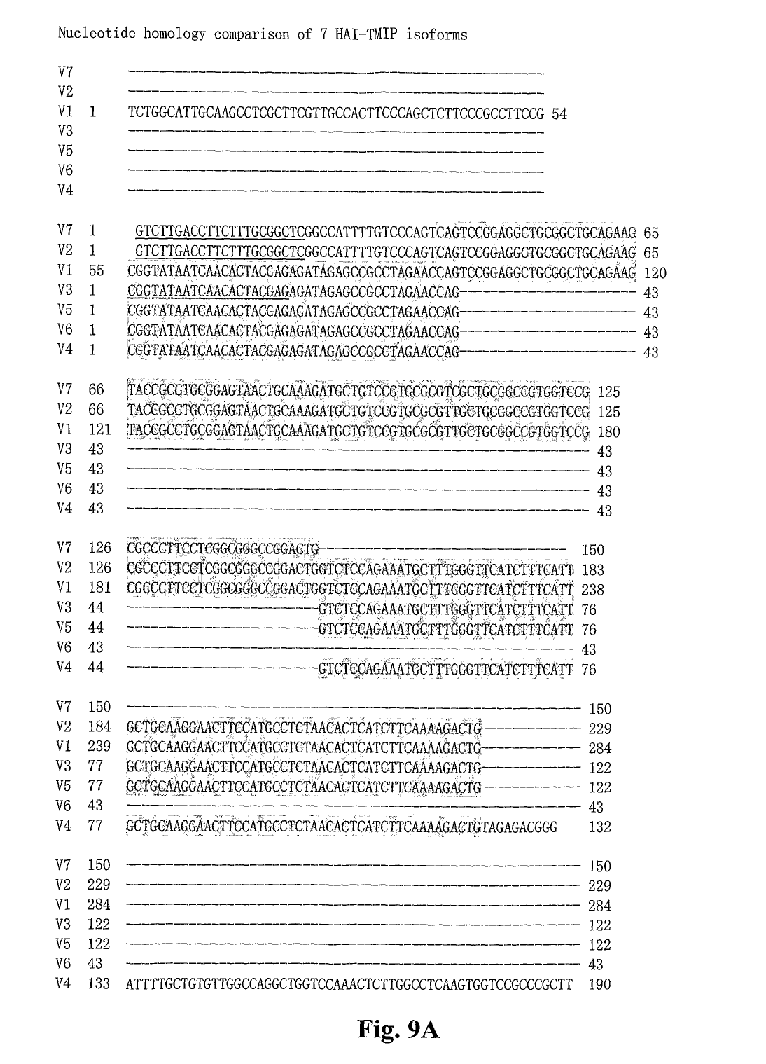

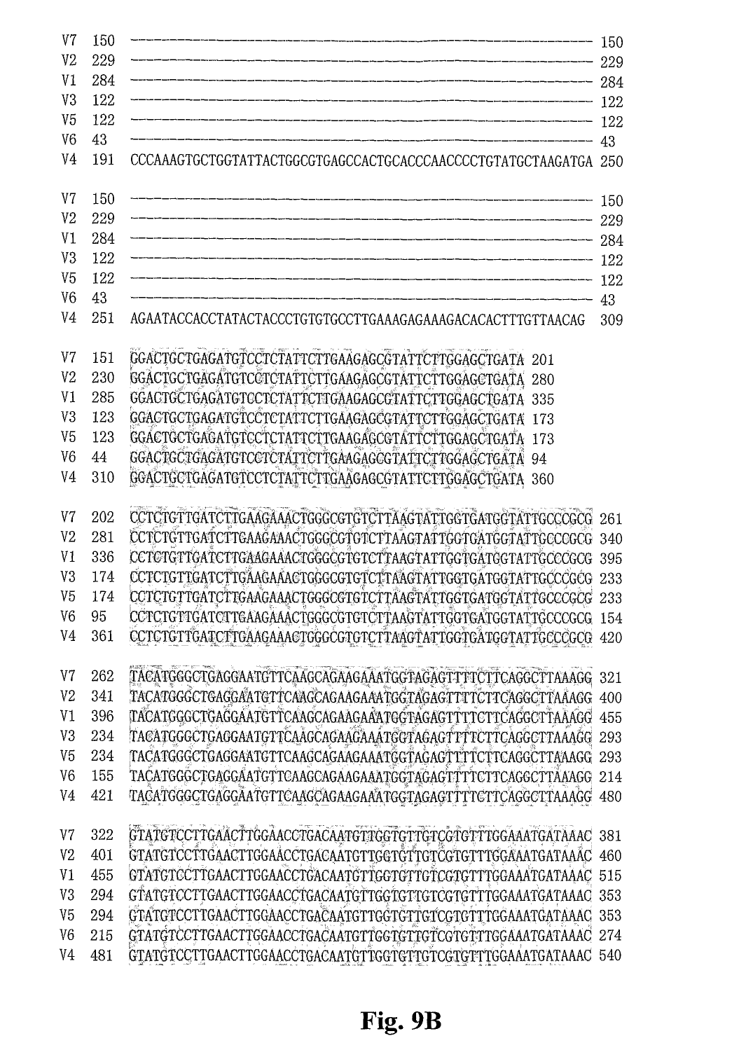

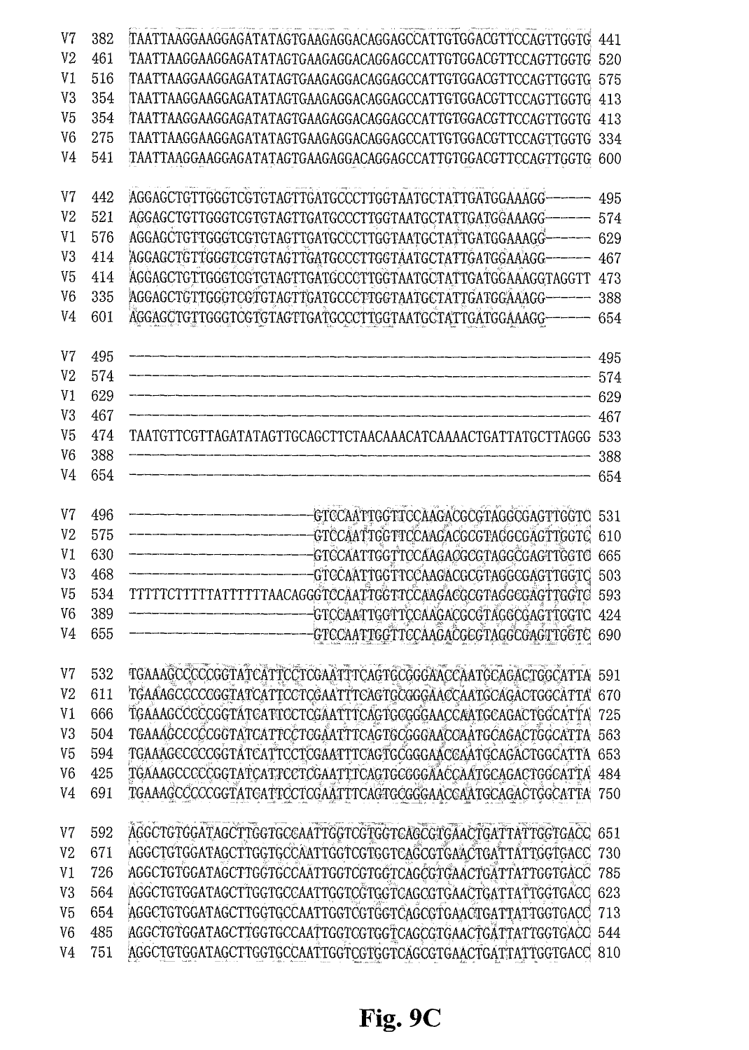

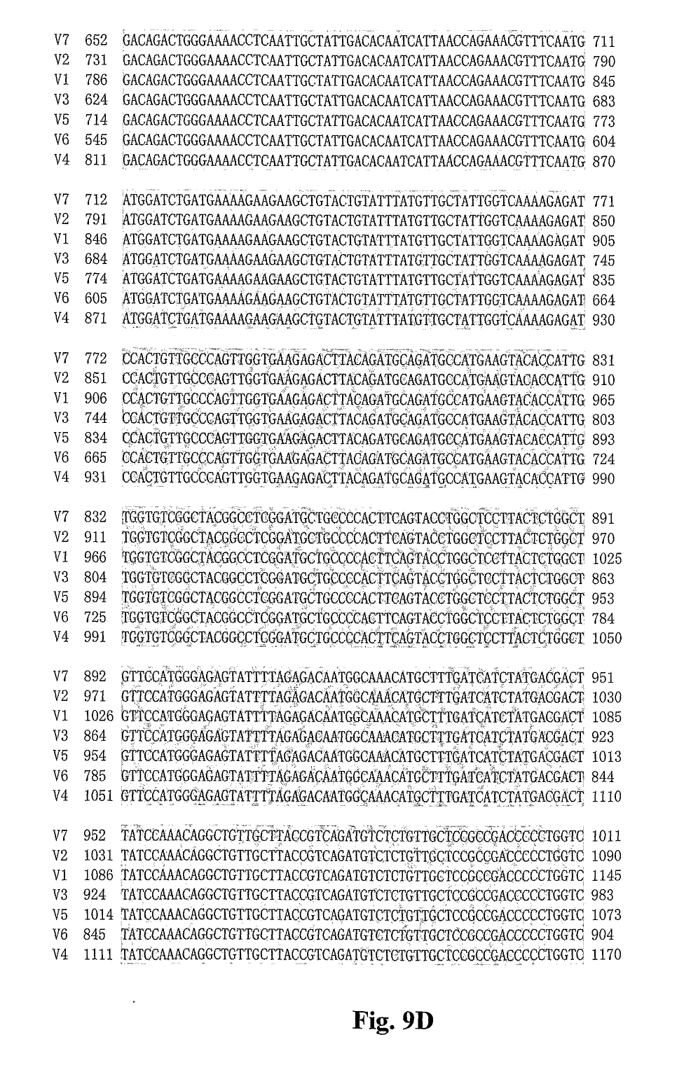

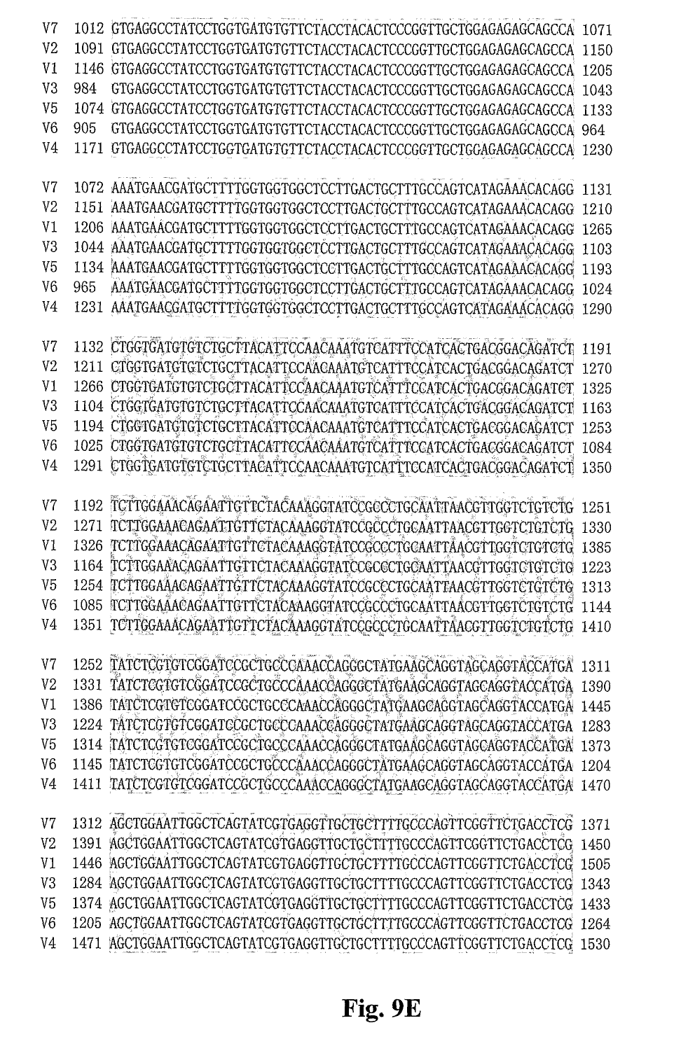

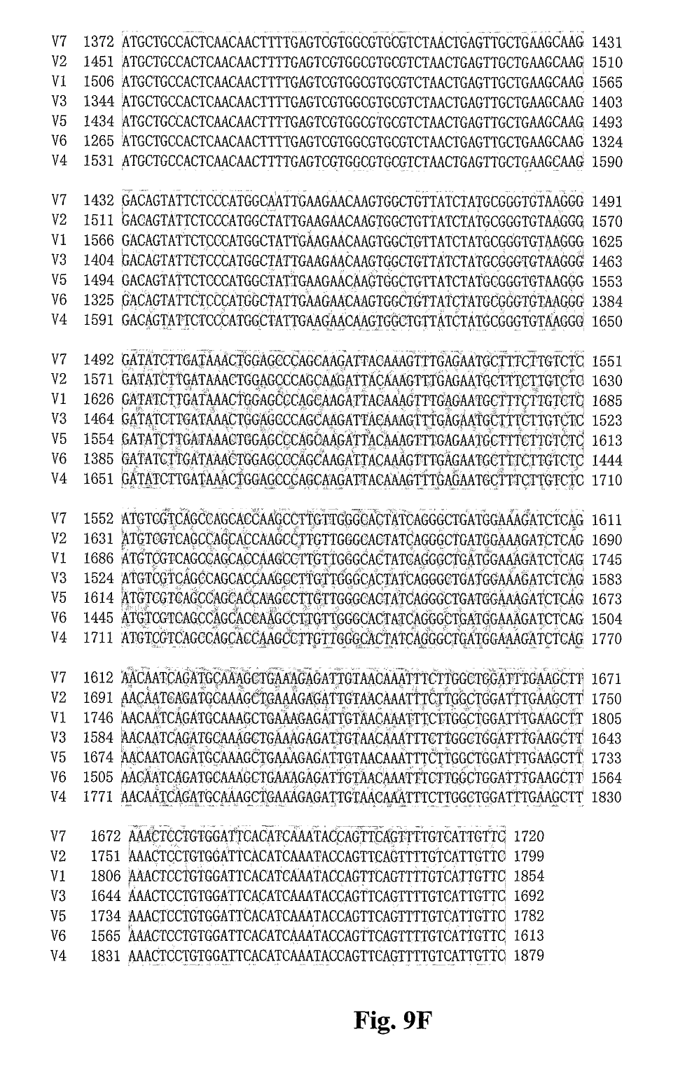

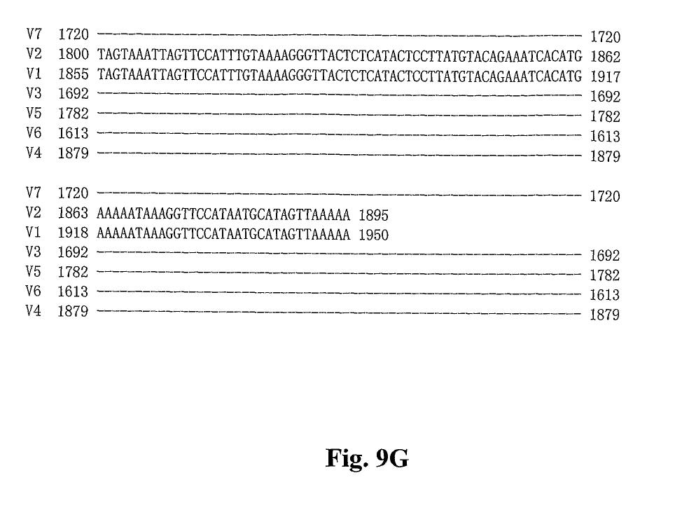

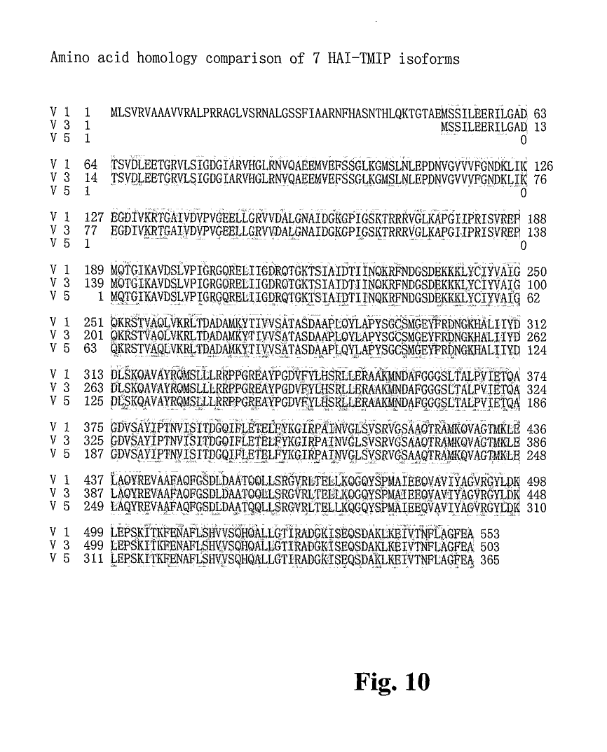

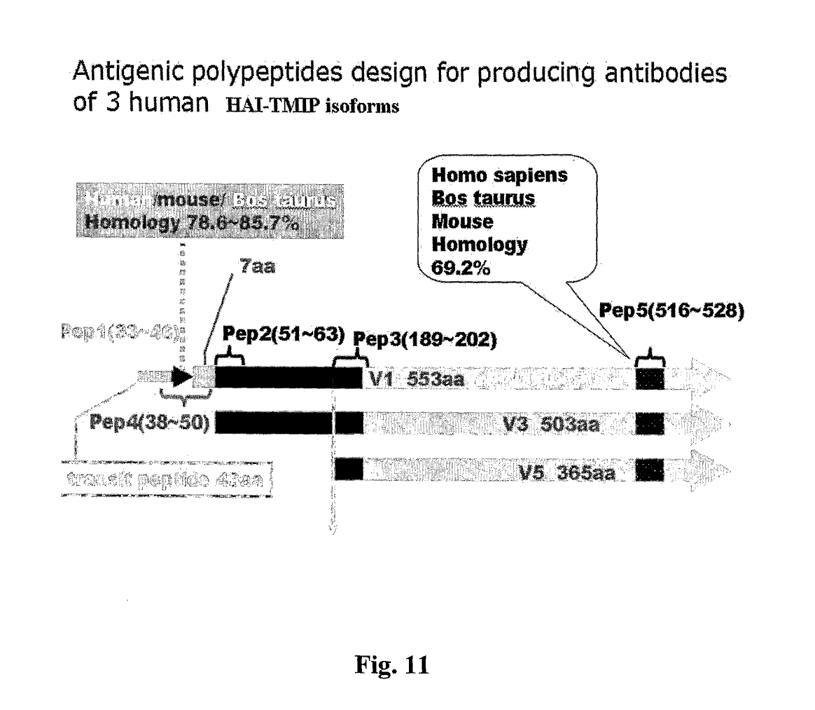

The present invention provides nucleic acids encoding isoforms of HAI-TMIP (e.g., HAI-TMIP variant 1, 2, 3, 4, 5, 6, or 7). In particular, the present invention provides nucleic acids encoding five isoforms of the HAI-TMIP, termed HAI-TMIP variant 3, HAI-TMIP variant 4, HAI-TMIP variant 5, HAI-TMIP variant 6, and HAI-TMIP variant 7. HAI-TMIP variant 3 differs from HAI-TMIP variant 1 as a result of a deletion of exon 2. HAI-TMIP variant 4 differs from HAI-TMIP variant 1 as a result of a deletion of exon 2 and the addition of an exon of 187 base pairs in the intron 3 position. HAI-TMIP variant 5 differs from HAI-TMIP variant 1 as a result of a deletion of exon 2 and the alternative splicing of intron 4 as an exon. HAI-TMIP variant 6 differs from HAI-TMIP variant 1 as a result of a deletion of exons 2 and 3. HAI-TMIP variant 7 differs from HAI-TMIP variant 2 as a result of truncation in exon 2. FIG. 9 depicts an alignment of the exons and introns of the seven HAI-TMIP variants, namely HAI-TMIP variants 1 to 7. FIG. 10 depicts a nucleotide sequence alignment of HAI-TMIP variants 1 to 7. FIG. 11 depicts an amino acid sequence alignment of HAI-TMIP variants 1, 3 and 5. HAI-TMIP variants 1 and 2 encode the same 553 amino acid sequence, and HAI-TMIP variants 3, 4, 6, and 7 encode the same 503 amino acid sequence.

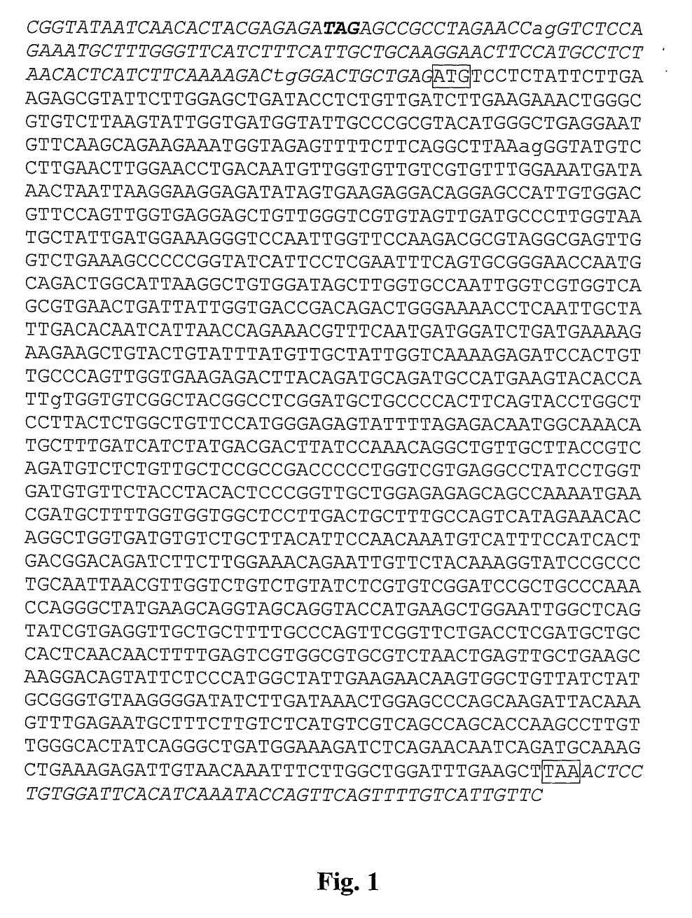

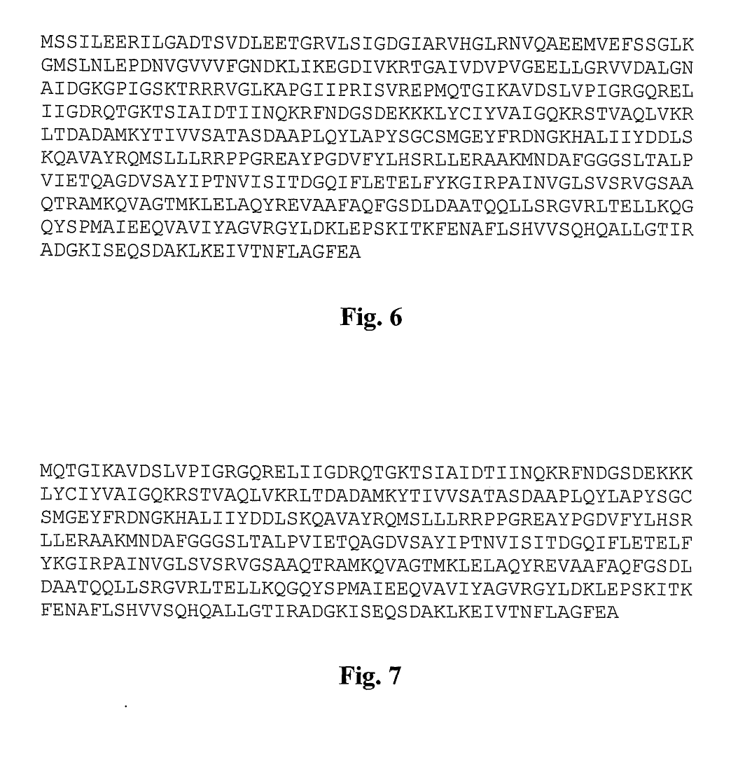

The present invention provides a nucleic acid sequence comprising: (a) one of the nucleotide sequences of HAI-TMIP variants 3 to 7 depicted in FIGS. 1, 2, 3, 4, and 5 (SEQ ID NOS:3, 4, 5, 6, and 7, respectively); (b) the coding region of one of the nucleotide sequences depicted in FIGS. 1, 2, 3, 4 and 5; (SEQ ID NOS:37, 38, 39, 40 and 41); (c) a fragment of one of the nucleotide sequences of HAI-TMIP variants 3 to 7 depicted in FIGS. 1, 2, 3, 4, and 5 (SEQ ID NOS:3, 4, 5, 6, and 7, respectively); (d) a fragment of the coding region of one of the nucleotide sequences depicted in FIGS. 1, 2, 3, 4 and 5 (SEQ ID NOS:37, 38, 39, 40 and 41); (e) a nucleotide sequence encoding one of the HAI-TMIP variants depicted in FIGS. 6 and 7 (SEQ ID NOS: 8 and 9); (f) a nucleotide sequence encoding a fragment of one of the HAI-TMIP variants depicted in FIGS. 6 and 7 (SEQ ID NOS:8 and 9); (g) any nucleotide sequence that hybridizes to the complement of one of the nucleotide sequences of HAI-TMIP variants 3 to 7 depicted in FIGS. 1, 2, 3, 4, and 5 (SEQ ID NOS:3, 4, 5, 6, and 7, respectively) under stringent conditions (in a specific embodiment, highly stringent conditions); (h) any nucleotide sequence that hybridizes to the complement of a nucleotide sequence encoding one of the HAI-TMIP variants depicted in FIGS. 6 and 7 (SEQ ID NOS:8 and 9); and/or (i) the complement of any of the nucleotide sequences recited in (a) through (h). As those skilled in the art will readily appreciate, an amino acid sequence encoded by a given nucleotide sequence may also be encoded by a number of degenerate nucleotide sequences which are apparent to those skilled in the art. Thus, the present invention includes degenerate variants of the sequences described in (a), (b), (c), (d), (h) and (i). In a specific embodiment, the nucleic acid sequences and nucleotide sequences disclosed in this Section 5.1 are purified.

The present invention provides nucleotide sequences encoding functionally active derivatives of HAI-TMIP variants 3 to 7. The present invention provides a nucleotide sequence that is at least 65%, preferably at least 70%, at least 75%, at least 80%, at least 85%, at least 90%, at least 95%, or at least 98% identical to one or more of the nucleotide sequences of (a) through (h). The present invention also provides a nucleotide sequence encoding a polypeptide that is at least 65%, preferably at least 70%, at least 75%, at least 80%, at least 85%, at least 90%, at least 95%, or at least 98% identical to one of the polypeptides encoded by the nucleotide sequences of (a), (b), (c), (d), (g) and (h).

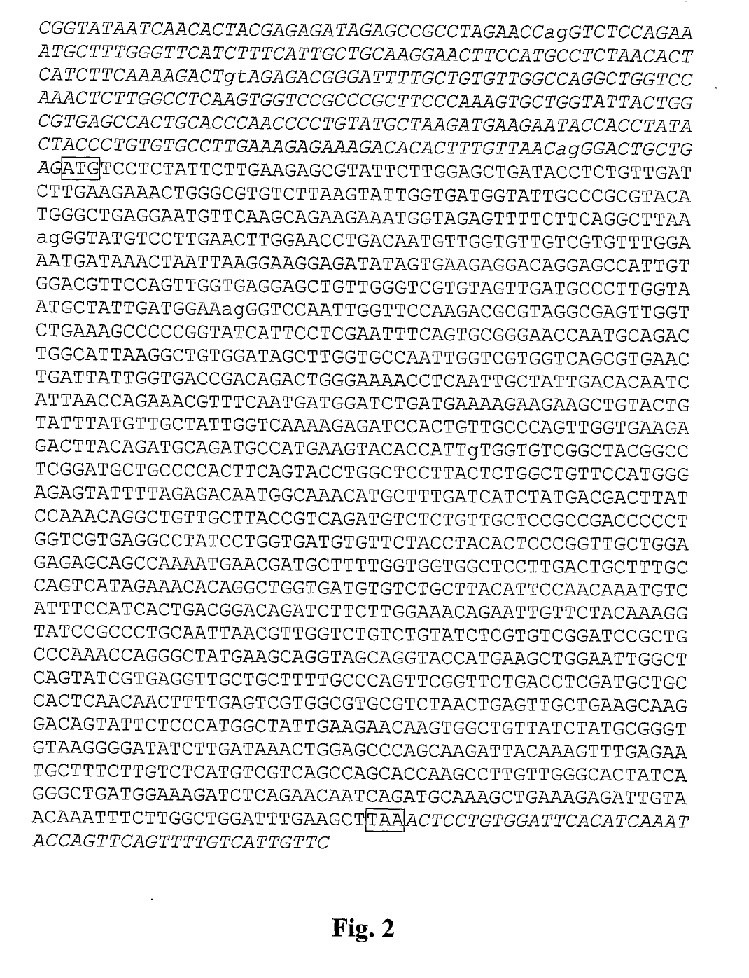

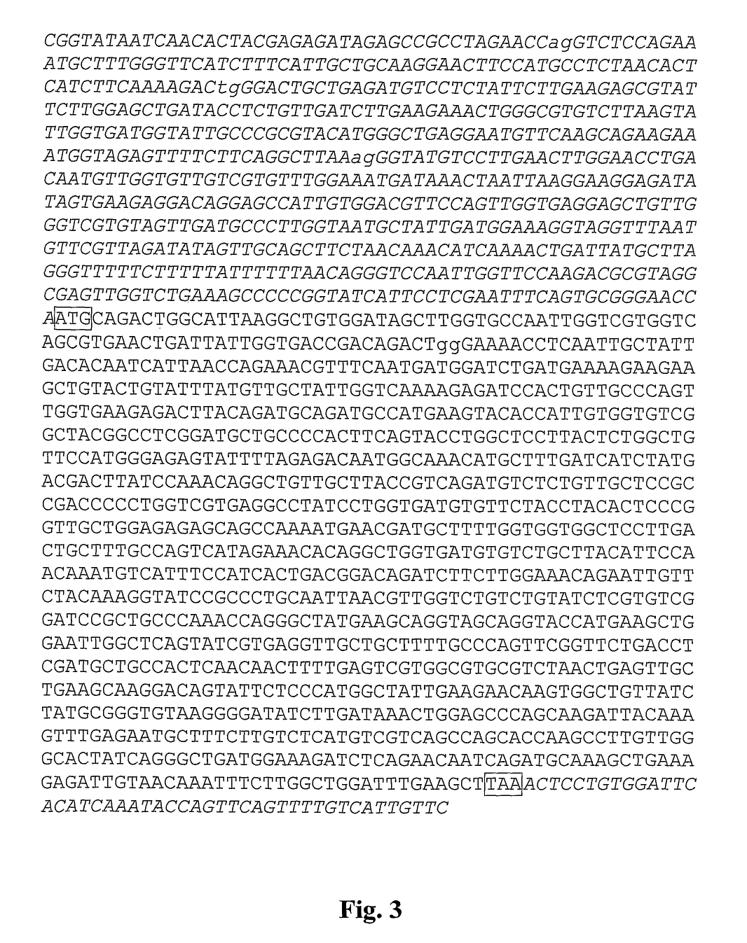

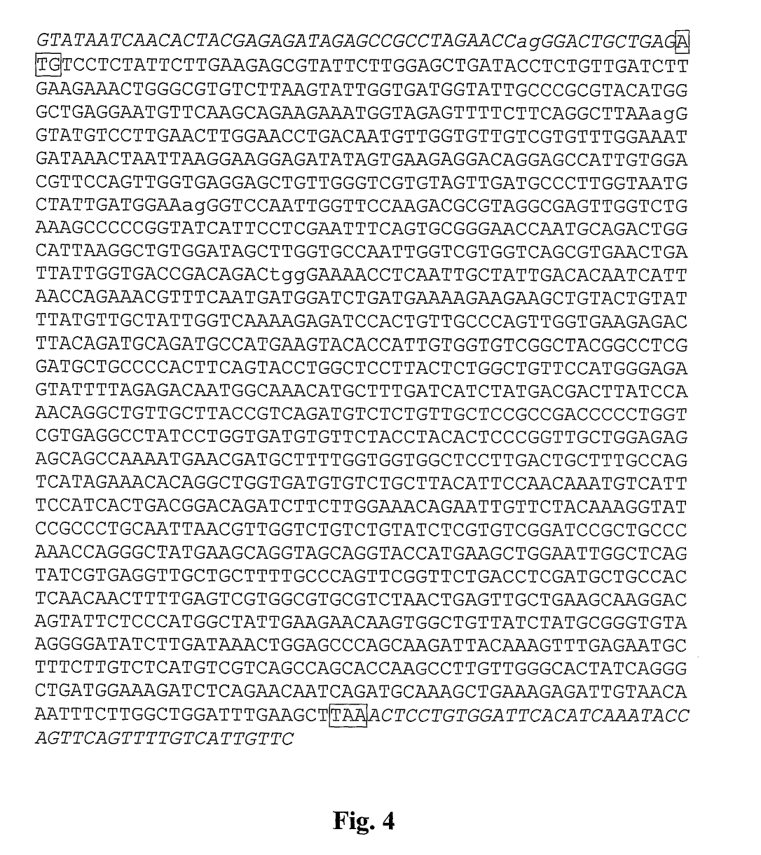

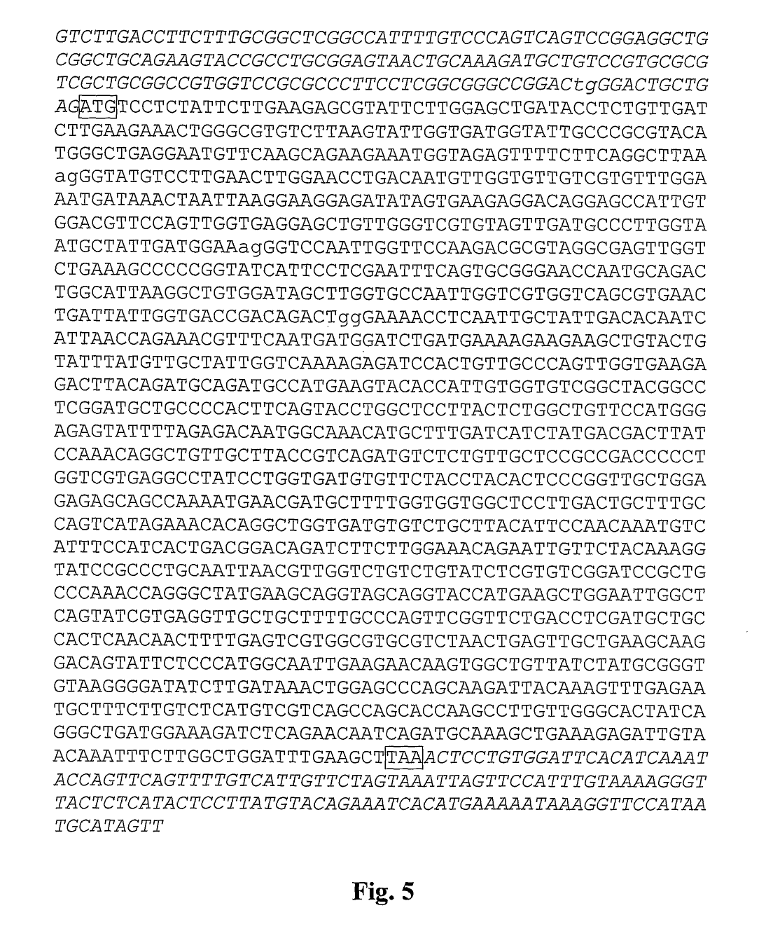

In a specific embodiment, the present invention provides a nucleic acid sequence comprising the nucleotide sequence of HAI-TMIP variant 3 depicted in FIG. 1 (SEQ ID NO:3 or 37), the complement thereof or a fragment thereof. In another embodiment, the present invention provides a nucleic acid sequence comprising the nucleotide sequence of HAI-TMIP variant 4 depicted in FIG. 2 (SEQ ID NO:4 or 38), the complement thereof or a fragment thereof. In another embodiment, the present invention provides a nucleic acid sequence comprising the nucleotide sequence of HAI-TMIP variant 5 depicted in FIG. 3 (SEQ ID NO:5 or 39), the complement thereof or a fragment thereof. In another embodiment, the present invention provides a nucleic acid sequence comprising the nucleotide sequence of HAI-TMIP variant 6 depicted in FIG. 4 (SEQ ID NO:6 or 40), the complement thereof or a fragment thereof. In another embodiment, the present invention provides a nucleic acid sequence comprising the nucleotide sequence of HAI-TMIP variant 7 depicted in FIG. 5 (SEQ ID NO:7 or 41), the complement thereof or a fragment thereof. In another embodiment, the present invention provides a nucleic acid sequence comprising a nucleotide sequence encoding the HAI-TMIP variant depicted in FIG. 6 (SEQ ID NO:8) or a fragment thereof. In yet another embodiment, the present invention provides a nucleic acid sequence comprising a nucleotide sequence encoding the HAI-TMIP variant depicted in FIG. 7 (SEQ ID NO:9) or a fragment thereof.

Fragments of the nucleotide sequences described herein are at least 10 nucleotides in length. In a specific embodiment, the fragments of the nucleotide sequences described herein are at least 15, at least 20, at least 25, at least 30, at least 35, at least 40, at least 45, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1200, at least 1500, at least 1600 or more contiguous nucleotides in length. In another embodiment, the fragments of the nucleotide sequences described herein are about 10 to about 50, about 10 to about 100, about 10 to about 150, about 10 to about 300, about 10 to about 500, about 10 to about 800 or about 10 to about 1000, about 10 to about 1200, about 10 to about 1500, about 200 to about 500, about 200 to about 800, about 200 to about 1000, about 200 to about 1200, about 200 to about 1500, about 500 to about 1000, or about 500 to about 1500 nucleotides in length. In another embodiment, the fragments of the nucleotide sequences described herein encode at least 10, at least 15, at least 20, at least 25, at least 50, at least 75, at least 100, at least 125, at least 150, at least 175, at least 200, at least 225, at least 250, at least 300, at least 325, at least 350 or more contiguous amino acid residues of a HAI-TMIP variant. In another embodiment, the fragments of the nucleotide sequences described herein encode about 10 to about 50, about 10 to about 100, about 10 to about 150, about 10 to about 200, about 10 to about 250, about 10 to about 300, about 50 to about 100, about 50 to about 150, about 50 to about 200, about 50 to about 250, or about 50 to about 300 amino acid residues in length. Fragments of the nucleotide sequences can refer to exons and/or introns of nucleotide sequences. In a specific embodiment, fragments of HAI-TMIP variants 3 to 7 are functionally active, i.e., they are functionally active fragments.

In certain embodiments, a fragment of a nucleotide sequence described herein comprises at least nucleotides 107 to 151 of SEQ ID NO: 7, preferably at least nucleotides 107 to 200 of SEQ ID NO:7, and more preferably at least nucleotides 107 to 250 of SEQ ID NO:7. In other embodiments, a fragment of a nucleotide sequence described herein comprises at least nucleotides 43 to 54 of SEQ ID NOS:3, 4, 5, and 6, preferably at least nucleotides 43 to 66 of SEQ ID NOS:3, 4, 5, and 6, and more preferably at least nucleotides 43 to 76 of SEQ ID NOS: 3, 4, 5, and 6. In other embodiments, a fragment of a nucleotide described herein comprises at least nucleotides 123 to 150 of SEQ ID NO:4, preferably at least nucleotides 123 to 175 of SEQ ID NO:4, at least nucleotides 123 to 200 of SEQ ID NO:4, at least nucleotides 123 to 225 of SEQ ID NO:4, at least nucleotides 123 to 250 of SEQ ID NO:4, at least nucleotides 123 to 275 of SEQ ID NO:4, or at least nucleotides 123 to 309 a SEQ ID NO:4. In other embodiments, a fragment of a nucleotide sequence described herein comprises at least nucleotides 468 to 500 of SEQ ID NO:5, preferably at least nucleotides 468 to 525 of SEQ ID NO:5 or at least nucleotides 468 to 557 of SEQ ID NO:5.

In a specific embodiment, the present invention provides a nucleic acid sequence comprising at least 10, preferably at least 15, at least 20, at least 25, at least 30, at least 35, at least 40, at least 45, at least 50 or more contiguous nucleotides of the nucleotide sequence of HAI-TMIP variant 7 depicted in FIG. 5 (SEQ ID NO:7) or the complement thereof, wherein the nucleic acid sequence comprises at least nucleotide residues 107 to 151 of SEQ ID NO:7 or the complement thereof. In another embodiment, the present invention provides a nucleic acid sequence comprising at least 10, preferably at least 15, at least 20, at least 25, at least 30, at least 35, at least 40, at least 45, at least 50 or more contiguous nucleotides of the nucleotide sequence of HAI-TMIP variants 3 to 6 depicted in FIGS. 1 to 4 (SEQ ID NOS:3, 4, 5, and 6) the complement thereof, wherein the nucleic acid sequence comprises at least nucleotides 43 to 70 of SEQ ID NOS: 3, 4, 5 or 6 or the complement thereof. In another embodiment, the present invention provides a nucleic acid sequence comprising at least 10, preferably at least 15, at least 20, at least 25, at least 30, at least 35, at least 40, at least 45, at least 50 or more contiguous nucleotides of the nucleotide sequence of HAI-TMIP variant 4 depicted in FIG. 2 (SEQ ID NO:4) the complement thereof, wherein the nucleic acid sequence comprises at least nucleotides 123 to 150 of SEQ ID NO:4 or the complement thereof. In yet another embodiment, the present invention provides a nucleic acid sequence comprising at least 10, preferably at least 15, least 20, at least 25, least 30 at least 35, at least 40, at least 45, at least 50 or more contiguous nucleotides of the nucleotide sequence of HAI-TMIP variant 5 depicted in FIG. 3 (SEQ ID NO:5) or the complement thereof, wherein the nucleic acid sequence comprises at least nucleotides 468 to 495 of SEQ ID NO:5 or the complement thereof.

The present invention provides a nucleotide sequence that hybridizes to the complement of one of the nucleotide sequences of HAI-TMIP variants 3 to 7 depicted in FIGS. 1, 2, 3, 4 and 5 (SEQ ID NOS:3, 4, 5, 6, 7, 37, 38, 39, 40 and 41) under stringent conditions (in a specific embodiment, highly stringent conditions). In a specific embodiment, the present invention provides a nucleotide sequence that hybridizes over its entire length to one of the nucleotide sequences of HAI-TMIP variants 3 to 7 depicted in FIGS. 1, 2, 3, 4 and 5 (SEQ ID NOS:3, 4, 5, 6, 7, 37, 38, 39, 40 and 41) under stringent conditions (in a specific embodiment, highly stringent conditions). In another embodiment, the present invention provides a nucleotide sequence that hybridizes to one of the nucleotide sequences of HAI-TMIP variants 3 to 7 depicted in FIGS. 1, 2, 3, 4 and 5 (SEQ ID NOS:3, 4, 5, 6, 7, 37, 38, 39, 40 and 41) under stringent conditions (in a specific embodiment, highly stringent conditions), wherein the nucleotide sequences that hybridize are the same length or about the same length as one of the nucleotide sequences of HAI-TMIP variants 3 to 7 depicted in FIGS. 1, 2, 3, 4, and 5 (SEQ ID NOS:3, 4, 5, 6, 7, 37, 38, 39, 40 and 41) and/or the protein encoded by the nucleotide sequence is functionally equivalent to an HAI-TMIP isoform.

The present invention provides a nucleotide sequence that hybridizes to the complement of the nucleotide sequence encoding one of the amino acid sequences depicted in FIGS. 6 and 7 (SEQ ID NOS:8 and 9) under stringent conditions (in a specific embodiment, highly stringent conditions). In a specific embodiment, the present invention provides a nucleotide sequence that hybridizes over its entire length to the nucleotide sequence encoding one of the amino acid sequences depicted in FIGS. 6 and 7 (SEQ ID NOS:8 and 9) under stringent conditions (in a specific embodiment, highly stringent conditions). In another embodiment, the present invention provides a nucleotide sequence that hybridizes to the nucleotide sequence encoding one of the amino acid sequences depicted in FIGS. 6 and 7 (SEQ ID NOS:8 and 9) under stringent conditions (in a specific embodiment, highly stringent conditions), wherein the nucleotide sequences that hybridize are the same length or about the same length as the nucleotide sequence encoding one of the amino acid sequences depicted in FIGS. 6 and 7 (SEQ ID NOS:8 and 9) and/or the protein encoded by the nucleotide sequence is functionally equivalent to an HAI-TMIP isoform.

The present invention provides nucleic acid sequences that encode fusion proteins, such as, e.g., IgG Fc fusion proteins, in which the nucleic acid sequences comprise a nucleotide sequence described herein, and a nucleotide sequence encoding a heterologous amino acid sequence (i.e., an amino acid not naturally found in conjunction with the amino acid sequence of an HAI-TMIP isoform described herein).

The present invention provides vectors comprising a nucleotide sequence described herein (e.g., a nucleotide sequence encoding HAI-TMIP variants 3 to 7). In a specific embodiment, the vectors comprise a nucleotide sequence described herein operatively linked to a regulatory element that directs the expression of the nucleotide sequence. The present invention provides host cells containing or comprising a nucleotide sequence described herein as well as host cells containing or comprising a vector comprising a nucleotide sequence described herein. Techniques well-known to one of skill in the art, such as electroporation, calcium phosphate precipitate and liposomes, may be used to transfect a host cell with a nucleotide sequence described herein. See, e.g., Section 5.5, infra, for a description of vectors, transfection techniques and host cells. Techniques well-known to one of skill in the art, such as immunoprecitation using antibodies immunospecific an HAI-TMIP or a functionally active fragment thereof, may be used to purify HAI-TMIP variants 3 to 7 or a functionally active fragment thereof. See Section 5.6, infra, for a description of methods for purifying proteinaceous agents.

### 5.2 Amino Acid Sequence of HAI-TMIPs

The present invention provides amino acid sequences of HAI-TMIP variants (e.g., HAI-TMIP variant, 1, 2, 3, 4, 5, 6, or 7) as well as fragments and derivatives thereof. In particular, the present invention provides amino acid sequences of HAI-TMIP variants 3 to 7 as well as functionally active fragments and functionally active derivatives thereof. In one embodiment, the present invention provides a purified protein encoded by a nucleotide sequence described in Section 5.1, supra. In a specific embodiment, the present invention provides a purified protein encoded by the nucleotide sequences depicted in FIGS. 1 to 5 (SEQ ID NOS: 3, 4, 5, 6, 7, 37, 38, 39, 40 and 41). In another embodiment, the present invention provides a purified protein having the amino acid sequence depicted in FIG. 6 (SEQ ID NO:8). In another embodiment, the present invention provides a purified protein having the amino acid sequence depicted in FIG. 7 (SEQ ID NO:9).

The present invention provides a purified protein encoded by a nucleotide sequence that hybridizes to one of the nucleotide sequence depicted in FIGS. 1 to 5 (SEQ ID NOS:3, 4, 5, 6, 7, 37, 38, 39, 40 and 41) under stringent conditions (in specific embodiments, highly stringent conditions). In a specific embodiment, the present invention provides a purified protein encoded by a nucleotide sequence that hybridizes over its entire length to one of the nucleotide sequences depicted in FIGS. 1 to 5 (SEQ ID NOS:3, 4, 5, 6, 7, 37, 38, 39, 40 and 41) under stringent conditions (in specific embodiments, highly stringent conditions). In another embodiment, the present invention provides a purified protein encoded by a nucleic acid sequence that hybridizes to one of the nucleotide sequences depicted in FIGS. 1 to 5 (SEQ ID NOS:3, 4, 5, 6, 7, 37, 38, 39, 40 and 41) under stringent conditions (in specific embodiments, highly stringent conditions), wherein the nucleic acid sequence is the same length or about the same length as one of the nucleotide sequences in FIGS. 1 to 5 (SEQ ID NOS:3, 4, 5, 6, 7, 37, 38, 39, 40 and 41) and/or the protein is functionally equivalent to an HAI-TMIP isoform.

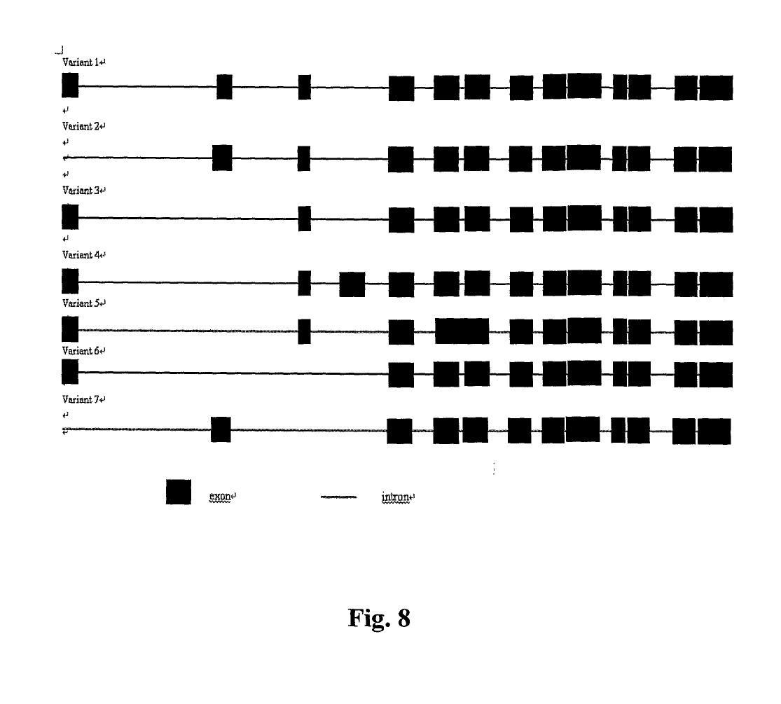

The present invention provides a purified protein comprising at least 10, preferably at least 15, at least 20, at least 25, at least 30, at least 35, at least 45, at least 50 or more contiguous residues of the amino acid sequence depicted in FIG. 8 or 9 (SEQ ID NO:8 or 9).

The present invention also provides a purified protein that is at least 65%, preferably at least 75%, at least 80%, at least 85%, at least 90%, at least 95%, at least 99%, at least 99.5% or at least 99.8% identical to the amino acid sequence depicted in FIG. 6 or 7 (SEQ ID NO:8 or 9). In a specific embodiment, the present invention provides a purified protein that is at least 80%, preferably at least 85%, at least 90%, at least 92%, at least 95%, at least 99%, at least 99.5% or at least 99.8% identical to the amino acid sequence depicted in FIG. 6 (SEQ ID NO:8), wherein the first 10 amino terminal amino acid residues of the purified protein are MSSILEERIL (SEQ ID NO:10). In another embodiment, the present invention provides a purified protein that is at least 80% preferably at least 85%, at least 90%, at least 92%, at least 95%, at least 99%, at least 99.5% or at least 99.8% identical to the amino acid sequence depicted in FIG. 6 (SEQ ID NO:8), wherein the first 10 amino terminal amino acid residues of the purified protein are MQTGIKAVDS (SEQ ID NO:11).

The present invention also provides fusion proteins comprising HAI-TMIP variants 3 to 7, a functionally active fragment or a functionally active derivative thereof and a heterologous amino acid sequence. In particular, the present invention provides fusion proteins comprising a purified protein described in this Section 5.2 and a heterologous amino acid sequence.

### 5.3 ATP Synthase Complexes

The present invention provides a protein complex comprising a HAI-TMIP variant (e.g., 1, 2, 3, 4, 5, 6, or 7), or fragment or derivative thereof. In particular, the present invention provides a protein complex comprising a HAI-TMIP variant 3, 4, 5, 6, or 7 or a functionally active derivative or a functionally active fragment thereof. The present invention provides a purified complex comprising a protein having the amino acid sequence of a purified protein described in Section 5.2, supra. In a specific embodiment, the present invention provides a protein complex comprising a protein having: (a) the amino acid sequence depicted in FIG. 6 or 7 (SEQ ID NO:8 or 9); (b) the amino acid sequence of a fragment of at least 10, preferably at least 15, at least at least 20, at least 25, at least 30, at least 35, at least 45, at least 50 or more contiguous residues of the amino acid sequence depicted in FIG. 8 or 9 (SEQ ID NO:8 or 9); (c) the amino acid sequence that is at least 65%, preferably at least 75%, at least 80%, at least 85%, at least 90%, at least 95%, at least 99%, at least 99.5% or at least 99.8% identical to the amino acid sequence depicted in FIG. 6 or 7 (SEQ ID NO:8 or 9).

The present invention provides a protein complex comprising a protein encoded by a nucleotide sequence described in Section 5.1, supra. In a specific embodiment, the present invention provides a protein complex comprising a protein encoded by: (a) one of the nucleotide sequences of HAI-TMIP variants 3 to 7 depicted in FIGS. 1, 2, 3, 4, and 5 (SEQ ID NOS:3, 4, 5, 6, 7, 37, 38, 39, 40 and 41); (b) a fragment of one of the nucleotide sequences of HAI-TMIP variants 3 to 7 depicted in FIGS. 1, 2, 3, 4, and 5 (SEQ ID NOS:3, 4, 5, 6, 7, 37, 38, 39, 40 and 41); (c) a nucleotide sequence encoding one of the HAI-TMIP variants depicted in FIGS. 6 and 7 (SEQ ID NOS: 8 and 9); (d) a nucleotide sequence encoding a fragment of one of the HAI-TMIP variants depicted in FIGS. 6 and 7 (SEQ ID NOS:8 and 9); (e) any nucleotide sequence that hybridizes to the complement of one of the nucleotide sequences of HAI-TMIP variants 3 to 7 depicted in FIGS. 1, 2, 3, 4, and 5 (SEQ ID NOS:3, 4, 5, 6, 7, 37, 38, 39, 40 and 41) under stringent conditions (in a specific embodiment, highly stringent conditions); (f) any nucleotide sequence that hybridizes to the complement of a nucleotide sequence encoding one of the HAI-TMIP variants depicted in FIGS. 6 and 7 (SEQ ID NOS:8 and 9); (g) a nucleotide sequence that is at least 65%, preferably at least 70%, at least 75%, at least 80%, at least 85%, at least 90%, at least 95%, or at least 98% identical to one or more of the nucleotide sequences of (a) through (f); (h) a nucleotide sequence encoding a polypeptide that is at least 65%, preferably at least 70%, at least 75%, at least 80%, at least 85%, at least 90%, at least 95%, or at least 98% identical to one of the polypeptides encoded by the nucleotide sequences of (a), (b), (e) and (f); and/or (i) the complement of any of the nucleotide sequences recited in (a) through (h). In one embodiment, the protein complex is an F₁ subcomplex of ATP synthase. In another embodiment, the protein complex is an F₁F₀ ATP synthase holoenzyme.

In some embodiments, a protein complex described in this Section 5.3 is the F₁ subcomplex of ATP synthase. In accordance with this embodiment, the protein complex may further comprise: (a) an ATP synthase β subunit or a functionally active derivative or a functionally active fragment thereof; (b) an ATP synthase γ subunit or a functionally active derivative or a functionally active fragment thereof; (c) an ATP synthase δ subunit or a functionally active derivative or a functionally active fragment thereof; and/or (d) an ATP synthase ε subunit or a functionally active derivative or a functionally active fragment thereof. Table 1, infra, provides the accession numbers of the nucleotide and amino acid sequences of the components of the human F₁ subcomplex of ATP synthase.

TABLE 1
GenBank Accession Nos. for Subunits of
Human F₁ Subcomplex of ATP Synthase

| | Nucleotide | Amino Acid |
|---|---|---|
| Name | Accession Nos. | Accession Nos. |
| Human ATP synthase | NM_001686 | NP_001677 |
| β subunit | BC016512 | |
| Human ATP synthase | NM_001001973 | NP_001001973 |
| γ subunit | NM_005174 | |
| Human ATP synthase | NM_001687 | NP_001678 |
| δ subunit | NM_001001975 | |
| | BC050458 | |
| | BC058927 | |
| Human ATP synthase | NM_001001977 | NP_008817 |
| ε subunit | BC070167 | |

In a specific embodiment, the present invention provides an F₁ subcomplex of an ATP synthase comprising: (a) an HAI-TMIP variant 3, 4, 5, 6, or 7 (SEQ ID NO: 8 or 9), an HAI-TMIP variant encoded by the nucleotide sequence depicted in FIG. 1, 2, 3, 4 or 5 (SEQ ID NO: 3, 4, 5, 6, 7, 37, 38, 39, 40 or 41), or a protein encoded by a nucleotide sequence that hybridizes to the nucleotide sequence depicted in FIG. 1, 2, 3, 4 or 5 (SEQ ID NO: 3, 4, 5, 6, 7, 37, 38, 39, 40 or 41) or its complement under stringent conditions (in specific embodiments, highly stringent conditions); and (b) one, two, three or all of the following: (i) ATP synthase β subunit (e.g., an Accession NO. in Table 1), an ATP synthase β subunit encoded by an ATP synthase β subunit nucleotide sequence (e.g., an Accession NO. in Table 1), or a protein encoded by a nucleotide sequence that hybridizes to the nucleotide sequence of an ATP synthase β subunit (e.g., an Accession NO. in Table 1) or its complement under stringent conditions (in specific embodiments, highly stringent conditions); (ii) ATP synthase γ subunit (e.g., an Accession NO. in Table 1), an ATP synthase γ subunit encoded by an ATP synthase γ subunit nucleotide sequence (e.g., an Accession NO. in Table 1), or a protein encoded by a nucleotide sequence that hybridizes to the nucleotide sequence of an ATP synthase γ subunit (e.g., an Accession NO. in Table 1) or its complement under stringent conditions (in specific embodiments, highly stringent conditions); (iii) ATP synthase δ subunit (e.g., an Accession NO. in Table 1), an ATP synthase δ subunit encoded by an ATP synthase δ subunit nucleotide sequence (e.g., an Accession NO. in Table 1), or a protein encoded by a nucleotide sequence that hybridizes to the nucleotide sequence of an ATP synthase δ subunit (e.g., an Accession NO. in Table 1) or its complement under stringent conditions (in specific embodiments, highly stringent conditions); and (iv) ATP synthases subunit (e.g., an Accession NO. in Table 1), an ATP synthase s subunit encoded by an ATP synthase s subunit nucleotide sequence (e.g., an Accession NO. in Table 1), or a protein encoded by a nucleotide sequence that hybridizes to the nucleotide sequence of an ATP synthase s subunit (e.g., an Accession NO. in Table 1) or its complement under stringent conditions (in specific embodiments, highly stringent conditions).

In accordance with the embodiment in the immediately preceding paragraph, the protein complex may further comprise one, two, three, four, five, more or all the following subunits of F₀ subcomplex: (i) B1 (e.g., an Accession NO. in Table 2), B1 encoded by a B1 nucleotide sequence (e.g., an Accession NO. in Table 2), or a protein encoded by a nucleotide sequence that hybridizes to the nucleotide sequence of B1 (e.g., an Accession NO. in Table 2) or its complement under stringent conditions (in specific embodiments, highly stringent conditions); (ii) C1 (e.g., an Accession NO. in Table 2), C1 encoded by a C1 nucleotide sequence (e.g., an Accession NO. in Table 2), or a protein encoded by a nucleotide sequence that hybridizes to the nucleotide sequence of C1 (e.g., an Accession NO. in Table 2) or its complement under stringent conditions (in specific embodiments, highly stringent conditions); (iii) C2 (e.g., an Accession NO. in Table 2), C2 encoded by a C2 nucleotide sequence (e.g., an Accession NO. in Table 2), or a protein encoded by a nucleotide sequence that hybridizes to the nucleotide sequence of C2 (e.g., an Accession NO. in Table 2) or its complement under stringent conditions (in specific embodiments, highly stringent conditions); (iv) C3 (e.g., an Accession NO. in Table 2), C3 encoded by a C3 nucleotide sequence (e.g., an Accession NO. in Table 2), or a protein encoded by a nucleotide sequence that hybridizes to the nucleotide sequence of C3 (e.g., an Accession NO. in Table 2) or its complement under stringent conditions (in specific embodiments, highly stringent conditions); (v) d (e.g., an Accession NO. in Table 2), d encoded by a d nucleotide sequence (e.g., an Accession NO. in Table 2), or a protein encoded by a nucleotide sequence that hybridizes to the nucleotide sequence of d (e.g., an Accession NO. in Table 2) or its complement under stringent conditions (in specific embodiments, highly stringent conditions); (vi) E (e.g., an Accession NO. in Table 2), E encoded by an E nucleotide sequence (e.g., an Accession NO. in Table 2), or a protein encoded by a nucleotide sequence that hybridizes to the nucleotide sequence of E (e.g., an Accession NO. in Table 2) or its complement under stringent conditions (in specific embodiments, highly stringent conditions); (vii) F2 (e.g., an Accession NO. in Table 2), F2 encoded by a F2 nucleotide sequence (e.g., an Accession NO. in Table 2), or a protein encoded by a nucleotide sequence that hybridizes to the nucleotide sequence of F2 (e.g., an Accession NO. in Table 2) or its complement under stringent conditions (in specific embodiments, highly stringent conditions); (viii) F6 (e.g., an Accession NO. in Table 2), F6 encoded by a F6 nucleotide sequence (e.g., an Accession NO. in Table 2), or a protein encoded by a nucleotide sequence that hybridizes to the nucleotide sequence of F6 (e.g., an Accession NO. in Table 2) or its complement under stringent conditions (in specific embodiments, highly stringent conditions); (ix) G (e.g., an Accession NO. in Table 2), G encoded by a G nucleotide sequence (e.g., an Accession NO. in Table 2), or a protein encoded by a nucleotide sequence that hybridizes to the nucleotide sequence of G (e.g., an Accession NO. in Table 2) or its complement under stringent conditions (in specific embodiments, highly stringent conditions); and/or (x) s (e.g., an Accession NO. in Table 2), s encoded by an s nucleotide sequence (e.g., an Accession NO. in Table 2), or a protein encoded by a nucleotide sequence that hybridizes to the nucleotide sequence of s (e.g., an Accession NO. in Table 2) or its complement under stringent conditions (in specific embodiments, highly stringent conditions).

In some embodiments, a protein complex described in this Section 5.3 is an F₁F₀ ATP synthase holoenzyme. In accordance with this embodiment, the protein complex may comprise: (a) HAI-TMIP variant 3, 4, 5, 6 or 7 (SEQ ID NO:8 or 9), an HAI-TMIP variant encoded by the nucleotide sequence depicted in FIG. 1, 2, 3, 4 or 5 (SEQ ID NO:3, 4, 5, 6, 7, 37, 38, 39, 40 or 41) or a protein encoded by a nucleotide sequence that hybridizes to the nucleotide sequence depicted in FIG. 1, 2, 3, 4 or 5 (SEQ ID NO:3, 4, 5, 6, 7, 37, 38, 39, 40 or 41) or its complement under stringent conditions (in specific embodiments, highly stringent conditions); (b) one, two, three or all of the subunits of F₁: (i) an ATP synthase β subunit or a functionally active derivative or a functionally active fragment thereof; (ii) an ATP synthase γ subunit or a functionally active derivative or a functionally active fragment thereof; (iii) an ATP synthase δ subunit or a functionally active derivative or a functionally active fragment thereof; and/or (iv) an ATP synthase s subunit or a functionally active derivative or a functionally active fragment thereof; and (c) one, two, three, four, more or all of the subunits of F₀: (i) B1 or a functionally active derivative or a functionally active fragment thereof; (ii) C1 or a functionally active derivative or a functionally active fragment thereof; (iii) C2 or a functionally active derivative or a functionally active fragment thereof; (iv) C3 or a functionally active derivative or a functionally active fragment thereof; (v) d or a functionally active derivative or a functionally active fragment thereof; (vi) E or a functionally active derivative or a functionally active fragment thereof; (vii) F2 or a functionally active derivative or a functionally active fragment thereof; (viii) F6 or a functionally active derivative or a functionally active fragment thereof; (ix) G or a functionally active derivative or a functionally active fragment thereof; and/or (x) s or a functionally active derivative or a functionally active fragment thereof. Table 1, supra, provides the accession numbers of the nucleotide and amino acid sequences of the components of the human F₁ subcomplex of ATP synthase. Table 2, infra, provides the accession numbers of the nucleotide and amino acid sequences of the components of the human F₀ subcomplex of ATP synthase.

TABLE 2
GenBank Accession Nos. for Subunits of
Human F₀ Subcomplex of ATP Synthase

| | Nucleotide Sequence | Amino Acid Sequence |
|---|---|---|
| Name | GenBank Accession Nos. | GenBank Accession Nos. |
| B1 | NM_001688 | NP_001679 |
| C1 | V1: NM_005175 | NP_005166 |
| | V2: NM_001002027 | NP_001002027 |
| C2 | V1: NM_001002031 | NP_001002031 |
| | V2: NM_005176 | NP_005167 |
| C3 | V2: NM_001689 | NP_001680 |
| | V3: NM_001002258 | NP_001002258 |
| D | V1: NM_006356 | NP_006347 |
| | V2: NM_001003785 | NP_001003785 |
| E | NM_007100 | NP_009031 |
| F2 | V1: NM_004889 | NP_004880 |
| | V2: NM_001003713 | NP_001003713 |
| | V3: NM_001003714 | NP_001003714 |
| | V4: NM_001039178 | NP_001034267 |
| F6 | V1: NM_001003703 | NP_001003703 |
| | V2: NM_001685 | NP_001676 |
| | V3: NM_001003696 | NP_001003696 |
| | V4: NM_001003697 | NP_001003697 |
| | V5: NM_001003701 | NP_001003701 |
| G | NM_006476 | NP_006467 |
| S | V1: NM_001003803 | NP_001003803 |
| | V2: NM_001003805 | NP_001003805 |
| | V3: NM_015684 | NP_056499 |

In specific embodiments, a protein complex described in this Section 5.3 has ATP synthase activity. In one embodiment, a protein complex described in this Section 5.3 catalyzes ATP synthesis when the electrochemical H+ gradient is favorable. In other embodiments, a protein complex described in this Section 5.3 hydrolyzes ATP.

In some embodiments, a protein complex described in this Section 5.3 is found in the mitochondria of human cells. In other embodiments, a protein complex described in this Section 5.3 is found at the cell surface of one or more of the following human cells: lymphocytes, hepatocytes, vascular endothelial cells and/or cancer cells (e.g., breast cancer cells). In other embodiments, a protein complex described in this Section 5.3 is a receptor for one or more of the following: apolipoprotein E-rich high-density lipoprotein, angiostatin and/or vasoconstrictor coupling factor 6. In certain embodiments, a protein complex described in this Section 5.3 is purified.

In certain embodiments, the invention provides protein complexes that comprise homologs or analogs of the human proteins of the complexes of the invention. Homologs or analogs of the components of a complex of the invention are at least 50%, at least 55%, at least 60%, at least 65%, at least 70%, at least 75%, at least 80%, at least 85%, at least 90%, at least 95%, at least 98%, at least 99% or at least 99.5% identical to a human protein of a complex of the invention. Derivatives can be, e.g., fusion proteins, mutant forms of the protein, or forms of the protein with chemical moieties linked to the protein. A fragment of a component of a complex of the invention is a portion of the protein component that maintains the ability of the component to be physically integrated into the complex.

In certain embodiments, the protein components of a complex of the invention are derived from the same species. In more specific embodiments, the protein components are all derived from human. In another specific embodiment, the protein components are all derived from a mammal.

In certain other embodiments, the protein components of a complex of the invention are derived from a non-human species, such as, but not limited to, cow, pig, horse, cat, dog, rat, mouse, a primate (e.g., a chimpanzee, a monkey such as a cynomolgous monkey). In certain embodiments, one or more components are derived from human and the other components are derived from a mammal other than a human to give rise to chimeric complexes.

In certain embodiments of the invention, at least two components of a complex of the invention are linked to each other via at least one covalent bond. A covalent bond between components of a complex of the invention increases the stability of the complex of the invention because it prevents the dissociation of the components. Any method known to the skilled artisan can be used to achieve a covalent bond between at least two components of the invention.

In specific embodiments, covalent cross-links are introduced between adjacent subunits. Such cross-links can be between the side chains of amino acids at opposing sides of the dimer interface. Any functional groups of amino acid residues at the dimer interface in combination with suitable cross-linking agents can be used to create covalent bonds between the protein components at the dimer interface. Existing amino acids at the dimer interface can be used or, alternatively, suitable amino acids can be introduced by site-directed mutagenesis.

In exemplary embodiments, cysteine residues at opposing sides of the dimer interface are oxidized to form disulfide bonds. See, e.g., Reznik et al., 1996, Nature Biotechnology 14:1007-1011, at page 1008. 1,3-dibromoacetone can also be used to create an irreversible covalent bond between two sulfhydryl groups at the dimer interface. In certain other embodiments, lysine residues at the dimer interface are used to create a covalent bond between the protein components of the complex. Crosslinkers that can be used to create covalent bonds between the epsilon amino groups of lysine residues are, e.g., but are not limited to, bis(sulfosuccinimidyl)suberate; dimethyladipimidate-2HD1; disuccinimidyl glutarate; N-hydroxysuccinimidyl 2,3-dibromoproprionate.

### 5.4 Fusion Proteins

One, two or more protein components of the complexes of the invention (e.g., HAI-TMIP variant 3, 4, 5, 6 or 7) can be fusion proteins comprising a peptide tag or other heterologous sequence. Also, an HAI-TMIP variant 1 or 2 can be fused to a heterologous sequence (such as a peptide tag). In certain embodiments, a leader peptide may also be fused to a protein component thereby facilitating the transport of the protein component into the endoplasmic reticulum (ER) for secretion.

In specific embodiments, at least two components of a complex of the invention are expressed as a fusion protein, i.e., fusion complexes. Any recombinant DNA technology known to the skilled artisan can be used to construct the DNA encoding the fusion complex. Care should be taken that the two or more open reading frames are cloned in frame with each other. Any method known to the skilled artisan can be used to express and purify the fusion protein. Exemplary methods are discussed herein. In certain, more specific embodiments, the two components that form the fusion protein are connected to each other via a linker peptide. Thus, the fusion complex is encoded by the open reading frame (ORF) for the first component protein, the ORF encoding the linker peptide, and the ORF encoding the second component protein. Without being bound by theory, the linker peptide retains the two components of the complex in close spatial proximity, thus increasing the rate of binding of the two components to each other and thereby stabilizing the complex of the invention.

In various embodiments, a fusion protein can be made by ligating a gene sequence encoding a protein component of a complex of the invention to the sequence encoding the peptide tag or the leader peptide in the proper reading frame. If genomic sequences are used, care should be taken to ensure that the modified gene remains within the same translational reading frame, uninterrupted by translational stop signals and/or spurious messenger RNA splicing signals.

In specific embodiments, the nucleotide sequence encoding a peptide tag is fused at its amino terminal to the carboxyl terminal of the ORF for the protein component. The precise site at which the fusion is made in the carboxyl terminal is not critical. For example, the nucleotide sequence encoding a peptide tag may replace part of the ORF encoding the protein. component. The optimal site can be determined by routine experimentation. In other embodiments, the nucleotide sequence encoding a peptide tag is fused at its carboxyl terminal to the amino terminal of the ORF for the protein component.

A variety of peptide tags known in the art may be used to generate fusion proteins of the protein components of a complex of the invention, such as but not limited to the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4; 220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Some peptide tags may afford the fusion protein novel structural properties, such as the ability to form multimers. Peptide tags that promote homodimerization or homopolymerization are usually derived from proteins that normally exist as homopolymers. Peptide tags such as the extracellular domains of CD8 (Shiue et al., 1988, J. Exp. Med. 168:1993-2005), or CD28 (Lee et al., 1990, J. Immunol. 145:344-352), or portions of the immunoglobulin molecule containing sites for interchain disulfide bonds, could lead to the formation of multimers. In certain embodiments, the formation of homodimers or homomultimers can interfere with the formation of a complex of the invention. If this is the case, peptide tags that do not promote the formation of homodimers or homomultimers should be used.

Other possible peptide tags are short amino acid sequences to which monoclonal antibodies are available, such as but not limited to the following well known examples, the FLAG epitope, the myc epitope at amino acids 408-439, the influenza virus hemaglutinin (HA) epitope. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

In certain embodiments, a combination of different peptide tags is used for the purification of the protein components of a complex of the invention or for the purification of a complex. In certain embodiments, at least one component has at least two peptide tags, e.g., a FLAG-tag and a His-tag. The different tags can be fused together or can be fused in different positions to the protein component. In the purification procedure, the different peptide tags are used subsequently or concurrently for purification. In certain embodiments, at least two different components are fused to a peptide tag, wherein the peptide tags of the two components can be identical or different. Using different tagged components for the purification of the complex ensures that only complex will be purified and minimizes the amount of uncomplexed protein components, such as monomers or homodimers.

A specific peptide tag is a non-variable portion of the immunoglobulin molecule.

Typically, such portions comprises at least the functional CH2 and CH3 domains of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based peptide tag may be obtained from IgG-1, -2, -3, or -4 subtypes, IgA, IgE, IgD, or IgM, but preferably IgG1. Preferably, a human immunoglobulin is used when the protein component is intended for in vivo use for humans. DNA sequences encoding immunoglobulin light or heavy chain constant regions are well-known or readily available from cDNA libraries. In a specific embodiment, such DNA sequences can be amplified using PCR. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labelling systems are available for the detection of immunoglobulins, the fusion protein of a protein component of a complex of the invention can readily be detected and quantified by a variety of immunological techniques known in the art, such as the use of enzyme-linked immunosorbent assay (ELISA), immunoprecipitation, fluorescence activated cell sorting (FACS), etc. Similarly, if the peptide tag is an epitope with readily available antibodies, such reagents can be used with the techniques mentioned above to detect, quantitate, and isolate the fusion protein component of a complex of the invention containing the peptide tag.

In a specific embodiment, a protein component is fused to the hinge, CH2 and CH3 domains of murine immunoglobulin G-1 (IgG-1) (Bowen et al., J. Immunol. 156:442-9). This peptide contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of a protein component of a complex of the invention from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816).

DNA sequences encoding desired peptide tag or leader peptide which are known or readily available from libraries or commercial suppliers are suitable in the practice of this invention.

### 5.5 Recombinant Expression of HAI-TMIP Variants and Complexes

A HAI-TMIP(s) and complexes comprising a HAI-TMIP can be generated by any technique known to one skilled in the art. In a specific embodiment, the protein components of the complexes described in Section 5.3, supra (e.g., HAI-TMIP variants described in Section 5.2, supra) and the complexes described in Section 5.4, supra, can be generated by any method known to the skilled artisan. In certain embodiments, the complexes can be generated by co-expressing the components of the complex in a cell and subsequently purifying the complex. In certain, more specific embodiments, the cell expresses at least one component of the complex by recombinant DNA technology. In other embodiments, the cells normally express the components of the complex. In certain other embodiments, the components of the complex are expressed separately, wherein the components can be expressed using recombinant DNA technology or wherein at least one component is purified from a cell that normally expresses the component. The individual components of the complex are incubated in vitro under conditions conducive to the binding of the components of a complex of the invention to each other to generate a complex of the invention.

If one or more of the components is expressed by recombinant DNA technology, any method known to the skilled artisan can be used to produce the recombinant protein. The nucleic and amino acid sequences of the component proteins of the protein complexes of the present invention are provided herein (see Tables 1 and 2; and SEQ ID NOS:1 to 10), and can be obtained by any method known in the art, e.g., by PCR amplification using synthetic primers hybridizable to the 3′ and 5′ ends of each sequence, and/or by cloning from a cDNA or genomic library using an oligonucleotide specific for each nucleotide sequence.

The protein components, either alone or in a complex, can be obtained by methods well known in the art for protein purification and recombinant protein expression. For recombinant expression of one or more of the proteins, the nucleotide sequence encoding the protein or a fragment thereof can be inserted into an appropriate expression vector, i.e., a vector that contains the necessary elements for the transcription and translation of the inserted protein coding sequence. The necessary transcriptional and translational signals can also be supplied by the native promoter of the component protein gene and/or flanking regions. Alternatively, the transcriptional and translational signals can be heterologous to the component protein gene.

A variety of host-vector systems may be utilized to express a protein coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In a specific embodiment, a complex of the present invention is obtained by expressing the coding sequences of the component proteins in the same cell, either under the control of the same promoter or separate promoters. In yet another embodiment, a derivative, fragment or homolog of a component protein is recombinantly expressed. Preferably the derivative, fragment or homolog of the protein forms a complex with the other components of the complex. In a specific embodiment, the protein components form a complex that binds to an antibody that recognizes (selectively or specifically) complex.

Any method available in the art can be used for the insertion of nucleotide sequences into a vector to construct expression vectors containing appropriate transcriptional/translational control signals and protein coding sequences. These methods may include in vitro recombinant DNA and synthetic techniques and in vivo recombinant techniques (genetic recombination). Expression of nucleotide sequences encoding a component protein, or a derivative, fragment or homolog thereof, may be regulated by a second nucleotide sequence so that the protein, or derivative, fragment or homolog thereof is expressed in a host transformed with the recombinant nucleotide sequence molecule(s). For example, expression of the proteins may be controlled by any promoter/enhancer known in the art. In specific embodiments, the promoter is not native to the genes for the component protein. In certain embodiments, the promoter used is a constitutive promoter. In other embodiments, the promoter used is an inducible promoter. In certain embodiments, the promoter used is a tissue-specific promoter.

Promoters that may be used to regulate the expression of a component protein include, but are not limited to, the SV40 early promoter (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42); prokaryotic expression vectors such as the β-lactamase promoter (VIIIa-Kamaroff et al., 1978, Proc. Natl. Acad. Sci. USA 75:3727-3731) or the tac promoter (DeBoer et al., 1983, Proc. Natl. Acad. Sci. USA 80:21-25; Gilbert et al., 1980, Scientific American 242:79-94); plant expression vectors comprising the nopaline synthetase promoter (Herrar-Estrella et al., 1984, Nature 303:209-213) or the cauliflower mosaic virus 35S RNA promoter (Garder et al., 1981, Nucleic Acids Res. 9:2871), and the promoter of the photosynthetic enzyme ribulose bisphosphate carboxylase (Herrera-Estrella et al., 1984, Nature 310:115-120); promoter elements from yeast and other fungi such as the Ga14 promoter (Johnston et al., 1987, Microbiol. Rev. 51:458-476), the alcohol dehydrogenase promoter (Schibler et al., 1987, Annual Review Genetics 21:237-257), the phosphoglycerol kinase promoter (Struhl et al., 1995, Annual Review Genetics 29:651-674-257; Guarente 1987, Annual Review Genetics 21:425-452), the alkaline phosphatase promoter (Struhl et al., 1995, Annual Review Genetics 29:651-674-257; Guarente 1987, Annual Review Genetics 21:425-452), and the following animal transcriptional control regions that exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald 1987, Hepatology 7:425-515); insulin gene control region which is active in pancreatic beta cells (Hanahan et al., 1985, Nature 315:115-122), immunoglobulin gene control region which is active in lymphoid cells (Grosschedl et al., 1984, Cell 38:647-658; Adams et al., 1985, Nature 318:533-538; Alexander et al., 1987, Mol. Cell Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell Biol. 5:1639-1648; Hammer et al., 1987, Science 235:53-58), alpha-1 antitrypsin gene control region which is active in liver (Kelsey et al., 1987, Genes and Devel. 1:161-171), beta globin gene control region which is active in myeloid cells (Mogram et al., 1985, Nature 315:338-340; Kollias et al., 1986, Cell 46:89-94), myelin basic protein gene control region which is active in oligodendrocyte cells of the brain (Readhead et al., 1987, Cell 48:703-712), myosin light chain-2 gene control region which is active in skeletal muscle (Sani 1985, Nature 314:283-286), and gonadotrophic releasing hormone gene control region which is active in gonadotrophs of the hypothalamus (Mason et al., 1986, Science 234:1372-1378).

In a specific embodiment, a vector is used that comprises a promoter operably linked to a nucleotide sequence encoding a component protein, or a fragment, derivative or homolog thereof, one or more origins of replication, and optionally, one or more selectable markers (e.g., an antibiotic resistance gene). In accordance with this embodiment, a promoter can be any promoter known to the skilled artisan. For example, the promoter may be a constitutive promoter, a tissue-specific promoter or an inducible promoter.

In another embodiment, an expression vector contains the coding sequence, or a portion thereof, of a component protein, and the coding sequence of a peptide tag, such as a GST-tag, His-tag, Flag-tag, strep-tag or GFP-tag. In a specific embodiment, an expression vector containing the coding sequence, or a portion thereof, of a protein component, is made by subcloning the gene sequences into the multiple cloning site of one of the three pGEX vectors (glutathione S-transferase (GST) expression vectors; Smith and Johnson, 1988, Gene 7:31-40) or the pQE-30 vector (6×His tag (His tag) vector; Qiagen, Hilden, Germany). Care should be taken that the nucleotide sequence encoding the protein component is in the same reading frame as the nucleotide sequence encoding the peptide tag (in this case, the GST- or His-tag) such that the protein component and the peptide tag (GST- or His-tag) are expressed as one fusion protein.

Expression vectors containing the sequences of interest can be identified by three general approaches: (1) nucleic acid hybridization, (2) presence or absence of “marker” gene function, and (3) expression of the inserted sequences. In the first approach, coding sequences can be detected by nucleic acid hybridization to probes comprising sequences homologous and complementary to the inserted sequences. For example, the T2 probe described, infra, may be used in a hybridization assay to detect HAI-TMIP variants. In the second approach, the recombinant vector/host system can be identified and selected based upon the presence or absence of certain “marker” functions (e.g., resistance to antibiotics, occlusion body formation in baculovirus, etc.) caused by insertion of the sequences of interest in the vector. For example, if a component protein gene, or portion thereof, is inserted within the marker gene sequence of the vector, recombinants containing the encoded protein or fragment will be identified by the absence of the marker gene function (e.g., loss of beta-galactosidase activity). In the third approach, recombinant expression vectors can be identified by assaying the expression of the component at the RNA (e.g., by Northern blot analysis and RT-PCR) and/or protein level (e.g., by Western blot and flow cytometry). Such assays can be based, for example, on the physical or functional properties of the interacting species in in vitro assay systems, e.g., formation of a complex comprising the protein or binding to an antibody. The expressed sequences can be detected using antibodies that are specifically directed to the expressed protein component. In certain embodiments, the expressed sequence is a fusion protein of the protein component and comprises a peptide tag, wherein the peptide tag can be visualized, such as a GFP tag.

Once recombinant component protein molecules are identified and the complexes or individual proteins purified, several methods known in the art can be used to propagate them. Using a suitable host system and growth conditions, recombinant expression vectors can be propagated and amplified in quantity. As previously described, the expression vectors or derivatives which can be used include, but are not limited to, human or animal viruses such as vaccinia virus or adenovirus; insect viruses such as baculovirus, yeast vectors; bacteriophage vectors such as lambda phage; and plasmid and cosmid vectors.

In addition, a host cell strain may be chosen that modulates the expression of the inserted sequences, or modifies or processes the expressed proteins in the specific fashion desired. Expression from certain promoters can be elevated in the presence of certain inducers; thus expression of the genetically-engineered component proteins may be controlled. Furthermore, different host cells have characteristic and specific mechanisms for the translational and post-translational processing and modification (e.g., glycosylation, phosphorylation, etc.) of proteins. Appropriate cell lines or host systems can be chosen to ensure that the desired modification and processing of the foreign protein is achieved. For example, expression in a bacterial system (e.g., E. coli) can be used to produce an unglycosylated core protein, while expression in mammalian cells ensures “native” glycosylation of a heterologous protein. Furthermore, different vector/host expression systems may effect processing reactions to different extents. Non-limiting examples of host cells include CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express a protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a component protein molecule. Such engineered cell lines may be particularly useful in screening and evaluation of compositions that interact directly or indirectly with a component protein.

A number of selection systems may be used, including but not limited to, the herpes simplex virus thymidine kinase (Wigler et al., 1977, Cell 11:223), hypoxanthineguanine phosphoribosyltransferase (Szybalska & Szyalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al., (eds.), Current Protocols in Molecular Biology, John Wiley & Sons, NY (1993); Ausubel et al., (eds), Short Protocols in Molecular Biology 5^{th} Edition, John Wiley & Sons, NY (2002); Kriegler, Gene Transfer and Expression, A Laboratory Manual, Stockton Press, NY (1990); and in Chapters 12 and 13, Dracopoli et al., (eds), Current Protocols in Human Genetics, John Wiley & Sons, NY (1994); Colberre-Garapin et al., 1981, J. Mol. Biol. 150:1, which are incorporated by reference herein in their entireties.

The expression levels of a protein can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing a protein is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

In other specific embodiments, a component protein or a fragment, homolog or derivative thereof, may be expressed as fusion or chimeric protein product comprising the protein, fragment, homolog, or derivative joined via a peptide bond to a heterologous protein sequence. Such chimeric products can be made by ligating the appropriate nucleic acid sequences encoding the desired amino acids to each other by methods known in the art, in the proper coding frame, and expressing the chimeric products in a suitable host by methods commonly known in the art. Alternatively, such a chimeric product can be made by protein synthetic techniques, e.g., by use of a peptide synthesizer. Chimeric genes comprising a nucleotide sequence encoding a fragment of a component protein fused to any heterologous protein-encoding sequences may be constructed.

In a specific embodiment, fusion proteins are provided that contain the interacting domains of the component proteins and, optionally, a peptide linker between the two domains, where such a linker promotes the interaction of the binding domains. These fusion proteins may be particularly useful where the stability of the interaction is desirable (due to the formation of the complex as an intra-molecular reaction), for example, in production of antibodies specific to the complex.

Protein component derivatives can be made by altering their sequences by substitutions, additions or deletions that provide for functionally equivalent molecules. Due to the degeneracy of nucleotide coding sequences, other DNA sequences that encode substantially the same amino acid sequence as a component gene or cDNA can be used in the practice of the present invention. These include but are not limited to nucleotide sequences comprising all or portions of the component protein gene that are altered by the substitution of different codons that encode a functionally equivalent amino acid residue within the sequence, thus producing a silent change. Likewise, the derivatives of the invention include, but are not limited to, those containing, as a primary amino acid sequence, all or part of the amino acid sequence of a component protein, including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

The protein component derivatives and analogs of the invention can be produced by various methods known in the art. The manipulations which result in their production can occur at the nucleotide or protein level. For example, the cloned gene sequences can be modified by any of numerous strategies known in the art (Sambrook et al., 1989, Molecular Cloning, A Laboratory Manual, 2d Ed., Cold Spring Harbor Laboratory Press, Cold Spring Harbor, N.Y.). The sequences can be cleaved at appropriate sites with restriction endonuclease(s), followed by further enzymatic modification if desired, isolated, and ligated in vitro. In the production of the gene encoding a derivative, homolog or analog of a component protein, care should be taken to ensure that the modified gene retains the original translational reading frame, uninterrupted by translational stop signals, in the gene region where the desired activity is encoded.

Additionally, the encoding nucleic acid sequence can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Any technique for mutagenesis known in the art can be used, including but not limited to, chemical mutagenesis and in vitro site-directed mutagenesis (Hutchinson et al., 1978, J. Biol. Chem 253:6551-6558), amplification with PCR primers containing a mutation, use of chimeric oligonucleotides, etc.

Once a recombinant cell expressing a component protein, or fragment or derivative thereof, is identified, the individual gene product or complex can be purified and analyzed. This is achieved by assays based on the physical and/or functional properties of the protein or complex, including, but not limited to, radioactive labelling of the product followed by analysis by gel electrophoresis, immunoassay, cross-linking to marker-labelled product, etc.

The component proteins and complexes may be purified by standard methods known in the art (either from natural sources or recombinant host cells expressing the complexes or proteins), including but not restricted to column chromatography (e.g., ion exchange, affinity, gel exclusion, reversed-phase high pressure, fast protein liquid, etc.), differential centrifugation, differential solubility, or by any other standard technique used for the purification of proteins. Functional properties may be evaluated using any suitable assay known in the art. For a more detailed description of purification procedures of the components and the complexes of the invention, see below.

Alternatively, once a component protein or its derivative, is identified, the amino acid sequence of the protein can be deduced from the nucleic acid sequence of the chimeric gene from which it was encoded. As a result, the protein or its derivative can be synthesized by standard chemical methods known in the art (e.g., Hunkapiller et al., 1984, Nature 310: 105-111).

Manipulations of component protein sequences may be made at the protein level. Included within the scope of the invention is a complex in which the component proteins or derivatives and analogs that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In specific embodiments, the amino acid sequences are modified to include a fluorescent label. In another specific embodiment, the protein sequences are modified to have a heterofunctional reagent; such heterofunctional reagents can be used to crosslink the members of the complex.

In addition, component proteins or fragments, analogs or derivatives thereof can be chemically synthesized. For example, a peptide corresponding to a fragment of a component protein, which comprises the desired domain or mediates the desired activity in vitro (e.g., complex formation) can be synthesized by use of a peptide synthesizer. Furthermore, if desired, non-classical amino acids or chemical amino acid analogs can be introduced as a substitution or addition into the protein sequence. Non-classical amino acids include, but are not limited to, the D-isomers of the common amino acids, α-amino isobutyric acid, 4-aminobutyric acid (4-Abu), 2-aminobutyric acid (2-Abu), 6-amino hexanoic acid (Ahx), 2-amino isobutyric acid (2-Aib), 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In cases where natural products are suspected of being mutant or are purified from new species, the amino acid sequence of a component protein purified from the natural source, as well as those expressed in vitro, or from synthesized expression vectors in vivo or in vitro, can be determined from analysis of the DNA sequence, or alternatively, by direct sequencing of the purified protein. Such analysis can be performed by manual sequencing or through use of an automated amino acid sequenator.

The protein components can also be analyzed by hydrophilicity analysis (Hopp and Woods, 1981, Proc. Natl. Acad. Sci. USA 78:3824-3828). A hydrophilicity profile can be used to identify the hydrophobic and hydrophilic regions of the proteins, and help predict their orientation in designing substrates for experimental manipulation, such as in binding experiments, antibody synthesis, etc. Secondary structural analysis can also be done to identify regions of the component proteins, or their derivatives, that assume specific structures (Chou and Fasman, 1974, Biochemistry 13:222-23). Manipulation, translation, secondary structure prediction, hydrophilicity and hydrophobicity profile predictions, open reading frame prediction and plotting, and determination of sequence homologies, etc., can be accomplished using computer software programs available in the art.

Other methods of structural analysis including, but not limited to, X-ray crystallography (Engstrom, 1974 Biochem. Exp. Biol. 11:7-13), mass spectroscopy and gas chromatography (Methods in Protein Science, J. Wiley and Sons, New York, 1997), and computer modeling (Fletterick and Zoller, eds., 1986, Computer Graphics and Molecular Modeling, In: Current Communications in Molecular Biology, Cold Spring Harbor Laboratory, Cold Spring Harbor Press, New York) can also be employed.

In certain embodiments, at least one component of the complex is generated by recombinant DNA technology and is a derivative of the naturally occurring protein. In certain embodiments, the derivative is a fusion protein, wherein the amino acid sequence of the naturally occurring protein is fused to a second amino acid sequence. The second amino acid sequence can be a peptide tag that facilitates the purification, immunological detection and identification of the protein (and in some embodiments, visualization of the protein). A variety of peptide tags with different functions and affinities can be used in the invention to facilitate the purification of the component or the complex comprising the component by affinity chromatography. In one embodiment, a peptide tag comprises the constant regions of an immunoglobulin or a fragment thereof. Other peptide tags that can be used with the invention include, but are not limited to, FLAG epitope or polyHistidine tag, e.g., Hisx6 tag. In other embodiments, the component is fused to a leader sequence to promote secretion of the protein component from the cell that expresses the protein component.

The methods described herein can be used to purify the individual components of the complex of the invention. The methods can also be used to purify the entire complex. Generally, the purification conditions as well as the dissociation constant of the complex will determine whether the complex remains intact during the purification procedure. Such conditions include, but are not limited to, salt concentration, detergent concentration, pH and redox-potential.

The protein components or the complex can be purified by any method known to the skilled artisan, including immunoprecipitation, ammonium sulfate precipitation, acid extraction, anion or cation exchange chromatography, phosphocellulose chromatography, immunoaffinity chromatography, hydroxyapatite chromatography, and lectin chromatography.

If at least one component of the complex comprises a peptide tag, methods for the purification of the complexes of the invention which are based on the properties of the peptide tag can be used. One approach is based on specific molecular interactions between a tag and its binding partner. The other approach relies on the immunospecific binding of an antibody to an epitope present on the tag. The principle of affinity chromatography well known in the art is generally applicable to both of these approaches. In a specific embodiment, the complex is purified using immunoprecipitation.

Described in Section 5.6 below are several methods based on specific molecular interactions of a tag and its binding partner for purification. The embodiments described in Section 5.6 may be used to recover and purify protein components of the complex separately or to recover and purify the complexes of the invention. Methods that do not require lowering pH or denaturing conditions are most preferred for purification of the complexes.

In certain embodiments, the components of the complex are co-expressed and the complex is purified from the expression system. In other embodiments, the individual components of a complex of the invention are expressed separately and the components are subsequently incubated under conditions conducive to the binding of the components of the complex to each other to generate the complex. In some embodiments, the protein components are purified before complex-formation. In other embodiments, the supernatants of cells that express the protein component (if the component is secreted) or cell lysates of cells that express the protein component (if, e.g., the component is not secreted) are combined first to give rise to the complex, and the complex is purified subsequently. Parameters affecting the ability of the protein components of the invention to bind to each other include, but are not limited to, salt concentration, detergent concentration, pH, and redox-potential. Once the complex has formed, the complex can be purified or concentrated by any method known to the skilled artisan. In certain embodiments, the complex is separated from the remaining individual components by filtration. The pore size of the filter should be such, that the individual components can still pass through the filter, but the complex does not pass through the filter. Other methods for enriching the complex include sucrose gradient centrifugation and chromatography.

### 5.6 Purification of Components and Complexes of the Invention

The complexes of the invention can be purified by any method known to the skilled artisan. The methods described for the purification of a complex may also be used to purify individual protein components. In certain embodiments, the complex is formed in the expression system itself, wherein the expression system can be, e.g., a cell or a cell-free expression system (such as a TNT® Coupled Reticulocyte Lysate System, which is commercially available from Promega Corporation, Madison Wis.). Once the protein components are expressed and the complex is formed, the complex is purified from the other components of the expression system and the individual protein components by any method known to the skilled artisan. If the expression system is a cell, the cell is lysed once the protein components are expressed and once the complex is formed, the protein complex of the invention is then purified from the lysate. In certain other embodiments, the protein components of a complex of the invention are expressed and purified individually and subsequently the purified components are combined to form the complex. The individual protein components can be purified by any method known to the skilled artisan.

In certain embodiments, the complex is purified via affinity chromatography using antibodies that are specific to the complex. In other embodiments, the complex is purified by performing subsequent purification steps wherein each step requires the presence of a different protein component in the complex to ensure that the purified complex is free of any monomeric protein components. Each individual purification step can be, e.g., based on the peptide tag of a protein component (for a more detailed description of the use of peptide tags in protein purification see below) or an affinity purification using antibodies specific to the protein component. Care should be taken that the antibodies to be used for the purification of the complex are not directed to epitopes that are located at the binding interface of the protein component.

In certain embodiments, a complex of the invention is purified via a protein tag that is fused to at least one of the protein components of the complex. In more specific embodiments, two protein components of a complex are fused to a peptide tag and one protein component is fused to a peptide tag different from the peptide tag to which the other protein component is fused. The complex is first purified via the one and subsequently via the other peptide tag to ensure that the purified complex is free from any monomeric protein components.

A method that is generally applicable to purifying a protein component that is fused to the constant regions of immunoglobulin or a complex that comprises a component that is fused to the constant regions of immunoglobulin is protein A affinity chromatography, a technique that is well known in the art. Staphylococcus protein A is a 42 kD polypeptide that binds specifically to a region located between the second and third constant regions of heavy chain immunoglobulins. Because of the Fc domains of different classes, subclasses and species of immunoglobulins, affinity of protein A for human Fc regions is strong, but may vary with other species. Subclasses that are less preferred include human IgG-3, and most rat subclasses. For certain subclasses, protein G (of Streptococci) may be used in place of protein A in the purification. Protein-A sepharose (Pharmacia or Biorad) is a commonly used solid phase for affinity purification of antibodies, and can be used essentially in the same manner for the purification of a protein component fused to an immunoglobulin Fc fragment. The protein component that is fused to the constant regions of immunoglobulin or a complex that comprises a component that is fused to the constant regions of immunoglobulin binds specifically to protein A on the solid phase, while the contaminants are washed away. Bound protein component that is fused to the constant regions of immunoglobulin or a complex that comprises a component that is fused to the constant regions of immunoglobulin can be eluted by various buffer systems known in the art, including a succession of citrate, acetate and glycine-HCl buffers which gradually lowers the pH. This method is less preferred if the recombinant cells also produce antibodies which will be copurified with the protein component that is fused to the constant regions of immunoglobulin or a complex that comprises a component that is fused to the constant regions of immunoglobulin. See, for example, Langone, 1982, J. Immunol. Meth. 51:3; Wilchek et al., 1982, Biochem. Intl. 4:629; Sjobring et al., 1991, J. Biol. Chem. 26:399; page 617-618, in Antibodies A Laboratory Manual, edited by Harlow and Lane, Cold Spring Harbor laboratory, 1988.

Alternatively, a polyhistidine tag may be used, in which case, the protein component that is fused to the polyhistidine tag or a complex that comprises a component that is fused to the polyhistidine tag can be purified by metal chelate chromatography. The polyhistidine tag, usually a sequence of six histidines, has a high affinity for divalent metal ions, such as nickel ions (Ni2+), which can be immobilized on a solid phase, such as nitrilotriacetic acid-matrices. Polyhistidine has a well characterized affinity for Ni2+-NTA-agarose, and can be eluted with either of two mild treatments: imidazole (0.1-0.2 M) will effectively compete with the resin for binding sites; or lowering the pH just below 6.0 will protonate the histidine sidechains and disrupt the binding. The purification method comprises loading the cell culture supernatant onto the Ni2+-NTA-agarose column, washing the contaminants through, and eluting the protein component that is fused to the polyhistidine tag or a complex that comprises a component that is fused to the polyhistidine tag with imidazole or weak acid. Ni2+-NTA-agarose can be obtained from commercial suppliers such as Sigma (St. Louis) and Qiagen. Antibodies that recognize the polyhistidine tag are also available which can be used to detect and quantitate the protein component that is fused to the polyhistidine tag or a complex that comprises a component that is fused to the polyhistidine tag.

Another exemplary peptide tag that can be used is the glutathione-S-transferase (GST) sequence, originally cloned from the helminth, Schistosoma japonicum. In general, a protein component-GST fusion or a complex comprising a protein component-GST fusion expressed in a host cell can be purified from the cell culture supernatant by absorption with glutathione agarose beads, followed by elution in the presence of free reduced glutathione at neutral pH. Denaturing conditions are not required at any stage during purification, and therefore, it may be desirable for the purification of the complex. Moreover, since GST is known to form dimers under certain conditions, dimeric protein components may be obtained. See, Smith, 1993, Methods Mol. Cell Bio. 4:220-229.

Another useful peptide tag that can be used is the maltose binding protein (MBP) of E. coli, which is encoded by the malE gene. The protein component-MBP fusion protein or the complex comprising a component-MPP fusion protein binds to amylose resin while contaminants are washed away. The bound modified protein component-MBP is eluted from the amylose resin by maltose. See, for example, Guan et al., 1987, Gene 67:21-30.

The second approach for purifying protein component fusion proteins is applicable to peptide tags that contain an epitope for which polyclonal or monoclonal antibodies are available. Various methods known in the art for purification of protein by immunospecific binding, such as immunoaffinity chromatography, and immunoprecipitation, can be used. See, for example, Chapter 13 in Antibodies A Laboratory Manual, edited by Harlow and Lane, Cold Spring Harbor laboratory, 1988; and Chapter 8, Sections I and II, in Current Protocols in Immunology, ed. by Coligan et al., John Wiley, 1991; the disclosure of which are both incorporated by reference herein.

A protein component of a complex of the invention can also be purified by immunoaffinity chromatography or immunoprecipitation using antibodies that are specific to the component. Likewise, a complex of the invention can be purified by immunoaffinity chromatography or immunoprecipitation using antibodies that bind to at least one of the components of the complex. In a specific embodiment, a complex of the invention can be purified by immunoaffinity chromatography or immunoprecipitation using antibodies that are specific to the complex.

### 5.7 Transgenic Animals

The present invention encompasses transgenic animals. Host cells comprising a nucleotide sequence encoding a protein component(s) can be used to produce nonhuman transgenic animals. For example, in one embodiment, a host cell is a fertilized oocyte or an embryonic stem cell into which a nucleotide sequence encoding a protein component(s) has been introduced. Such host cells can then be used to create non-human transgenic animals in which exogenous sequences encoding a protein described in Section 5.2, supra, have been introduced into their genome or homologous recombinant animals in which endogenous sequences encoding a protein component(s) have been altered. Such animals are useful for studying the function and/or activity of the protein component(s) and for identifying and/or evaluating modulators of protein activity. As used herein, a “transgenic animal” is a non-human animal, preferably a mammal (e.g., a rodent such as a rat or mouse), in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal (e.g., a mouse), in which an endogenous gene has been altered by homologous recombination between the endogenous gene and an exogenous DNA molecule introduced into a cell of the animal, e.g., an embryonic cell of the animal, prior to development of the animal.

A transgene animal of the invention can be created by introducing nucleotide sequence described in Section 5.1, supra, into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the protein component to particular cells. Methods for generating transgenic animals via embryo manipulation and microinjection, particularly animals such as mice, have become conventional in the art and are described, for example, in U.S. Pat. Nos. 4,736,866 and 4,870,009, 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create a homologous recombinant animal, a vector is prepared which contains at least a portion of a gene encoding a protein component into which a deletion, addition or substitution has been introduced to thereby alter, e.g., functionally disrupt, the gene. In a specific embodiment, the vector ig designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (, e.g., Li et al. (1992) Cell 69:915). The selected cells are then injected into a blastocyst of an animal (e.g., a mouse) to form aggregation chimeras (, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 35:910-913 and PCT Publication NOS. WO 97/07668 and WO 97/07669.

### 5.8 Antibodies Immunospecific for HAI-TMIP

Variants and Complexes

The present invention provides antibodies that immunospecifically bind to a protein described in Section 5.2, supra. In a specific embodiment, the present invention provides antibodies that immunospecifically bind to HAI-TMIP variant 1, 2, 3, 4, 5, 6 or 7. In certain embodiments, HAI-TMIP variant 3, 4, 5, 6 or 7, or a fragment thereof is used as an immunogen to produce such antibodies. In some embodiments, an immunogen which comprises an epitope unique to HAI-TMIP variant 1, 2, 3, 4, 5, 6 or 7 is used to produce such antibodies. In a specific embodiment, one or more of the following peptides are used to produce antibodies that bind to an HAI-TMIP variant:

| | |
|---|---|
| peptide 1 ARNFHASNTHLQKTC | (SEQ ID NO: 25) |
| peptide 2 MSSILEERILGADC | (SEQ ID NO: 26) |
| peptide 3 MQTGIKAVDSLVPC | (SEQ ID NO: 27) |
| peptide 4 CASNTHLQKTGTAE | (SEQ ID NO: 28) |
| peptide 5 CVSQHQALLGTIRA | (SEQ ID NO: 29) |

Peptides 1 and 4 can be used as an immunogen to generate antibodies that bind to HAI-TMIP variants 1 and 2. Peptide 2 can be used as an immunogen to generate antibodies that bind to HAI-TMIP variants 1, 2, 3, 4, 6 and 7 (and not HAI-TMIP variant 5). Peptides 3 and 5 can be used as an immunogen to generate antibodies that bind to HAI-TMIP variants 1, 2, 3, 4, 5, 6 and 7.

In a specific embodiment, the peptide used as an immunogen is peptide 2.

In a specific embodiment, an antibody immunospecifically binds to HAI-TMIP variant 3, 4, 5, 6 or 7 but not to HAI-TMIP variant 1 or 2. In certain embodiments of the invention, the affinity of an antibody that binds to HAI-TMIP variant 3, 4, 5, 6 or 7 is higher than the affinity of the antibody for HAI-TMIP variants 1 and/or 2. In certain embodiments of the invention, the affinity of an antibody that binds to HAI-TMIP variant 3, 4, 5, 6 or 7 is at least 2 times, at least 5 times, at least 10 times, at least 100 times, at least 1,000 times, at least 10,000 times or at least 100,000 times higher than the affinity of the antibody for HAI-TMIP variants 1 and/or 2. In certain embodiments of the invention, the affinity of an antibody that binds to HAI-TMIP variant 3, 4, 5, 6 or 7 is at about 2 to about 5 times, about 2 to about 10 times, about 5 to about 10 times, about 10 to about 25 times, about 10 to about 50 times, or about 10 to about 100 times higher than the affinity of the antibody for HAI-TMIP variants 1 and/or 2. In accordance with these embodiments, the affinity of the antibody may be determined utilizing methods described herein or known in the art (e.g., a plasma resonance assay such as the BIAcore® assay).

The present invention provides an antibody that immunospecifically binds to HAI-TMIP variant 3, 4, 6 or 7 but not to HAI-TMIP variants 1, 2 and/or 5. In certain embodiments, the affinity of an antibody that binds to HAI-TMIP variant 3, 4, 6 or 7 is higher than the affinity of the antibody for HAI-TMIP variants 1, 2 and/or 5. In certain embodiments of the invention, the affinity of an antibody that binds to HAI-TMIP variant 3, 4, 6 or 7 is at least 2 times, at least 5 times, at least 10 times, at least 100 times, at least 1,000 times, at least 10,000 times or at least 100,000 times higher than the affinity of the antibody for HAI-TMIP variants 1, 2 and/or 3. In certain embodiments of the invention, the affinity of an antibody that binds to HAI-TMIP variant 3, 4, 6 or 7 is at about 2 to about 5 times, about 2 to about 10 times, about 5 to about 10 times, about 10 to about 25 times, about 10 to about 50 times, or about 10 to about 100 times higher than the affinity of the antibody for HAI-TMIP variants 1, 2 and/or 5. In accordance with these embodiments, the affinity of the antibody may be determined utilizing methods described herein or known to one of skill in the art (e.g., a plasmon resonance assay such as the BIAcore®).

The present invention provides an antibody that immunospecifically binds to HAI-TMIP variant 1, 2, 3, 4, 6 or 7 but not to HAI-TMIP variant 5. In certain embodiments of the invention, the affinity of an antibody that binds to HAI-TMIP variant 1, 2, 3, 4, 6 or 7 is higher than the affinity of the antibody for HAI-TMIP variants 5. In certain embodiments of the invention, the affinity of an antibody that binds to HAI-TMIP variant 1, 2, 3, 4, 6 or 7 is at least 2 times, at least 5 times, at least 10 times, at least 100 times, at least 1,000 times, at least 10,000 times or at least 100,000 times higher than the affinity of the antibody for HAI-TMIP variant 5. In certain embodiments of the invention, the affinity of an antibody that binds to HAI-TMIP variant 1, 2, 3, 4, 6 or 7 is at about 2 to about 5 times, about 2 to about 10 times, about 5 to about 10 times, about 10 to about 25 times, about 10 to about 50 times, or about 10 to about 100 times higher than the affinity of the antibody for HAI-TMIP variant 5. In accordance with these embodiments, the affinity of the antibody may be determined utilizing methods described herein or known in the art (e.g., a plasma resonance assay such as the BIAcore® assay).

The present invention provides an antibody that immunospecifically binds to HAI-TMIP variant 5 but not to HAI-TMIP variants 1, 2, 3, 4, 6 and/or 7. In certain embodiments, the affinity of an antibody that binds to HAI-TMIP variant 5 is higher than the affinity of the antibody for HAI-TMIP variants 1, 2, 3, 4, 6 and/or 7. In certain embodiments of the invention, the affinity of an antibody that binds to HAI-TMIP variant 5 is at least 2 times, at least 5 times, at least 10 times, at least 100 times, at least 1,000 times, at least 10,000 times or at least 100,000 times higher than the affinity of the antibody for HAI-TMIP variants 1, 2, 3, 4, 6 and/or 7. In certain embodiments of the invention, the affinity of an antibody that binds to HAI-TMIP variant 5 is about 2 to about 5 times, about 2 to about 10 times, about 5 to about 10 times, about 10 to about 25 times, about 10 to about 50 times, or about 10 to about 100 times higher than the affinity of the antibody for HAI-TMIP variants 1, 2, 3, 4, 6 and/or 7. In accordance with these embodiments, the affinity of the antibody may be determined utilizing methods described herein or known to one of skill in the art (i.e., a plasmon resonance assay such as the BIAcore® assay).

In a specific embodiment, an antibody that immunospecifically binds to HAI-TMIP variant 1, 2, 3, 4, 5, 6 or 7 has a kₒₙ rate (antibody (Ab)+antigen (Ag)→Ab−Ag) of 10⁵ M⁻¹s⁻¹ or more, 5×10⁵ M⁻¹s⁻¹ or more, 10⁶ M⁻¹ₛ⁻¹ or more, 5×10⁶ M⁻¹s⁻¹, or more, 10⁷ M⁻¹s⁻¹ or more, 5×10⁷ M⁻¹s⁻¹ or more, 10⁸ M⁻¹s⁻¹ or more, 5×10⁸ M⁻¹s⁻¹ or more. In certain embodiments, an antibody binds to HAI-TMIP variant 3, 4, 5, 6 or 7 with a kₑₙ about 2 to about 10 times, about 10 to about 100 times, about 100 to about 1,000 times, about 1,000 to about 10,000 times, or about 10,000 to about 100,000 times higher than the antibody binds to HAI-TMIP variant 1 and/or 2. In other embodiments, an antibody binds to HAI-TMIP variant 3, 4, 6 or 7 with a kₒₙ about 2 to about 10 times, about 10 to about 100 times, about 100 to about 1,000 times, about 1,000 to about 10,000 times, or about 10,000 to about 100,000 times higher than the antibody binds to HAI-TMIP variant 1, 2 and/or 5. In other embodiments, an antibody immunospecifically binds to HAI-TMIP variant 1, 2, 3, 4, 6 or 7 with a kₒₙ about 2 to about 10 times, about 10 to about 100 times, about 100 to about 1,000 times, about 1,000 to about 10,000 times, or about 10,000 to about 100,000 times higher than the antibody binds to HAI-TMIP variant 5. In other embodiments, an antibody binds to HAI-TMIP variant 5 with a kₒₙ about 2 to about 10 times, about 10 to about 100 times, about 100 to about 1,000 times, about 1,000 to about 10,000 times, or about 10,000 to about 100,000 times higher than the antibody binds to HAI-TMIP variant 1, 2, 3, 4, 6 and/or 7. In accordance with these embodiments, the kₒₙ may be determined using a plasmon resonance assay such as the BIAcore® assay.

In another embodiment, an antibody that immunospecifically binds to HAI-TMIP variant 1, 2, 3, 4, 5, 6 or 7 has a k_{off} rate (antibody (Ab)+antigen (Ag)→Ab−Ag) of 5×10⁻³ s⁻¹ or less, 10⁻³ s⁻¹ or less, 5×10 s⁻¹ or less, 10 s⁻¹ or less, 5×10⁻⁵ s⁻¹ or less, 10⁻⁵ s⁻¹ or less, 5×10⁻⁶ s⁻¹ or less, 10⁻⁶ s⁻¹ or less, 5×10⁻⁷ s⁻¹ or less, 10⁻⁷ s⁻¹ or less, 5×10⁻⁸ s⁻¹ or less, 10⁻⁸ s⁻¹ or less, 5×10⁻⁹ s⁻¹ or less, 10⁻⁹ s⁻¹ or less. In a specific embodiment, the k_{off} rate of the antibody is determined by a plasmon resonance assay (e.g., a BIAcore® assay).

In another embodiment, an antibody that immunospecifically binds to HAI-TMIP variant 1, 2, 3, 4, 5, 6 or 7 has an affinity constant or K_{d} (kₒₙ/k_{off}) of at least 10⁶ M⁻¹, preferably at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, or at least 10¹¹ M⁻¹. In another embodiment, an antibody that immunospecifically binds to HAI-TMIP variant 1, 2, 3, 4, 5, 6 or 7 has a dissociation constant or K_{d} (k_{off}/kₒₙ) of 5×10⁻⁵ M or less, le M or less, 5×10⁻⁶ M or less, 10⁻⁶ M or less, 5×10⁻⁷ M or less, 10⁻⁷ M or less, 5×10⁻⁸M or less, 10⁻⁸ M or less, 5×10⁻⁶M or less, 10⁻⁹ M, or less than 5×10⁻¹⁰ M or less. In accordance with these embodiments, the Ka and Kd of the antibody maybe determined utilizing methods described herein or known to one of skill in the art (e.g., a plasmon resonance assay such as the BIAcore® assay).

The present invention provides antibodies that immunospecifically bind to a complex comprising a HAI-TMIP variant (e.g., HAI-TMIP variant 1, 2, 3, 4, 5, 6 or 7). In a specific embodiment, the present invention provides antibodies that immunospecifically bind to a complex of the invention comprising a purified protein described in Section 5.2, supra (e.g., HAI-TMIP variant 3, 4, 5, 6 or 7). In certain embodiments, such antibodies are produced using a complex of the invention as the immunogen, wherein the protein components of the complex are covalently linked to each other. In certain embodiments of the invention, the affinity of an antibody that binds to a complex of the invention is higher than the affinity of the antibody to any of the components of the complex individually. In certain embodiments of the invention, the affinity of an antibody that binds to a complex of the invention is at least 2 times, at least 5 times, at least 10 times, at least 100 times, at least 1,000 times, at least 10,000 times or at least 100,000 times higher than the affinity of the antibody to any of the components of the complex individually. In certain embodiments of the invention, the affinity of an antibody that binds to a complex of the invention is about 2 to about 5 times, about 2 to about 10 times, about 5 to about 10 times, about 10 to about 25 times, about 10 to about 50 times, or about 10 to about 100 times higher than the affinity of the antibody to any of the components of the complex individually. In a specific embodiment, the antibody immunospecific to the complex and the antibody does not bind the individual protein components of the complex. The binding affinity of an antibody to an antigen, such as the complex or a protein component, can be determined by any method described herein (e.g., a plasmon resonance assay such as the BIAcore® assay) or known to the skilled artisan (, e.g., van Cott et al., 1992, Real-time biospecific interaction analysis of antibody reactivity to peptides from the envelope glycoprotein, gp160, of HIV-1, J Immunol Methods 146(2):163-76).

In a specific embodiment, an antibody that immunospecifically binds to a complex of the invention has a kₒₙ rate (antibody (Ab)+antigen (Ag)→Ab+Ag) of 10⁵ M⁻¹s⁻¹ or more, 5×10⁵ M⁻¹s⁻¹ or more, 10⁶ M⁻¹s⁻¹ or more, 5×10⁶ M⁻¹s⁻¹ or more, 10⁷M⁻¹s⁻¹ or more, or 10⁸ M⁻¹s⁻¹ or more. In certain embodiments, an antibody binds to a complex of the invention with a kₒₙ about 2 to about 10 times, about 10 to about 100 times, about 100 to about 1,000 times, about 1,000 to about 10,000 times, or about 10,000 to about 100,000 times higher than the antibody binds to individual protein components of the complex. In accordance with these embodiments, the kₒₙ may be determined using a plasmon resonance assay such as the BIAcore® assay.

In another embodiment, an antibody that immunospecifically binds to a complex of the invention has a k_{off} rate (antibody (Ab)+antigen (Ag)←Ab−Ag) of 5×10⁻³ s⁻¹ or less, 10⁻³s⁻¹ or less, 5×10⁻⁴ s⁻¹ or less, 10⁻⁴ s⁻¹ or less, 5×10⁻⁵ s⁻¹ or less, le s⁻¹ or less, 5×10⁻⁶ s⁻¹ or less, 10⁻⁶ s⁻¹ or less, 5×10 s⁻¹ or less, 10⁻⁷ s⁻¹ or less, 5×10⁻⁸ s⁻¹ or less, 10⁻⁸ s⁻¹ or less, 5×10⁻⁹ s⁻¹ or less, 10⁻⁹ s⁻¹ or less. In a specific embodiment, the k_{off} rate of the antibody is determined by a plasmon resonance assay such as the BIAcore® assay. In another embodiment, an antibody that immunospecifically binds to a complex of the invention has an affinity constant or Kₐ (kₒₙ/k_{off}) of at least 10⁶ M⁻¹, preferably at least 5×10⁶ M⁻¹, at least 10⁷ M⁻¹, at least 5×10⁷M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, or at least 10¹¹ M⁻¹. In another embodiment, an antibody that immunospecifically binds to a complex of the invention has a dissociation constant or Kₐ (k_{off}/kₒₙ) of 5×10⁻⁵ M or less, 10⁻⁵ M or less, 5×10⁻⁶ M or less, 10″⁶ M or less, 5×10⁻⁷M or less, le M or less, 5×10⁻⁸ M or less, 10⁻⁸ M or less, 5×10⁻⁶M or less, 10⁻⁹ M, or less than 5×10¹⁰ M or less.

The present invention provides antibodies that bind to a HAI-TMIP variant (e.g., HAI-TMIP variant 1, 2, 3, 4, 5, 6 or 7) and prevent the binding of the HAI-TMIP variant to other components of the F₁ ATP synthase subcomplex. In a specific embodiment, the invention provides antibodies that bind to human HAI-TMIP variant (e.g., HAI-TMIP variant 1, 2, 3, 4, 5, 6 or 7) and prevent the binding of the HAI-TMIP variant to the ATP synthase subunit of the F₁ ATP synthase subcomplex. The present invention provides antibodies that bind to a HAI-TMIP variant (e.g., HAI-TMIP variant 1, 2, 3, 4, 5, 6 or 7) and prevent the binding of the F₁ ATP synthase subcomplex or F₀F₁ ATP synthase holoenzyme to its ligand. In some embodiments, such antibodies inhibit or reduce the activity of the F₁ ATP synthase subcomplex or F₀F₁ ATP synthase holoenzyme. In other embodiments, such antibodies increase the activity of the F₁ ATP synthase subcomplex or F₀F₁ ATP synthase holoenzyme. Non-limiting examples of such ligands include apolipoprotein E-rich high density lipoprotein, angiostatin, Vγ9Vδ2 T cell receptors and coupling factor 6. Methods for assessing whether an antibody prevents the binding of the F₁ ATP synthase subcomplex or F₀F₁ ATP synthase holoenzyme to its ligand can be measured using techniques well-known in the art and described herein (e.g., an immunoassay such as an ELISA).

The present invention provides antibodies that bind to a HAI-TMIP variant (e.g., HAI-TMIP variant 1, 2, 3, 4, 5, 6 or 7) and decrease the activity of the F₁ ATP synthase subcomplex or F₀F₁ ATP synthase holoenzyme. In some embodiments, such antibodies increase the binding of the F₁ ATP synthase subcomplex or F₀F₁ ATP synthase holoenzyme to its ligand. The present invention also provides antibodies that increase the activity of the F₁ ATP synthase subcomplex or F₀F₁ ATP synthase holoenzyme.

Antibodies of the invention include, but are not limited to, monoclonal antibodies, multispecific antibodies, diabodies, triabodies, tetrabodies, human antibodies, humanized antibodies, camelised antibodies, chimeric antibodies, single-chain Fvs (scFv), single chain antibodies, single domain antibodies, Fab fragments, F(ab′) fragments, F(ab′)₂ fragments, disulfide-linked Fvs (sdFv), and anti-idiotypic (anti-Id) antibodies (including, e.g., anti-Id antibodies to antibodies of the invention), intrabodies, and epitope-binding fragments of any of the above. In a specific embodiment, the antibodies of the invention are isolated.

### 5.8.1 Antibodies with Increased Half Lives

The present invention provides for antibodies that bind to a HAI-TMIP variant (e.g., HAI-TMIP variant 1, 2, 3, 4, 5, 6 or 7), which have an extended half-life in vivo. In one embodiment, the present invention provides for antibodies that immunospecifically bind to a protein described in Section 5.2, supra, which have an extended half-life in vivo. In a specific embodiment, the present invention provides antibodies that immunospecifically bind to a protein described in Section 5.2, supra, which have a half-life in a subject, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

The present invention provides for antibodies that bind to a complex comprising a HAI-TMIP variant (e.g., HAI-TMIP variant 1, 2, 3, 4, 5, 6 or 7), which have an extended half-life in vivo. In one embodiment, the present invention also provides for antibodies that immunospecifically bind to a protein complex described in Section 5.3, supra, which have an extended half-life in vivo. In a specific embodiment, the present invention provides antibodies that immunospecifically bind to a protein complex described in Section 5.3, supra, which have a half-life in a subject, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; International Publication No. WO 02/060919; and U.S. Pat. No. 6,277,375, each of which is incorporated herein by reference in its entirety.

Further, antibodies can be conjugated to albumin in order to make the antibody more stable in vivo or have a longer half life in vivo. The techniques are well-known in the art, see, e.g., International Publication Nos. WO 93/15199, WO 93/15200, and WO 01/77137; and European Patent No. EP 413,622, all of which are incorporated herein by reference.

### 5.8.2 Antibody Conjugates

The present invention provides antibodies that bind to a HAI-TMIP variant (e.g., HAI-TMIP variant 1, 2, 3, 4, 5, 6 or 7), and antibodies that bind to a complex comprising a HAI-TMIP variant (e.g., HAI-TMIP variant 1, 2, 3, 4, 5, 6 or 7) recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In one embodiment, the present invention provides of antibodies that immunospecifically bind to a protein described in Section 5.2, supra, and antibodies that immunospecifically bind a protein complex described in Section 5.3, supra, recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antigen-binding fragment of an antibody (e.g., a Fab fragment, Fd fragment, Fv fragment, F(ab)₂ fragment, a V11 domain, a VH CDR, a VL domain or a VL CDR) and a heterologous protein, polypeptide, or peptide. In a specific embodiment, the heterologous protein, polypeptide, or peptide that the antibody is fused to is useful for targeting the antibody to specific cell types. For example, an antibody that immunospecifically binds to a cell surface receptor expressed by a particular cell type may be fused or conjugated to an antibody. Methods for fusing or conjugating proteins, polypeptides, or peptides to an antibody are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references are incorporated herein by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding an antibody may be recombined with one or more components, motifs, sections, parts, domains, fragments, etc. of one or more heterologous molecules.

Moreover, the antibodies can be fused to marker sequences, such as a peptide to facilitate purification. In specific embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Zn, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and nonradioactive paramagnetic metal ions.

The present invention further encompasses uses of antibodies conjugated to a therapeutic moiety. An antibody may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN 1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate); HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

In a specific embodiment, an antibody is conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, interferon-α, interferon-β, interferon-γ, nerve growth factor, platelet derived growth factor, tissue plasminogen activator, an apoptotic agent, e.g., TNF-α, INF-β, AIM I (, International Publication No. WO 97/33899), AIM II (, International Publication No. WO 97/34911), Fas Ligand (Takahashi et al., 1994, J. Immunol., 6:1567-1574), and VEGF (, International Publication No. WO 99/23105), an anti-angiogenic agent, e.g., angiostatin, endostatin or a component of the coagulation pathway (e.g., tissue factor); or a biological response modifier such as, for example, a cytokine (e.g., interferon gamma (“IFN-γ”), interleukin-1 (“IL-1”), interleukin-2 (“IL-2”), interleukin-4 (“IL-4”), interleukin-5 (“IL-5”), interleukin-6 (“IL-6”), interleukin-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”), a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid. fibrinopeptides A and B from the a and (3 chains of fibrinogen, fibrin monomer).

Moreover, an antibody can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹L, ¹³¹Yb, ¹³¹Ho, ¹³¹Sm, to polypeptides or any of those listed supra. In certain embodiments, the macrocyclic chelator is 1,4,7,10-tetraazacyclododecane-N,N,N″,N″′-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabelled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

An antibody can also be conjugated to a second antibody to form an antibody heteroconjugate as described by Segal in U.S. Pat. No. 4,676,980, which is incorporated herein by reference in its entirety. In a specific embodiment, two or more antibodies are cross-linked to each to produce a bispecific or multispecific antibody.

The therapeutic moiety or drug conjugated to an antibody should be chosen to achieve the desired prophylactic or therapeutic effect(s) for a particular disease or disorder in a subject. A clinician or other medical personnel should consider the following when deciding on which therapeutic moiety or drug to conjugate to an antibody: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.9 Methods for Producing Antibodies

Antibodies that bind to an antigen can be produced by any method known in the art for the synthesis of antibodies, in particular, by chemical synthesis or preferably, by recombinant expression techniques. In some embodiments, an immunogen which comprises an epitope unique to a protein component is used to generate an antibody immunospecific for the component. In specific embodiments, the following peptides are used as immunogens to produce antibodies that distinguish HAI-TMIP variants:

| | |
|---|---|
| peptide 1 ARNFHASNTHLQKTC | (SEQ ID NO: 25) |
| peptide 2 MSSILEERILGADC | (SEQ ID NO: 26) |
| peptide 3 MQTGIKAVDSLVPC | (SEQ ID NO: 27) |
| peptide 4 CASNTHLQKTGTAE | (SEQ ID NO: 28) |
| peptide 5 CVSQHQALLGTIRA | (SEQ ID NO: 29) |

Peptides 1 and 4 can be used as an immunogen to generate antibodies that bind to HAI-TMIP variants 1 and 2. Peptide 2 can be used to generate antibodies that bind to HAI-TMIP variants 1, 2, 3, 4, 6 and 7 (but not to HAI-TMIP variant 5). Peptides 3 and 5 can be used as an immunogen to generate antibodies that bind to HAI-TMIP variants 1, 2, 3, 4, 5, 6 and 7. In other embodiments, an antigen comprising an epitope unique to a protein component is used to screen for antibodies immunospecific for the component.

Polyclonal antibodies immunospecific for an antigen can be produced by various procedures well-known in the art. For example, the antigen (i.e., a complex of the invention or a component of a complex of the invention) can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Köhler & Milstein, 1975, Nature 256:495-497; Pasqualini & Arap, 2004, PNAS USA 101:257-259; Steinitz et al., 1977, Nature 269:420-422; Vollmers et al., 1989, Cancer Res. 49:2471-2476; Vollmers & Brandlein, 2002 Hum. Antibodies 11(4):131-142; Harlow et al., Antibodies: A Laboratory Manual, (Cold Spring Harbor Laboratory Press, 2^{nd} ed. 1988); Hammerling, et al., in: Monoclonal Antibodies and T Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labelled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/01134; Griffiths et al., 1994, EMBO J 13:3245-3260; Winter et al., 1994, Annu. Rev. Immunol. 12:433-455; Liv et al., 2004, Cancer Res. 64:704-710; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJR1 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1a promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology.

The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65 93.

For a detailed discussion of methods for producing human antibodies and human monoclonal antibodies and protocols for producing such antibodies, e.g., Tomizuka et al., 2000 PNAS USA 97:722-727; Davis et al., 2004, Methods Mol. Biol. 248:191-200; Lagerkvist et al., 1995, Biotechniques 18:862-869; Babcook et al., 1996 PNAS USA 93:7843-7848; International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415, which are incorporated herein by reference in their entirety.

A humanized antibody is an antibody which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′).sub.2, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. A humanized antibody can be produced using variety of techniques known in the art, including but not limited to, CDR-grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see e.g., European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973, each of which is incorporated herein by its entirety by reference), chain shuffling (see e.g., U.S. Pat. No. 5,565,332, which is incorporated herein in its entirety by reference), and techniques disclosed in, e.g., U.S. Pat. No. 6,407,213, U.S. Pat. No. 5,766,886, International Publication No. WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s-5977s (1995), Couto et al., Cancer Res. 55(8):1717 22 (1995), Sandhu J S, Gene 150(2):409 10 (1994), and Pedersen et al., J. Mol. Biol. 235(3):959 73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Diabodies, tribodies, and tetrabodies can be produced by techniques known to one of skill in the art. See, e.g., Kipriyanov, 2002, Methods Mol. Biol. 178:317-331; Todorovska et al., 2001 J. Immunol. Methods 248:47-66; and Poljak et al., 1994, Structure 2:1121-1123, each of which are incorporated herein by reference in their entirety, for methods for producing diabodies, triabodies and tetrabodies. Single domain antibodies can also be produced by techniques known to one of skill in the art. For a description of techniques to produce single domain antibodies, see, e.g., Holliger & Hudson, 2005 Nat. Biotechnol. 23:1126-1136, Riechmann et al., 1999, J. Immunol. Methods 231:25-38; and Dick, 1990, BMJ 300:659-600, each of which is incorporated herein by reference in its entirety.

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

In one embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (scFv). The scFv typically comprises a single peptide with the sequence V_{H}-linker-V_{L} or V_{L}-linker-V_{H}. The linker is chosen to permit the heavy chain and light chain to bind together in their proper conformational orientation (see, for example, Huston et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 3.

TABLE 3

| Sequence | SEQ ID NO. |
|---|---|
| (Gly Gly Gly Gly Ser)₃ | SEQ ID NO: 43 |
| Glu Ser Gly Arg Ser Gly Gly Gly Gly | SEQ ID NO: 44 |
| Ser Gly Gly Gly Gly Ser | |
| Glu Gly Lys Ser Ser Gly Ser Gly Ser | SEQ ID NO: 45 |
| Glu Ser Lys Ser Thr | |
| Glu Gly Lys Ser Ser Gly Ser Gly Ser | SEQ ID NO: 46 |
| Glu Ser Lys Ser Thr Gln | |
| Glu Gly Lys Ser Ser Gly Ser Gly Ser | SEQ ID NO: 47 |
| Glu Ser Lys Val Asp | |
| Gly Ser Thr Ser Gly Ser Gly Lys Ser | SEQ ID NO: 48 |
| Ser Glu Gly Lys Gly | |
| Lys Glu Ser Gly Ser Val Ser Ser Glu | SEQ ID NO: 49 |
| Gln Leu Ala Gln Phe Arg Ser Leu Asp | |
| Glu Ser Gly Ser Val Ser Ser Glu Glu | SEQ ID NO: 50 |
| Leu Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J. Can. Res. 76:1132-5; Ratner et al., 1985, Nature 313:277-84). Examples of localization signals include, but are not limited to, those sequences disclosed in Table 4.

TABLE 4

| Localization | Sequence | SEQ ID NO. |
|---|---|---|
| endoplasmic reticulum | Lys Asp Glu Leu | SEQ ID NO: 51 |
| endoplasmic reticulum | Asp Asp Glu Leu | SEQ ID NO: 52 |
| endoplasmic reticulum | Asp Glu Glu Leu | SEQ ID NO: 53 |
| endoplasmic reticulum | Gln Glu Asp Leu | SEQ ID NO: 54 |
| endoplasmic reticulum | Arg Asp Glu Leu | SEQ ID NO: 55 |
| Nucleus | Pro Lys Lys Lys Arg Lys Val | SEQ ID NO: 56 |
| Nucleus | Pro Gln Lys Lys Ile Lys Ser | SEQ ID NO: 57 |
| Nucleus | Gln Pro Lys Lys Pro | SEQ ID NO: 58 |
| Nucleus | Arg Lys Lys Arg | SEQ ID NO: 59 |
| Nucleus | Lys Lys Lys Arg Lys | SEQ ID NO: 60 |
| nucleolar region | Arg Lys Lys Arg Arg Gln Arg Arg Arg Ala | SEQ ID NO: 61 |
| | His Gln | |
| nucleolar region | Arg Gln Ala Arg Arg Asn Arg Arg Arg Arg | SEQ ID NO: 62 |
| | Trp Arg Glu Arg Gln Arg | |
| nucleolar region | Met Pro Leu Thr Arg Arg Arg Pro Ala Ala | SEQ ID NO: 63 |
| | Ser Gln Ala Leu Ala Pro Pro Thr Pro | |
| Endosomal compartment | Met Asp Asp Gln Arg Asp Leu Ile Ser Asn | SEQ ID NO: 64 |
| | Asn Glu Gln Leu Pro | |
| mitochondrial matrix | Met Leu Phe Asn Leu Arg Xaa Xaa Leu Asn | SEQ ID NO: 65 |
| | Asn Ala Ala Phe Arg His Gly His Asn Phe | |
| | Met Val Arg Asn Phe Arg Cys Gly Gln Pro | |
| | Leu Xaa | |
| Peroxisome | Ala Lys Leu | SEQ ID NO: 66 |
| trans Golgi network | Ser Asp Tyr Gln Arg Leu | SEQ ID NO: 67 |
| plasma membrane | Gly Cys Val Cys Ser Ser Asn Pro | SEQ ID NO: 68 |
| plasma membrane | Gly Gln Thr Val Thr Thr Pro Leu | SEQ ID NO: 69 |
| plasma membrane | Gly Gln Glu Leu Ser Gln His Glu | SEQ ID NO: 70 |
| plasma membrane | Gly Asn Ser Pro Ser Tyr Asn Pro | SEQ ID NO: 71 |
| plasma membrane | Gly Val Ser Gly Ser Lys Gly Gln | SEQ ID NO: 72 |
| Plasma membrane | Gly Gln Thr Ile Thr Thr Pro Leu | SEQ ID NO: 73 |
| Plasma membrane | Gly Gln Thr Leu Thr Thr Pro Leu | SEQ ID NO: 74 |
| Plasma membrane | Gly Gln Ile Phe Ser Arg Ser Ala | SEQ ID NO: 75 |
| Plasma membrane | Gly Gln Ile His Gly Leu Ser Pro | SEQ ID NO: 76 |
| Plasma membrane | Gly Ala Arg Ala Ser Val Leu Ser | SEQ ID NO: 77 |
| Plasma membrane | Gly Cys Thr Leu Ser Ala Glu Glu | SEQ ID NO: 78 |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol. Biol. 291:1129-34).

Further, the antibodies that immunospecifically bind to a complex of the invention or a protein described in Section 5.2, supra, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

### 5.9.1 Nucleotide Sequences Encoding an Antibody

The invention provides nucleic acid sequences comprising a nucleotide sequence encoding an antibody that binds to a HAI-TMIP or a complex comprising a HAI-TMIP. In one embodiment, the invention provides nucleic acid sequences comprising a nucleotide sequence encoding an antibody that binds to a complex of the invention or a protein described in Section 5.2, supra. In a specific, such nucleic acid sequences are isolated. The invention also encompasses nucleic acid sequences that hybridize under high stringency, intermediate or lower stringency hybridization conditions, e.g., as defined supra, to nucleic acid sequences that encode an antibody of the invention.

The nucleic acid sequence may be obtained, and the nucleotide sequence of the nucleic acid sequence determined, by any method known in the art. The nucleotide sequence of antibodies immunospecific for a desired antigen can be obtained, e.g., from the literature or a database such as GenBank. Such a nucleic acid sequence encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a nucleic acid sequence encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (, e.g., Chothia et al., 1998, J. Mol. Biol. 278: 457-479 for a listing of human framework regions). Preferably, the nucleic acid sequence generated by the combination of the framework regions and CDRs encodes an antibody that immunospecifically binds to a particular antigen. In a specific embodiment, one or more amino acid substitutions may be made within the framework regions, and in certain embodiments, the amino acid substitutions improve binding of the antibody to its antigen. Additionally, such methods may be used to make amino acid substitutions or deletions of one or more variable region cysteine residues participating in an intrachain disulfide bond may be made to generate antibody molecules lacking one or more intrachain disulfide bonds. Other alterations to the nucleic acid sequence are encompassed by the present invention and within the skill of the art.

### 5.9.2 Recombinant Expression of an Antibody

Recombinant expression of an antibody that binds to a complex of the invention or a protein described in Section 5.2, supra, requires construction of an expression vector containing a nucleic acid sequence that encodes the antibody. Once a nucleic acid sequence encoding an antibody molecule, heavy or light chain of an antibody, or fragment thereof (preferably, but not necessarily, containing the heavy or light chain variable domain) of the invention has been obtained, the vector for the production of the antibody molecule may be produced by recombinant DNA technology using techniques well-known in the art. Thus, methods for preparing an antibody by expressing a nucleic acid sequence encoding the antibody are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a nucleic acid sequence encoding an antibody, or a heavy or light chain thereof, or fragment thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to a complex of the invention or a protein described in Section 5.2, supra, is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies may be fused to heterologous polypeptide sequences described herein or otherwise known in the art to facilitate purification.

### 5.10 Use of Antibodies

The antibodies described in Section 5.8 can be used with any method known to the skilled artisan. In certain embodiments, an antibody of the invention is used to detect or quantify HAI-TMIP variant (e.g., HAI-TMIP variant 1, 2, 3, 4, 5, 6, or 7), or a complex comprising a HAI-TMIP variant (e.g., a complex of the invention). To this end, Western blot analyses, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, or fluorescent immunoassays can be performed using an antibody of the invention.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody which recognizes the antigen to the cell lysate, incubating for a period of time (e.g, 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody which recognizes the antigen) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols, e.g., Ausubel et al., eds, 1994, Current Protocols in Molecular Biology, Vol. 1, John Wiley & Sons, Inc., New York at 10.8.1.

ELISAs comprise preparing antigen, coating the well of a microtiter plate (e.g., a 96 well microtiter plate) with the antigen, adding a primary antibody (which recognizes the antigen) conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the primary antibody) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs, e.g., Ausubel et al, eds, 1994, Current Protocols in Molecular Biology, Vol. 1, John Wiley & Sons, Inc., New York at 11.2.1.

The antibodies described in Section 5.8 can be used to modulate the binding of the F₁ ATP synthase subcomplex and/or F₀F₁ ATP synthase holoenzyme to its ligand. Non-limiting examples of such ligands include angiostatin, apolipoprotein E-rich high density lipoprotein, coupling factor 6 and Vδ9Vδ2 T cell receptors. The antibodies described in Section 5.8 can be used to modulate the binding of HAI-TMIP variant 3, 4, 5, 6 and/or 7 to angiostatin, apolipoprotein E-rich high density lipoprotein, coupling factor 6 and/or Vγ9Vδ2 T cell receptors. Antibodies that modulate the binding of F₁ ATP synthase subcomplex, F₀F₁ ATP synthase holoenzyme or a component thereof to a ligand can identified using techniques well-known in the art or described herein.

In a specific embodiment, the present invention provides a method of modulating the binding of a complex of the invention to angiostatin, the method comprising contacting cell expressing a complex of the invention with an antibody that binds to HAI-TMIP variant 3, 4, 5, 6 and/or 7, or a complex described herein. In another embodiment, the present invention provides a method of modulating the binding of a complex of the invention to apolipoprotein E-rich high density lipoprotein, the method comprising contacting cell expressing a complex of the invention with an antibody that binds to HAI-TMIP variant 3, 4, 5, 6 and/or 7, or a complex described herein. In another embodiment, the present invention provides a method of modulating the binding of a complex of the invention to coupling factor 6, the method comprising contacting cell expressing a complex of the invention with an antibody that binds to HAI-TMIP variant 3, 4, 5, 6 and/or 7, or a complex described herein. In another embodiment, the present invention provides a method of modulating the binding of a complex of the invention to Vγ9Vδ2 T cell receptors, the method comprising contacting cell expressing a complex of the invention with an antibody that binds to HAI-TMIP variant 3, 4, 5, 6 and/or 7, or a complex described herein. In accordance with these embodiments, the antibody preferably binds to HAI-TMIP variant 3, 4, 5, 6 or 7, or a complex described herein.

The antibodies described in Section 5.8 can be used to modulate the proliferation of various cells, such as, for example, cancer cells and endothelial cells. Antibodies that modulate the proliferation of cells can identified using techniques well-known in the art or described herein. In a specific embodiment, the invention provides a method of reducing the number of cancer cells, the method comprising contacting a cancer cell with an antibody that binds to HAI-TMIP variant 3, 4, 5, 6 and/or 7, or a complex described herein and reduces or prevents the formation of a complex of the invention, destabilizes a complex of the invention and/or inhibits or reduces the ATP synthase activity of a complex of the invention. In another embodiment, the invention provides a method of reducing the number of endothelial cells, the method comprising contacting an endothelial cell with an antibody that binds to HAI-TMIP variant 3, 4, 5, 6 and/or 7, or a complex described herein and reduces or prevents the formation of a complex of the invention, destabilizes a complex of the invention and/or inhibits or reduces the ATP synthase activity of a complex of the invention. In another embodiment, the invention provides a method of increasing the number of endothelial cells, the method comprising contacting an endothelial cell with an antibody that binds to HAI-TMIP variant 3, 4, 5, 6 and/or 7, or a complex described herein and increases the ATP synthase activity of a complex of the invention. In accordance with these embodiments, the antibody preferably immunospecifically binds to HAI-TMIP variant 3, 4, 5, 6 and/or 7 or a complex described herein. Further, in accordance with these embodiments, the cell population contacted with the antibody changes by at least 10%, preferably at least 25%, at least 45%, at least 50%, at least 60%, at least 70%, at least 80%, at least 85%, at least 90% relative to the same type of cell population contacted with a negative control as measured by an assay described herein or known to one of skill in the art.

The antibodies described in Section 5.8, supra, can be used to modulate HDL endocytosis. Antibodies that modulate HDL endocytosis can identified using techniques well-known in the art, e.g., the techniques described in Martinez et al., 2003, Nature 421:75-79. In a specific embodiment, the invention provides a method of increasing HDL endocytosis, the method comprising contacting a cell expressing a complex of the invention with an antibody that binds to HAI-TMIP variant 3, 4, 5, 6 and/or 7, or a complex described herein and activates ATP synthase activity of a complex of the invention. In accordance with this embodiment, the antibody preferably immunospecifically binds to HAI-TMIP variant 3, 4, 5, 6 and/or 7 or a complex described herein.

The antibodies described in Section 5.8, supra, can be used to modulate the immune response of Vδ9Vδ2 cells. Antibodies that modulate the immune response of Vε9Vδ2 cells can identified using techniques well-known in the art, e.g., the techniques described in Scotet et al., 2005, Immunity 22:71-80.

The antibodies described in Section 5.8, supra, can be used to prevent, treat and/or manage a disorder described herein below. The HAI-TMIP is overexpressed on cancer cells, including breast cancer cells, colon cancer cells, rectum cancer cells, kidney cancer cells, lung cancer cells, liver cancer cells, ovarian cancer cells, uterine cancer cells and prostate cancer cells. Accordingly, in a specific embodiment, antibodies described in Section 5.8, supra, can be used to prevent, treat and/or manage cancer.

The antibodies described in Section 5.8, supra, can also be used to screen for, detect and diagnose cancer. The antibodies described in Section 5.8, supra, can be used alone or in combination with other techniques to screen, detect and diagnose cancer. The antibodies described in Section 5.8, supra, can be used to confirm the diagnosis of cancer. The antibodies of the invention can be used prognose cancer, monitor the progression of cancer, and monitor the efficacy of a therapy. Techniques for detecting and diagnosing cancer using antibodies are well-known in the art and described briefly in Section 5.19.

Further, the antibodies described in Section 5.8, supra, can be used in standard immunoassays.

### 5.11 Screening Assays

The present invention provides methods for identifying compounds that modulate: (i) the expression of a protein or RNA product of a nucleotide sequence encoding a HAI-TMIP variant (e.g., a nucleotide sequence described in Section 5.1, supra); (ii) the activity of a HAI-TMIP variant (e.g., a protein described in Section 5.2, supra); (iii) the formation of a complex comprising a HAI-TMIP variant (e.g., a complex of the invention); (iv) stabilization of a complex comprising a HAI-TMIP variant (e.g., a complex of the invention); and/or (v) the activity of a complex comprising a HAI-TMIP variant (e.g, a complex of the invention). In a specific embodiment, high-throughput assays are used in the methods described herein. In another embodiment, the compounds assessed for the functions and activities described herein are screened in pools. Once a positive pool has been identified, the individual compounds of that pool are tested separately. In certain embodiments, the pool size is at least 2, at least 5, at least 10, at least 25, at least 50, at least 75, at least 100, at least 150, at least 200, at least 250, at least 500 compounds, or 5-25, 5 to 50, 25 to 100, 50 to 200, 50 to 500, 200 to 1,000 or 200 to 2,000 compounds.

### 5.11.1 Screening Assays for Modulators of HAI-TMIP Variants

The present invention provides methods of identifying compounds that bind to the products of the nucleotide sequences of a HAI-TMIP variant (e.g., the nucleotide sequences described in Section 5.1, supra). The present invention also provides methods for identifying compounds that modulate the expression and/or activity of the products of the nucleotide sequences of a HAI-TMIP variant (e.g., the nucleotide sequences described in Section 5.1, supra). The compounds identified via such methods are useful as lead compounds in the development of compositions for use in diagnosing a disease, monitoring the progression of a disease, determining the prognosis of a disease, and determining the efficacy of a therapy. The compounds identified via such methods are also useful as lead compounds in the development of prophylactic and therapeutic compositions for prevention, treatment, management and/or amelioration of a disease described herein or a symptom thereof. Such methods are particularly useful in that the effort and great expense involved in testing potential prophylactics and therapeutics in vivo is efficiently focused on those compounds identified via the in vitro and ex vivo methods described herein.

In certain embodiments, compounds are screened in pools. Once a positive pool has been identified, the individual molecules of that pool are tested separately. In certain embodiments, the pool size is at least 2, at least 5, at least 10, at least 25, at least 50, at least 75, at least 100, at least 150, at least 200, at least 250, or at least 500 compounds.

In certain embodiments of the invention, the screening method further comprises determining the structure of the candidate compound. The structure of a candidate compound can be determined by any technique known to the skilled artisan. Exemplary methods are described in Section 5.13.

The screening assay described in Sections 5.11.1.1 to 5.11.1.3 can be applied to identify compounds that bind to other ATP synthase subunits and variants (including HAI-TMIP variants 1 and 2), disrupt the interaction between cellular molecules and other ATP synthase subunits and variants (including HAI-TMIP variants 1 and 2), and/or modulate the expression and/or activity of other ATP synthase subunits and variants (including HAI-TMIP variants 1 and 2). The screening assays in Section 11.2 can also be applied to complexes comprising other ATP synthase subunits and variants (including complexes comprising HAI-TMIP variants 1 and 2). One of skill in the art would be able to modify such assays appropriately.

### 5.11.1.1 Screening Assays for Compounds that Bind to a HAI-TMIP Variant

The present invention provides a method of identifying a compound that binds to HAI-TMIP variant 3, 4, 5, 6 or 7, the method comprising: (a) contacting a cell expressing a protein described in Section 5.2, supra, with a test compound under conditions that permit the protein and test compound to interact and form a complex; and (b) detecting the formation of the protein-test compound complex formed. The present invention also provides a method for identifying a compound that binds to HAI-TMIP variant 3, 4, 5, 6 or 7, the method comprising: (a) contacting a cell expressing a RNA product transcribed from a nucleotide sequence described in Section 5.1, supra, with a test compound under conditions that permit the RNA product and test compound to interact and form a complex; and (b) detecting the formation of the RNA product-test compound complex formed. In accordance with the invention, the cell expressing the protein or RNA product can be, e.g., a yeast cell or a cell of mammalian origin.

The present invention provides a method of identifying a compound that binds to HAI-TMIP variant 3, 4, 5, 6 or 7, the method comprising: (a) contacting a protein described in Section 5.2, supra, with a test compound under conditions that permit the protein and test compound to interact and form a complex; and (b) detecting the formation of the protein-test compound complex formed. The present invention also provides a method for identifying a compound that binds to HAI-TMIP variant 3, 4, 5, 6 or 7, the method comprising: (a) contacting a RNA product transcribed from a nucleotide sequence described in Section 5.1, supra, with a test compound to interact and form a complex; and (b) detecting the formation of the RNA product-test compound complex formed. Techniques well-known in the art can be used to determine the formation of a complex between a test compound and a protein or RNA product.

The test compound may be labelled, directly or indirectly, with a detectable label, (e.g., radioisotope or enzymatic label) such that binding of the test compound to the protein product or RNA product can be determined by detecting the labelled compound in a complex. For example, test compounds can be labelled with ¹²⁵I, ³⁵S, ¹⁴C, or ³H, either directly or indirectly, and the radioisotope detected by direct counting of radioemmission or by scintillation counting. The test compounds can be enzymatically labelled with, for example, horseradish peroxidase, alkaline phosphatase, or luciferase, and the enzymatic label detected by determination of conversion of an appropriate substrate to product. Alternatively, the protein or RNA product may be labelled, directly or indirectly, with a radioisotope or enzymatic label such that binding of the protein or RNA product to the test compound can be determined by detecting the labelled compound in a complex.

In some embodiments of the above assay methods of the present invention, it may be desirable to immobilize a RNA product or a test compound to facilitate separation of complexed from uncomplexed forms of the RNA product, the compound or both, as well as to accommodate automation of the assay. In more than one embodiment of the above assay methods of the present invention, it may be desirable to immobilize either a protein or a test compound to facilitate separation of complexed from uncomplexed forms of the protein, the compound or both, as well as to accommodate automation of the assay. Binding of a test compound to a RNA product or protein can be accomplished in any vessel suitable for containing the reactants. Examples of such vessels include microtiter plates, test tubes, and microcentrifuge tubes. In one embodiment, a fusion protein comprising a protein described in Section 5.2 and a domain that allows the protein to be bound to a matrix is used in the assay methods. For example, glutathione-S-transferase (GST) fusion proteins can be adsorbed onto glutathione sepharose beads (Sigma Chemical; St. Louis, Mo.) or glutathione derivatized microtiter plates, which are then combined with the test compound and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding of a protein described in Section 5.2 can be determined using standard techniques.

Other techniques for immobilizing proteins on matrices can also be used in the screening assays of the invention. For example, either a protein or a test compound can be immobilized utilizing conjugation of biotin and streptavidin. A biotinylated protein or a test compound can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with a protein described in Section 5.2 can be derivatized to the wells of the plate, and protein trapped in the wells by antibody conjugation. Methods for detecting such complexes, in addition to those described above for the GST-immobilized complexes, include immunodetection of complexes using antibodies reactive with a protein described in Section 5.2, supra, as well as enzyme-linked assays which rely on detecting an enzymatic activity associated with a protein described in Section 5.2, or a test compound.

In a specific embodiment, the protein or RNA product is immobilized and the test compound is labelled. In another embodiment, the test compound is immobilized and the protein or RNA product is labelled.

In some embodiments of the above assay methods of the present invention, the step of contacting the protein and test compound are conducted in an aqueous solution and the altered physical properties of the protein-test compound complex is used to separate complex from non-complexed protein and test compound. In some embodiments of the above assay methods of the present invention, the step of contacting the RNA product and test compound are conducted in an aqueous solution and the altered physical properties of the RNA product-test compound complex is used to separate complex from non-complexed RNA product and test compound. Methods that can be used for the physical separation of protein-test compound complex or RNA product-test compound complex from unbound protein or unbound RNA product and unbound test compound include, but are not limited to, electrophoresis, fluorescence spectrometry, FRET assay, surface plasmon resonance, scintillation, proximity assay, structure-activity relationships (SAR) by NMR spectroscopy, size exclusion chromatography, affinity chromatography, and nanoparticle aggregation. See, e.g., U.S. publication No. 2004/0219545, which is incorporated herein by reference in its entirety, for similar assays and methods for separating complex based on physical properties. See Section 5.11.2 for a brief description of how a FRET assay may be conducted.

The interaction or binding of a protein described in Section 5.2 to a test compound can also be determined using such proteins as “bait proteins” in a two-hybrid assay or three hybrid assay (, e.g., U.S. Pat. No. 5,283,317; Zervos et al. (1993) Cell 72:223 232; Madura et al. (1993) J. Biol. Chem. 268:12046 12054; Bartel et al. (1993) Bio/Techniques 14:920-924; Iwabuchi et al. (1993) Oncogene 8:1693 1696; and International Publication No. WO 94/10300).

Once a compound has been identified as binding to a protein or RNA product of a nucleotide sequence described in Section 5.1, supra, the specificity and affinity of the compound for a HAI-TMIP variant RNA product or protein can be determined using techniques known in the art, such as immunoassays (e.g., ELISAs) and surface plasmon resonance.

### 5.11.1.2 Screening Assays to Identify Agents that Disrupt the Interaction of a HAI-TMIP Variant and a Cellular Molecule

The protein and RNA products of the nucleotide sequences described in Section 5.1, supra, in vivo, interact with a variety of cellular macromolecules, such as nucleotide sequences and proteins. Non-limiting examples of cellular macromolecules that a HAI-TMIP variant interacts with in vivo include, are but not limited to, an ATP synthase β subunit, an ATP synthase γ subunit, an ATP synthase δ subunit, an ATP synthase a subunit, angiostatin, apolipoprotein E-rich high-density lipoprotein, and coupling factor 6. For purposes of this discussion, such cellular macromolecules are referred to herein as “binding partners”. Compounds that disrupt the interaction between a protein or RNA product of a nucleotide sequence described in Section 5.1, supra, and a cellular macromolecule can be useful in regulating the activity of a HAI-TMIP variant.

The basic principle of the assay systems used to identify compounds that interfere with the interaction between a protein or RNA product of a nucleotide sequence described in Section 5.1, supra, and its binding partner(s) involves preparing a reaction mixture containing the protein or RNA product and the binding partner under conditions and for a time sufficient to allow the two to interact and bind, thus forming a complex. In order to test a compound for inhibitory activity, the reaction mixture is prepared in the presence and absence of the test compound. The test compound can be initially included in the reaction mixture, or can be added at a time subsequent to the addition of the protein or RNA product and its binding partner. Control reaction mixtures are incubated without the test compound or with a placebo. The formation of any complexes between the protein or RNA product and the binding partner is then detected. The formation of a complex in the control reaction, but not in the reaction mixture containing the test compound, indicates that the compound interferes with the interaction of the protein or RNA product and the interactive binding partner. Additionally, complex formation within reaction mixtures containing the test compound and a protein or RNA product of a specific HAI-TMIP variant can also be compared to complex formation within reaction mixtures containing the test compound and a protein or RNA product of a different HAI-TMIP variant. This comparison can be important in those cases wherein it is desirable to identify compounds that disrupt interactions of a specific HAI-TMIP variant but not other HAI-TMIP isoforms.

The assay for compounds that interfere with the interaction of a protein or RNA product and its binding partners can be conducted in a heterogeneous or homogeneous format.

Heterogeneous assays involve anchoring either the protein or RNA product, or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between a protein or RNA product and the binding partners, e.g., by competition, can be identified by conducting the reaction in the presence of the test substance; i.e., by adding the test compound to the reaction mixture prior to or simultaneously with the protein or RNA product and binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the protein or RNA product, or the binding partner, is anchored onto a solid surface, while the non-anchored species is labelled, either directly or indirectly. In practice, microliter plates are conveniently utilized. The anchored species can be immobilized by non-covalent or covalent attachments. Non-covalent attachment can be accomplished simply by coating the solid surface with a solution of the protein or RNA product or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labelled, the detection of label immobilized on the surface indicates that complexes were formed. Where the non-immobilized species is not pre-labelled, an indirect label can be used to detect complexes anchored on the surface; e.g., using a labelled antibody specific for the initially non-immobilized species (the antibody, in turn, can be directly labelled or indirectly labelled with a labelled anti-Ig antibody). Depending upon the order of addition of reaction components, test compounds which inhibit complex formation or which disrupt preformed complexes can be detected.

Alternatively, the reaction can be conducted in an aqueous phase in the presence or absence of the test compound, the reaction products separated from unreacted components, and complexes detected; e.g., using an immobilized antibody specific for one of the binding components to anchor any complexes formed in solution, and a labelled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of a protein or RNA product of a nucleotide sequence described in Section 5.1, supra, and its binding partner is prepared in which either the protein or RNA product, or its binding partner is labelled, but the signal generated by the label is quenched due to complex formation. The addition of a test substance that competes with and displaces one of the species from the preformed complex will result in the generation of a signal above background. In this way, test substances which disrupt protein or RNA product/binding partner interaction can be identified.

### 5.11.1.3 Screening Assays for Compounds that Modulate the Expression and/or Activity of a HAI-TMIP Variant

The present invention provides a method for identifying a compound that modulates the expression of HAI-TMIP variant 3, 4, 5, 6 or 7, the method comprising: (a) contacting a cell expressing a protein described in Section 5.2, supra, with a test compound; (b) determining the amount of the protein present in (a); and (c) comparing the amount in (a) to that present in a corresponding cell that has not been contacted with the test compound or has been contacted with a control compound, so that if the amount of the protein is altered relative to the amount in the control, a compound that modulates expression of HAI-TMIP variant 3, 4, 5, 6 or 7 is identified. In a specific embodiment, the expression level of the protein is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the expression level in the control as determined utilizing an assay described herein (e.g., an immunoassay) or an assay well known to one of skill in the art.

The present invention provides a method for identifying a compound that modulates the expression of HAI-TMIP variant 3, 4, 5, 6 or 7, the method comprising: (a) contacting a cell expressing a RNA product transcribed from a nucleotide sequence described in Section 5.1, supra, with a test compound; (b) determining the amount of the RNA product present in (a); and (c) comparing the amount in (a) to that present in a corresponding cell that has not been contacted with the test compound or has been contacted with a control compound, so that if the amount of the protein is altered relative to the amount in the control, a compound that modulates expression of HAI-TMIP variant 3, 4, 5, 6 or 7 is identified. In a specific embodiment, the expression level of the RNA product is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the expression level in the control as determined utilizing an assay described herein (e.g., RT-PCR or Northern blot analysis) or an assay well known to one of skill in the art.

The cells utilized in the cell-based assays described herein can be engineered to express a protein or RNA product of a nucleotide sequence described in Section 5.1, supra, utilizing techniques known in the art. See, e.g., Section 5.5, supra, and Section III entitled “Recombinant Expression Vectors and Host Cells” of U.S. Pat. No. 6,245,527, which is incorporated herein by reference. Alternatively, cells that endogenously express a protein or RNA product of a nucleotide sequence described in Section 5.1, supra, can be used.

The present invention also provides a method for identifying a compound that modulates the expression of HAI-TMIP variant 3, 4, 5, 6 or 7, the method comprising: (a) contacting a cell-free extract with a nucleotide sequence described in Section 5.1, supra, and a test compound; (b) determining the amount of the protein or RNA product of the nucleotide sequence present in (a); and (c) comparing the amount(s) in (a) to that present to a corresponding control that has not been contacted with the test compound or has been contacted with a control compound, so that if the amount of the protein or RNA product is altered relative to the amount in the control, a compound that modulates expression of HAI-TMIP variant 3, 4, 5, 6 or 7 is identified. In a specific embodiment, the expression level of the protein or RNA product is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the expression level in the control sample determined by utilizing an assay described herein (e.g., a microarray or RT-PCR) or an assay well known to one of skill in the art.

In certain embodiments, the amount of RNA product transcribed from a nucleotide sequence described in Section 5.1, supra, is determined, in other embodiments, the amount of protein encoded by a nucleotide sequence described in Section 5.1, supra, is determined, while in still other embodiments, the amount of RNA and protein product is determined. Standard methods and compositions for determining the amount of RNA or protein can be utilized. Such methods and compositions are described in detail below.

Reporter gene-based assays may also be conducted to identify a compound that modulates the expression of a HAI-TMIP isoform. In a specific embodiment, the present invention provides a method for identifying a compound that modulates the expression of HAI-TMIP variant 3, 4, 5, 6 or 7, the method comprising: (a) contacting a compound with a cell expressing a reporter gene construct comprising a reporter gene operably linked to a regulatory element of HAI-TMIP variant 3, 4, 5, 6 or 7 (e.g., a promoter/enhancer element); (b) measuring the expression of said reporter gene; and (c) comparing the amount in (a) to that present in a corresponding cell that has not been contacted with the test compound or has been contacted with a control compound, so that if the amount of expressed reporter gene is altered relative to the amount in the control cell, a compound that modulates expression of HAI-TMIP variant 3, 4, 5, 6 or 7 is identified. In another embodiment, the present invention provides a method for identifying a compound that modulates the expression of HAI-TMIP variant 3, 4, 5, 6 or 7, the method comprising: (a) contacting a compound with a cell-free extract and a reporter gene construct comprising a reporter gene operably linked to a regulatory element of HAI-TMIP variant 3, 4, 5, 6 or 7 (e.g., a promoter/enhancer element); (b) measuring the expression of said reporter gene; and (c) comparing the amount in (a) to that present in a corresponding control that has not been contacted with the test compound or has been contacted with a control compound, so that if the amount of expressed reporter gene is altered relative to the amount in the control, a compound that modulates expression of HAI-TMIP variant 3, 4, 5, 6 or 7 is identified.

Any reporter gene well-known to one of skill in the art may be used in reporter gene constructs used in accordance with the methods of the invention. Reporter genes refer to a nucleotide sequence encoding a RNA transcript or protein that is readily detectable either by its presence (by, e.g., RT-PCR, Northern blot, Western Blot, ELISA, etc.) or activity. Non-limiting examples of reporter genes are listed in Table 3, infra. Reporter genes may be obtained and the nucleotide sequence of the elements determined by any method well-known to one of skill in the art. The nucleotide sequence of a reporter gene can be obtained, e.g., from the literature or a database such as GenBank. Alternatively, a nucleotide sequence encoding a reporter gene may be generated from nucleic acid from a suitable source. If a clone containing a nucleotide sequence encoding a particular reporter gene is not available, but the sequence of the reporter gene is known, a nucleotide sequence encoding the reporter gene may be chemically synthesized or obtained from a suitable source (e.g., a cDNA library, or a cDNA library generated from, or nucleic acid, preferably poly A+ RNA, isolated from, any tissue or cells expressing the reporter gene) by PCR amplification. Once the nucleotide sequence of a reporter gene is determined, the nucleotide sequence of the reporter gene may be manipulated using methods well-known in the art for the manipulation of nucleotide sequences, e.g., recombinant DNA techniques, site directed mutagenesis, PCR, etc. (, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate reporter genes having a different amino acid sequence, for example to create amino acid substitutions, deletions, and/or insertions.

TABLE 5
Reporter Genes and the Properties of the Reporter Gene Products

| Reporter Gene | Protein Activity & Measurement |
|---|---|
| CAT (chloramphenicol | Transfers radioactive acetyl groups to |
| acetyltransferase) | chloramphenicol or detection by thin |
| | layer chromatography and autoradiography |
| GAL (beta-galactosidase) | Hydrolyzes colorless galactosides to |
| | yield colored products. |
| GUS (beta-glucuronidase) | Hydrolyzes colorless glucuronides to |
| | yield colored products. |
| LUC (luciferase) | Oxidizes luciferin, emitting photons |
| GFP (green fluorescent | Fluorescent protein without substrate |
| protein) | |
| SEAP (secreted alkaline | Luminescence reaction with suitable |
| phosphatase) | substrates or with substrates that |
| | generate chromophores |
| HRP (horseradish peroxidase) | In the presence of hydrogen oxide, |
| | oxidation of 3,3′,5,5′-tetramethylbenzidine |
| | to form a colored complex |
| AP (alkaline phosphatase) | Luminescence reaction with suitable |
| | substrates or with substrates that |
| | generate chromophores |

In accordance with the invention, cells that naturally or normally express one or more, all or any combination of HAI-TMIP variants 3, 4, 5, 6, and 7 can be used in the methods described herein. Alternatively, cells can be engineered to express one or more, all or any combination of HAI-TMIP variants 3, 4, 5, 6, and 7, or a reporter gene using techniques well-known in the art and used in the methods described herein. Examples of such techniques include, but are not to, calcium phosphate precipitation (, e.g., Graham & Van der Eb, 1978, Virol. 52:546), dextran-mediated transfection, calcium phosphate mediated transfection, polybrene mediated transfection, protoplast fusion, electroporation, encapsulation of the nucleic acid in liposomes, and direct microinjection of the nucleic acid into nuclei.

In a specific embodiment, the cells used in the methods described herein are lymphocytes (T or B lymphocytes) or endothelial cells. In another embodiment, the cells used in the methods described herein are immortalized cell lines derived from a source, e.g., a tissue. In another embodiment, the cells used in the methods described herein are cancer cells (e.g., breast cancer cells such as MDA-MB231 and MCF-7, and melanoma cells such as MDA-MB-435).

Any cell-free extract that permits the translation, and optionally but preferably, the transcription, of a nucleic acid can be used in accordance with the methods described herein. The cell-free extract may be isolated from cells of any species origin. For example, the cell-free translation extract may be isolated from human cells, cultured mouse cells, cultured rat cells, Chinese hamster ovary (CHO) cells, Xenopus oocytes, rabbit reticulocytes, wheat germ, or rye embryo (e.g., Krieg & Melton, 1984, Nature 308:203 and Dignam et al., 1990 Methods Enzymol. 182:194-203). Alternatively, the cell-free translation extract, e.g., rabbit reticulocyte lysates and wheat germ extract, can be purchased from, e.g., Promega, (Madison, Wis.). In a preferred embodiment, the cell-free extract is an extract isolated from human cells. In a specific embodiment, the human cells are cancer cells (e.g., breast cancer cells such as MDA-MB231 and MCF-7, and melanoma cells such as MDA-MB-435), lymphocytes, or endothelial cells.

Once a compound that modulates the expression of a protein or RNA product of a nucleotide sequence described in Section 5.1, supra, is identified, the specificity of the compound for a protein or RNA product of a particular HAI-TMIP variant can be assessed using techniques well-known in the art or described herein.

In addition to the ability to modulate the expression levels of RNA and/or protein products of a nucleotide sequence described in Section 5.1, supra, it may be desirable, at least in certain instances, that compounds modulate the activity of the protein product. Thus, the present invention provides methods of identifying compounds modulate the activity of HAI-TMIP variant 3, 4, 5, 6 or 7. Such methods can comprise: (a) contacting a cell expressing a protein described in Section 5.2, supra, with a test compound; (b) determining the activity level of the protein; and (c) comparing the activity level to that in a corresponding cell that has not been contacted with the test compound or has been contacted with a control compound, so that if the level of activity in (a) is altered relative to the level of activity in the control cell, a compound that modulates the activity of HAI-TMIP variant 3, 4, 5, 6 or 7 is identified. In a specific embodiment, the activity level(s) is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the activity level in the control as determined by utilizing an assay described herein (e.g., an immunoassay) or an assay well known to one of skill in the art.

The present invention provides methods of identifying compounds modulate the activity of HAI-TMIP variant 3, 4, 5, 6 or 7, the methods comprising: (a) contacting a cell-free extract with a nucleotide sequence encoding a protein described in Section 5.2, supra, and a test compound; (b) determining the activity level of the protein; and (c) comparing the activity level to that in a corresponding control that has not been contacted with the test compound or has been contacted with a control compound, so that if the level of activity in (a) is altered relative to the level of activity in the control, a compound that modulates the activity of HAI-TMIP variant 3, 4, 5, 6 or 7 is identified. In a specific embodiment, the activity level(s) is altered by 5%, 10%, 15%, 25%, 30%, 40%, 50%, 5 to 25%, 10 to 30%, at least 1 fold, at least 1.5 fold, at least 2 fold, 4 fold, 5 fold, 10 fold, 25 fold, 1 to 10 fold, or 5 to 25 fold relative to the activity level in the control as determined by utilizing an assay described herein (e.g., immunoassay) or an assay well known to one of skill in the art.

The affect of a test compound on the activity of HAI-TMIP variant 3, 4, 5, 6 or 7 can be determined, e.g., by measuring the ability of HAI-TMIP variant 3, 4, 5, 6 or 7 to bind to an ATP synthase β subunit. Standard techniques well-known in the art and described herein can be used to determine the ability of the HAI-TMIP variant to bind to an ATP synthase β subunit.

The affect of a test compound on the activity of HAI-TMIP variant 3, 4, 5, 6 or 7 can be determined, e.g., by measuring ATP synthase activity using standard enzyme assay protocols.) For example, the following assay can be conducted: Quiescent, confluent cells expressing a protein described in Section 5.2, supra, in 24 well plates are washed and equilibrated into media containing potassium phosphate. Cells are treated with a test compound for a period of time (e.g., 1 hour) at 37° C. All of the cells are then incubated with 0.1 Ci ₃₂P and 0.1 Ci (50 M) [2,8-₃H]-ADP (NEN Life Sciences) for a period of time (e.g., 1 minute). Supernatants are removed and centrifuged before assaying for ATP production by thin layer chromatography (TLC) or by firefly luciferase assay.

The bioluminescent luciferase assay to measure ATP production can be conducted using techniques well-known in the art. Briefly, aliquots of cellular supernatants from cell-surface ATP assays are analyzed using the ATP bioluminescence assay kit (Sigma Chemical, St. Louis, Mo.). In this firefly luciferin-luciferase reaction, only ATP is readily detected since the enzymatic reaction of firefly luciferase to oxidize luciferin is specific for ATP relative to all other nucleotides. Samples are injected with the ATP assay mixture and recordings were made in a LuminoskanRS (LabSystems, Franklin, Mass.). The response in a given sample or standard is quantified as area under the peak of the response and averaged for duplicate determinations. Data are expressed as picomoles of ATP produced per cell based on standards determined under the same conditions with each experiment.

The dual label radioactive thin layer chromatography assay to measure ATP production can be conducted using techniques well-known in the art. Briefly, supernatants are obtained as described above. Cell pellets are obtained after washing wells with media and lysing with lysis buffer. Aliquots of supernatant and cell pellet are applied to microcrystalline cellulose PEI plates (Anal. Tech., Newark, Del.) along with an authentic [³²P]ATP standard (0.025 Ci). Plates are developed. Dried spots containing [³²P]ATP, [³H]ATP, and [³H]ADP are detected by sodium iodide and phosphoimaging on a STORM 850 (Molecular Dynamics, Sunnyvale, Calif.). Areas corresponding in R_{f} value to co-chromatographed authentic [³²P]ATP standard are scraped off the plate and their radioactivity determined in a liquid scintillation analyzer (Packard BioScience Co., Meridan, Conn.).

The selectivity of a test compound for cell surface F₁ ATP synthase, in particular, for the alpha subunit thereof, as compared to mitochondrial F₁ ATP synthase, can be easily assessed. Compounds which, by virtue of their physicochemical properties, cannot diffuse across cellular membranes (and that are not natural or artificial ligands for cell transporters) can be considered selective for cell surface F₁ ATP synthase. For example, compounds that bind cell surface F₁ ATP synthase but are positively charged can thereby be prevented from diffusing across membranes.

### 5.11.2 Modulators of Complex

The present invention provides methods for screening for compounds that bind to, modulate the amount of, or activity of a complex of the present invention. In one embodiment of the invention, the method for screening for a compound that modulates directly or indirectly the function, activity or formation of a complex of the present invention comprises exposing said complex, or a cell or organism containing the complex machinery, to one or more compounds under conditions conducive to modulation; and determining the amount of, activity of, or identities of the protein components of said complex, wherein a change in said amount, activity, or identities relative to said amount, activity or identities in the absence of said compounds indicates that the compounds modulate function, activity or formation of said complex. Such screening assays can be carried out using cell-free and cell-based methods that are commonly known in the art.

In certain embodiments, the method for identifying a modulator of the formation or stability of a complex of the invention can be carried out in vitro, particularly in a cell-free system. In certain, more specific embodiments, the complex is purified. In certain embodiments the test compound is purified.

In a specific embodiment, screening can be carried out by contacting the library members with a complex immobilized on a solid phase, and harvesting those library members that bind to the complex. Examples of such screening methods, termed “panning” techniques, are described by way of example in Parmley and Smith, 1988, Gene 73:305 318; Fowlkes et al., 1992, BioTechniques 13:422-427; International Patent Publication No. WO 94/18318 (which are incorporated by reference herein); and in references cited hereinabove.

In one embodiment, compounds that modulate (i.e., antagonize or agonize) complex activity or formation can be screened for using a binding inhibition assay, wherein compounds are screened for their ability to modulate formation of a complex under aqueous, or physiological, binding conditions in which complex formation occurs in the absence of the compound to be tested. Compounds that promote the formation of complexes are identified as activators of complex formation. Compounds that completely interfere with or block the formation of complexes are identified as inhibitors of complex formation. In an exemplary embodiment, the binding conditions are, for example, but not by way of limitation, in an aqueous salt solution of 10-250 mM NaCl, 5-50 mM Tris-HCl, pH 5-8, and 0.5% Triton X-100 or other detergent that improves specificity of interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur. Particular complexes can be assayed using routine protein binding assays to determine optimal binding conditions for reproducible binding.

In certain embodiments, assays can be carried out using recombinant cells expressing the protein components of a complex, to screen for molecules that bind to, or interfere with, or promote complex activity or formation. In certain embodiments, at least one of the protein components is expressed in the recombinant cell as fusion protein, wherein the protein component is fused to a peptide tag to facilitate purification and subsequent quantification and/or immunological visualization and quantification.

A particular aspect of the present invention relates to identifying molecules that inhibit or promote formation or degradation of a complex of the present invention, e.g., using the method described for isolating the complex and identifying members of the complex using the TAP assay described in WO 00/09716 and Rigaut et al., 1999, Nature Biotechnology 17:1030-1032, which are each incorporated by reference in their entireties.

In a specific embodiment, a FRET cell-based assay may be conducted by microinjecting or transfecting a first subunit of a human F₁ ATP synthase subcomplex (e.g., HAI-TMIP variant 3, 4, 5, 6 or 7) labelled with a fluorophore and a second, different subunit of a human F₁ ATP synthase subcomplex (e.g., human ATP synthase β subunit) labelled with a quencher into a cell and contacting the cell with a test compound, and measuring the fluorescence of the human F₁ ATP synthase subcomplex by, e.g., fluorescence microscopy or a fluorescence emission detector such as a Viewlux or Analyst. Preferably, the cell microinjected or transfected is deficient in one or more of the subunits of the human F₁ ATP synthase subcomplex. Any methods known to the skilled artisan can be used to remove the expression and/or function of one or more subunits of the human F₁ ATP synthase subcomplex from the cell. In a specific embodiment, RNAi is used to transiently remove one or more of the subunits of the human F₁ ATP synthase subcomplex. The formation of the human F₁ ATP synthase subcomplex from the labelled subunits will result in a reduction in the fluorescence detectable. A compound that inhibits or reduces the formation of the human F₁ ATP synthase subcomplex will increase the fluorescence detectable signal relative to a negative control (e.g., PBS). A compound that enhances the formation of the human F₁ ATP synthase subcomplex will decrease the fluorescence detectable relative to a negative control (e.g., PBS).

Alternatively, a FRET cell-based assay may be conducted by microinjecting a first subunit of a human F₁ ATP synthase subcomplex (e.g., HAI-TMIP variant 3, 4, 5, 6 or 7) labelled with a fluorescent donor moiety and a second, different subunit of a human F₁ ATP synthase subcomplex (e.g., ATP synthase β subunit) labelled with a fluorescent acceptor moiety into a cell and contacting the cell with a compound, and measuring the fluorescence of the human F₁ ATP synthase subcomplex by, e.g., fluorescence microscopy or a fluorescence emission detector such as a Viewlux or Analyst. The formation of the human F₁ ATP synthase subcomplex will result in the production of a detectable fluorescent signal by the fluorescent donor moiety and fluorescent acceptor moiety at the wavelength of the fluorescent donor moiety. A compound that inhibits or reduces the formation of the human F₁ ATP synthase subcomplex will reduce the fluorescence emission of the fluorescent acceptor moiety at the wavelength of the fluorescent donor moiety relative to a negative control (e.g., PBS). A compound that enhances the formation of the human F₁ ATP synthase subcomplex will increase the fluorescence emission of the fluorescent acceptor moiety at the wavelength of the fluorescent donor moiety relative to a negative control (e.g., PBS). In a preferred embodiment, a negative control (e.g., PBS or another agent that is known to have no effect on the cleavage of the substrate) and a positive control (e.g., an agent that is known to have an effect on the cleavage of the substrate) are included in the FRET cell-based assays described herein.

In certain embodiments, the compound and the cell are incubated for at least 0.2 hours, 0.25 hours, 0.5 hours, 1 hour, 2 hours, 3 hours, 4 hours, 5 hours, 6 hours, 8 hours, 10 hours, 12 hours, 18 hours, or at least 1 day.

Methods for labelling a subunit of a human F₁ ATP synthase subcomplex with a fluorescent acceptor moiety, a fluorescent donor moiety and/or quencher are well-known in the art (, e.g., U.S. Pat. Nos. 6,472,156, 6,451,543, 6,348,322, 6,342,379, 6,323,039, 6,297,018, 6,291,201, 6,280,981, 5,843,658, and 5,439,797, the disclosures of which are incorporated by reference in their entirety).

In a specific embodiment, a FRET cell-free assay is conducted by contacting a first subunit of a human F₁ ATP synthase subcomplex (e.g., HAI-TMIP variant 3, 4, 5, 6 or 7) labelled with a fluorophore and a second, different subunit of a human F₁ ATP synthase subcomplex (e.g., ATP synthase β subunit) labelled with a quencher with a test compound in vitro under conditions conducive to the formation of the F₁ ATP synthase subcomplex, and measuring the fluorescence of the human F₁ ATP synthase subcomplex by, e.g., a fluorescence emission detector such as a Viewlux or Analyst. The formation of the human F₁ ATP synthase subcomplex from the labelled subunits will result in a reduction in the fluorescence detectable. A compound that inhibits or reduces the formation of the human F₁ ATP synthase subcomplex will enhance the production of detectable fluorescent signal relative to the absence of the compound or the presence of a negative control (e.g., PBS). A compound that enhances the formation of the human F₁ ATP synthase subcomplex will reduce or inhibit the fluorescence detectable relative to the absence of the compound or a negative control (e.g., PBS).

Alternatively, a FRET cell-free assay may be conducted by contacting a first subunit of a human F₁ ATP synthase subcomplex (e.g., HAI-TMIP variant 3, 4, 5, 6 or 7) labelled with a fluorophore and a second, different subunit of a human F₁ ATP synthase subcomplex (e.g., ATP synthase β subunit) labelled with a fluorescent acceptor moiety with a compound in vitro under conditions conducive to the formation of the F₁ ATP synthase subcomplex, and measuring the fluorescence of the human F₁ ATP synthase subcomplex by, e.g., a fluorescence emission detector such as a Viewlux or Analyst. The formation of the human F1 ATP synthase subcomplex will result in the production of a detectable fluorescent signal by the fluorescent donor moiety and fluorescent acceptor moiety at the wavelength of the fluorescent donor. A compound that inhibits or reduces the formation of the human F₁ ATP synthase subcomplex will reduce the fluorescence emission of the fluorescent acceptor moiety at the wavelength of the fluorescent donor moiety relative to the absence of the compound or the presence of a negative control (e.g., PBS). A compound that enhances the formation of the human F₁ ATP synthase subcomplex will increase the fluorescence emission of the fluorescent acceptor moiety at the wavelength of the fluorescent donor moiety relative to the absence of the compound or the presence of a negative control (e.g., PBS). In a preferred embodiment, a negative control (e.g., PBS or another agent that is known to have no effect on the cleavage of the substrate) and a positive control (e.g., an agent that is known to have an effect on the cleavage of the substrate) are included in the FRET cell-free assays described herein.

The affect of a test compound on the activity of an F₁F₀ ATP synthase complex of the invention can be determined by, e.g., conducting the assay described in Section 5.11.2 for ATP synthesis or ATP hydrolysis. The affect of a test compound on the activity of an F₁ ATP synthase subcomplex can be determined by measuring ATP hydrolysis using techniques well-known in the art. For example, activities can be measured in triplicate by monitoring reaction rates in a time period from 3 to 6 minutes using an ATP regenerating system at 25° C. (50 mM potassium phosphate buffer, pH 7.0, containing 100 mM KCl, 5 mM MgCl₂, 2.5 mM phosphoenolpyruvate, 2 mM ATP, 0.2 mM NADH, 0.1 mg/ml pyruvate kinase, and 0.1 mg/ml lactate dehydrogenase). Protein concentrations can be determined using the BCA Protein Assay Kit from Pierce, with bovine serum albumin as a standard.

### 5.12 Compounds

Any agent known in the art can be tested for its ability to modulate (increase or decrease) the expression and/or activity of a HAI-TMIP (e.g., HAI-TMIP variant 1, 2, 3, 4, 5, 6 or 7), or a complex comprising a HAI-TMIP (e.g., a complex of the present invention) as detected by a change in the amount and/or activity of HAI-TMIP variant or the ATP synthase complex. By way of example, a change in the amount of the complex can be detected by detecting a change in the amount of the complex that can be isolated from a cell expressing the complex machinery. In other embodiments, a change in signal intensity (e.g., when using FRET) in the presence of a compound compared to the absence of the compound indicates that the compound is a modulator of complex formation. For identifying a compound that modulates complex activity, a test compound can be directly provided to a cell expressing the complex, or, in the case of candidate proteins, can be provided by providing their encoding nucleic acids under conditions in which the nucleic acids are recombinantly expressed to produce the candidate proteins within the cell expressing the complex machinery, the complex is then purified from the cell and the purified complex is assayed for activity using methods well known in the art, not limited to those described, supra.

In certain embodiments, the invention provides screening assays using libraries for compounds which modulate, e.g., inhibit, antagonize, or agonize, the amount and/or activity of a HAI-TMIP variant (e.g., HAI-TMIP variant 1, 2, 3, 4, 5, 6 or 7), or a complex comprising a HAI-TMIP variant (e.g., a complex of the invention). Libraries screened using the methods of the present invention can comprise a variety of types of compounds. Examples of libraries that can be screened in accordance with the methods of the invention include, but are not limited to, peptoids; random biooligomers; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; carbohydrate libraries; and small molecule libraries (preferably, small organic molecule libraries). In some embodiments, the compounds in the libraries screened are nucleic acid or peptide molecules. In a non-limiting example, peptide molecules can exist in a phage display library. In other embodiments, the types of compounds include, but are not limited to, peptide analogs including peptides comprising non-naturally occurring amino acids, e.g., D-amino acids, phosphorous analogs of amino acids, such as α-amino phosphoric acids and α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose. Libraries of polypeptides or proteins can also be used in the assays of the invention. The chemical libraries that can be screened include peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate compounds can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or non-constrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922 10926; Erb et al., 1994, Proc. Natl. Acad. Sci. USA 91:11422 11426; Houghten et al., 1992, Biotechniques 13:412; Jayawickreme et al., 1994, Proc. Natl. Acad. Sci. USA 91:1614 1618; or Salmon et al., 1993, Proc. Natl. Acad. Sci. USA 90:11708 11712. PCT Publication No. WO 93/20242 and Brenner and Lerner, 1992, Proc. Natl. Acad. Sci. USA 89:5381 5383 describe “encoded combinatorial chemical libraries,” that contain oligonucleotide identifiers for each chemical polymer library member.

In a specific embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non naturally occurring amino acids, non-peptide structures, and peptides containing a significant fraction of γ carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367 9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142.

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non classical amino acids or cyclic peptides. Non-classical amino acids include but are not limited to the D-isomers of the common amino acids, α-amino isobutyric acid, 4-aminobutyric acid, Abu, 2-amino butyric acid; γ-Abu, ε-Ahx, 6-amino hexanoic acid; Aib, 2-amino isobutyric acid; 3-amino propionic acid; ornithine; norleucine; norvaline, hydroxyproline, sarcosine, citrulline, cysteic acid, t-butylglycine, t butylalanine, phenylglycine, cyclohexylalanine, β-alanine, designer amino acids such as β-methyl amino acids, Cα-methyl amino acids, Nα-methyl amino acids, fluoro-amino acids and amino acid analogs in general. Furthermore, the amino acid can be D (dextrorotary) or L (levorotary).

In a specific embodiment, fragments and/or analogs of complexes of the invention, or protein components thereof, especially peptidomimetics, are screened for activity as competitive or non-competitive inhibitors of complex activity or formation.

In another embodiment of the present invention, combinatorial chemistry can be used to identify modulators of HAI-TMIP variant 3, 4, 5, 6 or 7, or a complex of the invention. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See, e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (e.g., the protein complexes of the present invention and protein components thereof.) The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify complex modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

In a specific embodiment, small organic molecule libraries including, but not limited to, benzodiazepines, isoprenoids, thiazolidinones, metathiazanones, pyrrolidines, morpholino compounds, and benzodiazepines. In another embodiment, the combinatorial libraries comprise peptoids; random bio-oligomers; benzodiazepines; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; or carbohydrate libraries. Combinatorial libraries are themselves commercially available (see, e.g., ComGenex, Princeton, N.J.; Asinex, Moscow, Ru, Tripos, Inc., St. Louis, Mo.; ChemStar, Ltd, Moscow, Russia; 3D Pharmaceuticals, Exton, Pa.; Martek Biosciences, Columbia, Md.; etc.).

In a specific embodiment, the library is preselected so that the compounds of the library are more amenable for cellular uptake. For example, compounds are selected based on specific parameters such as, but not limited to, size, lipophilicity, hydrophilicity, and hydrogen bonding, which enhance the likelihood of compounds getting into the cells. In another embodiment, the compounds are analyzed by three-dimensional or four-dimensional computer computation programs.

The compound library for use in accordance with the methods of the present invention may be synthesized. There is a great interest in synthetic methods directed toward the creation of large collections of small organic compounds, or libraries, which could be screened for pharmacological, biological or other activity. The synthetic methods applied to create vast combinatorial libraries are performed in solution or in the solid phase, i.e., on a solid support. Solid phase synthesis makes it easier to conduct multi step reactions and to drive reactions to completion with high yields because excess reagents can be easily added and washed away after each reaction step. Solid phase synthesis also tends to improve isolation, purification and screening. However, the more traditional solution phase chemistry supports a wider variety of organic reactions than solid phase chemistry.

Combinatorial compound libraries of the present invention may be synthesized using the apparatus described in U.S. Pat. No. 6,190,619 to Kilcoin et al., which is hereby incorporated by reference in its entirety. U.S. Pat. No. 6,190,619 discloses a synthesis apparatus capable of holding a plurality of reaction vessels for parallel synthesis of multiple discrete compounds or for combinatorial libraries of compounds.

In one embodiment, the combinatorial compound library can be synthesized in solution. The method disclosed in U.S. Pat. No. 6,194,612 to Boger et al., which is hereby incorporated by reference in its entirety, features compounds useful as templates for solution phase synthesis of combinatorial libraries. The template is designed to permit reaction products to be easily purified from unreacted reactants using liquid/liquid or solid/liquid extractions. The compounds produced by combinatorial synthesis using the template will preferably be small organic molecules. Some compounds in the library may mimic the effects of non-peptides or peptides. In contrast to solid phase synthesize of combinatorial compound libraries, liquid phase synthesis does not require the use of specialized protocols for monitoring the individual steps of a multistep solid phase synthesis (Egner et al., 1995, J. Org. Chem. 60:2652; Anderson et al., 1995, J. Org. Chem. 60:2650; Fitch et al., 1994, J. Org. Chem. 59:7955; Look et al., 1994, J. Org. Chem. 49:7588; Metzger et al., 1993, Angew. Chem., Int. Ed. Engl. 32:894; Youngquist et al., 1994, Rapid Commun. Mass Spect. 8:77; Chu et al., 1995, J. Am. Chem. Soc. 117:5419; Brummel 1994, Science 264:399; and Stevanovic et al., 1993, Bioorg. Med. Chem. Lett. 3:431).

Combinatorial compound libraries useful for the methods of the present invention can be synthesized on solid supports. In one embodiment, a split synthesis method, a protocol of separating and mixing solid supports during the synthesis, is used to synthesize a library of compounds on solid supports (see e.g., Lam et al., 1997, Chem. Rev. 97:41-448; Ohlmeyer et al., 1993, Proc. Natl. Acad. Sci. USA 90:10922-10926 and references cited therein). Each solid support in the final library has substantially one type of compound attached to its surface. Other methods for synthesizing combinatorial libraries on solid supports, wherein one product is attached to each support, will be known to those of skill in the art (see, e.g., Nefzi et al., 1997, Chem. Rev. 97:449-472).

As used herein, the term “solid support” is not limited to a specific type of solid support. Rather a large number of supports are available and are known to one skilled in the art. Solid supports include silica gels, resins, derivatized plastic films, glass beads, cotton, plastic beads, polystyrene beads, alumina gels, and polysaccharides. A suitable solid support may be selected on the basis of desired end use and suitability for various synthetic protocols. For example, for peptide synthesis, a solid support can be a resin such as p-methylbenzhydrylamine (pMBHA) resin (Peptides International, Louisville, Ky.), polystyrenes (e.g., PAM-resin obtained from Bachem Inc., Peninsula Laboratories, etc.), including chloromethylpolystyrene, hydroxymethylpolystyrene and aminomethylpolystyrene, poly (dimethylacrylamide)-grafted styrene co-divinyl-benzene (e.g., POLYHIPE resin, obtained from Aminotech, Canada), polyamide resin (obtained from Peninsula Laboratories), polystyrene resin grafted with polyethylene glycol (e.g., TENTAGEL or ARGOGEL, Bayer, Tubingen, Germany) polydimethylacrylamide resin (obtained from Milligen/Biosearch, California), or Sepharose (Pharmacia, Sweden).

In some embodiments of the present invention, compounds can be attached to solid supports via linkers. Linkers can be integral and part of the solid support, or they may be nonintegral that are either synthesized on the solid support or attached thereto after synthesis. Linkers are useful not only for providing points of compound attachment to the solid support, but also for allowing different groups of molecules to be cleaved from the solid support under different conditions, depending on the nature of the linker. For example, linkers can be, inter alia, electrophilically cleaved, nucleophilically cleaved, photocleavable, enzymatically cleaved, cleaved by metals, cleaved under reductive conditions or cleaved under oxidative conditions. In a preferred embodiment, the compounds are cleaved from the solid support prior to high throughput screening of the compounds.

In certain embodiments of the invention, the compound is a small molecule. In other embodiments of the invention, the compound is an antibody. In yet other embodiments of the invention, the compound is a naturally occurring cellular macromolecule.

### 5.13 Characterization of the Structure of Compounds

If a library comprises arrays or microarrays of compounds, wherein each compound has an address or identifier, the compound can be deconvoluted, e.g., by cross-referencing the positive sample to original compound list that was applied to the individual test assays.

If the library is a peptide or nucleic acid library, the sequence of the compound can be determined by direct sequencing of the peptide or nucleic acid. Such methods are well known to one of skill in the art.

A number of physico-chemical techniques can be used for the de novo characterization of compounds identified by the screening methods of the invention. Examples of such techniques include, but are not limited to, mass spectrometry, NMR spectroscopy, X-ray crystallography and vibrational spectroscopy.

Mass spectrometry (e.g., electrospray ionization (“ESI”), matrix-assisted laser desorption-ionization (“MALDI”), and Fourier-transformation cyclotron resonance (“FT-ICR”) can be used for elucidating the structure of a compound.

ESI mass spectrometry (“ESI-MS”) has been of greater utility for studying non-covalent molecular interactions because, unlike the MALDI process, ESI-MS generates molecular ions with little to no fragmentation (Xavier et al., 2000, Trends Biotechnol. 18(8):349-356). ESI-MS has been used to study the complexes formed by HIV Tat peptide and protein with the TAR RNA (Sannes-Lowery et al., 1997, Anal. Chem. 69:5130-5135).

Fourier-transformation cyclotron resonance (“FT-ICR”) mass spectrometry provides high-resolution spectra, isotope-resolved precursor ion selection, and accurate mass assignments (Xavier et al., 2000, Trends Biotechnol. 18(8):349-356). FT-ICR has been used to study the interaction of aminoglycoside antibiotics with cognate and non-cognate RNAs (Hofstadler et al., 1999, Anal. Chem. 71:3436-3440; and Griffey et al., 1999, Proc. Natl. Acad. Sci. USA 96:10129-10133). As true for all of the mass spectrometry methods discussed herein, FT-ICR does not require labelling a compound.

NMR spectroscopy is a valuable technique for determining the structure of a compound by qualitatively determining changes in chemical shift, specifically from distances measured using relaxation effects. SAR by NMR can be used to elucidate the structure of a compound.

Examples of NMR that can be used for the invention include, but are not limited to, one-dimensional NMR, two-dimensional NMR, correlation spectroscopy (“COSY”), and nuclear Overhauser effect (“NOE”) spectroscopy. Such methods of structure determination of compounds are well-known to one of skill in the art.

X-ray crystallography can be used to elucidate the structure of a compound. For a review of x-ray crystallography, e.g., Blundell et al., 2002, Nat Rev Drug Discov 1(1):45 54. The first step in x-ray crystallography is the formation of crystals. The formation of crystals begins with the preparation of highly purified and soluble samples. The conditions for crystallization is then determined by optimizing several solution variables known to induce nucleation, such as pH, ionic strength, temperature, and specific concentrations of organic additives, salts and detergent. Techniques for automating the crystallization process have been developed to automate the production of high-quality protein crystals. Once crystals have been formed, the crystals are harvested and prepared for data collection. The crystals are then analyzed by diffraction (such as multi circle diffractometers, high speed CCD detectors, and detector off set). Generally, multiple crystals must be screened for structure determinations.

Vibrational spectroscopy (e.g., infrared (IR) spectroscopy or Raman spectroscopy) can be used for elucidating the structure of a compound. Infrared spectroscopy measures the frequencies of infrared light (wavelengths from 100 to 10,000 nm) absorbed by the compound as a result of excitation of vibrational modes according to quantum mechanical selection rules which require that absorption of light cause a change in the electric dipole moment of the molecule. The infrared spectrum of any molecule is a unique pattern of absorption wavelengths of varying intensity that can be considered as a molecular fingerprint to identify any compound.

Infrared spectra can be measured in a scanning mode by measuring the absorption of individual frequencies of light, produced by a grating which separates frequencies from a mixed frequency infrared light source, by the compound relative to a standard intensity (double-beam instrument) or pre-measured (‘blank’) intensity (single beam instrument). In a preferred embodiment, infrared spectra are measured in a pulsed mode (“FT-IR”) where a mixed beam, produced by an interferometer, of all infrared light frequencies is passed through or reflected off the compound. The resulting interferogram, which may or may not be added with the resulting interferograms from subsequent pulses to increase the signal strength while averaging random noise in the electronic signal, is mathematically transformed into a spectrum using Fourier Transform or Fast Fourier Transform algorithms.

Raman spectroscopy measures the difference in frequency due to absorption of infrared frequencies of scattered visible or ultraviolet light relative to the incident beam. The incident monochromatic light beam, usually a single laser frequency, is not truly absorbed by the compound but interacts with the electric field transiently. Most of the light scattered off the sample will be unchanged (Rayleigh scattering) but a portion of the scatter light will have frequencies that are the sum or difference of the incident and molecular vibrational frequencies. The selection rules for Raman (inelastic) scattering require a change in polarizability of the molecule. While some vibrational transitions are observable in both infrared and Raman spectrometry, must are observable only with one or the other technique. The Raman spectrum of any molecule is a unique pattern of absorption wavelengths of varying intensity that can be considered as a molecular fingerprint to identify any compound.

Raman spectra are measured by submitting monochromatic light to the sample, either passed through or preferably reflected off, filtering the Rayleigh scattered light, and detecting the frequency of the Raman scattered light. An improved Raman spectrometer is described in U.S. Pat. No. 5,786,893 to Fink et al., which is hereby incorporated by reference.

Vibrational microscopy can be measured in a spatially resolved fashion to address single beads by integration of a visible microscope and spectrometer. A microscopic infrared spectrometer is described in U.S. Pat. No. 5,581,085 to Reffner et al., which is hereby incorporated by reference in its entirety. An instrument that simultaneously performs a microscopic infrared and microscopic Raman analysis on a sample is described in U.S. Pat. No. 5,841,139 to Sostek et al., which is hereby incorporated by reference in its entirety.

### 5.14 Biological Assays

The compounds identified in the assays described supra that modulate the expression and/or activity of HAI-TMIP variant 1, 2, 3, 4, 5, 6 or 7, or a complex of the invention (for convenience, sometimes referred to herein as a “lead” compound) can be further tested for biological activity. In one embodiment, the compounds are tested for biological activity in further assays and/or animal models. In another embodiment, the lead compound is used to design congeners or analogs. In another embodiment, mutagenesis studies can be conducted to assess the mechanism by which a lead'compound is modulating the expression and/or activity of HAI-TMIP variant 1, 2, 3, 4, 5, 6 or 7, or a complex of the invention.

### 5.14.1 Characteristics of Compound

Compounds identified in the assays described above may be characterized in a variety of ways well-known to one of skill in the art. In particular, compounds may be assayed for the ability to specifically or selectively bind to a HAI-TMIP variant (e.g, HAI-TMIP variant 3, 4, 5, 6, or 7), or a complex comprising a HAI-TMIP variant (e.g., a complex of the invention). Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Cwirla et al., 1990, Proc. Natl. Acad. Sci. USA 87:6378-6382; and Felici, 1991, J. Mol. Biol. 222:301-310) (each of these references is incorporated herein in its entirety by reference). Compounds that have been identified to specifically bind to a HAI-TMIP variant (e.g., HAI-TMIP variant 3, 4, 5, 6 or 7) or a complex of the invention can then be assayed for their affinity for a HAI-TMIP variant (e.g., HAI-TMIP variant 3, 4, 5, 6 or 7) or a complex of the invention.

The compounds may be assayed for specific binding to a HAI-TMIP variant (e.g., HAI-TMIP variant 1, 2, 3, 4, 5, 6 or 7) or a complex of the invention and cross-reactivity with other antigens by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The binding affinity of a compound to a HAI-TMIP variant (e.g., HAI-TMIP variant 1, 2, 3, 4, 5, 6 or 7) or a complex of the invention and the off-rate of such an interaction can be determined by competitive binding assays. One example of a competitive binding assay is a radioimmunoassay comprising the incubation of labelled HAI-TMIP or complex of the invention (e.g., ³H or ¹²⁵I) with the compound of interest in the presence of increasing amounts of unlabelled HAI-TMIP or complex of the invention, and the detection of the compound bound to the labeled HAI-TMIP or complex of the invention. The affinity of the compound for HAI-TMIP or complex of the invention and the binding off-rates can be determined from the data by scatchard plot analysis. Competition with a second compound can also be determined using radioimmunoassays. In this case, HAI-TMIP or complex of the invention is incubated with a compound conjugated to a label (e.g., ³H or ¹²⁵I) in the presence of increasing amounts of an unlabelled second compound.

In a specific embodiment, BIAcore kinetic analysis is used to determine the binding on and off rates of compounds to a HAI-TMIP variant (e.g., HAI-TMIP variant 1, 2, 3, 4, 5, 6 or 7) or a complex of the invention. BIAcore kinetic analysis comprises analyzing the binding and dissociation of a HAI-TMIP variant (e.g., HAI-TMIP variant 1, 2, 3, 4, 5, 6 or 7) or a complex of the invention from chips with immobilized compounds on their surface. A typical BIAcore kinetic study involves the injection of 250 uL of a compound reagent (e.g., a mAb or Fab) at varying concentration in HBS buffer containing 0.005% Tween-20 over a sensor chip surface, onto which has been immobilized a HAI-TMIP variant (e.g., HAI-TMIP variant 1, 2, 3, 4, 5, 6, or 7) or a complex of the invention. The flow rate is maintained constant at 75 uL/min. Dissociation data is collected for 15 min. or longer as necessary. Following each injection/dissociation cycle, the bound compound is removed from the antigen surface using brief, 1 min. pulses of dilute acid, typically 10-100 mM HCl, though other regenerants are employed as the circumstances warrant. More specifically, for measurement of the rates of association, kₒₙ, and dissociation, k_{off}, a HAI-TMIP variant (e.g., HAI-TMIP variant 1, 2, 3, 4, 5, 6, or 7) or a complex of the invention is directly immobilized onto the sensor chip surface through the use of standard amine coupling chemistries, namely the EDC/NHS method (EDC=N-diethylaminopropyl)-carbodiimide). Briefly, a 5-100 nM solution of the HAI-TMIP variant or complex of the invention in 10 mM NaOAc, pH4 or pH5 is prepared and passed over the EDC/NHS-activated surface until approximately 30-50 RU's worth of HAI-TMIP or complex are immobilized. Following this, the unreacted active esters are “capped” off with an injection of 1M Et-NH2. A blank surface, containing no antigen, is prepared under identical immobilization conditions for reference purposes. Once an appropriate surface has been prepared, a suitable dilution series of each one of the compound reagents is prepared in HBS/Tween-20, and passed over both the HAI-TMIP or complex and reference cell surfaces, which are connected in series. The range of compound concentrations that are prepared varies, depending on what the equilibrium binding constant, K_{D}, is estimated to be. As described above, the bound compound is removed after each injection/dissociation cycle using an appropriate regenerant.

The compound can also be assayed for their ability to inhibit the binding of a HAI-TMIP variant or a complex of the invention to its host cell receptor or ligand, respectively, using techniques known to those of skill in the art. For example, cells expressing an ATP can be contacted with a ligand (e.g., angiostatin) in the presence or absence of a compound and the ability of the compound to inhibit the ligand's binding can measured by, for example, flow cytometry or a scintillation assay. The ligand or the compound can be labelled with a detectable agent such as a radioactive label (e.g., ³²P, ³⁵S, and ¹²⁵I) or a fluorescent label (e.g., fluorescein isothiocyanate, rhodamine, phycoerythrin, phycocyanin, allophycocyanin, o-phthaldehyde and fluorescamine) to enable detection of an interaction between the HAI-TMIP variant or complex and its ligand. Alternatively, the ability of compounds to inhibit a HAI-TMIP variant or a complex of the invention from binding to its ligand can be determined in cell-free assays. For example, a HAI-TMIP variant or a complex of the invention can be contacted with a compound and the ability of the compound to inhibit the HAI-TMIP variant or complex of the invention from binding to its ligand can be determined. In specific embodiments, the compound is immobilized on a solid support and the HAI-TMIP variant or complex of the invention is labelled with a detectable agent. Alternatively, a HAI-TMIP variant or complex of the invention is immobilized on a solid support and the compound is labelled with a detectable agent. The HAI-TMIP variant or complex of the invention may be partially or completely purified (e.g., partially or completely free of other polypeptides) or part of a cell lysate. Further, a HAI-TMIP variant or a component of the complex may be a fusion protein comprising a HAI-TMIP variant, a derivative, analog or fragment thereof and a domain such as glutathionine-S-transferase. Alternatively, a HAI-TMIP variant can be biotinylated using techniques well known to those of skill in the art (e.g., biotinylation kit, Pierce Chemicals; Rockford, Ill.).

### 5.14.2 Phenotypic Readout

The compounds identified in the assays described supra (for convenience referred to herein as a “lead” compound) can be tested for biological activity using host cells containing or engineered to contain HAI-TMIP variant 1, 2, 3, 4, 5, 6 or 7, or a complex of the invention coupled to a functional readout system.

In one embodiment, the effect of a lead compound can be assayed by measuring the cell growth or viability of the target cell. Such assays can be carried out with representative cells of cell types involved in a disorder, e.g., cancer cells or endothelial cells. A lower level of proliferation or survival of the contacted cells indicates that the lead compound is effective to treat a condition in the patient characterized by uncontrolled cell growth. Alternatively, instead of culturing cells from a patient, a lead compound may be screened using cells of a tumor or malignant cell line or an endothelial cell line. Specific examples of cell culture models include, but are not limited to, for lung cancer, primary rat lung tumor cells (Swafford et al., 1997, Mol. Cell. Biol., 17:1366 1374) and large-cell undifferentiated cancer cell lines (Mabry et al., 1991, Cancer Cells, 3:53 58); colorectal cell lines for colon cancer (Park and Gazdar, 1996, J. Cell Biochem. Suppl. 24:131 141); multiple established cell lines for breast cancer (such as MDA-MB-231, MCF-7 and MCF-7/A; e.g., Hambly et al., 1997, Breast Cancer Res. Treat. 43:247 258; Gierthy et al., 1997, Chemosphere 34:1495 1505; Prasad and Church, 1997, Biochem. Biophys. Res. Commun. 232:14 19); a number of well characterized cell models for prostate cancer (Webber et al., 1996, Prostate, Part 1, 29:386 394; Part 2, 30:58 64; and Part 3, 30:136 142; Boulikas, 1997, Anticancer Res. 17:1471 1505); for genitourinary cancers, continuous human bladder cancer cell lines (Ribeiro et al., 1997, Int. J. Radiat. Biol. 72:11 20); organ cultures of transitional cell carcinomas (Booth et al., 1997, Lab Invest. 76:843 857) and rat progression models (Vet et al., 1997, Biochim. Biophys Acta 1360:39 44); melanoma cells (e.g., MDA-MB-435) and established cell lines for leukemias and lymphomas (Drexler, 1994, Leuk. Res. 18:919 927, Tohyama, 1997, Int. J. Hematol. 65:309 317). More specific examples of cell lines include the cancer cell line Huh7 (human hepatocellular carcinoma cell line) and the cancer cell line Caco-2 (a colon-cancer cell line). In certain embodiments, the effect of a lead compound on the growth and/or viability of a cancerous cell or a transformed cell is compared to the effect of such a compound on the growth and/or viability of non-cancerous, normal cells. Preferably, compounds that differentially affect the growth and/or viability of cancerous cells or transformed cells are chosen as anti-proliferative agents.

Many assays well-known in the art can be used to assess the survival and/or growth of a patient cell or cell line following exposure to a lead compound; for example, cell proliferation can be assayed by measuring Bromodeoxyuridine (BrdU) incorporation (, e.g., Hoshino et al., 1986, Int. J. Cancer 38, 369; Campana et al., 1988, J. Immunol. Meth. 107:79) or (³H) thymidine incorporation (, e.g., Chen, J., 1996, Oncogene 13:1395-403; Jeoung, J., 1995, J. Biol. Chem. 270:18367 73), by direct cell count, by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as Western blotting or immunoprecipitation using commercially available antibodies. mRNA can be quantitated using methods that are well known and routine in the art, for example, using northern analysis, RNase protection, and the polymerase chain reaction in connection with the reverse transcription. Cell viability can be assessed by using trypan-blue staining or other cell death or viability markers known in the art. In a specific embodiment, the level of cellular ATP is measured to determined cell viability. Differentiation can be assessed, for example, visually based on changes in morphology.

The lead compound can also be assessed for its ability to inhibit cell transformation (or progression to malignant phenotype) in vitro. In this embodiment, cells with a transformed cell phenotype are contacted with a lead compound, and examined for a change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc. (see Luria et al., 1978, General Virology, 3d Ed., John Wiley & Sons, New York, pp. 436-446).

Loss of invasiveness or decreased adhesion can also be assessed to demonstrate the anti-cancer effects of a lead compound. For example, an aspect of the formation of a metastatic cancer is the ability of a precancerous or cancerous cell to detach from primary site of disease and establish a novel colony of growth at a secondary site. The ability of a cell to invade peripheral sites reflects its potential for a cancerous state. Loss of invasiveness can be measured by a variety of techniques known in the art including, for example, induction of E cadherin mediated cell cell adhesion. Such E cadherin mediated adhesion can result in phenotypic reversion and loss of invasiveness (Hordijk et al., 1997, Science 278:1464-66).

Loss of invasiveness can further be examined by inhibition of cell migration. A variety of 2-dimensional and 3-dimensional cellular matrices are commercially available (Calbiochem Novabiochem Corp. San Diego, Calif.). Cell migration across or into a matrix can be examined using microscopy, time-lapsed photography or videography, or by any method in the art allowing measurement of cellular migration. In a related embodiment, loss of invasiveness is examined by response to hepatocyte growth factor (HGF). HGF-induced cell scattering is correlated with invasiveness of cells such as Madin Darby canine kidney (MDCK) cells. This assay identifies a cell population that has lost cell scattering activity in response to HGF (Hordijk et al., 1997, Science 278:1464-66).

Alternatively, loss of invasiveness can be measured by cell migration through a chemotaxis chamber (Neuroprobe/Precision Biochemicals Inc. Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated can then be correlated with invasiveness (see e.g., Ohnishi, T., 1993, Biochem. Biophys. Res. Commun. 193:518 25).

A lead compound can be assayed (both in vitro and in vivo) for its ability to modulate endothelial cell migration. Any assay known in the art can be used to measure endothelial cell migration. For example, migration can be evaluated in a Boyden chamber migration assay. Briefly, endothelial cells (e.g., smooth muscle cell) are added to the upper well of the chamber. Following cell attachment, one or more lead compounds added to the upper chamber. Cells are allowed to migrate to the lower chamber either with or without an attractant (e.g., PDGF) added to the medium of the lower chamber. Cells which migrated through to the lower chamber can be stained and counted.

A lead compound can be tested for its ability to modulate secretion of extracellular matrix molecules, such as fibronectin, or matrix metalloproteinases. Any method known in the art to assay for extracellular matrix molecule and matrix metalloproteinase production or secretion can be used to quantitate differences in in vitro or in vivo endothelial cells that have been either treated or untreated with lead compounds. For example, western or northern blot analysis, reverse transcription-polymerase chain reaction, or ELISA assays can be used to quantitate expression levels. The activity of matrix metalloproteinases can be assayed by any method known in the art including zymography (see e.g., Badier Commander, 2000, J. Pathol. 192:105-112).

In certain embodiments, a lead compound is tested for its effects, such as, but not limited to, cytotoxicity, altered gene expression, and altered morphology, on PBMCs (Peripheral Blood Mononuclear Cells).

### 5.14.3 Animal Models

The lead compounds identified in the assays described herein can be tested for biological activity using animal models. These include animals engineered to contain HAI-TMIP variant 1, 2, 3, 4, 5, 6 or 7, or a complex of the invention coupled to a functional readout system, such as a transgenic mouse. Such animal model systems include, but are not limited to, rats, mice, chicken, cows, monkeys, pigs, dogs, rabbits, etc. In a specific embodiment of the invention, a compound identified in accordance with the methods of the invention is tested in a mouse model system. Such model systems are widely used and well-known to the skilled artisan such as the SCID mouse model or transgenic mice.

The anti-angiogenic activity of a compound identified in accordance with the invention can be determined by using various experimental animal models of vascularized tumors. The anti-tumor activity of a compound identified in accordance with the invention can be determined by administering the compound to an animal model and verifying that the compound is effective in reducing the proliferation or spread of cancer cells in said animal model. An example of an animal model for human cancer in general includes, but is not limited to, spontaneously occurring tumors of companion animals (, e.g., Vail & MacEwen, 2000, Cancer Invest 18(8):781-92).

Examples of animal models for lung cancer include, but are not limited to, lung cancer animal models described by Zhang & Roth (1994, In Vivo 8(5):755-69) and a transgenic mouse model with disrupted p53 function (, e.g., Morris et al., 1998, J La State Med Soc 150(4):179-85). An example of an animal model for breast cancer includes, but is not limited to, a transgenic mouse that overexpresses cyclin D1 (, e.g., Hosokawa et al., 2001, Transgenic Res 10(5):471-8). An example of an animal model for colon cancer includes, but is not limited to, a TCRbeta and p53 double knockout mouse (, e.g., Kado et al., 2001, Cancer Res 61(6):2395-8). Examples of animal models for pancreatic cancer include, but are not limited to, a metastatic model of Panc02 murine pancreatic adenocarcinoma (, e.g., Wang et al., 2001, Int J Pancreatol 29(1):37-46) and nu-nu mice generated in subcutaneous pancreatic tumours (, e.g., Ghaneh et al., 2001, Gene Ther 8(3):199-208). Examples of animal models for non-Hodgkin's lymphoma include, but are not limited to, a severe combined immunodeficiency (“SCID”) mouse (, e.g., Bryant et al., 2000, Lab Invest 80(4):553-73) and an IgHmu-HOX11 transgenic mouse (, e.g., Hough et al., 1998, Proc Natl Acad Sci USA 95(23):13853-8). An example of an animal model for esophageal cancer includes, but is not limited to, a mouse transgenic for the human papillomavirus type 16 E7 oncogene (, e.g., Herber et al., 1996, J Virol 70(3):1873-81). Examples of animal models for colorectal carcinomas include, but are not limited to, Apc mouse models (, e.g., Fodde & Smits, 2001, Trends Mol Med 7(8):369-73 and Kuraguchi et al., 2000, Oncogene 19(50):5755-63).

In certain embodiments, the animal model is a model system for vascular wound healing, for degenerated, leisured or insured tissue. Models for wound healing include sores, lesions, ulcers and bedsores. The lead compounds of the invention can be tested for their ability to facilitate, promote and/or enhance the process of wound healing.

Examples of animal models for non-alcoholic fatty liver disease (NAFLD) include, but are not limited to, genetically obese ob/ob mice, and lipoatrophic mice, and normal rats fed choline-deficient, methionine-restricted diets (see, e.g., Koteish A 2002, Best Pract Res Clin Gastroenterol 16(5):679-690 and Anstee QM 2006 Int J Exp Pathol 87(1):1-16). Examples of animal models for atherosclenosis include, but are not limited to, low or high cholesterol diet in rabbits (Hakimoglu F., 2006 Atherosclerosis August 8), and apolipoprotein E knockout mice. (Behr-Roussel D. 2006, J Sex Med. Jul. 3(4):596-603.)

Examples of animal models for rheumatoid arthritis include, but not limited to, adjuvant arthritis (AA) and collagen type-II induced arthritis (CIA) both in Lewis rats and in DBA-1 mice (Kwasny-Krachin B., 2002, Amino Acids 23(4):419-426) and in spontaneously developing polyarthritis in MRL/1 mice (Abe C., 1995; Int J Tissue React. 17(5-6):175-180). Examples of animal models for Crohn's disease include, but not limited to, dextran sulfate sodium (DSS)-induced colitis in BALB/C mice (Araki Y., 2006; Int J Mol Med. 17(2):331-334) and trinitrobenzene sulfonic acid (TNBS)-induced colitis in mice (Arranz A., 2006; Ann N Y Acad Sci. 1070:129-134). Examples of animal models for focal/whole cerebral ischemia/reperfusion include, but not limited to, transient or permanent middle cerebral artery occlusion both in rats and mice (Haddad M., 2006; Br J Pharmacol. 149(1):23-30, and Lammer A., 2006; Eur J Neurosci. 23(10):2824-2828), and bilateral common carotid artery and middle cerebral artery occlusion in gerbils (Dhar A., 2006; Pharmacol Res. 54(4):311-316).

### 5.14.4 Toxicity

The toxicity and/or efficacy of a compound identified in accordance with the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). Cells and cell lines that can be used to assess the cytotoxicity of a compound identified in accordance with the invention include, but are not limited to, peripheral blood mononuclear cells (PBMCs), Caco-2 cells, and Huh7 cells. The dose ratio between toxic and therapeutic effects is the therapeutic index and it can be expressed as the ratio LD₅₀/ED₅₀. A compound identified in accordance with the invention that exhibits large therapeutic indices is preferred. While a compound identified in accordance with the invention that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of a compound identified in accordance with the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the compound that achieves a half-maximal inhibition of symptoms) as determined in cell culture. Such information can be used to more accurately determine useful doses in humans. Levels in plasma may be measured, for example, by high performance liquid chromatography.

### 5.15 Agents that Inhibit or Reduce Expression and/or Activity of a HAI-TMIP Variant

### 5.15.1 Antisense Oligonucleotides

In some embodiments of the present invention, the expression of a HAI-TMIP variant is reduced or inhibited by the use of antisense nucleic acids specific to the HAI-TMIP variant, and in some embodiments, as a result, the activity and formation of a complex of the invention is reduced or inhibited. In a specific embodiment of the present invention, the expression of HAI-TMIP variant 3, 4, 5, 6 or 7 is reduced or inhibited by the use of antisense nucleic acids specific to HAI-TMIP variant 3, 4, 5, 6 or 7, and in some embodiments, as a result, the activity and formation of a complex of the invention is reduced or inhibited. The present invention provides the therapeutic or prophylactic use of nucleic acids of at least six nucleotides that are antisense to a gene or cDNA encoding a component protein, or a portion thereof. An “antisense” nucleic acid as used herein refers to a nucleic acid capable of hybridizing to a portion of a component protein RNA (preferably mRNA) by virtue of some sequence complementarity. The antisense nucleic acid may be complementary to a coding and/or noncoding region of a component protein mRNA.

In a specific embodiment, the present invention is directed to a method for inhibiting or reducing the expression of HAI-TMIP variant (e.g., HAI-TMIP variant 1, 2, 3, 4, 5, 6 or 7) nucleic acid sequences, in a prokaryotic or eukaryotic cell, comprising providing the cell with an effective amount of a composition comprising an antisense nucleic acid of the component protein, or a derivative thereof, of the invention.

The antisense nucleic acids are of at least six nucleotides and are preferably oligonucleotides, ranging from 6 to about 200 nucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures, or derivatives or modified versions thereof, and either single-stranded or double-stranded. The oligonucleotide can be modified at the base moiety, sugar moiety, or phosphate backbone. The oligonucleotide may include other appending groups such as peptides, agents facilitating transport across the cell membrane (, e.g., Letsinger et al., 1989, Proc. Natl. Acad. Sci. U.S.A. 86:6553-6556; Lemaitre et al., 1987, Proc. Natl. Acad. Sci. 84:648-652; International Patent Publication No. WO 88/09810) or blood-brain barrier (, e.g., International Patent Publication No. WO 89/10134), hybridization-triggered cleavage agents (, e.g., Krol et al., 1988, BioTechniques 6:958-976), or intercalating agents (, e.g., Zon, 1988, Pharm. Res. 5:539-549).

In a specific aspect of the invention, an antisense oligonucleotide is provided, preferably as single-stranded DNA. The oligonucleotide may be modified at any position in its structure with constituents generally known in the art.

The antisense oligonucleotides may comprise at least one modified base moiety which is selected from the group including but not limited to 5 fluorouracil, 5 bromouracil, 5 chlorouracil, 5 iodouracil, hypoxanthine, xanthine, 4 acetylcytosine, 5 (carboxyhydroxylmethyl)uracil, 5 carboxymethylaminomethyl 2 thio-uridine, 5 carboxymethylaminomethyluracil, dihydrouracil, beta D galactosylqueosine, inosine, N6 isopentenyladenine, 1 methylguanine, 1 methylinosine, 2,2 dimethylguanine, 2 methyladenine, 2 methylguanine, 3 methylcytosine, 5 methylcytosine, N6 adenine, 7 methylguanine, 5 methylaminomethyluracil, 5 methoxyaminomethyl 2 thiouracil, beta D mannosylqueosine, 5N methoxycarboxymethyluracil, 5 methoxyuracil, 2 methyl-thio N6 isopentenyladenine, uracil 5 oxyacetic acid (v), wybutoxosine, pseudouracil, queosine, 2 thiocytosine, 5 methyl 2 thiouracil, 2 thiouracil, 4 thiouracil, 5 methyluracil, uracil 5 oxyacetic acid methylester, uracil 5 oxyacetic acid (v), 5 methyl 2 thiouracil, 3 (3 amino 3 N 2 carboxypropyl) uracil, (acp3)w, and 2,6 diaminopurine.

In another embodiment, the oligonucleotide comprises at least one modified sugar moiety selected from the group including, but not limited to, arabinose, 2 fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of the foregoing.

In yet another embodiment, the oligonucleotide is a 2-a-anomeric oligonucleotide. An a-anomeric oligonucleotide forms specific double-stranded hybrids with complementary RNA in which, contrary to the usual B units, the strands run parallel to each other (Gautier et al., 1987, Nucl. Acids Res. 15:6625-6641).

The oligonucleotide may be conjugated to another molecule, e.g., a peptide, hybridization-triggered cross-linking agent, transport agent, hybridization-triggered cleavage agent, etc.

Oligonucleotides of the invention may be synthesized by standard methods known in the art, e.g., by use of an automated DNA synthesizer (such as are commercially avail-able from Biosearch, Applied Biosystems, etc.). As examples, phosphorothioate oligo-nucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, the antisense oligonucleotides comprise catalytic RNAs, or ribozymes (, e.g., International Patent Publication No. WO 90/11364; Sarver et al., 1990, Science 247:1222-1225). In another embodiment, the oligonucleotide is a 2′-0-methylribonucleotide (Inoue et al., 1987, Nucl. Acids Res. 15:6131-6148), or a chimeric RNA-DNA analog (Inoue et al., 1987, FEBS Lett. 215:327-330).

In an alternative embodiment, the antisense nucleic acids of the invention are produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the component protein. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art to be capable of replication and expression in mammalian cells. Expression of the sequences encoding the antisense RNAs can be by any promoter known in the art to act in mammalian, preferably human, cells. Such promoters can be inducible or constitutive. Such promoters include, but are not limited to, the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of HAI-TMIP variant 3, 4, 5, 6 or 7. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a component protein RNA it may contain and still form a stable duplex (or triplex, as the case may be). One skilled in the art can ascertain a tolerable degree of mismatch by use of standard procedures to determine the melting point of the hybridized complex.

HAI-TMIP variant (e.g., HAI-TMIP 1, 2, 3, 4, 5, 6 or 7) antisense nucleic acids can be used to treat (or prevent) disorders of a cell type that expresses, or preferably overexpresses, HAI-TMIP variant (e.g., HAI-TMIP variant 1, 2, 3, 4, 5, 6 or 7), or a complex of the invention.

Cell types that express or overexpress HAI-TMIP variant 3, 4, 5, 6 or 7 can be identified by various methods known in the art. Such methods include, but are not limited to, hybridization with HAI-TMIP variant 1, 2, 3, 4, 5, 6 or 7-specific nucleic acids (e.g., by Northern blot hybridization, dot blot hybridization, or in situ hybridization), or by observing the ability of RNA from the cell type to be translated in vitro into the component protein by immunohistochemistry, Western blot analysis, ELISA, etc. In a preferred aspect, primary tissue from a patient can be assayed for protein expression prior to treatment, e.g., by immunocytochemistry, in situ hybridization, or any number of methods to detect protein or mRNA expression.

In some embodiments of the invention, compositions of the invention (See Section 5.17), comprise a HAI-TMIP variant (e.g., HAI-TMIP variant 1, 2, 3, 4, 5, 6 or 7) antisense nucleic acid in a pharmaceutically acceptable carrier.

### 5.15.2 RNA Interference

In certain embodiments, an RNA interference (RNAi) molecule is used to decrease the expression of a HAI-TMIP variant (e.g., HAI-TMIP variant 1, 2, 3, 4, 5, 6 or 7). In specific embodiments, an RNA interference (RNAi) molecule is used to decrease the expression of HAI-TMIP variant 3, 4, 5, 6 or 7. Non-limiting examples of miRNA oligonucleotide that can downregulate the expression of HAI-TMIP expression include those in Table 6 below.

TABLE 6

| | | Corresponding |
|---|---|---|
| | | Nucleotides |
| Name of | | of HAI-TMIP |
| mRNAi | Sequence | variant 1 |
| miRNA320 | GCTG AGAGGTATCAGCTCCAA | 320-340 |
| | GAATGTTTTGGCCACTGACTGA | |
| | CATTCTTGGCT GATACCTCTC | |
| | AGG | |
| | (SEQ ID NO: 31) | |
| miRNA483 | TGCT GCAA ACACGACAACAC | 483-503 |
| | CAACATGTTTTGGCCACTGACT | |
| | GACAT GTTGGTTGTCGTGTTT | |
| | GCAGG | |
| | (SEQ ID NO: 32) | |
| miRNA565 | TGC TGAACAGCTCCTCACCAA | 565-585 |
| | CTGGAGTTTTGGCCACTGACTG | |
| | ACTCCAGTTGGAGGAGCTGTTC | |
| | AGG | |
| | (SEQ ID NO: 33) | |
| miRNA591 | TGCT GTACCAAGGGCATCAAC | 591-611 |
| | TACACGTTTTGGCCACTGACTG | |
| | ACGTGTAGTTT GCCCTTGGTA | |
| | CAGG | |
| | (SEQ ID NO:34) | |
| miRNA755 | TGCTG AATCAGTTCACGCTGA | 755-775 |
| | CCACGGTTTTGGCCACTGACTG | |
| | ACCGTGGTCAGT GAACTGATT | |
| | CAGG | |
| | (SEQ ID NO: 35) | |

RNA interference (RNAi) is the ability of double-stranded RNA (dsRNA) to suppress the expression of a gene corresponding to its own sequence (, e.g., Cogoni and Macino, 2000, Genes Dev 10: 638-643, Guru, 2000, Nature 404, 804-808, Hammond et al., 2001, Nature Rev Gen 2: 110-119, Shi, 2003, Trends Genet. 19:9-12, U.S. Pat. No. 6,506,559, each incorporated by reference in their entireties herein). RNAi is also called post-transcriptional gene silencing or PTGS. Without being bound by theory, since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

The current models of the RNAi mechanism includes both initiation and effector steps (Hutvagner and Zamore, 2002, Curr Opin Genetics & Development 12:225-32; Hammond et al., 2001, Nature Rev Gen 2: 110-9, each incorporated by reference in their entireties herein). In the initiation step, input dsRNA is digested into 21-23 nucleotide small interfering RNAs (siRNAs), which have also been called “guide RNAs” (Sharp, 2001, Genes Dev 15: 485-490). Evidence indicates that siRNAs are produced when the enzyme Dicer, a member of the RNase III family of dsRNA-specific ribonucleases, processively cleaves dsRNA (introduced directly or via a transgene or virus) in an ATP-dependent, processive manner. Successive cleavage events degrade the RNA to 19-21 base pair duplexes (siRNAs), each with 2-nucleotide 3′ overhangs (Bernstein et al., 2001, Nature 409:363-366; Hutvagner and Zamore, 2002, Curr Opin Genetics & Development 12:225-232). In the effector step, the siRNA duplexes bind to a nuclease complex to form what is known as the RNA-induced silencing complex, or RISC. An ATP-depending unwinding of the siRNA duplex is required for activation of the RISC. The active RISC then targets the homologous transcript by base pairing interactions and cleaves the mRNA ˜12 nucleotides from the 3′ terminus of the siRNA. Although the mechanism of cleavage is at this date unclear, research indicates that each RISC contains a single siRNA and an RNase that appears to be distinct from Dicer (Hutvagner and Zamore, 2002, Curr Opin Genetics & Development 12:225-232). Because of the remarkable potency of RNAi in some organisms, an amplification step within the RNAi pathway has also been proposed. Amplification could occur by copying of the input dsRNAs, which would generate more siRNAs, or by replication of the siRNAs themselves. Alternatively or in addition, amplification could be effected by multiple turnover events of the RISC.

Elbashir and colleagues (Elbashir et al., 2001, Nature 411:494-498; Elbashir et al., 2001, EMBO 20:6877-6888) have suggested a procedure for designing siRNAs for inducing RNAi in mammalian cells. Briefly, find a 21 nucleotide sequence in the mRNA of interest that begins with an adenine-adenine (AA) dinucleotide as a potential siRNA target site. This strategy for choosing siRNA target sites is based on the observation that siRNAs with 3′ overhanging UU dinucleotides are the most effective. This is also compatible with using RNA pol III to transcribe hairpin siRNAs because RNA pol III terminates transcription at 4-6 nucleotide poly(T) tracts creating RNA molecules with a short poly(U) tail. Although siRNAs with other 3′ terminal dinucleotide overhangs have been shown to effectively induce RNAi, siRNAs with guanine residues in the overhang are not recommended because of the potential for the siRNA to be cleaved by RNase at single-stranded guanine residues. In addition to beginning with an AA dinucleotide, the siRNA target site should have a guanosine and cytidine residue percentage within the range of 30-70%. The chosen siRNA target sequence should then be subjected to a BLAST search against the EST database to ensure that only the desired gene is targeted. Various products are commercially available to aid in the preparation and use of siRNA (e.g., Ambion, Inc., Austin, Tex.).

Double-stranded (ds) RNA can be used to interfere with gene expression in mammals (Brummelkamp et al., Science 296:550-3, Krichevsky and Kosik, 2002, PNAS 99:11926-9, Paddison et al., 2002, PNAS 99:1443-8, Wiarmy & Zernicka-Goetz, 2000, Nature Cell Biology 2:70-75, European Patent 1144623, International Patent Publication Nos. WO 02/055693, WO 02/44321, WO 03/006,477; each incorporated by reference in their entireties herein).

### 5.15.3 Aptamers

In certain embodiments, the invention provides aptamers of a HAI-TMIP variant (e.g., HAI-TMIP variant 1, 2, 3, 4, 5, 6, or 7). In specific embodiments, the invention provides aptamers of HAI-TMIP variants 3, 4, 5, 6 and 7. As is known in the art, aptamers are macromolecules composed of nucleic acid (e.g., RNA, DNA) that bind tightly to a specific molecular target (e.g., HAI-TMIP variant 3, 4, 5, 6 or 7). A particular aptamer may be described by a linear nucleotide sequence and is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind to a HAI-TMIP variant (e.g., HAI-TMIP variant 1, 2, 3, 4, 5, 6 or 7) can be achieved through methods known in the art. For example, aptamers can be selected using the SELEX (Systematic Evolution of Ligands by Exponential Enrichment) method (Tuerk and Gold, 1990, Science 249:505-510, which is incorporated by reference herein in its entirety). In the SELEX method, a large library of nucleic acid molecules (e.g., 10.sup.15 different molecules) is produced and/or screened with the target molecule (e.g., HAI-TMIP variant 1, 2, 3, 4, 5, 6 or 7). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology, the entire teachings of which are incorporated herein by reference).

### 5.15.4 Antibodies

The present invention provides antibodies that immunospecifically bind to HAI-TMIP variant (e.g., HAI-TMIP variant 1, 2, 3, 4, 5, 6, or 7) and inhibits or reduces the binding/association of the subunit to other subunits of the ATP synthase. In a specific embodiment, the antibody immunospecifically binds to a HAI-TMIP variant (e.g., HAI-TMIP variant 1, 2, 3, 4, 5, 6, or 7) and inhibits or reduces its binding to other subunits of ATP synthase. The present invention also provides antibodies that immunospecifically bind to a HAI-TMIP variant (e.g., HAI-TMIP variant 1, 2, 3, 4, 5, 6 or 7) and inhibit or reduce the formation, stability and/or activity of the F₁ ATP synthase subcomplex or F₀F₁ ATP synthase holoenzyme. In a specific embodiment, the present invention provides antibodies that immunospecifically bind to a HAI-TMIP variant (e.g., HAI-TMIP variant 1, 2, 3, 4, 5, 6 or 7) and inhibit or reduce the activity of the F₁ ATP synthase subcomplex or F₀F₁ ATP synthase holoenzyme. The present invention also provides antibodies that immunospecifically bind to a complex of the invention and inhibit or reduce the stability and/or activity of the complex. Methods for assessing whether an antibody inhibits or reduces the activity of F₁ ATP synthase subcomplex or F₀F₁ ATP synthase holoenzyme are well-known in the art and are described herein. In certain embodiments, the antibody competes with a ligand (e.g., angiostatin) of the HAI-TMIP variant or the complex for binding to the subunit or the complex.

The present invention also provides antibody conjugates that inhibit or reduce the binding/association of ATP synthase subunits to each other. The present invention also provides antibody conjugates that inhibit or reduce the formation, stability and/or activity of F₁ ATP synthase subcomplex or F₀F₁ ATP synthase holoenzyme. In a specific embodiment, the antibody conjugate comprising an antibody that immunospecifically binds to a HAI-TMIP variant (e.g., HAI-TMIP variant 1, 2, 3, 4, 5, 6 or 7) or a complex of the invention, and a drug, modifier or toxin, such as presented in Section 5.8.2.

In some embodiments, a composition of the invention comprises an antibody described in this Section 5.15.4, and a pharmaceutically acceptable carrier.

### 5.16 Agents that Increase Expression and/or Activity of a HAI-TMIP Variant

In some embodiments of the invention, the nucleotide sequences of a HAI-TMIP variant (e.g., HAI-TMIP variant 1, 2, 3, 4, 5, 6, or 7) are used in gene therapy to increase the level of expression of the HAI-TMIP variant (e.g., HAI-TMIP variant 1, 2, 3, 4, 5, 6, or 7). In specific embodiments of the invention, the nucleotide sequences described in Section 5.1, supra, are used in gene therapy to increase the level of expression of HAI-TMIP variant 3, 4, 5, 6 or 7. Any of the methods for gene therapy available in the art can be used according to the present invention.

The present invention provides antibodies that immunospecifically bind to a HAI-TMIP variant and increase the binding/association of the subunit to other subunits of the ATP synthase. In a specific embodiment, the antibody immunospecifically binds to HAI-TMIP variant 3, 4, 5, 6 or 7 and increases its binding to other subunits of the ATP synthase. The present invention also provides antibodies that immunospecifically bind to a HAI-TMIP variant and increases the formation, stability and/or activity of the F₁ ATP synthase subcomplex or F₀F₁ ATP synthase holoenzyme. In a specific embodiment, the present invention provides antibodies that immunospecifically bind to HAI-TMIP variant 3, 4, 5, 6 or 7 and increase the formation, stability and/or activity of the F₁ ATP synthase subcomplex or F₀F₁ ATP synthase holoenzyme. The present invention also provides antibodies that immunospecifically bind to a complex of the invention and inhibit or reduce the stability and/or activity of the complex. Methods for assessing whether an antibody increases the activity of F₀F₁ ATP synthase holoenzyme are well-known in the art and are described herein. In certain embodiments, the antibody competes with a ligand (e.g., angiostatin) of the HAI-TMIP variant or the complex for binding to the subunit or the complex.

In some embodiments, a composition of the invention comprises an antibody described in this Section 5.16, and a pharmaceutically acceptable carrier.

### 5.17 Pharmaceutical Compositions, Routes of Administration & Dosage

The present invention provides compositions comprising a carrier and one the following or a combination of two or more of the following: (i) a nucleotide sequence described in Section 5.1, supra; (ii) a protein described in Section 5.2, supra; (iii) a complex of the invention; (iv) an antibody that immunospecifically binds to a HAI-TMIP variant (e.g, HAI-TMIP variant 1, 2, 3, 4, 5, 6 or 7), an F₁ ATP synthase subcomplex or an F₀F₁ ATP synthase holoenzyme (e.g., a complex of the invention); (v) a compound that modulates the expression of a HAI-TMIP variant (e.g., HAI-TMIP variant 1, 2, 3, 4, 5, 6 or 7); (v) a compound that modulates that activity of a HAI-TMIP variant (e.g., HAI-TMIP variant 1, 2, 3, 4, 5, 6 or 7); (vi) a compound that modulates the formation of an F₁ ATP synthase subcomplex or an F₀F₁ ATP synthase holoenzyme (e.g., a complex of the invention); and/or (vii) a compound that modulates the ATP synthase activity of an F₁ ATP synthase subcomplex or an F₀F₁ ATP synthase holoenzyme (e.g., a complex of the invention). In a preferred embodiment, the compositions are pharmaceutical compositions. In accordance with this embodiment, the pharmaceutical compositions are preferably sterile and in suitable form for the intended method of administration or use. In certain embodiments, the pharmaceutical compositions of the invention comprise a pharmaceutically acceptable carrier, and in other embodiments, the pharmaceutical compositions further comprise one or more other prophylactic or therapeutic agents. The invention encompasses the use of the compositions of the invention in the prevention, treatment and/or management of a disorder described herein.

The compositions of the invention include, but are not limited to, bulk drug compositions useful in the manufacture of pharmaceutical compositions (e.g., impure or non-sterile compositions) and pharmaceutical compositions (i.e., compositions that are suitable for administration to a subject) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is contained in or administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Compounds identified using the methods of the invention or a pharmaceutically acceptable salt thereof, complexes of the invention, proteins described in Section 5.2, supra, nucleotide sequences described in Section 5.1, supra, antibodies that immunospecifically bind to HAI-TMIP variant 1, 2, 3, 4, 5, 6 or 7, or a complex of the invention, antisense oligonucleotides, aptamers, and RNAi can be administered to a patient in need thereof, preferably a mammal in need thereof, more preferably a human in need thereof, to prevent, treat and/or manage a disorder described herein. In this section, compounds identified using the methods of the invention or a pharmaceutically acceptable salt thereof, complexes of the invention, a HAI-TMIP variant (e.g., a protein described in Section 5.2, supra), nucleotide sequences of a HAI-TMIP variant (e.g., the nucleotide sequences described in Section 5.1, supra), antibodies that immunospecifically bind to a HAI-TMIP variant (e.g., HAI-TMIP variant 1, 2, 3, 4, 5, 6 or 7), or an F₁ ATP synthase subcomplex or an F₀F₁ ATP synthase holoenzyme (e.g., a complex of the invention), antisense oligonucleotides, aptamers, and RNAi are collectively referred to as compound to be used with the therapeutic and prophylactic methods of the invention. In a specific embodiment, a compound to be used with the prophylactic methods of the invention is administered to a patient, preferably a mammal, more preferably a human, as a preventative measure against disorder. In another embodiment, a compound to be used with the therapeutic and prophylactic methods of the invention is administered to a patient, preferably a mammal, more preferably a human, to treat and/or manage a disorder described herein.

When administered to a patient, the compound to be used with the therapeutic and prophylactic methods of the invention is preferably administered as component of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the compound and pharmaceutically acceptable salts thereof.

Routes of administration include but are not limited to intradermal, intramuscular, intraperitoneal, intravenous, subcutaneous, intranasal, epidural, oral, sublingual, intranasal, intracerebral, oral, intra-arterial, intrasynovial, intrathecal, intravaneous, intravaginal, transdermal, rectal, inhalation, and topical, particularly to the ears, nose, eyes, or skin. The mode of administration is left to the discretion of the practitioner. In most instances, administration will result in the release of the compound or a pharmaceutically acceptable salt thereof into the bloodstream.

In specific embodiments, it may be desirable to administer the compound to be used with the therapeutic and prophylactic methods of the invention locally. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce the compound to be used with the therapeutic and prophylactic methods of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound to be used with the therapeutic and prophylactic methods of the invention can be formulated as a suppository, with traditional binders and vehicles such as triglycerides.

In another embodiment, the compound to be used with the therapeutic and prophylactic methods of the invention can be delivered in a vesicle, in particular a liposome (see Langer, 1990, Science 249:1527 1533; Treat et al., in Liposomes in the Therapy of Infectious Disease and Cancer, Lopez-Berestein and Fidler (eds.), Liss, New York, pp. 353 365 (1989); Lopez Berestein, ibid., pp. 317 327; see generally ibid.).

In yet another embodiment, the compound to be used with the therapeutic and prophylactic methods of the invention can be delivered in a controlled release system (, e.g., Goodson, in Medical Applications of Controlled Release, supra, vol. 2, pp. 115 138 (1984)). Other controlled-release systems discussed in the review by Langer, 1990, Science 249:1527 1533 may be used. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of a target RNA of the compound or a pharmaceutically acceptable salt thereof, thus requiring only a fraction of the systemic dose.

In certain embodiments, the compound to be administered to a subject is a nucleotide sequence (e.g., a nucleotide sequence described in Section 5.1, supra, antisense, or RNAi). Delivery of the nucleotide sequences into a subject may be either direct, in which case the subject is directly exposed to the nucleotide sequence or nucleotide sequence-carrying vectors, or indirect, in which case, cells are first transformed with the nucleic acids in vitro, then transplanted into the subject. These two approaches are known, respectively, as in vivo or ex vivo gene therapy. For general reviews of the methods of gene therapy, see Loise, 2006, Methods Mol Biol. 333:201-26; Lawson, 2006, Methods Mol Biol. 333:175-200; Baum et al., 2006, Mol Ther. 13(6):1050-63; Ghosh et al., 2006, Appl Biochem Biotechnol. 133(1):9-29; Loewen et al., 2005, Adv Biochem Eng Biotechnol. 99:169-91; Grieger et al., 2005, Adv Biochem Eng Biotechnol. 99:119-45; Mastrobattista et al., 2006, Nat Rev Drug Discov. 25(2):115-21. Methods commonly known in the art of recombinant DNA technology which can be used are described in Ausubel et al. (eds.), Current Protocols in Molecular Biology, John Wiley & Sons, NY (1993); Ausubel et al. (eds), Short Protocols in Molecular Biology, 5^{th} edition, John Wiley & Sons, NY (2002); Balbas et al., (eds), Recombinant Gene Expression: Reviews and Protocols, 2^{nd} edition, Humana Press Inc., NJ (2004); Bartlett et al. (eds), PCR Protocols, 2^{nd} edition, Humana Press Inc., NJ (2003); and Kriegler, Gene Transfer and Expression, A Laboratory Manual, Stockton Press, NY (1990).

Compositions comprising the compound to be used with the therapeutic and prophylactic methods of the invention (“compound compositions”) can additionally comprise a suitable amount of a pharmaceutically acceptable carrier so as to provide the form for proper administration to the patient.

Compound compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington's Pharmaceutical Sciences, Alfonso R. Gennaro, ed., Mack Publishing Co. Easton, Pa., 19^{th} ed., 1995, pp. 1447 to 1676, incorporated herein by reference.

In a preferred embodiment, the compound to be used with the therapeutic and prophylactic methods of the invention is formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, and the like. Such vehicles are preferably of pharmaceutical grade. Typically, compositions for intravenous administration comprise sterile isotonic aqueous buffer. Where necessary, the compositions may also include a solubilizing agent.

In another embodiment, the compound to be used with the therapeutic and prophylactic methods of the invention can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the compound to be used with the therapeutic and prophylactic methods of the invention is to be administered by infusion, it can be dispensed, for example, with an infusion bottle containing sterile pharmaceutical grade water or saline. Where the compound to be used with the therapeutic and prophylactic methods of the invention is administered by injection, an ampoule of sterile water for injection or saline can be provided so that the ingredients may be mixed prior to administration.

The amount of a compound to be used with the therapeutic and prophylactic methods of the invention that will be effective in the treatment of a particular disease will depend on the nature of the disease, and can be determined by standard clinical techniques. In addition, in vitro or in vivo assays may optionally be employed to help identify optimal dosage ranges. The precise dose to be employed will depend on a variety of factors including the compound employed, the age of the subject, the subject's body weight, the subject's general health, the subject's diet, the route of administration of the compound employed, the frequency of administration of the compound employed, the rate of excretion of the compound employed, the disease, the seriousness of the disease and the drug combination, and should be decided according to the judgment of the practitioner and each patient's circumstances. However, suitable dosage ranges for oral administration are generally about 0.001 milligram to about 500 milligrams of a compound or a pharmaceutically acceptable salt thereof per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound is administered, or if a compound is administered with a therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

The compound and pharmaceutically acceptable salts thereof are preferably assayed in vitro and in vivo, for the desired therapeutic or prophylactic activity, prior to use in humans. For example, in vitro assays can be used to determine whether it is preferable to administer the compound, a pharmaceutically acceptable salt thereof, and/or another therapeutic agent. Animal model systems can be used to demonstrate safety and efficacy.

Exemplary doses of proteins, polypeptides, peptides, fusion proteins and complexes encompassed by the invention is 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg and 0.25 mg/kg, 0.0001 and 0.15 mg/kg, 0.0001 and 0.10 mg/kg, 0.001 and 0.5 mg/kg, 0.01 and 0.25 mg/kg, 0.01 and 0.10 mg/kg or 0.1 and 10 mg/kg of the patient's body weight.

### 5.18 Prophylactic and Therapeutic Uses

### 5.18.1 Disorders Associated with Aberrant Angiogenesis

The present invention also provides methods for reducing or preventing angiogenesis, the methods comprising administering to a subject in need thereof one or more inhibitors. The present invention also provides methods of preventing, treating and/or managing a disorder characterized by, associated with or caused by aberrant angiogenesis, said methods comprising administering to a subject in need thereof one or more inhibitors. In one embodiment, the present invention methods for preventing, treating and/or managing a disorder associated with aberrant angiogenesis said methods comprising administering to a subject one or more inhibitors of HAI-TMIP variant 1, 2, 3, 4, 5, 6, and/or 7, or a complex of the invention. Non-limiting examples of inhibitors include those described in Section 5.15. In a specific embodiment, the inhibitor is a compound that prevents or reduces the binding of a complex of the invention to its ligand and inhibits ATP synthase activity. In certain embodiments, the inhibitor reduces angiogenesis in an angiogenesis model, described herein or known to one of skill in the art, by at least 15%, preferably at least 50%, at least 75%, at least 90%, or at least 95% relative to a negative control (e.g., PBS). In a specific embodiment, the inhibitor reduces angiogenesis in a chick choroallantoic membrane (CAM) model or retina angiogenesis model by at least 25%, preferably at least 50%, at least 75%, at least 90%, or at least 95% relative to a negative control.

Disorders encompassed by the methods of the present invention that are characterized by, associated with or caused by aberrant angiogenesis include, but are not limited to, cancers, asthma, ischemia, atherosclerosis, scleroderma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retinopathy of prematurity, retrolental fibroplasias, diabetic neovascularization, peptic ulcer, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In a specific embodiment, the disorder characterized by, associated with or caused by aberrant angiogenesis is a disorder involving cells (e.g., endothelial cells) that overexpress HAI-TMIP variant 1, 2, 3, 4, 5, 6 and/or 7. In certain embodiments, the cells involved in the disorder express 2 times, 5 times, 10 times, 25 times, or 2 fold, 5 fold or 10 fold more HAI-TMIP variant 1, 2, 3, 4, 5, 6 and/or 7 than the same cell type found in a healthy, disorder-free subject.

The invention also provides methods of preventing, treating and/or managing a disorder characterized by, associated with or caused by aberrant angiogenesis, said methods comprising administering to a subject in need thereof one or more inhibitors and one or more other therapies (e.g., prophylactic or therapeutic agents). In a specific embodiment, the other therapies are currently being used, have been used or are known to be useful in the prevention, treatment and/or management of a disorder characterized by, associated with or caused by aberrant angiogenesis. The therapies (e.g., prophylactic or therapeutic agents) of the combination therapies of the invention can be administered sequentially or concurrently.

In a specific embodiment, the combination therapies of the invention comprise a compound identified in accordance with the invention and at least one other therapy that has the same mechanism of action as said compound. In another specific embodiment, the combination therapies of the invention comprise a compound identified in accordance with the methods of the invention and at least one other therapy (e.g., prophylactic or therapeutic agent) which has a different mechanism of action than said compound. In one embodiment, the combination therapies of the present invention improve the prophylactic or therapeutic effect of a compound of the invention by functioning together with the compound to have an additive or synergistic effect. In another embodiment, the combination therapies of the present invention reduce the side effects associated with the therapies (e.g., prophylactic or therapeutic agents).

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In specific embodiment, the present invention provides methods of preventing, treating and/or managing a disorder characterized by, associated with or caused by aberrant angiogenesis, said methods comprising administering to a subject in need thereof a pharmaceutical composition comprising an inhibitor. In accordance with the invention, the pharmaceutical composition may also comprise one or more other prophylactic or therapeutic agents.

An inhibitor may be used as a first, second, third, fourth or fifth line of therapy to prevent, treat and/or manage a disorder characterized by, associated with or caused by aberrant angiogenesis. In certain embodiments, a subject administered an inhibitor in accordance with the methods of the invention has been diagnosed with a disorder characterized by, associated with or caused by aberrant angiogenesis. In some embodiments, a subject administered an inhibitor in accordance with the methods of the invention is prone or predisposed to a disorder characterized by, associated with or caused by aberrant angiogenesis. In some embodiments, a subject administered an inhibitor in accordance with the methods of the invention has received or is receiving another therapy. Non-limiting examples of the other therapy include surgery, chemotherapy, radiation therapy, hormonal therapy, biological therapy and immunotherapy.

In certain embodiments, a subject administered an inhibitor in accordance with the methods of the invention is refractory to conventional therapies for a disorder characterized by, associated with or caused by aberrant angiogenesis. For example, a cancer may be determined to be refractory to a therapy means when at least some significant portion of the cancer cells are not killed or their cell division arrested in response to the therapy. Such a determination can be made either in vivo or in vitro by any method known in the art for assaying the effectiveness of therapy on cancer cells, using the art-accepted meanings of “refractory” in such a context. In a specific embodiment, a cancer is refractory where the number of cancer cells has not been significantly reduced, or has increased.

In more specific embodiments, a subject administered an inhibitor in accordance with the methods of the invention is refractory to existing single agent therapies for a disorder characterized by, associated with or caused by aberrant angiogenesis. In some embodiments, a subject administered an inhibitor in accordance with the methods of the invention is refractory to therapies other than an inhibitor but is no longer on those therapies.

In some embodiments, a subject administered an inhibitor in accordance with the methods of the invention is immunosuppressed by reason, e.g., of having previously undergone other therapies. In alternative embodiments, a subject administered an inhibitor in accordance with the methods of the invention is immunocompetent.

In some embodiments, a subject administered an inhibitor in accordance with the methods of the invention has experienced or is experiencing an adverse reaction to another therapy. In other embodiments, a subject administered an inhibitor in accordance with the methods of the invention is susceptible to an adverse reaction to another therapy.

In some embodiments, a subject administered an inhibitor in accordance with the methods of the invention is in remission. In some embodiments, a subject administered an inhibitor in accordance with the methods of the invention had a disorder characterized by, associated with or caused by aberrant angiogenesis but is now disorder-free. In other embodiments, a subject administered an inhibitor in accordance with the methods of the invention is has recurrence of a disorder characterized by, associated with or caused by aberrant angiogenesis.

In some embodiments, a subject administered an inhibitor in accordance with the methods of the invention is a non-human animal such as a pet, farm animal, and lab animal. In other embodiments, a subject administered an inhibitor in accordance with the methods of the invention is a human. In a specific embodiment, a subject administered an inhibitor in accordance with the methods of the invention is a human subject with other conditions. In another embodiment, a subject administered an inhibitor in accordance with the methods of the invention is a human infant. In another embodiment, a subject administered an inhibitor in accordance with the methods of the invention is a human child. In another embodiment, a subject administered an inhibitor in accordance with the methods of the invention is a human adult. In yet another embodiment, a subject administered an inhibitor in accordance with the methods of the invention is an elderly human.

### 5.18.2 Cancer

The present invention provides methods for preventing or reducing the proliferation of a cancer cell, the methods comprising contacting an inhibitor with the cancer cell. In a specific embodiment, the inhibitor prevents or reduces cancer cell proliferation by at least 10%, preferably by at least 25%, at least 30%, at least 50%, at least 75%, at least 80%, at least 85%, at least 90%, at least 90% or at least 95% relative to a negative control (e.g., PBS), as measured by an assay described herein or known to one of skill in the art. The invention also provides methods for inducing apoptosis of cancer cells, the methods comprising contacting an inhibitor with the cancer cells. In a specific embodiment, the inhibitor induces at least 10%, preferably at least 25%, at least 50%, at least 75%, at least 80%, at least 85%, at least 90%, or at least 95% of the cancer cells to undergo apoptosis as measured by an assay described herein or known to one of skill in the art.

The present invention provides methods of preventing, treating and/or managing cancer, said methods comprising administering to a subject in need thereof one or more inhibitors. In one embodiment, the present invention provides methods for preventing, treating and/or managing cancer, said methods comprising administering to a subject in need thereof one or more inhibitors of HAI-TMIP variant 1, 2, 3, 4, 5, 6 and/or 7. In a specific embodiment, the cancer being prevented, treated and/or managed involves cancer cells that overexpress HAI-TMIP variant 4, 5, 6, and/or 7. In certain embodiments, the inhibitor is an antibody that immunospecifically binds to HAI-TMIP variant 1, 2, 3, 4, 5, 6 or 7.

The invention also provides methods of preventing, treating and/or managing cancer, said methods comprising administering to a subject in need thereof one or more inhibitors and one or more other therapies (e.g., prophylactic or therapeutic agents). In a specific embodiment, the other therapies are currently being used, have been used or are known to be useful in the prevention, treatment and/or management of cancer. Non-limiting examples of other therapies that can be administered to a subject in combination with an inhibitor are provided in Section 5.18.2.1. The therapies (e.g., prophylactic or therapeutic agents) of the combination therapies of the invention can be administered sequentially or concurrently.

The present invention provides methods of preventing, treating and/or managing cancer, said methods comprising administering to a subject in need thereof one or more activators. In one embodiment, the present invention provides methods of preventing, treating and/or managing cancer, said methods comprising administering to a subject in need thereof one or more activators of HAI-TMIP variant 3, or a complex comprising such a variant In a specific embodiment, the activator is an antibody that immuno specifically binds to HAI-TMIP 3. The invention also provides methods of preventing, treating and/or managing cancer, said methods comprising administering to a subject in need thereof one or more activators of HAI-TMIP variant 3, and one or more other cancer therapies.

In a specific embodiment, the combination therapies of the invention comprise a compound identified in accordance with the invention and at least one other therapy that has the same mechanism of action as said compound. In another specific embodiment, the combination therapies of the invention comprise a compound identified in accordance with the methods of the invention and at least one other therapy (e.g., prophylactic or therapeutic agent) which has a different mechanism of action than said compound. In one embodiment, the combination therapies of the present invention improve the prophylactic or therapeutic effect of a compound of the invention by functioning together with the compound to have an additive or synergistic effect. In another embodiment, the combination therapies of the present invention reduce the side effects associated with the therapies (e.g., prophylactic or therapeutic agents).

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In a specific embodiment, the present invention provides methods of preventing, treating and/or managing cancer, said methods comprising administering to a subject in need thereof a pharmaceutical composition comprising an inhibitor. In another embodiment, the present invention provides methods of preventing, treating and/or managing cancer, said methods comprising administering a pharmaceutical composition comprising an activator. In accordance with the invention, such pharmaceutical compositions may also comprise one or more other prophylactic or therapeutic agents.

An inhibitor or activator may be used as a first, second, third, fourth or fifth line of therapy to prevent, treat and/or manage cancer.

### 5.18.2.1 Other Therapies

Therapeutic or prophylactic agents include, but are not limited to, peptides, polypeptides, proteins, fusion proteins, nucleic acid molecules, small molecules, mimetic agents, synthetic drugs, inorganic molecules, and organic molecules. Any agent or therapy (e.g., chemotherapies, radiation therapies, hormonal therapies, and/or biological therapies/immunotherapies) which is known to be useful, or which has been used or is currently being used for the prevention, treatment and/or management of cancer can be used in combination with an inhibitor in accordance with the invention described herein.

In certain embodiments, the anti-cancer agent is an immunomodulatory agent, such as a chemotherapeutic agent. In certain other embodiments, the anti-cancer agent is an immunomodulatory agent other than a chemotherapeutic agent. In other embodiments, the anti-cancer agent is not an immunomodulatory agent. In specific embodiments, the anti-cancer agent is an anti-angiogenic agent. In other embodiments, the anti-cancer agent is not an anti-angiogenic agent. In specific embodiments, the anti-cancer agent is an anti-inflammatory agent. In other embodiments, the anti-cancer agent is not an anti-inflammatory agent.

In particular embodiments, the anti-cancer agent is, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandomate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; Eph inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-nl; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432 which are incorporated herein by reference in their entirety); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

Other examples of anti-cancer agents include, but are not limited to, angiogenesis inhibitors, topoisomerase inhibitors and immunomodulatory agents (such as chemotherapeutic agents and non-therapeutic immunomodulatory agents, including but not limited to, anti-T cell receptor antibodies (e.g., anti-CD4 antibodies (e.g., cM-T412 (Boeringer), IDEC-CE9.1.RTM. (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-alpha. antibodies, anti-IL-1 beta. antibodies, anti-IL-6 antibodies, anti-IL-8 antibodies (e.g., ABX-IL-8 (Abgenix)), and anti-IL-12 antibodies)); CTLA4-immunoglobulin; soluble cytokine receptors (e.g., the extracellular domain of a TNF-alpha. receptor or a fragment thereof, the extracellular domain of an IL-1 beta. receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, TNF-alpha., TNF-beta., interferon (IFN)-alpha, IFN-beta, IFN-gamma, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-alpha antibodies, and anti-IFN-gamma antibodies), and antibodies that immunospecifically bind to tumor-associated antigens (e.g., HERCEPTIN®).

The invention also encompasses administration of an inhibitor in combination with radiation therapy comprising the use of x-rays, gamma rays and other sources of radiation to destroy the cancer cells. In specific embodiments, the radiation therapy is administered as external beam radiation or teletherapy wherein the radiation is directed from a remote source. In other preferred embodiments, the radiation therapy is administered as internal therapy or brachytherapy wherein a radiaoactive source is placed inside the body close to cancer cells or a tumor mass.

In specific embodiments, patients with breast cancer are administered a prophylactically or therapeutically effective amount of an inhibitor in combination with the administration of a prophylactically or therapeutically effective amount of one or more other agents useful for breast cancer therapy including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), Herceptin®, tamoxifen, the combination of tamoxifen and cytotoxic chemotherapy. In certain embodiments, patients with metastatic breast cancer are administered a prophylactically or therapeutically effective amount of one or more inhibitors in combination with the administration of an effective amount of taxanes such as docetaxel and paclitaxel.

In other embodiments, a prophylactically or therapeutically effective amount of an inhibitor is administered in combination with the administration of a prophylactically or therapeutically effective amount of taxanes plus standard doxorubicin and cyclophosphamide for adjuvant treatment of node-positive, localized breast cancer.

In specific embodiments, patients with prostate cancer are administered a prophylactically or therapeutically effective amount of an inhibitor of the invention in combination with the administration of a prophylactically or therapeutically effective amount of one or more other agents useful for prostate cancer therapy including but not limited to: external-beam radiation therapy, interstitial implantation of radioisotopes (i.e., 12, palladium, Iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel.

In specific embodiments, patients with ovarian cancer are administered a prophylactically or therapeutically effective amount of an inhibitor in combination with a prophylactically or therapeutically effective amount of one or more other agents useful for ovarian cancer therapy including but not limited to: intraperitoneal radiation therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. It is contemplated that a prophylactically or therapeutically effective amount of an inhibitor is administered in combination with the administration Taxol for patients with platinum-refractory disease. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors. In specific embodiments, patients with bone sarcomas are administered a prophylactically or therapeutically effective amount of an inhibitor in combination with a prophylactically or therapeutically effective amount of one or more other agents useful for bone sarcoma therapy including but not limited to: doxorubicin, ifosfamide, cisplatin, high-dose methotrexate, cyclophosphamide, etoposide, vincristine, dactinomycin, and surgery.

In specific embodiments, patients with tumor metastatic to bone are administered a prophylactically or therapeutically effective amount of an inhibitor in combination with a prophylactically or therapeutically effective amount of one or more other agents useful for bone metastatic tumor therapy including but not limited to: agents or therapies used in treatment of underlying malignancy (non-limiting examples are hormone inhibitors for prostate or breast cancer metastasized to bone and surgery), radiotherapy (non-limiting examples are strontium 89 and samarium 153, which are bone-seeking radionuclides that can exert antitumor effects and relieve symptoms), and bisphosphonates.

Cancer therapies and their dosages, routes of administration and recommended usage are known in the art and have been described in such literature as the Physician's Desk Reference (60^{th} ed., 2006).

### 5.18.2.2 Types of Cancers

Specific examples of cancers that can be prevented, treated and/or managed by the methods encompassed by the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma.

In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). It is also contemplated that cancers caused by aberrations in apoptosis can also be treated by the methods and compositions of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

In a specific embodiment, the cancer prevented, treated and/or managed in accordance with the methods of the invention involves cancer cells expressing (preferably overexpressing) a HAI-TMIP variant, such as HAI-TMIP variant 1, 2, 3, 4, 5, 6, and/or 7. In another embodiment, the cancer prevented, treated and/or managed in accordance with the methods of the invention is benign. In another embodiment, the cancer prevented, treated and/or managed in accordance with the methods of the invention is metastatic. In a particular embodiment, the cancer prevented, treated and/or managed in accordance with the methods of the invention is breast cancer. In another embodiment, the cancer prevented, treated and/or managed in accordance with the methods of the invention is metastatic breast cancer. The breast cancer may be estrogen-dependent or estrogen-independent. In some embodiments, the breast cancer is estrogen-dependent. In other embodiments, the breast cancer is estrogen-independent. In other embodiments, the cancer is not breast cancer. In some embodiments, the cancer is not prostate cancer, colon cancer, and/or leukemia (e.g., acute myelogenous leukemia).

### 5.18.2.3 Patient Population

In certain embodiments, a subject administered an inhibitor in accordance with the methods of the invention has been diagnosed with cancer. In some embodiments, a subject administered an inhibitor in accordance with the methods of the invention is prone or predisposed to cancer. In some embodiments, a subject administered an inhibitor in accordance with the methods of the invention has received or is receiving another therapy. Non-limiting examples of the other therapy include surgery, chemotherapy, radiation therapy, hormonal therapy, biological therapy and immunotherapy. More specific examples are provided above in Section 5.18.2.2.

In certain embodiments, a subject administered an inhibitor in accordance with the methods of the invention is refractory to conventional therapies for cancer. For example, a cancer may be determined to be refractory to a therapy means when at least some significant portion of the cancer cells are not killed or their cell division arrested in response to the therapy. Such a determination can be made either in vivo or in vitro by any method known in the art for assaying the effectiveness of therapy on cancer cells, using the art-accepted meanings of “refractory” in such a context. In a specific embodiment, a cancer is refractory where the number of cancer cells has not been significantly reduced, or has increased.

In more specific embodiments, a subject administered an inhibitor in accordance with the methods of the invention is refractory to existing single agent therapies for cancer. In some embodiments, a subject administered an inhibitor in accordance with the methods of the invention is refractory to therapies other than an inhibitor but is no longer on those therapies.

In some embodiments, a subject administered an inhibitor in accordance with the methods of the invention is immunosuppressed by reason, e.g., of having previously undergone other therapies. In alternative embodiments, a subject administered an inhibitor in accordance with the methods of the invention is immunocompetent.

In some embodiments, a subject administered an inhibitor in accordance with the methods of the invention has experienced or is experiencing an adverse reaction to another therapy. In other embodiments, a subject administered an inhibitor in accordance with the methods of the invention is susceptible to an adverse reaction to another therapy.

In some embodiments, a subject administered an inhibitor in accordance with the methods of the invention is in remission. In some embodiments, a subject administered an inhibitor in accordance with the methods of the invention had cancer but is now cancer-free. In other embodiments, a subject administered an inhibitor in accordance with the methods of the invention is has recurrence of cancer.

In some embodiments, a subject administered an inhibitor in accordance with the methods of the invention is a non-human animal such as a pet, farm animal, and lab animal. In other embodiments, a subject administered an inhibitor in accordance with the methods of the invention is a human. In a specific embodiment, a subject administered an inhibitor in accordance with the methods of the invention is a human subject with other conditions. In another embodiment, a subject administered an inhibitor in accordance with the methods of the invention is a human infant. In another embodiment, a subject administered an inhibitor in accordance with the methods of the invention is a human child. In another embodiment, a subject administered an inhibitor in accordance with the methods of the invention is a human adult. In yet another embodiment, a subject administered an inhibitor in accordance with the methods of the invention is an elderly human.

### 5.18.3 Promotion of Angiogenesis

The present invention provides methods for promoting or increasing angiogenesis, the methods comprising administering to a subject in need thereof one or more activators. Accordingly, the present invention provides methods of preventing, treating and/or managing a condition in which the promotion of angiogenesis would be beneficial, said methods comprising administering to a subject in need thereof one or more activators. Non-limiting examples of activators include those described in Section 5.16. In certain embodiments, an activator increases angiogenesis by at least 25%, preferably at least 50%, at least 75%, at least 90%, or at least 95% in an angiogenesis model described herein or known to one of skill in the art. In a specific embodiment, an activator increases angiogenesis by at least 25%, preferably at least 50%, at least 75%, at least 90%, or at least 95% in a CAM assay or retinal angiogenesis model.

Conditions in which the promotion of angiogenesis is desirable include, but are not limited to, conditions involving wounds, conditions involving occluded vessels, conditions in which organs and/or tissues have insufficient vascularization, and conditions such as spinal cord injuries that may benefit from vascularization. Specific disorders which can be prevented, treated and/or managed in accordance with methods the invention by administering an activator include, but are not limited to, ischemic heart disease, peripheral vascular disease, thromboembolic disease, stroke, vasculititis (Buerger's disease, Wegener's granulomatosis, and Giant Cell Arteritis), and surface ulcers involving the vascular endothelium (e.g., diabetic, haemophiliac, and varicose ulcers). Activators can also be used to promote wound healing, repair vascular damage following myocardial infarction, and to promote angiogenesis following surgical incisions, transplantation, and grafting (e.g., skin grafting and vascular grafting). Non-limiting examples of wounds include lacerations, incisions, and penetrations of blood vessels.

The invention also provides methods of preventing, treating and/or managing a condition in which the promotion of angiogenesis would be beneficial, said methods comprising administering to a subject in need thereof one or more activators and one or more other therapies (e.g., prophylactic or therapeutic agents). In a specific embodiment, the other therapies are currently being used, have been used or are known to be useful in the prevention, treatment and/or management of a disorder characterized by, associated with or caused by aberrant angiogenesis. The therapies (e.g., prophylactic or therapeutic agents) of the combination therapies of the invention can be administered sequentially or concurrently.

In a specific embodiment, the combination therapies of the invention comprise a compound identified in accordance with the invention and at least one other therapy that has the same mechanism of action as said compound. In another specific embodiment, the combination therapies of the invention comprise a compound identified in accordance with the methods of the invention and at least one other therapy (e.g., prophylactic or therapeutic agent) which has a different mechanism of action than said compound. In one embodiment, the combination therapies of the present invention improve the prophylactic or therapeutic effect of a compound of the invention by functioning together with the compound to have an additive or synergistic effect. In another embodiment, the combination therapies of the present invention reduce the side effects associated with the therapies (e.g., prophylactic or therapeutic agents).

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In specific embodiment, the present invention provides methods of preventing, treating and/or managing a condition in which the promotion of angiogenesis would be beneficial, said methods comprising administering to a subject in need thereof a pharmaceutical composition comprising an activator. In accordance with the invention, the pharmaceutical composition may also comprise one or more other prophylactic or therapeutic agents.

An activator may be used as a first, second, third, fourth or fifth line of therapy to prevent, treat and/or manage a disorder characterized by, associated with or caused by aberrant angiogenesis. In certain embodiments, a subject administered an activator in accordance with the methods of the invention has been diagnosed with a condition in which the promotion of angiogenesis would be beneficial. In some embodiments, a subject administered an activator in accordance with the methods of the invention is prone or predisposed to a condition in which the promotion of angiogenesis would be beneficial. In some embodiments, a subject administered an activator in accordance with the methods of the invention has received or is receiving another therapy. Non-limiting examples of the other therapies include cholesterol retulating drugs (e.g., Lipitor), angiogenic agents, hormonal therapy, biological therapy and immunotherapy.

In certain embodiments, a subject administered an activator in accordance with the methods of the invention is refractory to conventional therapies for a condition in which the promotion of angiogenesis would be beneficial. In more specific embodiments, a subject administered an activator in accordance with the methods of the invention is refractory to existing single agent therapies for a condition in which the promotion of angiogenesis would be beneficial. In some embodiments, a subject administered an activator in accordance with the methods of the invention is refractory to therapies other than an activator but is no longer on those therapies.

In some embodiments, a subject administered an activator in accordance with the methods of the invention is immunosuppressed by reason of having previously undergone other therapies. In alternative embodiments, a subject administered an activator in accordance with the methods of the invention is immunocompetent.

In some embodiments, a subject administered an activator in accordance with the methods of the invention has experienced or is experiencing an adverse reaction to another therapy. In other embodiments, a subject administered an activator in accordance with the methods of the invention is susceptible to an adverse reaction to another therapy.

In some embodiments, a subject administered an activator in accordance with the methods of the invention is a non-human animal such as a pet, farm animal, and lab animal. In other embodiments, a subject administered an activator in accordance with the methods of the invention is a human. In a specific embodiment, a subject administered an activator in accordance with the methods of the invention is a human subject with other conditions. In another embodiment, a subject administered an activator in accordance with the methods of the invention is a human infant. In another embodiment, a subject administered an activator in accordance with the methods of the invention is a human child. In another embodiment, a subject administered an activator in accordance with the methods of the invention is a human adult. In yet another embodiment, a subject administered an activator in accordance with the methods of the invention is an elderly human.

### 5.18.4 Disorders Involving Lipoprotein Metabolism

The alpha and beta chains of ATP synthase have been identified as receptors for apolipoprotein E-enriched high-density lipoprotein (HDL) (Beisiegel et al., 1988, Arteriosclerosis 8:288-297 and Mahley et al., 1989, Arterisclerosis 9:114-118) and the beta chain of ATP synthase, probably associated with the alpha chain of ATP synthase, has been shown to be present in the heptocyte plasma membrane. HDL, unlike low-density lipoprotein (LDL), is beneficial to a subject's health. An increase in plasma HDL level has been shown to be inversely related to the occurrence of heart disease (Martinez et al., 2004, Cell. Mol. Life Sci., 61:2343-2360) and so a low plasma HDL level is an important risk factor of atherosclerosis. It has also been shown that plasma HDL has anti-inflammatory and anti-atherosclerosis activities.

The major role of HDL is to remove cholesterol from peripheral tissues, including atherosclerotic lesions, and to deliver it to the liver for elimination in a process is referred to as “reverse cholesterol transport” (Martinez et al., 2004, Cell. Mol. Life Sci., 61:2343-2360). As part of this process, HDL endocytosis in heptocytes is an important step. It has been shown that HDL is internalized in heptocytes, via the formation of clathrin-coated vesicles, following the engagement of the high affinity binding sites, i.e., the beta chain of ATP synthase (Martinez et al., 2004, Cell. Mol. Life. Sci., 61:2343-2360). The binding of HDL to the beta subunit of ATP synthase has been proposed as an upstream event in HDL endocytosis (Martinez et al., 2004, Cell. Mol. Life Sci., 61:2343-2360).

Based, in part, upon the role of ATP synthase in HDL endocytosis, the present invention, in one aspect, provides methods for modulating HDL endocytosis, and thus, methods of modulating the elimination of cholesterol by the liver, by modulating the expression and/or activity of a HAI-TMIP variant (e.g., HAI-TMIP variant 1, 2, 3, 4, 5, 6 and/or 7), or the formation, stability and/or activity of an F₀F₁ ATP synthase holoenzyme or an F₁ ATP synthase subcomplex.

In a specific embodiment, the present invention provides methods for increasing high density lipoprotein (HDL) endocytosis, the methods comprising administering to a subject in need thereof one or more activators. In another embodiment, the present invention also provides methods for decreasing cholesterol in the blood, the methods comprising administering to a subject in need thereof one or more activators. In another aspect, the present invention, based, in part, upon the importance of HDL, provides methods for preventing, treating and/or managing a disorder characterized by, associated with or caused by elevated levels of cholesterol. In a specific embodiment, the present invention provides methods of preventing, treating and/or managing a disorder characterized by, associated with or caused by elevated levels of cholesterol in the blood, said methods comprising administering to a subject in need thereof one or more activators. The term “elevated levels of cholesterol”, as used herein, refers to a higher level of cholesterol than recommended by a health care professional. Currently, recommended levels of total blood cholesterol are less than 200 mg/dl. Non-limiting examples of activators include those described in Section 5.16.

Disorders encompassed by the methods of the present invention that are characterized by, associated with or caused by elevated levels of cholesterol in the blood include, but are not limited to, cardiovascular disease, type 2 diabetes, coronary heart disease, stroke, pancreatitis, hyperlipidemia, obesity, atherosclerosis and gout.

The invention also provides methods of preventing, treating and/or managing a disorder characterized by, associated with or caused by elevated levels of cholesterol in the blood, said methods comprising administering to a subject in need thereof one or more activators and one or more other therapies (e.g., prophylactic or therapeutic agents). In a specific embodiment, the other therapies are currently being used, have been used or are known to be useful in the prevention, treatment and/or management of a disorder characterized by, associated with or caused by elevated levels of cholesterol in the blood. The therapies (e.g., prophylactic or therapeutic agents) of the combination therapies of the invention can be administered sequentially or concurrently.

In a specific embodiment, the combination therapies of the invention comprise a compound identified in accordance with the invention and at least one other therapy that has the same mechanism of action as said compound. In another specific embodiment, the combination therapies of the invention comprise a compound identified in accordance with the methods of the invention and at least one other therapy (e.g., prophylactic or therapeutic agent) which has a different mechanism of action than said compound. In one embodiment, the combination therapies of the present invention improve the prophylactic or therapeutic effect of a compound of the invention by functioning together with the compound to have an additive or synergistic effect. In another embodiment, the combination therapies of the present invention reduce the side effects associated with the therapies (e.g., prophylactic or therapeutic agents).

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In specific embodiment, the present invention provides methods of preventing, treating and/or managing a disorder characterized by, associated with or caused by elevated levels of cholesterol in the blood, said methods comprising administering to a subject in need thereof a pharmaceutical composition comprising an activator. In accordance with the invention, the pharmaceutical composition may also comprise one or more other prophylactic or therapeutic agents.

An activator may be used as a first, second, third, fourth or fifth line of therapy to prevent, treat and/or manage a disorder characterized by, associated with or caused by elevated levels of cholesterol in the blood. In certain embodiments, a subject administered an activator in accordance with the methods of the invention has been diagnosed with a disorder characterized by, associated with or caused by elevated levels of cholesterol in the blood. In some embodiments, a subject administered an activator in accordance with the methods of the invention is prone or predisposed to a disorder characterized by, associated with or caused by elevated levels of cholesterol in the blood. In some embodiments, a subject administered an activator in accordance with the methods of the invention has received or is receiving another therapy. Non-limiting examples of the other therapies include surgery, cholesterol regulating drugs (e.g., Lipitor), insulin, anti-angiogenic agents, biological therapy and immunotherapy.

In certain embodiments, a subject administered an activator in accordance with the methods of the invention is refractory to conventional therapies for a disorder characterized by, associated with or caused by elevated levels of cholesterol in the blood. In more specific embodiments, a subject administered an activator in accordance with the methods of the invention is refractory to existing single agent therapies for a disorder characterized by, associated with or caused by elevated levels of cholesterol in the blood. In some embodiments, a subject administered an activator in accordance with the methods of the invention is refractory to therapies other than an activator but is no longer on those therapies.

In some embodiments, a subject administered an activator in accordance with the methods of the invention is immunosuppressed by reason of having previously undergone other therapies. In alternative embodiments, a subject administered an activator in accordance with the methods of the invention is immunocompetent.

In some embodiments, a subject administered an activator in accordance with the methods of the invention has experienced or is experiencing an adverse reaction to another therapy. In other embodiments, a subject administered an activator in accordance with the methods of the invention is susceptible to an adverse reaction to another therapy.

In some embodiments, a subject administered an activator in accordance with the methods of the invention is a non-human animal such as a pet, farm animal, and lab animal. In other embodiments, a subject administered an activator in accordance with the methods of the invention is a human. In a specific embodiment, a subject administered an activator in accordance with the methods of the invention is a human subject with other conditions. In another embodiment, a subject administered an activator in accordance with the methods of the invention is a human infant. In another embodiment, a subject administered an activator in accordance with the methods of the invention is a human child. In another embodiment, a subject administered an activator in accordance with the methods of the invention is a human adult. In yet another embodiment, a subject administered an activator in accordance with the methods of the invention is an elderly human.

In a specific embodiment, a subject administered an activator in accordance with invention has experienced or is experiencing a myocardial infarction. In another embodiment, a subject administered an activator in accordance with invention is a human with a blood LDL cholesterol level of 130 to 159 mg/dL. In another embodiment, a subject administered an activator in accordance with the invention is a human with a blood LDL cholesterol level of 160 to 189 mg/dL. In another embodiment, a subject administered an activator in accordance with the invention is a human with a blood LDL cholesterol level of 190 mg/dL or more. In another embodiment, a subject administered an activator in accordance with the invention is a human with a blood triglyceride level of 150 mg/dL or more. In another embodiment, a subject administered an activator in accordance with the invention is a human with a blood total cholesterol level of 200 to 239 mg/dL. In a subject administered an activator in accordance with the invention is a human with a blood total cholesterol level of 240 mg/dL or more.

The β subunit of ATP synthase has been shown to bind to enterostatin, a peptide released from procolipase during fat digestion (Berger et al., 2004, Physiol. Behav 83:623-630). Enterostatin has been shown to restrict fat intake and to prevent the overconsumption of fat (Berger et al., 2004, Physiol Behav 83:623-630).

The present invention provides methods of modulating energy metabolism, said methods comprising to a subject in need thereof an inhibitor or activator, alone or in combination with other therapies. In a specific embodiment, an activator is administered to a subject to prevent, treat and/or manage obesity. In another embodiment, an activator is administered to a subject to prevent, treat and/or manage type 2 diabetes. In yet another embodiment, an inhibitor is administered to prevent, treat and/or manage disorders associated with excessive weight loss.

### 5.18.5 Hypertension

It has been shown that an antibody specific for the β subunit of ATP synthase can suppress coupling factor 6-induced increases in blood pressure (Osani et al., 2005, Hypertension 46:1140-1146). The present invention provides methods for modulating blood pressure in a subject, said methods comprising administering to a subject in need thereof a modulator. The present invention also provides methods of preventing, treating and/or managing hypertension, said methods comprising administering to a subject in need thereof one or more inhibitors. Non-limiting examples of inhibitors include those described in Section 5.15. The invention also provides methods of preventing, treating and/or managing hypertension, said methods comprising administering to a subject in need thereof one or more inhibitors and one or more other therapies (e.g., prophylactic or therapeutic agents). In a specific embodiment, the other therapies are currently being used, have been used or are known to be useful in the prevention, treatment and/or management of hypertension. The therapies (e.g., prophylactic or therapeutic agents) of the combination therapies of the invention can be administered sequentially or concurrently.

In a specific embodiment, the combination therapies of the invention comprise a compound identified in accordance with the invention and at least one other therapy that has the same mechanism of action as said compound. In another specific embodiment, the combination therapies of the invention comprise a compound identified in accordance with the methods of the invention and at least one other therapy (e.g., prophylactic or therapeutic agent) which has a different mechanism of action than said compound. In another embodiment, the combination therapies of the present invention improve the prophylactic or therapeutic effect of a compound of the invention by functioning together with the compound to have an additive or synergistic effect. The combination therapies of the present invention reduce the side effects associated with the therapies (e.g., prophylactic or therapeutic agents).

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In specific embodiment, the present invention provides methods of preventing, treating and/or managing hypertension, said methods comprising administering to a subject in need thereof a pharmaceutical composition comprising an inhibitor. In accordance with the invention, the pharmaceutical composition may also comprise one or more other prophylactic or therapeutic agents.

An inhibitor may be used as a first, second, third, fourth or fifth line of therapy to prevent, treat and/or manage hypertension. In certain embodiments, a subject administered an inhibitor in accordance with the methods of the invention has been diagnosed with hypertension. In some embodiments, a subject administered an inhibitor in accordance with the methods of the invention is prone or predisposed to hypertension. In some embodiments, a subject administered an inhibitor in accordance with the methods of the invention has received or is receiving another therapy. Non-limiting examples of the other therapy include biological therapy and drugs.

In certain embodiments, a subject administered an inhibitor in accordance with the methods of the invention is refractory to conventional therapies for hypertension. In more specific embodiments, a subject administered an inhibitor in accordance with the methods of the invention is refractory to existing single agent therapies for hypertension. In some embodiments, a subject administered an inhibitor in accordance with the methods of the invention is refractory to therapies other than an inhibitor but is no longer on those therapies.

In some embodiments, a subject administered an inhibitor in accordance with the methods of the invention is immunosuppressed by reason of having previously undergone other therapies. In alternative embodiments, a subject administered an inhibitor in accordance with the methods of the invention is immunocompetent.

In some embodiments, a subject administered an inhibitor in accordance with the methods of the invention has experienced or is experiencing an adverse reaction to another therapy. In other embodiments, a subject administered an inhibitor in accordance with the methods of the invention is susceptible to an adverse reaction to another therapy.

In some embodiments, a subject administered an inhibitor in accordance with the methods of the invention is a non-human animal such as a pet, farm animal, and lab animal. In other embodiments, a subject administered an inhibitor in accordance with the methods of the invention is a human. In a specific embodiment, a subject administered an inhibitor in accordance with the methods of the invention is a human subject with other conditions. In another embodiment, a subject administered an inhibitor in accordance with the methods of the invention is a human infant. In another embodiment, a subject administered an inhibitor in accordance with the methods of the invention is a human child. In another embodiment, a subject administered an inhibitor in accordance with the methods of the invention is a human adult. In yet another embodiment, a subject administered an inhibitor in accordance with the methods of the invention is an elderly human.

### 5.19 Diagnostic and Monitoring Techniques

Although the methods described in this Section 5.19 are described with respect to a HAI-TMIP variant, the methods described herein can be applied to other components of the F₁ ATP synthase subcomplex and the F₀F₁ ATP synthase holoenzyme. The present invention provides methods for detecting and/or diagnosing cancer in a subject, the method comprising detecting the amount of a HAI-TMIP gene product in a sample from a subject and comparing the amount of HAI-TMIP gene product in the sample to the amount of HAI-TMIP gene product in one or more control samples, or to a predetermined reference range, wherein the amount of HAI-TMIP gene product in the sample from the subject relative to the control sample or reference range indicates whether cancer is detected and/or diagnosed. In one embodiment, cancer is detected and/or diagnosed if there is an equivalent or greater amount of HAI-TMIP gene product in the sample from the subject relative to the amount of HAI-TMIP gene product in a positive control sample or to a predetermined reference range for a subject or a population of subjects having cancer. In another embodiment, an equivalent or decreased amount of HAI-TMIP gene product (e.g., HAI-TMIP variant 4, 5, 6, and/or 7 gene product) in the sample from the subject relative to the amount of HAI-TMIP gene product in a negative control sample, or to a predetermined reference range for a subject or a population of healthy or cancer-free subjects, indicates a negative diagnosis for cancer. In accordance with these embodiments, the HAI-TMIP gene product is, in certain embodiments, the HAI-TMIP variant 4, 5, 6 or 7 gene product.

In a specific embodiment, the present invention provides methods for detecting and/or diagnosing cancer in a subject, the method comprising detecting the amount of HAI-TMIP variant 3 gene product in a sample from a subject and comparing the amount of HAI-TMIP variant 3 gene product in the sample to the amount of HAI-TMIP gene product in one or more control samples, or to a predetermined reference range, wherein the amount of HAI-TMIP variant 3 gene product in the sample from the subject relative to the control sample or reference range indicates whether cancer is detected and/or diagnosed. In one embodiment, cancer is detected and/or diagnosed if there is an equivalent or less than the amount of HAI-TMIP variant 3 gene product in the sample from the subject relative to the amount of HAI-TMIP variant 3 gene product in a positive control sample or to a predetermined reference range for a subject or a population of subjects having cancer. In another embodiment, an equivalent or increased amount of HAI-TMIP variant 3 gene product in the sample from the subject relative to the amount of HAI-TMIP variant 3 gene product in a negative control sample, or to a predetermined reference range for a subject or a population of healthy or cancer-free subjects, indicates a negative diagnosis for cancer.

Non-limiting examples of a positive control sample include a sample from a subject with the cancer of interest and a sample from a population of subjects with the cancer. In a specific embodiment, the positive control sample is from a subject or a population of subjects manifesting the early stages of the cancer of interest. In certain embodiments, the amount of HAI-TMIP gene product in the sample from the subject is compared to the amount of HAI-TMIP gene product in a negative control sample and a positive control sample in order to detect or diagnose cancer. Non-limiting examples of a negative control sample include a sample from a healthy or cancer-free subject or a sample from a population of healthy or cancer-free subjects.

The present invention provides methods for predicting the onset of cancer, the methods comprising measuring the amount of HAI-TMIP gene product in a sample from the subject and comparing the amount of HAI-TMIP gene product in the sample to the amount of HAI-TMIP gene product in a control sample, or to a predetermined reference range. In one embodiment, the subject's sample is compared to a negative control sample, or to a predetermined reference range for a healthy or cancer-free subject, and cancer is predicted if there is an increase in the amount of HAI-TMIP gene product (e.g., HAI-TMIP variant 4, 5, 6, and/or 7) in the sample from the subject relative to the amount of HAI-TMIP gene product in the control sample, or to the predetermined reference range. In another embodiment, the subject's sample is compared to a positive control sample, or to a predetermined reference range for a subject with the cancer, and the cancer is predicted if there is an equivalent or greater amount of HAI-TMIP gene product (e.g., HAI-TMIP variant 4, 5, 6, and/or 7) in the sample from the subject relative to the amount of HAI-TMIP gene product in the control sample, or to the predetermined reference range. In accordance with these embodiments, the HAI-TMIP gene product is, in certain embodiments, the HAI-TMIP variant 4, 5, 6, and/or 7 gene product.

In a specific embodiment, the present invention provides methods for predicting the onset of cancer, the methods comprising measuring the amount of HAI-TMIP variant 3 gene product in a sample from the subject and comparing the amount of HAI-TMIP variant 3 gene product in the sample to the amount of HAI-TMIP variant 3 gene product in a negative control sample, or to a predetermined reference range for a healthy or cancer-free subject or population or such subjects, wherein the cancer is predicted if there is a decrease in the amount of HAI-TMIP variant 3 gene product in the sample from the subject relative to the amount of HAI-TMIP variant 3 gene product in the control sample, or to the predetermined reference range. In another embodiment, the present invention also provides methods for predicting the onset of cancer, the methods comprising measuring the amount of HAI-TMIP variant 3 gene product in a sample from the subject and comparing the amount of HAI-TMIP variant 3 gene product in the sample to the amount of HAI-TMIP gene product in a positive control sample, or to a predetermined reference range for a subject or population of subjects with the cancer, wherein the cancer is predicted if there is an equivalent or decreased amount of HAI-TMIP variant 3 gene product in the sample from the subject relative to the amount of HAI-TMIP variant 3 gene product in the control sample, or to the predetermined reference range.

In a specific embodiment, the positive control is from a subject or a population of subjects manifesting early stages of the cancer of interest. In certain embodiments, the amount of HAI-TMIP gene product in the sample from the subject are compared to the amount of HAI-TMIP gene product in a negative control sample and a positive control sample in order to predict the onset of cancer.

In a specific embodiment, the subject sample is from a subject predisposed, either genetically, environmentally or both, to the cancer of interest. In another specific embodiment, once a subject is predicted to develop the cancer of interest, routine assays to diagnose or predict the onset of the cancer are conducted; the subject is routinely monitored for the onset of the cancer utilizing the methods described herein and/or known to one of skill in the art; and/or the subject is administered prophylactic therapy.

The present invention provides methods for monitoring the progression of cancer in a subject, the methods comprising measuring the amount of HAI-TMIP gene product in a sample from the subject and comparing the amount of HAI-TMIP gene product in the sample to the amount of HAI-TMIP gene product in a control sample, or to a predetermined reference range, wherein the amount of HAI-TMIP gene product in the sample from the subject relative to the control, or to the predetermined reference range indicates whether the cancer has progressed. In a specific embodiment, the control sample is an initial or earlier sample(s) from the subject, and the predetermined reference range is the amount of HAI-TMIP gene product present in an initial or earlier sample(s) from the subject. In accordance with this embodiment, an increase in the amount of HAI-TMIP gene product (e.g., HAI-TMIP variant 4, 5, 6, and/or 7) in the sample from the subject relative to the control sample, or to the predetermined reference range, in some embodiments, indicates that the cancer has progressed, and a decrease or no change in the amount of HAI-TMIP gene product in the sample from the subject relative to the control, or the predetermined reference range indicates, in some embodiments, that the cancer has not progressed. In other embodiments, a decrease or no change in the amount of HAI-TMIP gene product (e.g., HAI-TMIP variant 3) in the sample from the subject relative to the control sample or predetermined reference range indicates that the cancer has not progressed, and an increase in the amount of the HAI-TMIP variant relative to the control sample or predetermined reference range indicates that the disease has progressed. In another embodiment, the control sample is from a subject or a population of subjects with an early stage of the cancer, a moderate stage of the cancer and/or a late stage of the cancer. In another embodiment, more than one control sample is used in accordance with the methods.

In a specific embodiment, the present invention provides methods for monitoring the progression of cancer in a subject, the methods comprising measuring the amount of HAI-TMIP gene product in a sample from the subject and comparing the amount of HAI-TMIP gene product in the sample to the amount of HAI-TMIP gene product in a sample from a subject or population of subjects with an early stage of the cancer, or to a predetermined reference range for a subject with an early stage of the cancer, wherein (i) a decrease in the amount HAI-TMIP gene product in the sample from the subject relative to the control, or to the predetermined reference range indicates that the cancer has not progressed or has regressed; (ii) an equivalent amount of HAI-TMIP gene product in the sample from the subject relative to the control, or to the predetermined reference range indicates that the cancer has not progressed; and (iii) an increase in the amount of HAI-TMIP gene product in the sample from the subject relative to the control or to the predetermined reference range indicates or suggests that the cancer may have progressed.

In another embodiment, the present invention provides methods for monitoring the progression of cancer in a subject, the methods comprising measuring the amount of HAI-TMIP gene product in a sample from the subject and comparing the amount of HAI-TMIP gene product in the sample to the amount of HAI-TMIP gene product in a sample from a subject or population of subjects with a moderate stage of the cancer, or to a predetermined reference range for a subject with a moderate stage of the cancer, wherein: (i) a decrease in the amount HAI-TMIP gene product in the sample from the subject relative to the control, or to the predetermined reference range indicates that the cancer has not progressed or has regressed; (ii) an equivalent amount of HAI-TMIP gene product in the sample from the subject relative to the control, or to the predetermined reference range indicates that the cancer has not progressed; and (iii) an increase in the amount of HAI-TMIP gene product in the sample from the subject relative to the control or to the predetermined reference range indicates or suggests that the cancer may have progressed.

In another embodiment, the present invention provides methods for monitoring the progression of cancer in a subject, the methods comprising measuring the amount of HAI-TMIP gene product in a sample from the subject and comparing the amount of HAI-TMIP gene product in the sample to the amount of HAI-TMIP gene product in a sample from a subject or population of subjects with a late stage of the cancer, or to a predetermined reference range for a subject with a late stage of the cancer, wherein: (i) a decrease in the amount HAI-TMIP gene product in the sample from the subject relative to the control, or to the predetermined reference range indicates that the cancer has not progressed or has regressed; (ii) an equivalent amount of HAI-TMIP gene product in the sample from the subject relative to the control, or to the predetermined reference range indicates that the cancer has not progressed; and (iii) an increase in the amount of HAI-TMIP gene product in the sample from the subject relative to the control or to the predetermined reference range indicates or suggests that the cancer may have progressed.

The present invention provides methods for monitoring the efficacy of a therapy in a subject, the methods comprising measuring the amount of HAI-TMIP gene product in a sample from the subject and comparing the amount of HAI-TMIP gene product in the sample to the amount of HAI-TMIP gene product in a control sample, or to a predetermined reference range, wherein the amount of HAI-TMIP in the sample from the subject relative to the control sample, or to the predetermined reference range indicates whether the therapy has been efficacious. In a specific embodiment, the control sample is a sample from the subject prior to initiation of the therapy, and the predetermined reference range is the amount of HAI-TMIP gene product in a sample from the subject prior to initiating the therapy. In accordance with this embodiment, a decrease in the amount of HAI-TMIP gene product (e.g., HAI-TMIP variant 4, 5, 6 and/or 7 gene product) relative to the control sample, or to the predetermined reference range indicates, in some embodiments, that the therapy is being efficacious. Alternatively, in accordance with this embodiment, an increase in the amount of HAI-TMIP gene product (e.g., HAI-TMIP variant 3 gene product) in sample relative to the control sample, or the predetermined reference range indicates, in some embodiments, that the therapy is being efficacious. In another embodiment, the control sample is a sample from a healthy or cancer-free subject or a population of healthy or cancer-free subjects and/or a sample from a subject or a population of subjects with the cancer where the therapy has been efficacious, or the predetermined reference range is the amount of HAI-TMIP gene product in a sample from a healthy or cancer-free subject or a population of healthy or cancer-free subjects and/or a sample from a subject or a population of subjects with the cancer where the therapy has been efficacious. In accordance with this embodiment, an equivalent amount of HAI-TMIP gene product (e.g., HAI-TMIP variant 3, 4, 5, 6 and/or 7 gene product) in the sample from the subject relative to the control sample or predetermined reference range indicates, in some embodiments, that the therapy is being efficacious.

The present invention provides methods for detecting or diagnosing cancer in a subject, the methods comprising measuring the amount of HAI-TMIP gene product in the subject and comparing the amount of HAI-TMIP gene product in the subject to a predetermined reference range for a healthy or cancer-free subject, wherein a difference in the amount of HAI-TMIP gene product indicates whether cancer is detected or diagnosed. In a specific embodiment, cancer is detected or diagnosed if there is an increase in the amount of HAI-TMIP gene product (e.g., HAI-TMIP variant 4, 5, 6 and/or 7 gene product) in the subject relative to the predetermined reference range. In some embodiments, cancer is detected or diagnosed if there is a decrease in the amount of HAI-TMIP variant 3 gene product relative to a predetermined reference range for a healthy or cancer-free subject. The present invention also provides methods for detecting or diagnosing cancer in a subject, the methods comprising measuring the amount of HAI-TMIP gene product (e.g., HAI-TMIP variant 4, 5, 6 and/or 7 gene product) in the subject and comparing the amount of HAI-TMIP gene product in the subject to a predetermined reference range for a subject with the cancer, wherein a difference in the amount of HAI-TMIP gene product indicates whether cancer is detected or diagnosed. In a specific embodiment, cancer is detected or diagnosed if there is an equivalent or greater amount of HAI-TMIP gene product (e.g., HAI-TMIP variant 4, 5, 6 and/or 7 gene product) in the subject relative to the predetermined reference range.

The present invention provides methods for predicting the onset of cancer in a subject, the methods comprising measuring the amount of HAI-TMIP in the subject and comparing the amount of HAI-TMIP gene product in the subject to a predetermined reference range for a healthy or cancer-free subject, wherein a difference in the amount of ATP synthase gene product indicates whether the onset of cancer is predicted. In a specific embodiment, cancer is predicted if there is an increase in the amount of HAI-TMIP gene product (e.g., HAI-TMIP variant 4, 5, 6 and/or 7 gene product) in the subject relative to the predetermined reference range. In some embodiments, cancer is predicted if there is a decrease in the amount of HAI-TMIP variant 3 gene product relative to a predetermined reference range for a healthy or cancer-free subject.

The present invention also provides methods for predicting the onset of cancer in a subject, the methods comprising measuring the amount of HAI-TMIP gene product in the subject and comparing the amount of HAI-TMIP gene product in the subject to a predetermined reference range for a subject with the cancer, wherein a difference in the amount of HAI-TMIP gene product indicates whether the onset of cancer is predicted. In a specific embodiment, cancer is predicted if there is an equivalent or greater amount of HAI-TMIP gene product (e.g., HAI-TMIP variant 4, 5, 6, or 7) in the subject relative to the predetermined reference range. In a specific embodiment, the subject is predisposed, either genetically, environmentally or both, to the cancer of interest. In another specific embodiment, once a subject is predicted to develop the cancer of interest, routine assays to diagnose or predict the onset of the cancer are conducted; the subject is routinely monitored for the onset of the cancer utilizing the methods described herein and/or known to one of skill in the art; and/or the subject is administered prophylactic therapy.

The present invention provides methods for monitoring the progression of cancer in a subject, the methods comprising measuring the amount of HAI-TMIP gene product in the subject and comparing the amount of HAI-TMIP gene product in the subject or to a predetermined reference range, wherein the amount of HAI-TMIP gene product in the subject relative to the predetermined reference range indicates whether the cancer has progressed. In a specific embodiment, the predetermined reference range is the amount of HAI-TMIP gene product present in an initial or earlier sample(s) from the subject. In accordance with this embodiment, an increase in the amount of HAI-TMIP gene product (e.g., HAI-TMIP variant 4, 5, 6 and/or 7 gene product) in the subject relative to the predetermined reference range indicates, in some embodiments, that the cancer has progressed, and a decrease or no change in the amount of HAI-TMIP gene product in the subject relative to the predetermined reference range indicates, in some embodiments, that the cancer has not progressed. In some embodiments, a decrease in the amount of HAI-TMIP variant 3 relative to the predetermined reference range indicates that the cancer has progressed.

In a specific embodiment, the present invention provides methods for monitoring the progression of cancer in a subject, the methods comprising measuring the amount of HAI-TMIP gene product in the subject and comparing the amount of HAI-TMIP gene product in the subject to a predetermined reference range for a subject with an early stage of the cancer, wherein (i) a decrease in the amount HAI-TMIP gene product in the subject relative to the predetermined reference range indicates that the cancer has not progressed or has regressed; (ii) an equivalent amount of HAI-TMIP gene product in the subject relative to the predetermined reference range indicates that the cancer has not progressed; and (iii) an increase in the amount of HAI-TMIP gene product in the subject relative to the predetermined reference range indicates or suggests that the cancer may have progressed.

In another embodiment, the present invention provides methods for monitoring the progression of cancer in a subject, the methods comprising measuring the amount of HAI-TMIP gene product in the subject and comparing the amount of HAI-TMIP gene product in the subject to a predetermined reference range for a subject with a moderate stage of the cancer, wherein: (i) a decrease in the amount HAI-TMIP gene product in the subject relative to the predetermined reference range indicates that the cancer has not progressed or has regressed; (ii) an equivalent amount of HAI-TMIP gene product in the subject relative to the predetermined reference range indicates that the cancer has not progressed; and (iii) an increase in the amount of HAI-TMIP gene product in the subject relative to the predetermined reference range indicates or suggests that the cancer may have progressed.

In another embodiment, the present invention provides methods for monitoring the progression of cancer in a subject, the methods comprising measuring the amount of HAI-TMIP gene product in the subject and comparing the amount of HAI-TMIP gene product in the subject to a predetermined reference range for a subject with a late stage of the cancer, wherein: (i) a decrease in the amount HAI-TMIP gene product in the subject relative to the predetermined reference range indicates that the cancer has not progressed or has regressed; (ii) an equivalent amount of HAI-TMIP gene product in the subject relative to the predetermined reference range indicates that the cancer has not progressed; and (iii) an increase in the amount of HAI-TMIP gene product in the subject relative to the predetermined reference range indicates or suggests that the cancer may have progressed.

The present invention provides methods for monitoring the efficacy of a therapy in a subject, the methods comprising measuring the amount of HAI-TMIP gene product in the subject and comparing the amount of HAI-TMIP gene product in the subject to a predetermined reference range, wherein the amount of HAI-TMIP in the sample from the subject relative to the predetermined reference range indicates whether the therapy has been efficacious. In a specific embodiment, the predetermined reference range is the amount of HAI-TMIP gene product in a sample from the subject prior to initiating the therapy. In accordance with this embodiment, a decrease in the amount of HAI-TMIP gene product (e.g., HAI-TMIP variant 4, 5, 6 and/or 7 gene product) relative to the predetermined reference range indicates, in some embodiments, that the therapy is being efficacious. In other embodiments, an increase in the amount of HAI-TMIP variant 3 gene product relative to the predetermined reference range indicates that the therapy is being efficacious. In another embodiment, the predetermined reference range is the amount of HAI-TMIP gene product in a healthy or cancer-free subject or a population of healthy or cancer-free subjects and/or the amount of HAI-TMIP gene product in a subject or a population of subjects with the cancer where the therapy has been efficacious. In accordance with this embodiment, an equivalent amount of HAI-TMIP gene product (e.g., HAI-TMIP variant 3, 4, 5, 6 and/or 7 gene product) in the subject relative to the predetermined reference range indicates, in some embodiments, that the therapy is being efficacious.

The present invention also provides methods for detecting and/or diagnosing cancer in a subject, the methods comprising detecting the amount of a complex of the invention and comparing the amount of the complex in the sample or subject to a control sample or predetermined reference range, wherein a difference in the amount of complex in the subject sample or in the subject relative to the control sample of predetermined reference indicates whether cancer is detected and/or diagnosed. In some embodiments, the complex comprises HAI-TMIP variant 4, 5, 6 or 7 and the control sample or predetermined reference range is from a healthy or cancer-free subject or population of subjects. In accordance with this embodiment, an increase in the amount of complex relatives to the control sample or predetermined refers indicates a positive diagnosis/detection of cancer. In other embodiments, the complex comprises HAI-TMIP variant 3 and the control sample or predetermined reference range is from a healthy or cancer-free subject. In accordance with this embodiment, a decrease in the amount of HAI-TMIP variant 3 indicates a positive diagnosis/detection of cancer. In yet other embodiments, the complex comprises HAI-TMIP variant 4, 5, 6 or 7 and the control sample or predetermined reference range is from a cancer subject or population of subjects. In accordance with this embodiment, an equivalent or greater amount of the complex relative to the control sample or predetermined reference range indicates a positive diagnosis of cancer.

The present invention provides methods for monitoring the progression of cancer in a subject, the methods comprising detecting the amount of a complex of the invention in a subject or a sample from a subject and comparing the amount in the subject or sample to a control sample or predetermined reference range, wherein a difference in the amount of complex in the subject or subject's sample relative to the control sample indicates whether the cancer has progressed. In certain embodiments, the control sample or predetermined reference range is from the subject at an earlier time point and the complex comprises HAI-TMIP 4, 5, 6 or 7. In accordance with this embodiments, an increase in the amount of complex indicates the progression of the cancer and a decrease indicates the cancer has not progressed. In other embodiments, the control sample or predetermined reference range is from the subject at an earlier time point and the complex comprises HAI-TMIP variant 3. In accordance with this embodiment, an equivalent amount or an increase in the amount of complex indicates that the cancer has not progressed. In yet other embodiments, the control sample or predetermined reference range are from a healthy subject or cancer-free subject or population subjects and an amount similar to the control sample or predetermined reference indicates an improvement.

The present invention provides methods for monitoring the efficacy of a therapy in a subject, the methods comprising detecting the amount of a complex of the invention in a subject or a sample from a subject and comparing the amount in the subject or sample to a control sample or predetermined reference range, wherein a difference in the amount of complex in the subject or subject's sample relative to the control sample indicates whether the therapy is efficacious. In certain embodiments, the control sample or predetermined reference range is from the subject at an earlier time point and the complex comprises HAI-TMIP 4, 5, 6 or 7. In accordance with this embodiments, an increase in the amount of complex indicates that therapy is not efficacious and a decrease indicates the therapy is a efficacious. In other embodiments, the control sample or predetermined reference range is from the subject at an earlier time point and the complex comprises HAI-TMIP variant 3. In accordance with this embodiment, an increase in the amount of complex indicates that the therapy is efficacious. In yet other embodiments, the control sample and predetermined reference range are from a healthy subject or cancer-free subject or population of subjects and an amount similar to the control sample or predetermined reference indicates that the therapy is efficacious.

The amount of HAI-TMIP gene product in a sample from a subject can be determined by measuring HAI-TMIP RNA, preferably HAI-TMIP mRNA, protein or protein activity. In one embodiment, the amount of HAI-TMIP RNA is detected and/or measured. In accordance with this embodiment, the amount of HAI-TMIP RNA itself can be detected or measured, or the HAI-TMIP RNA can be reverse transcribed into cDNA and the cDNA can be detected or measured. In a preferred embodiment, the amount of HAI-TMIP mRNA is detected and/or measured. In another embodiment, the amount of HAI-TMIP protein is detected and/or measured. HAI-TMIP RNA, protein and protein activity can be detected and measured by techniques well-known in the art, and by techniques described herein.

In accordance with the methods of the invention, the amount of HAI-TMIP gene product can be determined in vitro or in vivo, or both. In one embodiment, a sample from a subject is used to determine the amount of HAI-TMIP gene product expressed by a subject. In alternative embodiment, the amount of HAI-TMIP gene product is determined by measuring the amount of HAI-TMIP gene product in the subject.

Any semi-quantitative or quantitative technique for measuring the amount of a gene product in a sample can be used to measure the amount of HAI-TMIP gene product or complex according to the methods of this invention. The amount of the HAI-TMIP gene product can be determined by measuring the amount of either nucleic acid or protein, or both. For example, the amount of a particular nucleic acid in a sample can be determined by hybridization with a complementary nucleic acid probe which is detectably labelled, as in the standard protocols for Northern and Southern hybridization or as in a nuclease protection assay. Alternatively, the amount of a particular nucleic acid in a sample can be determined by a polymerase chain reaction based assay or by microarray analysis. The amount of a particular protein in a sample can be determined, e.g., by any number of antibody-based assays such as immunoprecipitation, enzyme linked immunoassay, radioimmunoassay, flow cytometry, or Western hybridization techniques.

In accordance with the methods of this invention, the amount of HAI-TMIP gene product or complex is either the absolute or relative amount, including but not limited to values representing abundances or abundance ratios. In a specific embodiment, the expression of the HAI-TMIP gene product is measured using a high throughput technique, such as a polymerase chain reaction based technique, a flow cytometry based technique, or a technique utilizing nucleic acid or protein microarrays.

In a particular embodiment, the amount of HAI-TMIP gene product is measured in vitro in a sample from a subject according to a method comprising the steps of: (a) measuring the amount of HAI-TMIP gene product in the sample using one or more HAI-TMIP-binding agents; and (b) comparing the amount of the HAI-TMIP gene product in the sample with the amount of HAI-TMIP gene product in a control sample or a predetermined reference range. The particular control or predetermined reference range used will vary.

In one embodiment, a HAI-TMIP RNA product in a sample from a subject is detected and/or measured by steps comprising: (a) contacting the sample (which may or may not have been modified after obtaining it from that subject) with a HAI-TMIP-binding agent (e.g., a probe) which binds to the HAI-TMIP RNA product under conditions that permit the agent to bind to the HAI-TMIP RNA product; and (b) detecting and/or measuring the binding of the HAI-TMIP RNA product to the agent. In a specific embodiment, the agent is an antisense ribonucleic acid of HAI-TMIP. In one embodiment, the agent is labelled with a detectable label and the amount of bound agent is determined by techniques well-known.

In one embodiment, a HAI-TMIP RNA product in a sample from a subject is detected and/or measured by steps comprising: (a) producing a product complementary to the HAI-TMIP RNA product using primers complementary to the HAI-TMIP RNA product; and (b) detecting and/or measuring the product produced in step (a). In another embodiment, a HAI-TMIP RNA product in a sample from a subject is detected and/or measured by steps comprising: (a) reverse transcribing the HAI-TMIP RNA product into cDNA using appropriate primers (e.g., olig o dT primers); (b) amplifying the cDNA using primers complementary to the cDNA; and (c) detecting and/or measuring the amplified cDNA. In a specific embodiment, the amplification is performed by means of a polymerase chain reaction and the amplified HAI-TMIP cDNA is detected using techniques known to one of skill in the art, for example, by visualization on an ethidium bromide stained gel, or similar gel containing a detectable label having the property of intercalation with nucleic acids, or by including a labelled deoxynucleic acid triphosphate in the reaction mixture, or by southern hybridization.

In one embodiment of the invention, the HAI-TMIP gene product detected and/or measured is and HAI-TMIP protein. In a specific embodiment, the amount of HAI-TMIP protein in a sample from the subject is detected and/or measured by steps comprising: (a) contacting the sample with a HAI-TMIP-binding agent which binds to HAI-TMIP protein (e.g., an anti-HAI-TMIP antibody) under conditions that permit binding of the agent to the HAI-TMIP protein; and (b) detecting and/or measuring the binding of the HAI-TMIP protein to the agent. The HAI-TMIP-binding agent may be labelled or a labelled antibody which binds to the HAI-TMIP-binding agent may be used in accordance with the methods of the invention. Alternatively, the HAI-TMIP protein may be labelled or a labelled antibody which binds to the HAI-TMIP protein may be used in accordance with the methods of the invention. Techniques for labelling agents (e.g., antibodies) are known to one of skill in the art and include, for example, radioactive isotopes, fluorescent molecules, enzymes, and magnetic beads. Similar methods can be applied to the detection of a complex of the invention.

In another embodiment, HAI-TMIP protein is detected in a sample from a subject by steps comprising: (a) contacting the sample with a first HAI-TMIP-binding agent (e.g., an anti-HAI-TMIP antibody) bound to a solid support under conditions that permit binding of the agent to the HAI-TMIP protein; (b) contacting the solid support with a second HAI-TMIP-binding agent (e.g., an anti-HAI-TMIP antibody) under conditions that permit binding of the second agent to the HAI-TMIP protein; and (c) detecting the binding, if any, between the second agent and the HAI-TMIP protein. In accordance with this embodiment, the second HAI-TMIP-binding agent may be labelled or unlabelled. In one embodiment, the second HAI-TMIP-binding agent is labelled. In another embodiment, the second HAI-TMIP-binding agent is unlabelled and a labelled antibody which binds to the second agent is used for detection of bound HAI-TMIP-binding agent. Similar methods can be applied to the detection of a complex of the invention.

In one embodiment, binding of a HAI-TMIP-binding agent in tissue sections is used to detect HAI-TMIP localization or aberrant (e.g., high, low, absent) amounts of HAI-TMIP protein or RNA in the tissue. In a specific embodiment, binding of a HAI-TMIP-binding agent in tissue sections is used to detect HAI-TMIP expression on the surface of cells. In a specific embodiment, a HAI-TMIP-binding agent can be used to assay a patient tissue or serum sample for the presence of the HAI-TMIP where an aberrant level of HAI-TMIP is indicative of a cancer. Similar embodiments are applicable to a complex of the invention.

In a specific embodiment, the amount of a HAI-TMIP gene product is detected in vivo in a subject according to a method comprising the steps of: (a) administering to the subject an effective amount of a labelled HAI-TMIP-binding agent that immuno specifically binds to HAI-TMIP, and (b) detecting the labelled agent in the subject following a time interval sufficient to allow the labelled agent to concentrate at sites in the subject where the HAI-TMIP gene product is expressed. In accordance with this embodiment, the HAI-TMIP-binding agent is administered to the subject according to any suitable method in the art, for example, parenterally or intraperitoneally. In accordance with this embodiment, the effective amount of the agent is the amount which permits the detection of the agent in the subject. This amount will vary according to the particular subject, the label used, and the detection method employed. For example, it is understood in the art that the size of the subject and the imaging system used will determine the amount of labelled agent needed to detect the agent in a subject using an imaging means. In the case of a radiolabelled agent for a human subject, the amount of labelled agent administered is measured in terms of radioactivity, for example from about 5 to 20 millicuries of ⁹⁹Tc. The time interval following the administration of the labelled agent which is sufficient to allow the labelled agent to concentrate at sites in the subject where the HAI-TMIP gene product is expressed will vary depending on several factors, for example, the type of label used, the mode of administration, and the part of the subject's body that is imaged. In a particular embodiment, the time interval that is sufficient is 6 to 48 hours, 6 to 24 hours, or 6 to 12 hours. In another embodiment the time interval is 5 to 20 days or 5 to 10 days. The presence of the labelled HAI-TMIP-binding agent can be detected in the subject using imaging means known in the art. In general, the imaging means employed depends upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), and sonography. In a specific embodiment, the HAI-TMIP-binding agent is labelled with a radioisotope and is detected in the patient using a radiation responsive surgical instrument (Thurston et al., U.S. Pat. No. 5,441,050). In another embodiment, the HAI-TMIP-binding agent is labelled with a fluorescent compound and is detected in the patient using a fluorescence responsive scanning instrument. In another embodiment, the HAI-TMIP-binding agent is labelled with a positron emitting metal and is detected in the patient using positron emission-tomography. In yet another embodiment, the HAI-TMIP-binding agent is labelled with a paramagnetic label and is detected in a patient using magnetic resonance imaging (MRI). Similar methods can be applied to detecting a complex of the invention in a subject.

Non-limiting examples of types of cancers that can be detected, diagnosed, monitored, predicted and/or prognosed in accordance with the methods of the invention are provided in Section 5.18.2.2. In a particular embodiment, the cancer detected, diagnosed, monitored, predicted and/or prognosed in accordance with the methods of the invention is breast, prostate, colon, kidney, liver, lung, ovarian, uterine, rectum, skin, or esophageal cancer. In some embodiments, the cancer detected, diagnosed, monitored, predicted and/or prognosed is not prostate cancer, colon cancer and/or leukemia (e.g., acute myelogenous leukemia). In another embodiment, the cancer detected, diagnosed, monitored, predicted and/or prognosed in accordance with the methods of the invention is metastatic cancer. In a specific embodiment, the cancer detected, diagnosed, monitored, predicted and/or prognosed in accordance with the methods of the invention is metastatic breast, metastatic prostate, metastatic colon, metastatic kidney, metastatic liver, metastatic lung, metastatic ovarian, metastatic uterine, metastatic rectum, metastatic skin, or metastatic esophageal cancer.

In a specific embodiment, the efficacy of a therapy is monitored by determining the effect of a therapy (e.g., an agent) on the expression and/or activity of a HAI-TMIP gene product, and/or the formation, stabilization and/or activity of a complex of the invention. In a particular embodiment, the monitoring of the effect of the therapy on the expression and/or activity of a HAI-TMIP gene product is applied to basic drug screening, preclinical studies, clinical trials and during prophylactic or therapeutic regimens designed to treat, prevent or manage cancer.

Methods for monitoring the progression of a cancer can be performed at any point following the diagnosis of the cancer. Methods for monitoring the efficacy of a therapy can also be performed at any point following the administration of a therapy. In some instances, monitoring is performed biweekly, weekly, bimonthly, monthly, biannually, yearly or every two years. It is understood that the frequency of monitoring can, at least in part, depend upon the particular situation (e.g., the severity of the cancer, overall health of the subject, the particular therapy being administered, etc.). In one embodiment, monitoring the cancer is carried out by repeating the method for diagnosis at specific times following the initial diagnosis. In a particular embodiment, the method for diagnosis is repeated 1 month, two months, three months, four months, five months, or six months, one year, two years, three years, four years, or five years after initial diagnosis.

In particular embodiments, the sample is taken from the subject one month, two months, three months, six months, or one year prior to the time at which the progression of the cancer is monitored. In one embodiment, the control sample is a sample taken from the subject following diagnosis of the cancer. In a specific embodiment, the control sample is a sample taken from the subject within 1 week, 2 weeks, 3 weeks, 4 weeks, 5 weeks, 6 weeks, 7 weeks, 8 weeks, 9 weeks, or 10 weeks following the diagnosis of the cancer. In another specific embodiment, the control sample is a sample taken from the subject within 1 month, 2 months, 3 months, 4 months, 5 months, or 6 months following the diagnosis of the cancer. In a preferred embodiment, more than one control sample is taken from the subject at each of several time points in order to monitor the progression of the cancer.

In a particular embodiment, the sample from the subject is tested one week, two weeks, three weeks, four weeks, five weeks, six weeks, seven weeks, eight weeks, one month, two months, three months, four months, five months, six months, seven months, eight months, one year, two years, three years, four years, five years, six years, seven years, eight years, nine years, or ten years following the administration of one or more rounds of therapy.

In one embodiment of the methods of this invention, the detection, diagnosis, or monitoring is carried out by detecting the amount of HAI-TMIP RNA, preferably mRNA, in a tumor biopsy sample from the subject. In another embodiment, detection, diagnosis, or monitoring is carried out by detecting the amount of HAI-TMIP protein in a tumor biopsy sample from the subject. In another embodiment, detection, diagnosis, or monitoring is carried out by detecting the activity of HAI-TMIP in a tumor biopsy sample from the subject.

In one embodiment, the HAI-TMIP gene product is an HAI-TMIP variant 1, 2, 3, 4, 5, 6 or 7 gene product. In a specific embodiment, the HAI-TMIP gene product is an HAI-TMIP variant 1, 2, 3, 4, 5, 6 or 7 RNA product. In another embodiment, the HAI-TMIP gene product is an HAI-TMIP variant 1, 2, 3, 4, 5, 6 or 7 protein product.

In another embodiment, the invention provides a method for imaging and localizing primary tumors or metastases in vivo. In accordance with this embodiment, the cancer is detected, diagnosed, or monitored in a subject. In a preferred embodiment, the subject is an animal, preferably a mammal and most preferably a human. In accordance with one aspect of this embodiment, a tumor is detected using a HAI-TMIP-binding agent or an agent that immunospecifically binds to a complex of the invention (e.g., an antibody). In a particular embodiment, the agent is an anti-HAI-TMIP antibody which immunospecifically binds to a HAI-TMIP or a specific variant. In a specific embodiment, the agent is an anti-HAI-TMIP antibody which immunospecifically binds to HAI-TMIP variant 3, 4, 5, 6, and/or 7.

The present invention provides methods for detecting or diagnosing cancer (e.g., metastic cancer) in a subject, the methods comprising: (a) determining the amount of autoantibody that immunospecifically binds to a polypeptide described in Section 5.2 in the subject or a sample from the subject; and (b) comparing the amount of autoantibody in (a) to a corresponding control, wherein cancer is detected or diagnosed if there is an alteration in the amount of autoantibody in (a) relative to the amount of autoantibody in the control. The present invention also provides methods for detecting or diagnosing cancer (e.g., metastic cancer) in a subject, the methods comprising: (a) determining the amount of autoantibody that immunospecifically binds to a complex of the invention in the subject or a sample from the subject; and (b) comparing the amount of autoantibody in (a) to a corresponding control, wherein cancer is detected or diagnosed if there is an alteration in the amount of autoantibody in (a) relative to the amount of autoantibody in the control. In specific embodiments, an increase in the amount of autoantibody relative to a negative control (such as, a healthy, cancer-free subject or a sample from a healthy cancer-free subject) indicates that the subject has cancer. The amount of autoantibody can be determined in vitro or in vivo using techniques known to one of skill in the art. In a specific embodiment, a sample from the subject is obtained and an immunoassay, such as an ELISA, is used to determine the amount of autoantibody.

In accordance with the methods of this invention, the detection of one or more HAI-TMIP gene products, a complex of the invention or an autoantibody described herein may optionally be combined with detection of one or more additional predictive, prognostic or diagnostic indicators. The particular indicator will vary depending on the particular cancer suspected or present in the subject. In one embodiment, the prognostic indicator is selected from the group consisting of a Ras protein or clinically relevant mutant thereof, an ErbB2/c-neu protein, an epidermal growth factor receptor, an estrogen receptor, a p53 tumor suppressor protein or a clinically relevant mutant thereof, a retinoblastoma protein, or a c-myc protein. In another embodiment, the prognostic indicator is a nucleic acid corresponding to the protein product of a Raps protein or clinically relevant mutant thereof, an ErbB2/c-neu protein, an epidermal growth factor receptor, an estrogen receptor, a p53 tumor suppressor protein or a clinically relevant mutant thereof, a retinoblastoma protein, or a c-myc protein, or a polypeptide or fragment thereof having clinical significance as a prognostic indicator.

Samples from a subject used in accordance with the methods of the invention for predicting, detecting, diagnosing, prognosing and/or monitoring cancer in the subject include, but are not limited to, biological samples and samples derived from a biological sample. In certain embodiments, in addition to the biological sample itself or in addition to material derived from the biological sample such as cells, nucleic acids or proteins, the sample used in the methods of this invention comprises added water, salts, glycerin, glucose, an antimicrobial agent, paraffin, a chemical stabilizing agent, heparin, an anticoagulant, or a buffering agent. In certain embodiments, the biological sample is blood, serum, urine, or interstitial fluid. In another embodiment, the sample is a tissue sample. In a particular embodiment, the tissue sample is breast, colon, lung, liver, ovarian, pancreatic, prostate, renal, bone or skin tissue. In a specific embodiment, the tissue sample is a biopsy. The amount of biological sample taken from the subject will vary according to the type of biological sample and the method of the HAI-TMIP gene product detection to be employed.

In accordance with the methods of the invention, a sample derived from a biological sample is one in which the biological sample has been subjected to one or more pretreatment steps prior to the detection and/or measurement a HAI-TMIP gene product in the sample. In certain embodiments, a biological fluid is pretreated by centrifugation, filtration, precipitation, dialysis, or chromatography, or by a combination of such pretreatment steps. In other embodiments, a tissue sample is pretreated by freezing, chemical fixation, paraffin embedding, dehydration, permeablization, or homogenization followed by centrifugation, filtration, precipitation, dialysis, or chromatography, or by a combination of such pretreatment steps. In certain embodiments, the sample is pretreated by adjusting the concentration of protein or nucleic acid in the sample, by adjusting the pH or ionic strength of the sample, or by removing contaminating proteins, nucleic acids, lipids, or debris from the sample prior to the determination of the amount of the HAI-TMIP gene product in the sample according to the methods of this invention.

The samples for use in the methods of this invention may be taken from any animal subject, preferably mammal, most preferably a human. The subject from which a sample is obtained and utilized in accordance with the methods of this invention includes, without limitation, an asymptomatic subject, a subject manifesting or exhibiting 1, 2, 3, 4 or more symptoms of cancer, a subject clinically diagnosed as having cancer, a subject predisposed to cancer, a subject suspected of having cancer, a subject diagnosed as having cancer, a subject undergoing therapy for cancer, a subject that has been medically determined to be free of cancer (e.g., following therapy for the cancer), a subject that is managing cancer, or a subject that has not been diagnosed with cancer. In certain embodiments, the term “cancer-free,” as used herein, refers to a subject or subjects that are free from cancer. In other embodiments, the term refers to a subject or subjects free from any disorder.

In certain embodiments, a positive or negative control sample is a sample that is obtained or derived from a corresponding tissue or biological fluid as the sample to be analyzed in accordance with the methods of the invention.

### 5.20 Kits

The present invention provides kits that can be used in the above methods. In specific embodiments, the present invention provides kits for detecting and diagnosis a disorder described herein. In other embodiments, the present invention provides kits for determining the prognosis of a disorder described herein. In other embodiments, the present invention provides kits for monitoring the progression of a disorder described herein. In yet other embodiments, the present invention provides kits for determining the efficacy of a therapy. In a specific embodiment, the disorder is cancer. In a more specific embodiment, the cancer is breast cancer.

The present invention also provides a pharmaceutical pack or kit comprising one or more containers filled with reagents for detecting and/or measuring a HAI-TMIP gene product. In one embodiment, the pharmaceutical pack or kit optionally comprises instructions for the use of the reagents provided for detecting and/or measuring a HAI-TMIP gene product. In another embodiment, the pharmaceutical pack or kit optionally comprises a notice in the form prescribed by a governmental agency regulating the manufacture, use or sale of pharmaceuticals or biological products, which notice reflects approval by the agency of manufacture, use or sale for human administration.

In an embodiment, the pharmaceutical pack or kit comprises in one or more containers a HAI-TMIP-binding agent. In a particular embodiment, the agent is a HAI-TMIP antibody that selectively or immunospecifically binds to a HAI-TMIP. In another embodiment, the agent is a HAI-TMIP antibody that selectively or immunospecifically binds to HAI-TMIP variant 3, 4, 6 and 7 or HAI-TMIP variant 5.

For nucleic acid micoarray kits, the kits generally comprise probes attached to a solid support surface. In one such embodiment, probes can be either oligonucleotides or longer length probes including probes ranging from 150 nucleotides in length to 800 nucleotides in length. The probes may be labelled with a detectable label. The microarray kits may comprise instructions for performing the assay and methods for interpreting and analyzing the data resulting from the performance of the assay. The kits may also comprise hybridization reagents and/or reagents necessary for detecting a signal produced when a probe hybridizes to a HAI-TMIP nucleic acid sequence (e.g., a nucleotide sequence of Section 5.1, supra). Generally, the materials and reagents for the microarray kits are in one or more containers. Each component of the kit is generally in its own a suitable container.

For RT-PCR kits, the kits generally comprise pre-selected primers specific for particular RNA products (e.g., an exon(s), an intron(s), an exon junction(s), and an exon-intron junction(s)) of a nucleotide sequence of a HAI-TMIP (e.g., a nucleotide sequence of Section 5.1, supra). The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for a nucleotide sequence of a HAI-TMIP (e.g., a nucleotide sequence of Section 5.1, supra). The probes may or may not be labelled with a detectable label (e.g., a fluorescent label). Each component of the RT-PCR kit is generally in its own suitable container. Thus, these kits generally comprise distinct containers suitable for each individual reagent, enzyme, primer and probe. Further, the RT-PCR kits may comprise instructions for performing the assay and methods for interpreting and analyzing the data resulting from the performance of the assay.

For Quantitative PCR, the kits generally comprise pre-selected primers specific for a HAI-TMIP nucleic acid sequences. The Quantitative PCR kits may also comprise enzymes suitable for amplifying nucleic acids (e.g., polymerases such as Taq), and deoxynucleotides and buffers needed for the reaction mixture for amplification. The Quantitative PCR kits may also comprise probes specific for the nucleic acid sequences associated with or indicative of a condition. The probes may or may not be labelled with a fluorophore. The probes may or may not be labelled with a quencher molecule. In some embodiments, the Quantitative PCR kits also comprise components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the assay and methods for interpreting and analyzing the data resulting from the performance of the assay.

Examples of probes and primers specific for HAI-TMIP variants 1 to 7 that can be included in the kits are provided below:

Probe 1: T1:
(exon 2; SEQ ID NO: 22)
5′-FAM-AGAGCCGCCTAGAACCAGTCCG-TAMRA-3′
Probe 2: T2:
(exon 5; SEQ ID NO: 23)
5′-FAM-CAAGGGCATCAACTACACGACCCA-TAMRA-3′

Primers:

| | |
|---|---|
| ATPa-P1: | |
| 5′ CGGTATAATCAACACTACGAGAG 3′ | (SEQ ID NO: 12) |
| ATPa-P11: | |
| 5′ CAAAGCATTTCTGGAGACcaGT 3′ | (SEQ ID NO: 15) |
| ATPa-P3: | |
| 5′ GTCTTGACCTTCTTTGCGGCTC 3′ | (SEQ ID NO: 14) |
| ATPa-P19: | |
| 5′ GGAACCAATTGGACCctTTC 3′ | (SEQ ID NO: 19) |
| ATPa-P13: | |
| 5′ GGCCGGACtgGGACT 3′ | (SEQ ID NO: 16) |
| ATPa-P14: | |
| 5′ TAGAGCCGCCTAGAACCagGGA 3′ | (SEQ ID NO: 17) |
| ATPa-P15 | |
| 5′ GAAAGACACACTTTGTTAACAGGGA 3′ | (SEQ ID NO: 18) |
| ATPa-P16: | |
| 5′ ACACTCATCTTCAAAAGACTGGGA 3′ | (SEQ ID NO: 24) |
| ATPa-P20 | |
| 5′ GAAGCTGCAACTATATCTAACGAAC 3′ | (SEQ ID NO: 20) |
| ATPa-P21: | |
| 5′ AGAGCCGCCTAGAACCAGGTC 3′ | (SEQ ID NO: 21) |

TABLE 7

| | | | Length | |
|---|---|---|---|---|
| | | | (PCR | |
| Variant | Primers | Probe | fragment ) | Tm |
| Variant 1 | ATPα -P1/ATPα -P11 | T1 | 168 bp | |
| Variant 2 | ATPα -P3/ATPα -P11 | T1 | 168 bp | 68° C. |
| Variant 3 | ATPα -P21/ATPα -P19 | T2 | 459 bp | 68° C. |
| Variant 4 | ATPα -P15/ATPα -P19 | T2 | 382 bp | 61.8° C. |
| Variant 5 | ATPα -P16/ATPα -P20 | T2 | 400 bp | 68° C. |
| Variant 6 | ATPα -P14/ATPα -P19 | T2 | 389 bp | 68° C. |
| Variant 7 | ATPα -P13/ATPα -P19 | T2 | 370 bp | |

For antibody based kits, the kit can comprise, for example: (1) a first antibody (which may or may not be attached to a solid support) which binds to a HAI-TMIP protein (e.g., HAI-TMIP variant 3, 4, 5, 6 or 7); and, optionally, (2) a second, different antibody which binds to either the HAI-TMIP protein, or the first antibody and is conjugated to a detectable label (e.g., a fluorescent label, radioactive isotope or enzyme). In a specific embodiment, the peptide, polypeptide or protein of interest is associated with or indicative of a condition (e.g., a disease). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay.

The present invention also provides kits comprising, in one or more containers, one or more RNAi provided in Table 6, supra. These kits may comprise instructions for using the RNAi.

The present invention further provides kits comprising, in one or in one containers, peptides 1 to 5 described in Section 5.8, supra.

### 5.21 Articles of Manufacture

The present invention also encompasses a finished packaged and labelled pharmaceutical product. This article of manufacture includes the appropriate unit dosage form in an appropriate vessel or container such as a glass vial or other container that is hermetically sealed. The pharmaceutical product may be formulated in single dose vials. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intranasal, or topical delivery.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses and monitoring procedures.

In a specific embodiment, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises an activator and wherein said packaging material includes instruction means which indicate that said antibody can be used to prevent, treat, and/or ameliorate a disorder described herein by administering specific doses and using specific dosing regimens as described herein. In another embodiment, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises an inhibitor and wherein said packaging material includes instruction means which indicate that said antibody can be used to prevent, treat, and/or ameliorate a disorder described herein by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises an activator or an inhibitor and the other pharmaceutical agent comprises a second, different prophylactic or therapeutic, and wherein said packaging material includes instruction means which indicate that said agents can be used to treat, prevent and/or manage a disorder described herein by administering specific doses and using specific dosing regimens as described herein.

The present invention provides that the adverse effects that may be reduced or avoided by the methods of the invention are indicated in informational material enclosed in an article of manufacture for use in preventing, treating and/or managing a disorder described herein. Adverse effects that may be reduced or avoided by the methods of the invention include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation.

Further, the information material enclosed in an article of manufacture for use in preventing, treating and/or managing disorder described herein can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### 6. Examples

### 6.1 Example 1

### Identification of HAI-TMIP Variants

The techniques below were used to identify HAI-TMIP variants 3 to 7.

### 6.1.1 Materials & Methods

### 6.1.1.1 Cell Lines

The cell lines used in the methods to identify the HAI-TMIP variants were MDA-MB-231 (a highly invasive breast cancer cell line), MCF-10F (an immortalized human breast epithelial cell line), MDA-MB-435 (a melanoma cell line), MCF-7 (a human breast adenocarcinoma cell line), and MCF-7/ADR (an adriamycin-resistant variant of the MCF-7 cell line).

### 6.1.1.2 Total RNA Extraction from Cell Line

1 ml of TRIZOL Reagent was added to a dish (about 10 cm²) to lyse cells. The homogenized samples were incubated for 5 minutes at 15 to 30° C., then added 0.2 ml of chloroform. Tubes were vigorously shaken by hand for 15 seconds and then incubated at 15 to 30° C. for 2 to 3 minutes. The samples were centrifuged at no more than 12,000×g for 15 minutes at 2 to 8° C. Following centrifugation, the colorless upper aqueous phase was transferred to a RNase-free 1.5 ml tube and 0.5 ml of isopropyl alcohol was added. The samples were incubated at 15 to 30° C. for 10 minutes and centrifuged at no more than 12,000×g for 10 minutes at 2 to 8° C. The supernatant was removed and the RNA pellet was washed once with 1 ml 75% ethanol, mixed by vortexing and centrifuged at no more than 7,500×g for 5 minutes at 2 to 8° C. The RNA pellet was then dried.

### 6.1.1.3 First-Strand cDNA Synthesis

Approximately 1 to 5 μg of total RNA, 1 μl of oligo(dT)₂O (50 μM), and 1 μl 10 mM dNTP mix was added to a RNase-free microcentrifuge tube and sterile, distilled water was added to bring the volume up to 13 μl. The mixture was heated at 65° C. for 5 minutes and incubated on ice for at least 1 minute. The contents of the tube were briefly centrifugation and 4 μl 5× First-Strand Buffer, 1 μl 0.1 M DTT, 1 μl RNaseOUT and 1 μl of SuperScript. III RT (200 units/μl) were added to the tube. The contents of the tube were mixed by pipetting gently up and down and the tube was incubated at 50° C. for approximately 30 to 60 minutes.

### 6.1.1.4 PCR

PCR was performed in a final volume of 50 μl of a reaction mix containing 0.1 mM each dNTP, 1.5 mM MgCl₂, PCR buffer without magnesium, 0.25 μM each primer for the investigation of HAI-TMIP splice variants, and 2 units of Taq DNA polymerase. Reaction mixtures were heated for 4 minutes at 94° C., followed by 30 cycles of amplification. Each amplification cycle consisted of denaturation at 94° C. for 30 seconds, annealing between 55 and 68° C., and extension at 72° C. for 2 minutes. The following primers were designed for the investigation of HAI-TMIP splice variants:

ATPa-P1:
(SEQ ID NO: 12)
5′ CGG TAT AAT CAA CAC TAC GAG AG 3′
ATPa-P2:
(SEQ ID NO: 13)
5-GAA CAA TGA CAA AAC TGA ACT GG-3′
ATPa-P3
(SEQ ID NO: 14)
5′-GTCTTGACCTTCTTTGCGGCTC-3′

Primers ATPa-P1 and ATPa-P2 were used to amplify variant 1, variant 3, variant 4, variant 5, variant 6. Primers ATPa-P3/ATPa-P2 were used to amplify variant 2 and variant 7.

### 6.1.1.5 Cloning and Sequencing

Gel-purified PCR fragments were linked to linearized pGEM-T. E. coli was transformed with the pGEM-T vectors containing the PCR fragments and white clones were picked for DNA sequencing. SP6/T7 primers were used to sequence, and sequencing were performed by ABI PRISM 3730.

### 6.1.1.6 Southern Blot

Genomic DNA was isolated from cultured cells according to the manuals of Axygen Genomic DNA kits and quantified by measuring A260. 10 μg DNA was digested with Ecorl and BamH I, then run it in 0.8% agarose TAE gel, transferred to charged Nylon membrane, immobilized by baking at 80° C. for 2 hours. The probes to distinguish the different isoforms were labelled with Digoxin-dUTP. Prehybrization was carried out in 5×SSC, SxDeharts', 0.5% SDS,0.1% sarcosle and 100 μg/ml denatured sheared salmon DNA, colored by NBT/BCIP.

Exons were predicted using the NCBI LocusLink program.

### 6.1.1.7 Northern Blot

Total RNA was isolated from breast tumor sample, quantified and 10 microgram of total RNA to electrophoresis. ECL kits were used to detect the specific mRNA bands on the immobilized membrane.

### 6.1.2 Results

HAI-TMIP variants 3 to 7 were identified from the cell lines indicated in Table 8, infra.

The nucleotide sequence of HAI-TMIP variant 3 is depicted in FIG. 1. The nucleotide sequence is 1692 base pairs. The open reading frame for HAI-TMIP variant 3 begins at base pair 134 and ends at base pair 1643.

The nucleotide sequence of HAI-TMIP variant 4 is depicted in FIG. 2. The nucleotide sequence is 1879 base pairs. The open reading frame for HAI-TMIP variant begins at base pair 321 and ends at base pair 1830.

The nucleotide sequence of HAI-TMIP variant 5 is depicted in FIG. 3. The nucleotide sequence is 1782 base pairs. The open reading frame for HAI-TMIP variant 5 begins at base pair 638 and ends at base pair 1733.

The nucleotide sequence of HAI-TMIP variant 6 is depicted in FIG. 4. The nucleotide sequence is 1613 base pairs. The open reading frame for HAI-TMIP variant 6 begins at base pair 55 and ends at base pair 1564.

The nucleotide sequence of HAI-TMIP variant is depicted in FIG. 5. The nucleotide sequence is 1720 base pairs. The open reading frame for HAI-TMIP variant begins at base pair 162 and ends at base pair 1671.

An alignment of the nucleotide sequences of HAI-TMIP variants 1 to 7 is depicted in FIG. 9. The exon and intron structure of HAI-TMIP variants 1 to 7 is depicted in FIG. 8. HAI-TMIP variant 1 comprises 13 exons. HAI-TMIP variant 2 comprises 12 exons. HAI-TMIP variants 1 and 2 encode the same amino acid sequence. The difference between HAI-TMIP variants 1 and 2 is that exon 1 of HAI-TMIP variant 1 is deleted in HAI-TMIP variant 2 and exon 2 in HAI-TMIP variant 2 is 42 base pairs longer than exon 2 in HAI-TMIP variant 1. The difference between HAI-TMIP variants 1 and 3 is that exon 2 of HAI-TMIP variant 1 is deleted in HAI-TMIP variant 3. The difference between HAI-TMIP variants 1 and 4 is that exon 2 of HAI-TMIP variant 1 is deleted in HAI-TMIP variant 4 and HAI-TMIP variant 4 contains an exon that is 187 base pairs in the intron 3 position of HAI-TMIP variant I. The difference between HAI-TMIP variants 1 and 5 is that exon 2 of HAI-TMIP variant 1 is deleted in HAI-TMIP variant 5 and intron 4 is alternatively spliced as an exon in HAI-TMIP variant 5. The difference between HAI-TMIP variants 1 and 6 is that exons 2 and 3 of HAI-TMIP variant 1 are deleted in HAI-TMIP variant 6. The difference between HAI-TMIP variants 2 and 7 is that exon 2 of HAI-TMIP variant 2 is deleted in HAI-TMIP variant 7.

The predicted amino acid sequence for HAI-TMIP variants 3, 4, 6 and 7 are identical and is depicted in FIG. 6. The amino acid sequence for HAI-TMIP variants 3, 4, 6, and 7 is 503 amino acid residues in length. The predicted amino acid sequence for HAI-TMIP variant 5 is depicted in FIG. 7 and is 365 amino acid residues in length. An alignment of the amino acid sequences of HAI-TMIP variants 1, 3 and 5 is depicted in FIG. 10. The amino acid sequence of HAI-TMIP variant 1 comprises the amino acid sequences of HAI-TMIP variants 3 and 5. The amino acid sequence of HAI-TMIP variant 3 comprises the amino acid sequence of HAI-TMIP variant 5.

TABLE 8

| | | | cDNA |
|---|---|---|---|
| | | | Sequence |
| | Source Material | | Length |
| Variant | (Cell lines) | Primers | (bp) |
| Variant 1 | GenBank No | ATPa-P1/ATPa-P2 | |
| | NM_001001937 | | |
| | (SEQ ID NO: 1) | | |
| Variant 2 | GenBank No NM_004046 | ATPa-P3/ATPa-P2 | |
| | (SEQ ID NO: 2) | | |
| | MCF-10F, MDA-MB-231, | | |
| | MDA-MB-435, MCF-7, | | |
| | MCF-7/ADR | | |
| Variant 3 | MCF-7/ADR (12/14) | ATPa-P1/ATPa-P2 | 1692 |
| Variant 4 | MCF-7/ADR (12/14) | ATPa-P1/ATPa-P2 | 1879 |
| Variant 5 | MCF 10F(1/2) | ATPa-P1/ATPa-P2 | 1782 |
| Variant 6 | MCF 10F(1/2) | ATPa-P1/ATPa-P2 | 1613 |
| Variant 7 | MCF-7/ADR (1/2) | ATPa-P3/ATPa-P2 | 1720 |

### 6.2 Example 2

### Development of Antibodies

This example provides methods for producing antibodies that immunospecifically bind to HAI-TMIP variants 3, 4, 5, 6 and 7.

### 6.2.1 Development of Murine-Derived Monoclonal Antibodies

### 6.2.1.1 Antigens

HAI-TMIP variants 3 to 7 were recombinantly expressed in E. coli and five (5) peptides covering HAI-TMIP variants 3 to 7 were chemically synthesized using commercially available methods. The sequences of the five peptides are provided in FIG. 11.

### 6.2.1.2 Immunization

Balb/c mice were immunized with an antigen in complete or incomplete Freuds adjuvent (Sigma). Then mouse with highest titer against antigen was selected and sacrificed by bleeding. Spleen cells of sacrificed mouse were fused to partner cells (sp2/0, ATCC) at ratio of 2:1 by 50% PEG(Sigma). Fused cells in medium containing 10% FBS and HAT (Sigma) were seeded in 96-well microplates, at approximately 10000 cells/well. Supernatant of wells with clones growing were screened by indirect ELISA and positive wells were subcloned 2-3 times.

### 6.2.2 Development of Rabbit-Derived Monoclonal Antibodies

### 6.2.2.1 Antigens

HAI-TMIP variants 3 to 7 were recombinantly expressed in E. coli and five (5) peptides covering HAI-TMIP variants 3 to 7 were chemically synthesized using commercially available methods. The sequences of the five peptides are provided in FIG. 11.

### 6.2.2.2 Immunization

Rabbits were primary immunized with an antigen in complete Freuds adjuvent (CFA, Sigma) and boosted by antigen in incomplete Freuds adjuvent (IFA, Sigma). Titers of immunized rabbits serum were tested before fusion and the rabbit with highest titer against antigen was sacrificed by bleeding. Spleen cells of sacrificed rabbit were fused to partner cells (240E1-1-2) at ratio of 2:1 by 50% PEG (Sigma). Fused cells in medium containing 10% FBS and HAT(Sigma) were seeded in 96-well microplates, at approximately 20000 spleen cells/well. Supernatant of wells with clones growing were screened by indirect ELISA and positive wells were subcloned 2-3 times.

### 6.2.3 Development of Human-Derived Monoclonal Antibodies by Hetro-Hybridoma

The anti-agglutinated human blood was obtained from the blood bank of Changhai Hospital, Shanghai. Blood positive in an anti-HAI-TMIP antigen reaction by ELISA was selected as the source of T/B cells. Generally, the peripheral blood mononuclear cells (PBMC) are initially separated upon Ficoll-Paque (Amersham Pharmacia Biosciences) and then the PBMCs are further refined through the second step of Ficoll-Paque (Amersham Pharmacia Biosciences). Finally the B cells were obtained for the following experiments. CD19-positive cells and CD4-positive cells were isolated from PBMCs by a human CD4 and CD19 selection kit (Miltenyi Biotec GmbH), respectively, and mixed to make a B cell/T cell. B/T cells were cultured in complete RPMI medium 1640 (Invitrogen), which contain 10% heat-inactivated human serum AB, 2 mM L-glutamine, 0.1 mM nonessential amino acids, 1 mM sodium pyruvate, and 55 μM 2-mercaptoethanol (Invitrogen).

Purified B cells were fused with partner cells (K6H6/B5,ATCC) by electrofusion (Eppendorf) at ratio of 1:1.Fused cells in RPMI medium 1640 containing 10% FBS and HAT(Sigma) were seeded in 96-well microplates at 5000 cells/well. Seeded wells containing viable hybridoma cells were screened by indirect ELISA after 2-3 weeks and positive wells were subcloned 2-3 times.

### 6.2.4 Development of Human-Derived Monoclonal Antibodies by Human-Human Hybridoma

B cells were isolated as described in Section 6.2.1.3. Purified B cells were fused with partner cells (Karpas 707 cells, gift from Prof. Karpas) by electrofusion (Eppendorf) at ratio of 1:1. Fused cells in RPMI medium 1640 containing 10% FBS, ouabain and HAT(Sigma) were seeded in 96-well microplates at 5000 cells/well. Seeded wells containing viable hybridoma cells were screened by indirect ELISA after 2-3 weeks and positive wells were subcloned 2-3 times.

### 6.2.5 Development of Human-Derived Monoclonal Antibodies by Immortalized B Cells

B cells were isolated as described in Section 6.2.1.3. Supernatant of cultured B958 was used as the source of EBV. Three (3) days before transforming, feeder cells were prepared from bone marrow of Balb/c mice and seeded in 96-well microplates. Then, purified B cells in RPMI medium 1640 were seeded in 96-well microplates containing feeder cells at approximately 3-5 cells per well. 100 ul/well of EBV from B958 cells supernatant was simultaneously added into microplates. Every 4 days, half supernatant of the wells was changed with fresh completed medium until the appearance of colony of B cells was visible under microscope. Wells containing viable B cells were screened by indirect ELISA after 3-4 weeks and positive wells were subcloned 2-3 times.

### 6.2.6 Development of Monoclonal Antibodies by Phage Display

DNA sequences encoding VH and VL domains were amplified from animal cDNA libraries (e.g., human or murine cDNA libraries of affected tissues). The DNA encoding the VH and VL domains were recombined together with an scFv linker by PCR and cloned into a phagemid vector. The vector was electroporated into E. coli and the E. coli was infected with helper phage. Phage used in these methods was typically filamentous phage including fd and M13 and the VH and VL domains were usually recombinantly fused to either the phage gene III or gene VIII. Phage expressing an antigen binding domain that binds to a particular antigen was selected or identified with antigen, e.g., using labelled antigen or antigen bound or captured to a solid surface or bead.

### 6.2.7 Indirect ELISA Screening

Medium was replaced weekly, and HAT selection was maintained until completion of antigen reactivity screening. For the identification of antigen-reacting monoclonal antibodies, ELISA-based screenings were performed. Briefly, microliter plates were coated at room temperature for 6 hours with 1 μg per well of HAI-TMIP antigen diluted in coating buffer (50 mM carbonate-bicarbonate, pH 9.6). Plates were then blocked with blocking buffer (PBS containing 3% BSA and 0.05% Tween 20) for 2 hours at room temperature. Plates were washed once with washing buffer (PBS containing 0.05% Tween 20), and 100 μl supernatant from screened well was transferred into the ELISA plates. The binding reaction was carried out at room temperature for 2 hours. Subsequently, plates were washed 4 times, and 100 μl of horseradish peroxidase-conjugated goat anti-human IgG+M (R&D) diluted 1:10,000 in binding buffer (PBS containing 1% BSA and 0.05% Tween 20) was added, and reactions are carried out at room temperature for 1 hour. Finally, plates were washed 4 times, and 100 μl of the substrate of horseradish peroxidase (Sigma, USA) per well was added for 10 min. Reactions were stopped by adding 50 μl of 1 N sulfuric acid per well, and the absorbance was determined at 450 mu.

### 6.2.8 Preservation of the Positive Clones

The positive clones after ELISA have been carefully preserved in −80° C. and/or liquid nitrogen at −196° C. In some case, the positive clones have been further subcloned.

### 6.3 Example 3

### Breast Cancer Diagnosis

This example demonstrates the correlation between the expression of HAI-TMIP variants and breast cancer.

### 6.3.1 Materials & Methods

### 6.3.1.1 Genomic DNA copies of Different Variants on Different Cell Lines by PCR Cell Lines

The cell lines used in the methods to identify the HAI-TMIP variants were MDA-MB-231 (a highly invasive breast cancer cell line), MCF-10F (an immortalized human breast epithelial cell line), MDA-MB-435 (a melanoma cell line), MCF-7 (a human breast adenocarcinoma cell line), and MCF-7/ADR (an adriamycin-resistant variant of the MCF-7 cell line).

### Genomic DNA Extraction from Cell Line

Genomic DNA extraction Kit from Tiangen was used to extract genomic DNA. Briefly, genemic DNA was extracted by lysing cells, transferring the DNA to a centrifuge filter, eluting the DNA with TE and measuring the DNA by an ultraviolet-photometer.

### PCR

Primers was designed at 16042 (upstream AGGCTTGAACTCCTGGGCTCA) (SEQ ID NO: 42) and 16779 (downstream AATCCTTCCTCCCACCCCTGA) (SEQ ID NO: 30) of human ATP synthase genomic (gi:559316). 30 ng genomic DNA was used as template to amplify ATP synthase DNA, denatured at 94° C. 5 min, then 94° C. 30s, 68° C. 30s, 72° C. 30s, 35 cycles. Validation by real-time PCR(Sybgreen I).

### 6.3.1.2 Expression of Different Variants on Breast Tumor Tissues and Normal Breast Tissues by Real-Time RT-PCR

### Total RNA Extraction from Breast Tumor and Normal Breast Tissues

1 ml of TRIZOL Reagent was added to a dish (about 10 cm²) to lyse tissue cells. The homogenized samples were incubated for 5 minutes at 15 to 30° C., then added 0.2 ml of chloroform. Tubes were vigorously shaken by hand for 15 seconds and then incubated at 15 to 30° C. for 2 to 3 minutes. The samples were centrifuged at no more than 12,000×g for 15 minutes at 2 to 8° C. Following centrifugation, the colorless upper aqueous phase was transferred to a RNase-free 1.5 ml tube and 0.5 ml of isopropyl alcohol was added. The samples were incubated at 15 to 30° C. for 10 minutes and centrifuged at no more than 12,000×g for 10 minutes at 2 to 8° C. The supernatant was removed and the RNA pellet was washed once with 1 ml 75% ethanol, mixed by vortexing and centrifuged at no more than 7,500×g for 5 minutes at 2 to 8° C. The RNA pellet was then dried.

### First-Strand cDNA Synthesis

Approximately 1 to 5 μg of total RNA, 1 μl of oligo(dT)₂O (50 μM), and 1 μl 10 mM dNTP mix was added to a RNase-free microcentrifuge tube and sterile, distilled water was added to bring the volume up to 13 μl. The mixture was heated at 65° C. for 5 minutes and incubated on ice for at least 1 minute. The contents of the tube were briefly centrifugation and 4 μl 5× First-Strand Buffer, 1 μl 0.1 M DTT, 1 μl RNaseOUT and 1 μl of SuperScript. III RT (200 units/μl) were added to the tube. The contents of the tube were mixed by pipetting gently up and down and the tube was incubated at 50° C. for approximately 30 to 60 minutes.

### Real-Time PCR

PCR was performed in a final volume of 25 μl of a reaction mix containing 0.2 mM each dNTP, 2.5 mM MgCl₂, PCR buffer without magnesium, 0.2 μM each primer and 0.4 μM probe for the investigation of HAI-TMIP splice variants, 1.25 units of Taq DNA polymerase and 2.5 μl cDNA templates for each plasmid containing the splice variant to be quantified in molecular concentration ladder ranging from 107 to 103, or 1 μg cDNA templates for each sample to be quantified, such as cDNA extracted from MCF-10F, MCF-7, and MCF-7/ADR cell lines. Reaction mixtures were heated for 4 minutes at 94° C., followed by 45 cycles of amplification. Each amplification cycle consisted of denaturation at 94° C. for 25 seconds, annealing between 63 and 68.5° C. for 25 seconds, and extension at 72° C. for 25 seconds. The standard curve was drawn according to the CT value of the plasmid templates, and the copy number of the sample templates was calculated according to its CT value in the standard curve. The following primers were designed for the investigation of HAI-TMIP splice variants:

ATPa-P1:
(SEQ ID NO: 12)
5′ CGG TAT AAT CAA CAC TAC GAG AG 3′
ATPa-P2:
(SEQ ID NO: 13)
5-GAA CAA TGA CAA AAC TGA ACT GG-3
ATPa-P3
(SEQ ID NO: 14)
5′-GTCTTGACCTTCTTTGCGGCTC-3′

Primers ATPa-P1 and ATPa-P2 were used to amplify variant 1, variant 3, variant 4, variant 5, and variant 6. Primers ATPa-P3/ATPa-P2 were used to amplify variant 2 and variant 7.

Primers and probes for real-time PCR:

| Variants | Primers | probe | length |
|---|---|---|---|
| Variant 4 | ATPa-P15/ATPa-P19 | T2 | 382 bp |
| Variant 5 | ATPa-P16/ATPa-P20 | T2 | 402 bp |
| Variant 6 | ATPa-P14/ATPa-P19 | T2 | 378 bp |
| Variant 7 | ATPa-P13/ATPa-P19 | T2 | 369 bp |

Sequences of primers and probes for real-time PCR:

ATPa-P11:
(SEQ ID NO: 15)
5′-CAA AGC ATT TCT GGA GAC CAG T-3′
ATPa-P13:
(SEQ ID NO: 16)
5′-GGC CGG ACT GGG ACT-3′
ATPa-P14:
(SEQ ID NO: 17)
5′-TAG AGC CGC CTA GAA CCA GGG A-3′
ATPa-P15:
(SEQ ID NO: 18)
5′-GAA AGA CAC ACT TTG TTA ACA GGG A-3′
ATPa-P16:
(SEQ ID NO: 24)
5′-ACA CTC ATC TTC AAA AGA CTG GGA-3′
ATPa-P19:
(SEQ ID NO: 19)
5′-GGA ACC AAT TGG ACC CTT TC-3′
ATPa-P20:
(SEQ ID NO: 20)
5′-GAA GCT GCA ACT ATA TCT AAC GAA C-3′
Probe T2:
(SEQ ID NO: 23)
5′-FAM-CAAGGGCATCAACTACACGACCCA-TAMRA-3′.

### 6.3.1.3 Immunohistochemistry Analysis on Breast Cancer Tissue Microarray (TMA)

### Tissue Arrays and Patient Selection

Breast cancer tissue microarrays were purchased from Shanghai Hujing Biotech Co., Ltd, China and Cybrdi Inc., USA, which contained duplicates of 194 human breast cancer tissues and 15 normal breast tissues with a diameter of 1.0 mm. In addition, 13 atypical hyperplasia of breast tissues and 18 normal breast tissues were collected between 2004 and 2005 from the Shanghai Putuo Hospital. Selection of normal tissues was based on morphological criteria, and samples were taken from morphologically normal areas surrounding diseased tissue. Patients who received chemotherapy or radiotherapy before surgery were excluded. The samples of missing dots and invalid dots were also excluded. In this study 168 female breast cancer patients (range in age from 31 to 82, with a mean of 58 years), 13 atypical hyperplasia and 33 normal breast tissues were analyzed. All surgical material was fixed in 4% formalin and processed routinely. The tumors were classified according to the Pathological Tumor-Node-Metastasi (pTNM) system (sixth edition). The pathologic features examined included histological subtype, tumor size, primary tumor stage, and regional lymph node involvement. The microscopic slides from all specimens were reviewed by at least two pathologists. Table 9 summarizes the clinic pathological parameters of this study.

TABLE 9
Clinicopathological data and HAI-TMIP expression in 168 breast cancers

| | HAI-TMIP expression | | | | | | | |
|---|---|---|---|---|---|---|---|---|
| | Negative | | Moderate | | Strong | | Total | P value |
| Age | | | | | | | | |
| <50 | 5 | (7.0%) | 30 | (42.3%) | 36 | (50.7%) | 71 (100%) | No significant |
| ≧50 | 4 | (4.1%) | 31 | (32.0%) | 62 | (63.9%) | 97 (100%) | |
| Size | | | | | | | | |
| <2 cm | 2 | (13.3%) | 10 | (66.7%) | 3 | (20.0%) | 15 (100%) | P < 0.05 |
| ≧2 cm | 2 | (3.6%) | 24 | (42.8%) | 30 | (53.6%) | 56 (100%) | |
| Histological grade | | | | | | | | |
| Well differentiated | 3 | (0%) | 11 | (%) | 10 | (%) | 24 (100%) | P < 0.05 |
| Moderately differentiated | 6 | (5.3%) | 43 | (37.7%) | 65 | (57.0%) | 114 (100%) | |
| Poorly differentiated | 0 | (0.0%) | 3 | (15.0%) | 17 | (85.0%) | 20 (100%) | |
| TNM Stage | | | | | | | | |
| I | 2 | (22.2%) | 4 | (44.5%) | 3 | (33.3%) | 9 (100%) | P < 0.05 |
| II | 2 | (4.6%) | 24 | (54.5%) | 18 | (40.9%) | 44 (100%) | |
| III | 0 | (0.0%) | 3 | (23.1%) | 10 | (76.9%) | 13 (100%) | |
| Lymph node invasion | | | | | | | | |
| Positive | 3 | (5.9%) | 18 | (35.3%) | 30 | (58.8%) | 51 (100%) | No significant |
| Negative | 2 | (4.8%) | 11 | (26.2%) | 29 | (69.0%) | 42 (100%) | |

P value of chi-squared analysis.

### Immunohistochemical Staining and Evaluation of Tumor Specimens

Immunohistochemistry was performed on paraffin sections 5 μm thick tissues and breast cancer TMA. All slides were deparaffinized in xylene and rehydrated in graded alcohols. For antigen retrieval, slides were immersed in 0.01 M citrate buffer, pH 6.0, and boiled for 10 min in microwave oven. Endogenous peroxidase was blocked in 0.3% H₂O₂ in PBS for 20 min. To reduce nonspecific binding, sections were pre-incubated in normal goat serum. The anti-HAI-TMIP mouse monoclonal antibody were incubated overnight in a humidified chamber at 4° C. Incubation with anti-mouse peroxidase-conjugated antibody was done at room temperature for 30 min by a PowerVision Homo-Mouse IHC kit (ImmunoVision Technologies, Daly City, Calif.). Hematoxylin (Sigma) was used for counterstaining. Positive controls were included in each staining series. No significant staining was observed in the negative controls using isotype mouse IgG2b replacing the primary antibody.

HAI-TMIP immunopositivity was scored as follows: 0, no staining or sporadic staining in <5% of tumor cells; 1, weak and sporadic staining in 5%-25% of tumor cells; 2, weak staining in 26-50% of tumor cells; 3, strong diffuse cytoplasmic and member staining in 26-50% of tumor cells; and 4, strong, diffuse cytoplasmic staining and member in staining>50% of tumor cells. For statistical analysis, negative (O), intermediate (1+ and 2+) and strong (3+ and 4+) groups were created. Samples were evaluated under light microscopy by two independent pathologists without prior knowledge of the patients' clinical data.

The ER and PR data were offered by Shanghai Hujing Biotech Co., Ltd.

### Statistical Analysis

The breast tissue array data were converted to a PC and statistically analyzed using SPSS Version 10 for Windows (SPSS Inc., Chicago, Ill., USA). Correlation of the HAI-TMIP expression between the several different clinicopathological factors was calculated with the non-parametric Spearman correlation coefficient. Results with a P<0.05 were deemed statistically significant.

### 6.3.1.4 Detection of Autoantibodies to HAI-TMIP in Serum from Different Kinds of Cancers by Antigen-Mediated ELISA

Applied 0.1 ml of appropriately diluted HAI-TMIP to coat each of the 12 wells of the microtiter plate and incubated overnight at 4° C. or for 1 hour at 37° C. Removed coating solution and washed the wells of the microtiter plate three (3) times with washing buffer. Diluted serum from different kinds of cancers to be tested to an appropriate dilution in PBS or applied the culture supernatant without dilution. Pipetted 0.1 ml of the sample to be tested to each of the coated wells. Incubated the plate for 2 hours at room temperature and washed the wells three (3) times with washing buffer. Diluted the peroxidase labelled rabbit anti-mouse IgG 1:5,000 in washing buffer and added 0.1 ml of the enzyme labelled antibody to each well. Incubated the plate at room temperature for 15 minutes. Added 0.1 ml of the freshly prepared substrate to each well and incubated at room temperature for 10-15 minutes. Stopped the reaction by adding 50 μl of 3 N NaOH to each well.

### 6.3.2 Results

### 6.3.2.1 Real-Time PCR

Real-time PCR revealed that HAI-TMIP genomic DNA copies were amplified in 231 cell lines (≈200%) and 435 cell lines (≈300%), changed less in MCF-7 cell lines, MA cell lines and MR cell lines. Variant 4 of HAI-TMIP was lowly expressed in 10F cell lines, and variant 6 was highly expressed in MCF-7 cell lines, and variant 5 maybe lowly expressed in breast cell lines, relative to other cell lines, based on semi-quantitive PCR and real-time PCR.

### 6.3.2.2 Expression of Different Variants on Breast Tumor Tissues and Normal Tissues by Real-Time RT-PCR

HAI-TMIP variant 3 copy number in different sample templates from breast tumor tissues and normal breast tissues suggests that variant 3 is more abundant in normal breast tissues and has a relatively low expression in breast cancer tissues, and that its expression is down-regulated as the tumor progresses. Thus, HAI-TMIP variant 3 may have an anti-tumor role in normal samples, and its low expression may be the reason for tumor occurrence.

HAI-TMIP variant 4 was overexpressed in breast cancer tumor tissues and the overexpression of HAI-TMIP variant 4 is closely associated with advanced progression of tumor and metastasis.

HAI-TMIP variant 5 was overexpressed in breast tumor tissues and was also associated with drug resistance. HAI-TMIP subunit variant 6 was overexpressed in metastatic breast cancer. HAI-TMIP variant 7 was overexpressed in drug resistance breast tumors.

### 6.3.2.3 Immunohistochemistry & Tissue Microarray Analysis of HAI-TMIP

Staining results were obtained from all 234 cases. In all 33 normal breast epithelium, a weak to missing HAI-TMIP was detected at the cell membrane and in the cytoplasm. In 13 atypical hyperplasia of breast tissues, HAI-TMIP positive staining percentage was 23% (3/23). While the positive staining percentage on breast cancer tissues was 94.6% (159/168) (Table 10). Strong HAI-TMIP over-expression was detected in 98 breast cancer patients (58.3%), moderate staining in 61 patients (36.3%) and negative staining in 9 patients (5.3%). The cellular staining pattern was predominantly cellular membrane and cytoplasmic. The cytoplasmic immunoreaction was granular and appeared in epithelial cells. The expression of HAI-TMIP in breast cancer was significantly higher than in breast atypical hyperplasia and normal breast tissues. The analysis showed that the higher HAI-TMIP expression was significantly associated with tumor sizes, histological grades and stages (P<0.05). No statistically significant differences were found among cases with different ages or lymph node invasion (P>0.05) (Table 10).

TABLE 10
Comparation of HAI-TMIP Expression in Normal,
Atypical Breast Hyperplasia and Breast Cancer

HAI-TMIP expression

| | N | Negative | Positive | P value |
|---|---|---|---|---|
| Normal | 33 | 33 | 0 (0%) | <0.05 |
| Hyperplasia | 13 | 10 | 3 (23%) | |
| Breast Cancer | 168 | 9 | 159 (94.6%) | |

P value of chi-squared analysis.

### 6.3.2.4 Correlation of HAI-TMIP Protein Expression With ER Statrus, PR Status

The ER, PR status were obtained from 162 tumors. For ER expression: 59 tumors (36.4%) were classified as negative (0), 39 tumors (24.1%) as weakly positive (1+), 21 tumors (13.0%) as moderate positive (2+) and 43 (26.5%) as strongly positive (3+) as ER− positive. Tumors with a score of 0 and 1+were defined as ER-negative and with a score of 2+and 3+ as ER− positive. Sixty-four tumors (40.5%) showed a significant ER expression whereas 98 tumors (60.5%) were classified as negative. A total 71.9% of tumors classified as strongly HAI-TMIP-positive exhibited ER protein positive, weakly HAI-TMIP-positive classified tumors totaled only 23.4% of ER positive tumors, and 5.1% of the HAI-TMIP-negative tumors were ER negative. Statistical analysis revealed a significant relationship of elevated HAI-TMIP expression (P<0.05) with expression of ER (Table 11).

As for PR expression, tumors with a score of 0 and 1+were defined as PR-negative and with a score of 2+ and 3+ as PR− positive. Seventy-four tumors (45.7%) showed a significant PR expression whereas 88 tumors (54.3%) were classified as negative. A total 67.6% of tumors classified as strongly HAI-TMIP-positive exhibited PR protein positive, weakly HAI-TMIP-positive classified tumors totaled only 27.0% of PR positive tumors, and 5.7% of the HAI-TMIP-negative tumors were ER negative. No statistically significant differences were found between HAI-TMIP protein expression with PR status (P>0.05).

TABLE 11
Correlation between HAI-TMIP, ER and PR status

| | HAI-TMIP expression | | | | |
|---|---|---|---|---|---|
| | Negative | Weak | Strong | Total | P value |
| Estrogen receptor | | | | | |
| Positive (2+ and 3+) | 3 (4.7%) | 15 (23.4%) | 46 (71.9%) | 64 (100%) | P < 0.05 |
| Negative (0 and 1+) | 5 (5.1%) | 45 (45.9%) | 48 (49.0%) | 98 (100%) | |
| Progesterone receptor | | | | | |
| Positive (2+ and 3+) | 4 (5.4%) | 20 (27.0%) | 50 (67.6%) | 76 (100%) | No Significant |
| Negative (0 and 1+) | 5 (5.7%) | 40 (45.5%) | 43 (48.8) | 88 (100%) | |

P value of chi-squared analysis

### 6.3.2.5 Autoantibodies to HAI-TMIP in Serum of Different Kinds of Cancers

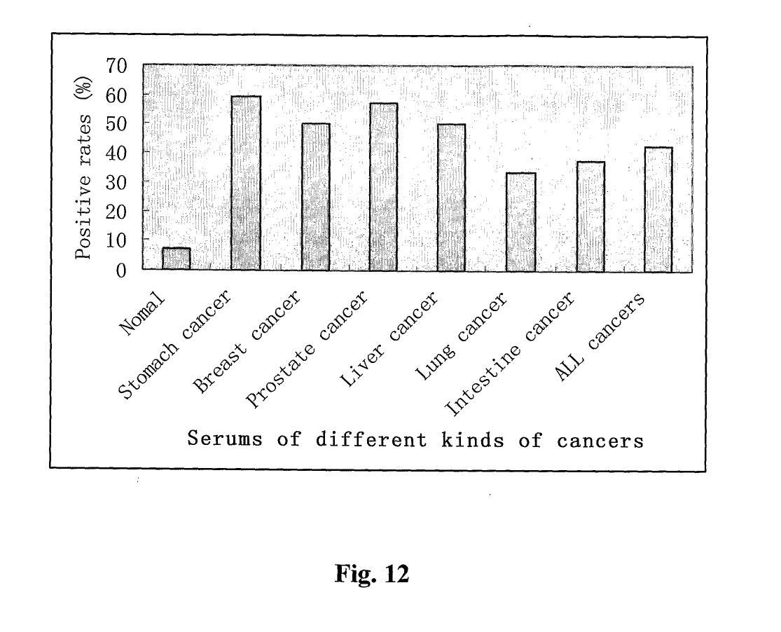

Results were obtained from all 96 cases. The percentage of positive autoantibodies of HAI-TMIP in cancer patients' serums were: 59.1% in stomach cancer, 50% in breast cancer, 57.1% in prostate cancer, 50% in liver cancer, 33.3% in lung cancer, 37.5% in intestine cancer, 42.7% in all cancers. While in 60 normal human serums, the percentage of positive autoantibodies of HAI-TMIP was 6.9% (FIG. 12). The percentage of positive autoantibodies of HAI-TMIP in patients' serums cancers were significantly higher than in normal human serums.

### 6.4 Example 4

### Involvement of HAI-TMIP Expression in Breast Cancer Progression and Metastasis

This example demonstrates that the HAI-TMIP is over-expressed in breast cancer and that an anti-HAI-TMIP antibody exerts a significant inhibitory effect on the proliferation and migration of breast cancer cells.

### 6.4.1 Materials & Methods

### 6.4.1.1 2-D liquid Chromatograph and MALDI-TOF/MS Analysis

ProteomeLab PF 2D two-dimensional liquid chromatography system (Beckman Coulter, Calif., USA). consists of 1^{st} dimension chromatofocusing separation based on pI and 2^{nd} dimension reverse-phase chromatography separation based on hydrophobicity. Chromatofocusing was carried out on the chromatofocusing column by mixing two buffers with different pH; Starting Buffer (pH 8.5), Eluention Buffer (pH 4.0) to create a linear pH gradient from 8.5 to 4.0, which is followed by a wash buffer comprising 1 M NaCl. Cell lysates (2 mg) of MHCC97-H and MHCC97-L, with different metastasis potentials, was injected onto the chromatofocusing column equilibrated for 130 minutes at 0.2 ml/min with the proprietary starting buffer included urea and a reducing compound at pH 8.5. Fractions were collected at 0.3-pH intervals and each fraction (200-500 μl) was sequentially analyzed by reverse-phase HPLC. Protein were separated on a non-porous C18 reverse phase column using 3.33% B/minute linear gradient in which solvent A was 0.1% aqueous trifluoroacetic acid and solvent B was 0.08% trifluoroacetic acid in acetronitrile at a flow rate of 0.75 ml/min. The different proteins were collected and digested by trypsin and conducted matrix-assisted laser desorption ionization time-of-flight (MALDI-TOF)/MS.

### 6.4.1.2 Immunofluorescence Analysis of Breast Cell Lines

Highly invasive breast cancer cell line, MDA-MB-231 and immortalized human breast epithelial cell line, MCF-10F were purchased from American Type Culture Collection (ATCC). MDA-MB-231 cells were cultured in DMEM medium supplemented with 10% fetal bovine serum (FBS). MCF10A cells were cultured in Ham's F12 medium supplemented with 10% FBS, and 20 μg/ml of epidermal growth factor at 37° C. and 5% CO₂. MDA-MB-231 and MCF-10F cells maintained on glass coverslips were stained with the anti-HAI-TMIP antibodies as described previously (Moser, T. L., Stack, M. S., Asplin, I., Enghild, J. J., H jrup, P., Everitt, L., Hubchak, S., Schnaper, H. W., and Pizzo, S. V. (1999) Proc. Natl. Acad. Sci. U.S.A. 96, 2811-2816; Moser, T. L., Kenan, D. J., Ashley, T. A., Roy, J. A., Goodman, M. D., Misra, U. K., Cheek, D. J., and Pizzo, S. V. (2001) Proc. Natl. Acad. Sci. U.S.A. 98, 6656-6661). Briefly, MDA-MB-231 and MCF-10F cells were washed with PBS several times before fixation with 4% paraformaldehyde. A sample was permeated with 100% ethanol for 5 min at room temperature as positive control. Cells were then incubated overnight with anti-HAI-TMIP mouse monoclonal antibody (1 μg/ml) at 4° C. and then washed them twice in PBS. The immunostaining was carried out for 1 h in the dark with antibody against mouse IgG2a conjugated to Alexa Fluor 488 (1:200, Molecular Probe) in staining buffer. Samples stained with the isotype mouse IgG2b antibody served as negative controls. The nucleuses were labelled with PI. After final washing, the cells were mounted and analyzed using a Zeiss LSM-410 (Switzerland) confocal microscope at a magnification of ×63.

### 6.4.1.3 Flow Cytometry Analysis of Breast Cell Lines

MDA-MB-231 and MCF-10F cells were incubated at 20° C. for 1 h in PBS, pH 7.4, containing 1% bovine serum albumin plus primary monoclonal antibodies in order to analyze cell-surface expression of HAI-TMIP. The cells were then washed in PBS plus 1% bovine serum albumin and incubated at 20° C. for 30 min with a goat antibody against mouse IgG2a conjugated to Alexa 488 (1:200) and washed three times. The isotype mouse IgG2b antibody served as negative controls. Propidium iodide was added to exclude the dead cells before analysis on a Coulter XL 4C flow cytometer (Beckman-Coulter).

### 6.4.1.4 Cell Proliferation Assay

MDA-MB-231 were plated at a density of 5,000 cells/well in DMEM media depleted of FCS overnight to allow the cells to become quiescent and MCF10F in Ham's F12 medium supplemented with 20 μg/ml of epidermal growth factor. Fresh media were added to the wells along with anti-HAI-TMIP antibody (0.5 μg/ml or 10 μg/ml). After 24 h, 10 μl CCK-8 (Dojindo, Kumamoto, Japan) were added to incubate for 2 h at 37° C. and the absorbance was measured on a Thermomax plate reader at a wavelength of 450 nm according to the manufacturer's specifications. The absorbance values were used to calculate the percent proliferation of the cells.

### 6.4.1.5 Cell Migration Assay

Migration assays were performed in Transwell membrane filter inserts in 24-well tissue culture plates (BD Labware, Bedford, Mass.) as described previously (Zhang, X. A., He, B., Zhou, B., and Liu, L. (2003) J. Biol. Chem. 278, 27319-27328). Briefly, the transwell membrane filter inserts that were placed in a 24-well tissue culture plate were 6.5 mm in diameter, 8-μm pore size, 10-nm thick polycarbonate membrane. The lower surface of the porous membrane was coated with either human plasma fibronectin (FN) (10 μg/ml) or mouse laminin (LN) (10 μg/ml) at 4° C. overnight, and then blocked with 0.1% heat-inactivated BSA (Calbiochem, San Diego, Calif.) at 37° C. for 45 min. MDA-MB-231 and MCF-10F cells were detached at 90% confluence with 2 mM EDTA/PBS, washed once in PBS, and re-suspended in serum-free DMEM containing 0.1% BSA. A 300 μl cell suspension was added to inserts at a density of 3×10⁴ cells/insert before anti-HAI-TMIP primary monoclonal antibody (5 μg/ml) and negative control mIgG2b (5 μg/ml) were incubated with cells for 30 min. Migration was allowed to proceed at 37° C. for 8 h. DMEM containing 1% FCS was added to the lower wells. Cells that did not migrate through the filters were removed using cotton swabs, and cells that migrated through the inserts were fixed and stained with Trypan Blue. The number of migrated cells per insert was counted under a light microscope at magnification×20. Experiments were carried out in triplicate and repeated at least three times. Data from several independent experiments were pooled and analyzed using a two-tailed, Student's t test.

### 6.4.2 Results

### 6.4.2.1 2-D Liquid Chromatograph and MALDI-TOF MS Analysis

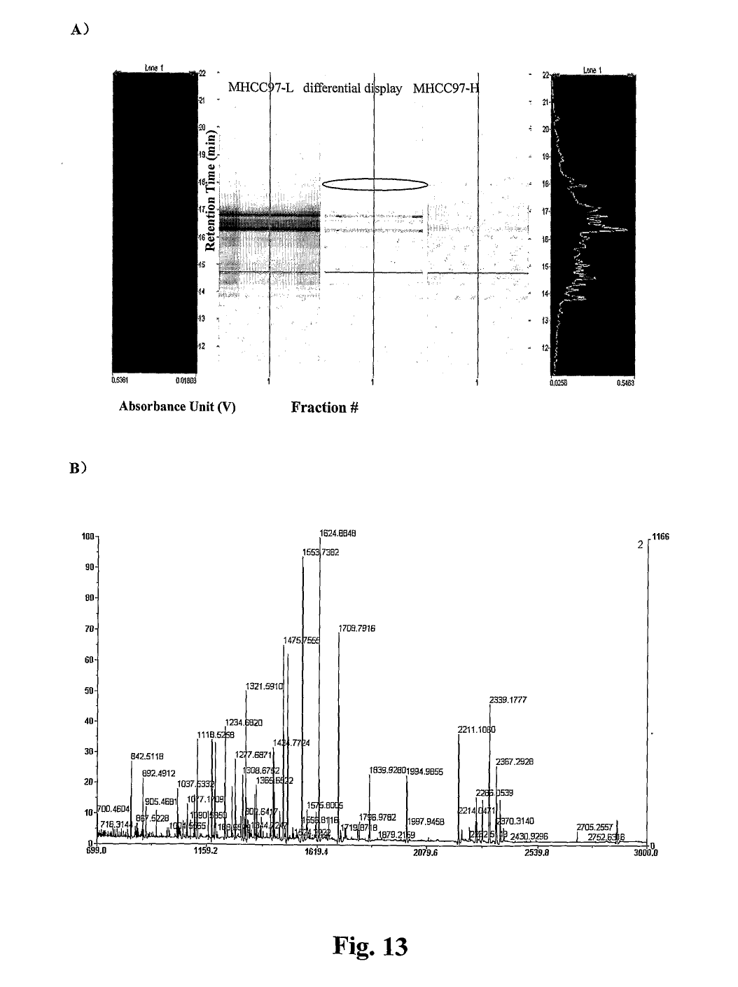

Image analysis of 2-D map revealed that 72 protein peaks showed significantly differential expression in MHCC97-H and MHCC97-L cells and 9 protein peaks were further identified by tryptic digestion, peptide mass fingerprinting and mass spectrometry. Five proteins were down-regulated while four proteins up-regulated in MHCC97-H cells include HAI-TMIP. FIG. 13 shows differential display map of HAI-TMIP between the MHCC97-H and MHCC97-L (FIG. 13A) and MALDI-TOF/MS tryptic peptide mass map of HAI-TMIP (FIG. 13B). MS-Digest search using the peptide mass fingerprint data indicated that 10 peptides were matched with peptides from HAI-TMIP, giving sequence coverage of 28% (126/450 AA's) of the protein. The amino acid sequence of this protein can be accessed through NCBI Protein Database under NCBI Accession No. 4757810.

### 6.4.2.2 Binding of the HAI-TMIP Antibody to the Surface of Breast Cancer Cells by Immunofluorescence Microscopy and Flow Cytometry,

To confirm the surface localization of the HAI-TMIP, breast cancer cells were analyzed by immunofluorescence microscopy and flow cytometry. Immunofluorescence microscopy confirmed the surface-associated immunoreactivity of HAI-TMIP antibody on MDA-MB-231 cell membrane. Control experiments were performed with isotype mouse IgG2b and normal breast cell line MCF-10F and there was no reaction on cell membrane. Permeabilized cells were performed as positive control and also reacted with anti-HAI-TMIP antibody.

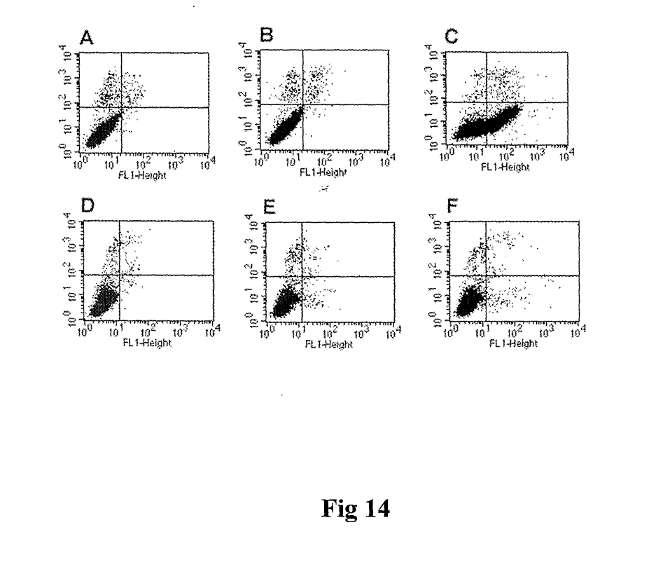

The surface localization of the HAI-TMIP on MDA-MB-231 cells was further confirmed by flow cytometry (FIG. 14C). Control experiments were performed with second antibody only (FIG. 14A) and isotype mouse IgG2b (FIG. 14B). The control cell flow cytometry studies were performed by MCF-10F cells (FIG. 14 D, E, F).

### 6.4.2.3 Antiproliferative Effect of Anti-HAI-TMIP Antibody on Breast Cancer Cells

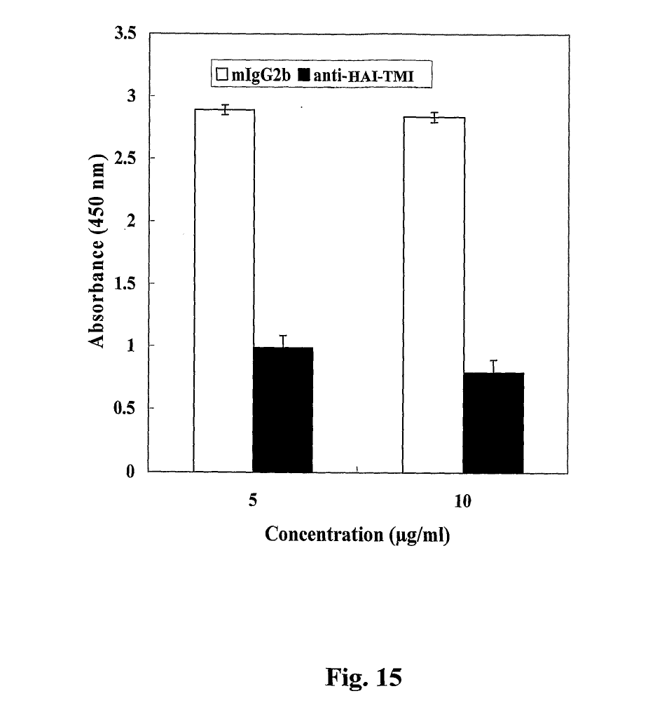

To gain insight into the function of HAI-TMIP, the anti-HAI-TMIP antibody was tested to determine if it can inhibit the growth of MDA-MB-231. The anti-HAI-TMIP antibody inhibited the MDA-MB-231 proliferation in a concentration-dependent manner. An unrelated isotype mouse IgG2b had no effect on inhibition (FIG. 15). In comparison, for MCF-10F cells treated with anti-HAI-TMIP antibody, no inhibition of cell proliferation was observed (data not shown).

### 6.4.2.4 Inhibition of MDA-MB-231 Migration by Anti-HAI-TMIP Antibody

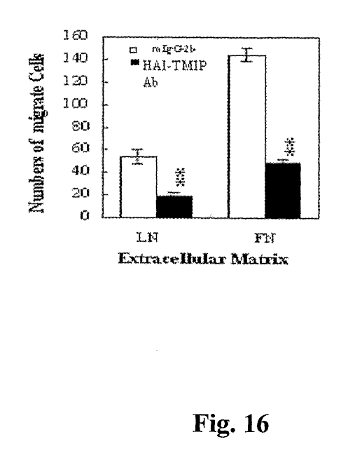

To investigate the effect of HAI-TMIP expression on cell motility, a metastatic breast cancer cell line MDA-MB-231 was tested for its directional motility on FN and LN in a transwell chemohaptotactic migration assay. As shown in FIG. 16, the ability of cells treated by anti-HAI-TMIP antibody to migrate toward FN and LN was remarkably reduced compared with that treated by isotype mouse IgG2b. The diminished chemohaptotactic cell migration in MDA-MB-231 cells was observed on both FN and LN-coated substrata toward 1% fetal calf serum (FCS). No migration difference was seen on MCF-10F cells (data not shown).

### 6.5 Example 5

### Apoptosis Induced by Adriamycin & a HAI-TMIP Antibody

### 6.5.1 Materials & Methods

### 6.5.1.1 Cytostasis Assay

Cell were plated at a density of 15,000 cells/well in DMEM media depleted of FCS with adriamycin alone or along with different concentrations of anti-HAI-TMIP antibody for 48 hours. The anti-proliferative activity was determined 48 hours later by the 3-(4,5-dimethylthiazol-2-yl)-2,5-diphenyltetrazolium bromide assay as described (Jordan et al, 1992, Neurooncol 14:19-35.). 100 μL of mixture of DMEM&CCK-8 (10:1) (Dojindo, Kumamoto, Japan) were incubated for 2 hours at 37° C. and the absorbance was quantitated on a Thermomax plate reader at OD450 nm according to the manufacturer's specifications. The absorbance values were used to calculate the percent amount of the cells.

### 6.5.1.2 Apoptosis Assay

Cells were incubated with adriamycin alone or along with different concentrations of anti-HAI-TMIP antibody. Cells were washed and resuspended in 100 μl of staining solution (containing annexin V fluorescein and PI in a HEPES buffer; Annexin-V-FITC Staining Kit; Becton.Dickinson). After incubation at room temperature for 20 minutes, cells were analyzed by flow cytometry. Annexin V binds to cells that express phosphatidylserine on the outer layer of the cell membrane, and PI stains the cellular DNA of cells with a compromised cell membrane. This allows for the discrimination of live cells (unstained with either fluorochrome) from apoptotic cells (stained only with annexin V) and necrotic cells (stained with both annexin V and PI).

### 6.5.2 Results

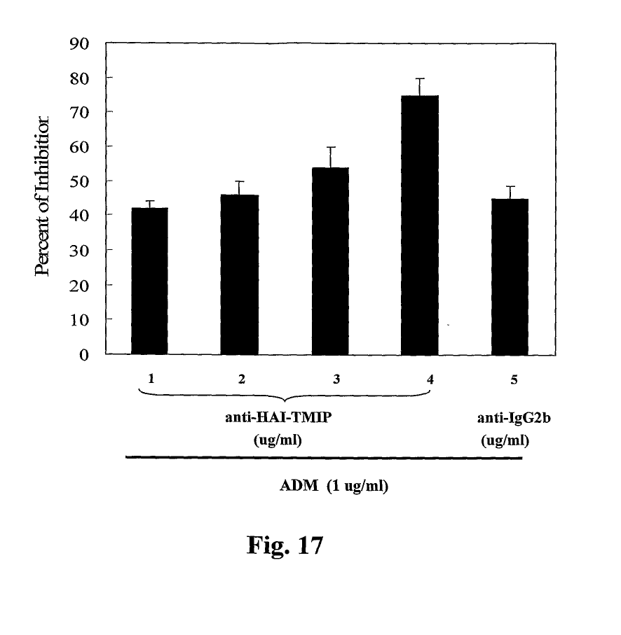

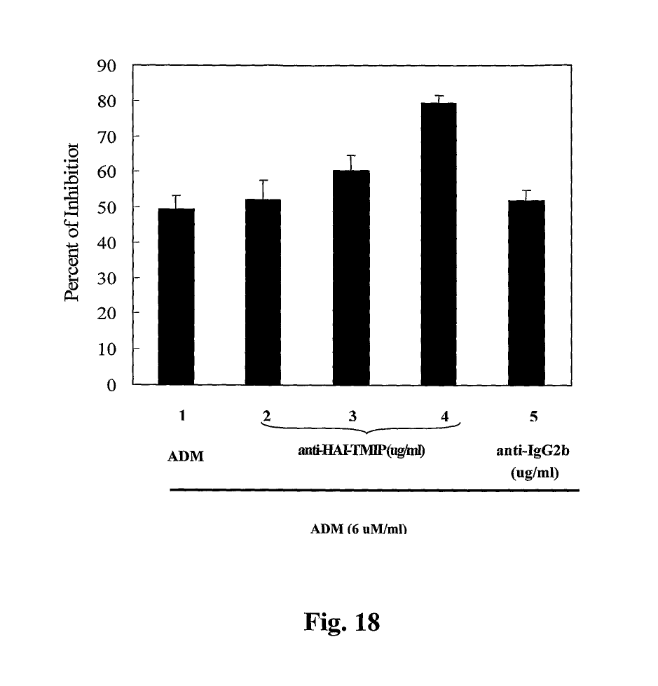

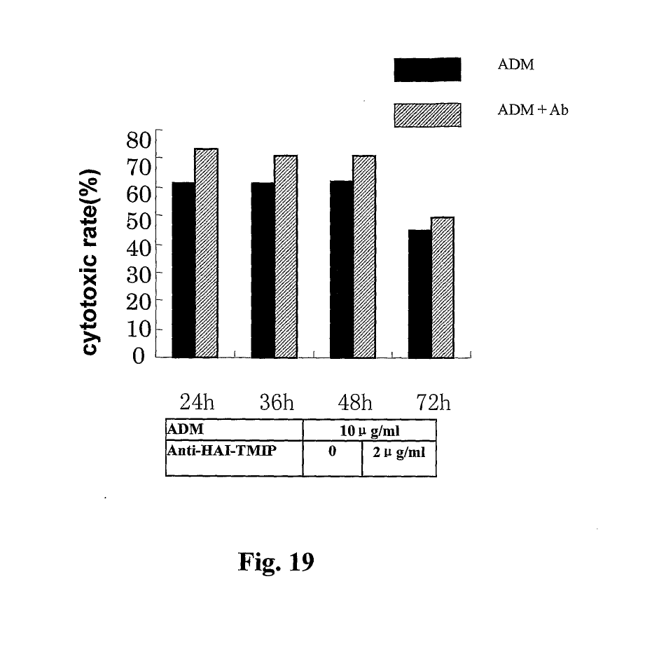

As shown in FIGS. 17-19, the combination of adriamycin (ADM) and a HAI-TMIP-antibody effectively suppressed the growth of MDA-MB-435, MCF-7 and MCF-7/ADR cells compared to ADM alone.

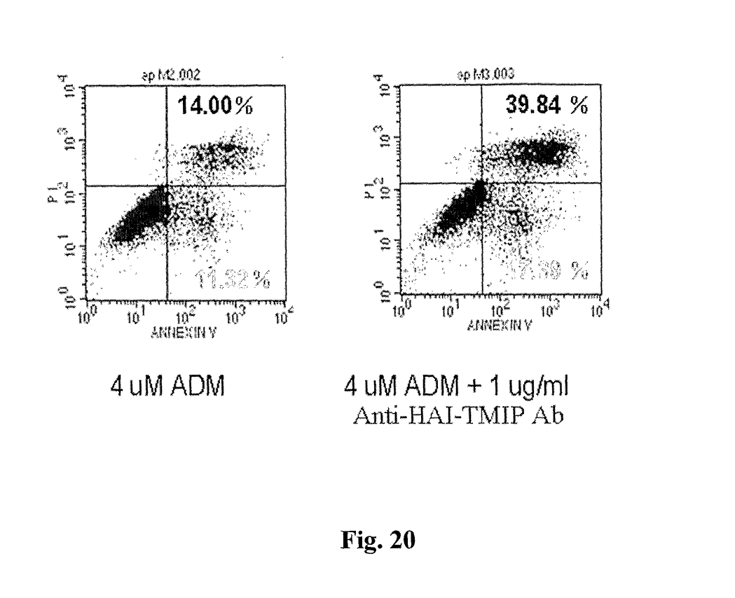

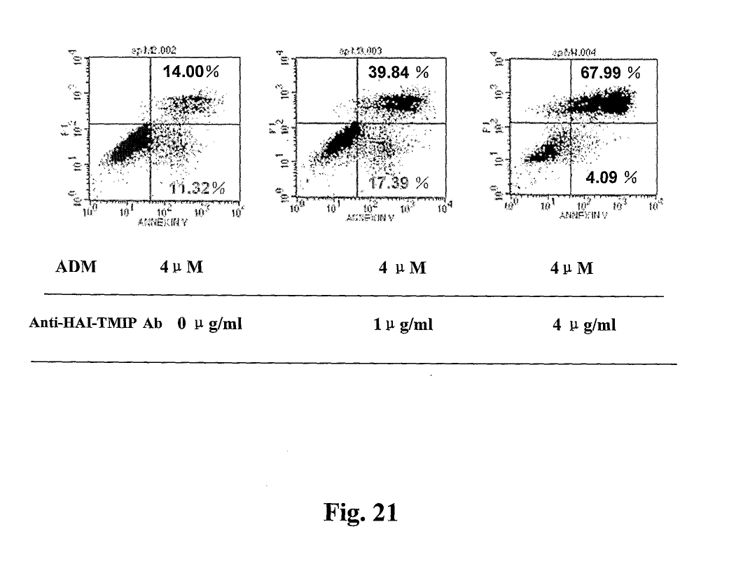

As shown in FIGS. 20 and 21, the combination of ADM and the HAI-TMIP antibody effectively induced apoptosis of MCF7 and MCF7/ADR compared to ADM alone.

### 6.6 Example 6

### RNAi of HAI-TMIP

### 6.6.1 Materials & Methods

Target sequences for miRNA were selected based on published optimization criteria (The miRNA user guide, available on the Invitrogen website for rnaidesigner under rnaiexpress). Sequences were analyzed using the BLAST program (available on the NCBI website) for short nearly exact matches to avoid non-specific binding.

Five different miRNA sequences corresponding to different nucleotides of the HAI-TMIP variant 1 sequence (National Center for Biotechnology Information (NCBI) GenBank NM 001001937) were chosen for initial transient test transfections, miRNA320 (GCTG AGAGGTA TCAGCTCCAAGAATGTTTTGGCCACTGACTGACATTC TTGGCT GATACCTCTCAGG (SEQ ID NO:31)) corresponding to nucleotides 320-340, miRNA483 (TGCT GCAA ACACGACAACACCAACATG TTTTGGCCACTGACTGACAT GTTGG TTGTCGTGTTTGCAGG (SEQ ID NO:32)) corresponding to nucleotides 483-503, miRNA565 (TGCTGAACAGCTCCTCACCAACTGGAGTTTTGGCCACTGACTGACTCCAGTTG GAGGAGCTGTTCAGG (SEQ ID NO:33)) corresponding to nucleotides 565-585, miRNA591 (TGCT GTACCAAGGGCATCAACTACACGTTTTGGCCACTGACTGACGTGTAGTT T GCCCTTGGTACAGG (SEQ ID NO:34)) corresponding to nucleotides 591-611, miRNA 755(TGCTG AATCAG TTCACGCTGACCACGGTTTTGGCCACTGACTGACCGTGGTCAGT GAACTGATTCAGG (SEQ ID NO:35) corresponding to nucleotides 755-775.

MCF-7 cells were obtained from the American Type Culture Collection (Manassas, Va., USA). 5×10⁴ MCF-7 cells were seeded into six-well plates. After 24 hours, cells were transfected with 100 Nm miRNA using either TKO transfection agent (Mirus Madison, Wis., USA) or Effectene transfection agent (Qiagen, Hilden, Germany) according to the manufacturer's instructions. Transfection efficiency was optimized using fluorescein-labelled miRNA against luciferase (Dharmacon Research) which is not expressed in mammalian cells. After 24 and 48 hours, cells were harvested for western blot and immunolocalization analyses.

The expression vector pSUPER (OligoEngine, Seattle, Wash., USA) was used to express miRNA against HAI-TMIP in MCF-7 cells. The miRNA2 sequence was used for stable transfection. The 19-nucleotide sequence separated by a nine-nucleotide non-complementary spacer (TTCAAGAGA (SEQ ID NO:36)) from the reverse complement of the same 19-nucleotide sequence was synthesized by MWG Biotech (Ebersberg, Germany). The sequence was inserted into the pSUPER backbone after digestion with Bg11I and HindIII. This vector (pSUPER-PTPt) was transformed into XL-10 Gold ultracompetent cells (Stratagene, La Jolla, Calif., USA). Several clones were obtained, and the correct vector sequence was verified by sequencing. MCF-7 cells were plated at 6×10⁵ cells on to 10-cm dishes. After 24 hours, cells were co-transfected with pSUPER-PTP and pRc/CMV (Invitrogen, Carlsbad, Calif., USA) using Effectene (Qiagen). As pSUPER does not contain a resistance gene for eukaryotic cell systems, co-transfection with pRc/CMV was performed to introduce the neomycin gene for selection. Two micrograms of plasmid DNA containing pSUPER-PTP and co-plasmid at a ratio of 5:1 or 10:1 were transfected. The empty vector pSUPER was mock transfected as control. Twenty-four hours after transfection, selection with 400 μg/mL G418 (Invitrogen) was initiated and continued for 3 weeks. To obtain stable cell lines, clonal cells were singled out and grown separately.

Total cell lysates were prepared using lysis buffer (50 mm HEPES pH 7.4, 1% Triton X-100, 10% glycerol, 150 mm NaCl, 1.5 mm MgCl₂, 1 mm EGTA supplemented with protease inhibitors). Lysates were cleared from cellular debris by centrifugation, and protein concentrations were determined using the bicinchoninic assay (Pierce, Rockford, Ill., USA). Proteins were separated by SDS-polyacrylamide electrophoresis on 5% gels, and transferred to a polyvinylidene difluoride membrane (Millipore, Bedford, Mass., USA). Loading of equal protein amounts was verified by Coomassie staining. Membranes were blocked with 1× Rotiblock (Roth, Karlsruhe, Germany) for 1 hour at room temperature, and probed overnight at 4° C. with 1 μg/mL HAI-TMIP monoclonal antibody (Invitrogen, Carlsbad, Calif., USA). Binding was detected using secondary horseradish peroxidase-conjugated goat anti-mouse IgG (Dianova, Hamburg, Germany). Immunoreactive bands were visualized using chemiluminescent SuperSignal substrate (Pierce). As control, blots were stripped and re-probed with a polyclonal antibody against the nuclear protein scaffold attachment factor-A (SAF-A).

### 6.6.2 Results

Five different miRNA oligonucleotides were detected to down-regulate HAI-TMIP expression in MCF7 cells by RT-PCR. All of these sequences targeted the 5′ region of the HAI-TMIP mRNA, upstream of the first splice site and expression of all three main splice variants was antagonized. This region corresponds to the extracellular N-terminal part of the HAI-TMIP protein. The expression of HAI-TMIP was decreased as analysed by western-blot.

### 6.7 Example 7

### Antibody Immunospecifically Binds to Tumor Tissues

This example demonstrates that an antibody raised against peptide 2 of HAI-TMIP immunospecifically binds to breast cancer tissues and hepatocellular carcinomar tissues. Peptide 2 (SEQ ID NO:26) as an immunogen generates antibodies that immunospecifically bind to HAI-TMIP variant 1, 2, 3, 4, 6 and 7 (but not to HAI-TMIP variant 5).

### 6.7.1 Materials & Methods

Biopsy specimens 3 or 4 mm in thickness were blocked and fixed for 18-24 hours in neutral buffered formalin. The tissues were then dehydrated in a series of alcohols and xylene, followed by infiltration by melted paraffin held at no more than 60° C. Sections (4-5 μm) were cut, mounted on Fisherbrand Superfrost/plus slides and stained using immunohistochemistry (1HC) with anti-peptide 2 antibody. Anti-peptide 2 IHC was performed manually. Sections were dewaxed in two xylene baths (5 minutes each), taken through a series of alcohols (100, 95, 75%), rehydrated in distilled water and then submitted to heat-induced antigen retrieval for 40 minutes in the presence of a citrate buffer. Sections were blocked and then incubated using a peptide 2 monoclonal antibody at 0.5 μg/ml concentration in a humidified chamber at 37° C. for 1 hour. Following incubation with biotinylated goat anti-mouse antibody for 30 minutes at 1:1000, peptide 2 was visualized with 3,3′-diaminobenzidine (DAB). Sections were then counterstained, dehydrated, cleared and mounted.

### 6.7.2 Results

The IHC results showed that expression of a protein(s) comprising peptide 2 was up-regulated within the malignant breast tissue sections compared with the nonmalignant breast tissue sections. Furthermore, the over-expression of a protein(s) comprising peptide 2 was mainly localized at thed cytoplasm of the cells and cell membranes. The same tendency was also observed on the hepatocellular tissue sections.

### 6.8 Example 8

### Breast Cancer Therapy

### 6.8.1 Murine Breast Cancer Therapeutic Regime

This example describes the mice breast cancer therapeutic regime by the inhibitors of HAI-TMIP.

4-6 week old, female, CD-1(nu/nu), athymic mice are inoculated subcutaneously in the mid-back regions with 5×10⁶ MCF-7cells. Prior to cell injection, all mice are primed with 17 β-estradiol applied s.q. to promote tumor growth. Five to ten animals are randomly assigned to each treatment group. Treatment with control antibody, cytotoxic chemotherapeutic drug, anti-HAI-TMIP antibody, or the combination is initiated 9-14 days post-xenograft inoculation at which time xenograft volumes measure approximately 50-100 mm³. Tumor volumes, calculated as the product of length, width and depth are monitored twice weekly by serial micrometer measurements by a single observer. Human myeloma IgG2b serves as the control antibody for these experiments and is administrated at the same dose and dose interval. For the breast cancer xenograft studied, MDA-MB-231, MDA-MB-435, MCF7/ADR, HCC1569, and ZR-75-1 are used.

### 6.8.2 Human Breast Cancer Therapeutic Regime

This example describes the human breast cancer therapeutic regime by using the inhibitors of HAI-TMIP.

The patient's cancer biopsy sample is stained positively for the expression of HAI-TMIP on the cancer cell surface by immunohistochemistry for the patient to be eligible for therapy. An anti-HAI-TMIP antibody (e.g., an antibody that immunospecifically binds to a HAI-TMIP variant) is administered to patients either intravenously or subcutaneously in a dose range from 0.05 to about 25 mg/kg. Patients receive at least 4 weekly doses. Tumor size is monitored by CAT scan or MRI prior to therapy and post therapy. Reduction of the tumor size is the primary indication of the antibody's efficacy. Tumor shrinkage by 50% or more is considered to be a partial response. Complete disappearance of the tumor is considered as a complete response. For breast cancer patients, CA15-3 level prior to therapy and post therapy is also monitored as a secondary indication of treatment efficacy.

### 7. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. An isolated nucleic acid molecule comprising:
a) the nucleotide sequence of SEQ ID NO: 1, 2, 3, 4, 5, 6, 7, 37, 38, 39, 40 or 41;
b) the nucleotide sequence which encodes a polypeptide comprising the amino acid sequence of SEQ ID NO:8 or 9;
c) a nucleotide sequence comprising nucleic acid residues 123 to 309 of SEQ ID NO:4;
d) a nucleotide sequence comprising nucleic acid residues 468 to 557 of SEQ ID NO:5;
e) a nucleotide sequence which hybridizes to the complement of the nucleotide sequence of SEQ ID NO: 1, 2, 3, 4, 5, 6 or 7;
f) a nucleotide sequence which hybridizes to the complement of a nucleotide sequence encoding a polypeptide comprising the amino acid sequence of SEQ ID NO:8 or 9; or
g) a nucleotide sequence that is at least 95% identical to the nucleotide sequence of SEQ ID NO: 1, 2, 3, 4, 5, 6 or 7.

2. An isolated polypeptide comprising:
h) the amino acid sequence of SEQ ID NO:8 or 9;
i) the amino acid sequence encoded by the nucleotide sequence of SEQ ID NO: 1, 2, 3, 4, 5, 6 or 7;
j) an amino acid sequence encoded by a nucleotide sequence that hybridizes over its full length to the nucleotide sequence of SEQ ID NO: 1, 2, 3, 4, 5, 6 or 7;
k) an amino acid sequence that is at least 90% identical to the amino acid sequence of SEQ ID NO: 8; or
l) an amino acid sequence that is at least 65% identical to the amino acid sequence of SEQ ID NO:5.

3. A vector comprising the nucleic acid molecule of claim 1.

4. The vector of claim 3 further comprising a nucleotide sequence which regulates the expression of a polypeptide encoded by the nucleic acid molecule.

5. A host cell comprising the nucleic acid molecule of claim 1.

6. A host cell comprising the vector of claim 3.

7-10. (canceled)

11. A fusion protein comprising a polypeptide encoded by the nucleic acid molecule of claim 1.

12. A fusion protein comprising the polypeptide of claim 2.

13. A purified complex comprising a polypeptide encoded by the nucleic acid molecule of claim 1.

14. A purified complex comprising the polypeptide of claim 2.

15-20. (canceled)

21. An antibody that immunospecifically binds to a polypeptide encoded by the nucleic acid molecule of claim 1.

22. An antibody that immunospecifically binds to the polypeptide of claim 2.

23-31. (canceled)

32. A pharmaceutical composition comprising a polypeptide encoded by the nucleic acid molecule of claim 1 and a pharmaceutically acceptable carrier or excipient.

33. A pharmaceutical composition comprising the polypeptide of claim 2 and a pharmaceutically acceptable carrier or excipient.

34. A pharmaceutical composition comprising the complex of claim 13 and a pharmaceutically acceptable carrier or excipient.

35-47. (canceled)

48. A method of preventing, treating and/or managing cancer, the method comprising administering to a subject in need thereof a compound that alters the expression of a polypeptide encoded by the nucleic acid molecule of claim 1.

49. A method of preventing, treating and/or managing cancer, the method comprising administering to a subject in need thereof a compound that alters the expression of the polypeptide of claim 2.

50. A method of preventing, treating and/or managing diabetes, hypertension, hyperlipidemia or obesity, the method comprising administering to a subject in need thereof a compound that alters the expression of a polypeptide encoded by the nucleic acid molecule of claim 1.

51. A method of preventing, treating and/or managing diabetes, hypertension, hyperlipidemia or obesity, the method comprising administering to a subject in need thereof a compound that alters the expression of the polypeptide of claim 2.

52-75. (canceled)

76. An isolated peptide having the amino acid sequence of:

| | |
|---|---|
| ARNFHASNTHLQKTC; | (SEQ ID NO: 25) |
| MSSILEERILGADC; | (SEQ ID NO: 26) |
| MQTGIKAVDSLVPC; | (SEQ ID NO: 27) |
| CASNTHLQKTGTAE; | (SEQ ID NO: 28) |
| or | |
| CVSQHQALLGTIRA. | (SEQ. ID NO: 29) |

77-106. (canceled)

107. A host cell comprising the vector of claim 4.

108. A pharmaceutical composition comprising the complex of claim 14 and a pharmaceutically acceptable carrier or excipient.


SEQUENCE LISTING (78 sequences)

SEQ ID NO: 1 - DNA, 1950 bp, homo sapiens
  human ATP synthase alpha subunit variant 1 (Genbank accession no NM_001001937)
tctggcattgcaagcctcgcttcgttgccacttcccagctcttcccgccttccgcggtataatcaacactacgagagatagagccgcctagaaccagtccggaggctgcggctgcagaagtaccgcctgcggagtaactgcaaagatgctgtccgtgcgcgttgctgcggccgtggtccgcgcccttcctcggcgggccggactggtctccagaaatgctttgggttcatctttcattgctgcaaggaacttccatgcctctaacactcatcttcaaaagactgggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttctagtaaattagttccatttgtaaaagggttactctcatactccttatgtacagaaatcacatgaaaaataaaggttccataatgcatagttaaaaa

SEQ ID NO: 2 - DNA, 1895 bp, homo sapiens
  human ATP synthase alpha subunit variant 2 (Genbank accession no NM_004046)
gtcttgaccttctttgcggctcggccattttgtcccagtcagtccggaggctgcggctgcagaagtaccgcctgcggagtaactgcaaagatgctgtccgtgcgcgttgctgcggccgtggtccgcgcccttcctcggcgggccggactggtctccagaaatgctttgggttcatctttcattgctgcaaggaacttccatgcctctaacactcatcttcaaaagactgggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttctagtaaattagttccatttgtaaaagggttactctcatactccttatgtacagaaatcacatgaaaaataaaggttccataatgcatagttaaaaa

SEQ ID NO: 3 - DNA, 1692 bp, homo sapiens
  Human angiostatin interacting and tumor metastasis involving protein, HAI-TMIP, variant 3 (cloned from cell line MCF-7/A)
cggtataatcaacactacgagagatagagccgcctagaaccaggtctccagaaatgctttgggttcatctttcattgctgcaaggaacttccatgcctctaacactcatcttcaaaagactgggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttc

SEQ ID NO: 4 - DNA, 1879 bp, homo sapiens
  Human angiostatin interacting and tumor metastasis involving protein, HAI-TMIP, variant 4 (cloned from cell line MCF-7/A)
cggtataatcaacactacgagagatagagccgcctagaaccaggtctccagaaatgctttgggttcatctttcattgctgcaaggaacttccatgcctctaacactcatcttcaaaagactgtagagacgggattttgctgtgttggccaggctggtccaaactcttggcctcaagtggtccgcccgcttcccaaagtgctggtattactggcgtgagccactgcacccaacccctgtatgctaagatgaagaataccacctatactaccctgtgtgccttgaaagagaaagacacactttgttaacagggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttc

SEQ ID NO: 5 - DNA, 1782 bp, homo sapiens
  Human angiostatin interacting and tumor metastasis involving protein, HAI-TMIP, variant 5 (cloned from cell line MCF-10F)
cggtataatcaacactacgagagatagagccgcctagaaccaggtctccagaaatgctttgggttcatctttcattgctgcaaggaacttccatgcctctaacactcatcttcaaaagactgggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaaggtaggtttaatgttcgttagatatagttgcagcttctaacaaacatcaaaactgattatgcttagggtttttctttttattttttaacagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttc

SEQ ID NO: 6 - DNA, 1613 bp, homo sapiens
  Human angiostatin interacting and tumor metastasis involving protein, HAI-TMIP, variant 6 (cloned from cell line MCF-10F)
cggtataatcaacactacgagagatagagccgcctagaaccagggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttc

SEQ ID NO: 7 - DNA, 1720 bp, homo sapiens
  Human angiostatin interacting and tumor metastasis involving protein, HAI-TMIP, variant 7 (cloned from cell line MCF-7/A)
gtcttgaccttctttgcggctcggccattttgtcccagtcagtccggaggctgcggctgcagaagtaccgcctgcggagtaactgcaaagatgctgtccgtgcgcgtcgctgcggccgtggtccgcgcccttcctcggcgggccggactgggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggcaattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttc

SEQ ID NO: 8 - protein, 503 aa, homo sapiens
  Human angiostatin interacting and tumor metastasis involving protein, HAI-TMIP, variant 3, 4, 6 & 7
MSSILEERILGADTSVDLEETGRVLSIGDGIARVHGLRNVQAEEMVEFSSGLKGMSLNLEPDNVGVVVFGNDKLIKEGDIVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 9 - protein, 365 aa, homo sapiens
  Human angiostatin interacting and tumor metastasis involving protein, HAI-TMIP, variant 5
MQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 10 - protein, 10 aa, homo sapiens
  N-terminal 10 aa fragment of human angiostatin interacting and tumor metastasis involving protein, HAI-TMIP, variant 3
MSSILEERIL

SEQ ID NO: 11 - protein, 10 aa, homo sapiens
  N-terminal 10 aa fragment of human angiostatin interacting and tumor metastasis involving protein, HAI-TMIP, variant 5
MQTGIKAVDS

SEQ ID NO: 12 - DNA, 23 bp, Artificial Sequence
  primer ATPa-P1
cggtataatcaacactacgagag

SEQ ID NO: 13 - DNA, 23 bp, Artificial Sequence
  primer ATPa-P2
gaacaatgacaaaactgaactgg

SEQ ID NO: 14 - DNA, 22 bp, Artificial Sequence
  primer ATPa-P3
gtcttgaccttctttgcggctc

SEQ ID NO: 15 - DNA, 22 bp, Artificial Sequence
  primer ATPa-P11
caaagcatttctggagaccagt

SEQ ID NO: 16 - DNA, 15 bp, Artificial Sequence
  primer ATPa-P13
ggccggactgggact

SEQ ID NO: 17 - DNA, 22 bp, Artificial Sequence
  primer ATPa-P14
tagagccgcctagaaccaggga

SEQ ID NO: 18 - DNA, 25 bp, Artificial Sequence
  primer ATPa-P15
gaaagacacactttgttaacaggga

SEQ ID NO: 19 - DNA, 20 bp, Artificial Sequence
  primer ATPa-P19
ggaaccaattggaccctttc

SEQ ID NO: 20 - DNA, 25 bp, Artificial Sequence
  primer ATPa-P20
gaagctgcaactatatctaacgaac

SEQ ID NO: 21 - DNA, 21 bp, Artificial Sequence
  primer ATPa-P21
agagccgcctagaaccaggtc

SEQ ID NO: 22 - DNA, 22 bp, Artificial Sequence
  probe T1
agagccgcctagaaccagtccg

SEQ ID NO: 23 - DNA, 24 bp, Artificial Sequence
  probe T2
caagggcatcaactacacgaccca

SEQ ID NO: 24 - DNA, 24 bp, Artificial Sequence
  primer ATPa-P16
acactcatcttcaaaagactggga

SEQ ID NO: 25 - protein, 15 aa, Artificial Sequence
  synthetic polypeptide 1 - to be used for producing antibodies to distinguish 3 human ATP synthase alpha subunit isoforms
ARNFHASNTHLQKTC

SEQ ID NO: 26 - protein, 14 aa, Artificial Sequence
  synthetic polypeptide 2 - to be used for producing antibodies to distinguish 3 human angiostatin interacting and tumor metastasis involving protein, HAI-TMIP, isoforms
MSSILEERILGADC

SEQ ID NO: 27 - protein, 14 aa, Artificial Sequence
  synthetic polypeptide 3 - to be used for producing antibodies to distinguish 3 human angiostatin interacting and tumor metastasis involving protein, HAI-TMIP, isoforms
MQTGIKAVDSLVPC

SEQ ID NO: 28 - protein, 14 aa, Artificial Sequence
  synthetic polypeptide 4 - to be used for producing antibodies to distinguish 3 human angiostatin interacting and tumor metastasis involving protein, HAI-TMIP, isoforms
CASNTHLQKTGTAE

SEQ ID NO: 29 - protein, 14 aa, Artificial Sequence
  synthetic polypeptide 5 - to be used for producing antibodies to distinguish 3 human angiostatin interacting and tumor metastasis involving protein, HAI-TMIP, isoforms
CVSQHQALLGTIRA

SEQ ID NO: 30 - DNA, 21 bp, Artificial Sequence
  downstream primer
aatccttcctcccacccctga

SEQ ID NO: 31 - DNA, 67 bp, Artificial Sequence
  miRNA 320
gctgagaggtatcagctccaagaatgttttggccactgactgacattcttggctgatacctctcagg

SEQ ID NO: 32 - DNA, 68 bp, Artificial Sequence
  miRNA 483
tgctgcaaacacgacaacaccaacatgttttggccactgactgacatgttggttgtcgtgtttgcagg

SEQ ID NO: 33 - DNA, 68 bp, Artificial Sequence
  miRNA 565
tgctgaacagctcctcaccaactggagttttggccactgactgactccagttggaggagctgttcagg

SEQ ID NO: 34 - DNA, 68 bp, Artificial Sequence
  miRNA 591
tgctgtaccaagggcatcaactacacgttttggccactgactgacgtgtagtttgcccttggtacagg

SEQ ID NO: 35 - DNA, 68 bp, Artificial Sequence
  miRNA 755
tgctgaatcagttcacgctgaccacggttttggccactgactgaccgtggtcagtgaactgattcagg

SEQ ID NO: 36 - DNA, 9 bp, Artificial Sequence
  nine-nucleotide non-complementary spacer
ttcaagaga

SEQ ID NO: 37 - DNA, 1512 bp, homo sapiens
  coding region of human angiostatin interacting and tumor metastasis involving protein, HAI-TMIP, variant 3
atgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaa

SEQ ID NO: 38 - DNA, 1512 bp, homo sapiens; identical to SEQ ID NO: 37
  coding region of human angiostatin interacting and tumor metastasis involving protein, HAI-TMIP, variant 4

SEQ ID NO: 39 - DNA, 1098 bp, homo sapiens
  coding region of human angiostatin interacting and tumor metastasis involving protein, HAI-TMIP, variant 5
atgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaa

SEQ ID NO: 40 - DNA, 1512 bp, homo sapiens; identical to SEQ ID NO: 37
  coding region of human angiostatin interacting and tumor metastasis involving protein, HAI-TMIP, variant 6

SEQ ID NO: 41 - DNA, 1512 bp, homo sapiens
  coding region of human angiostatin interacting and tumor metastasis involving protein, HAI-TMIP, variant 7
atgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggcaattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaa

SEQ ID NO: 42 - DNA, 21 bp, Artificial Sequence
  upstream primer
aggcttgaactcctgggctca

SEQ ID NO: 43 - protein, 15 aa, Unknown
  an example of linker peptide joining VH and VL domain
GGGGSGGGGSGGGGS

SEQ ID NO: 44 - protein, 15 aa, Unknown
  an example of linker peptide joining VH and VL domain
ESGRSGGGGSGGGGS

SEQ ID NO: 45 - protein, 14 aa, Unknown
  an example of linker peptide joining VH and VL domain
EGKSSGSGSESKST

SEQ ID NO: 46 - protein, 15 aa, Unknown
  an example of linker peptide joining VH and VL domain
EGKSSGSGSESKSTQ

SEQ ID NO: 47 - protein, 14 aa, Unknown
  an example of linker peptide joining VH and VL domain
EGKSSGSGSESKVD

SEQ ID NO: 48 - protein, 14 aa, Unknown
  an example of linker peptide joining VH and VL domain
GSTSGSGKSSEGKG

SEQ ID NO: 49 - protein, 18 aa, Unknown
  an example of linker peptide joining VH and VL domain
KESGSVSSEQLAQFRSLD

SEQ ID NO: 50 - protein, 16 aa, Unknown
  an example of linker peptide joining VH and VL domain
ESGSVSSEELAFRSLD

SEQ ID NO: 51 - protein, 4 aa, Unknown
  an example of localization signal peptide for endoplasmic reticulum
KDEL

SEQ ID NO: 52 - protein, 4 aa, Unknown
  an example of localization signal peptide for endoplasmic reticulum
DDEL

SEQ ID NO: 53 - protein, 4 aa, Unknown
  an example of localization signal peptide for endoplasmic reticulum
DEEL

SEQ ID NO: 54 - protein, 4 aa, Unknown
  an example of localization signal peptide for endoplasmic reticulum
QEDL

SEQ ID NO: 55 - protein, 4 aa, Unknown
  an example of localization signal peptide for nucleus
RDEL

SEQ ID NO: 56 - protein, 7 aa, Unknown
  an example of localization signal peptide for endoplasmic reticulum
PKKKRKV

SEQ ID NO: 57 - protein, 7 aa, Unknown
  an example of localization signal peptide for nucleus
PQKKIKS

SEQ ID NO: 58 - protein, 5 aa, Unknown
  a example of localization signal peptide for nucleus
QPKKP

SEQ ID NO: 59 - protein, 4 aa, Unknown
  an example of localization signal peptide for nucleus
RKKR

SEQ ID NO: 60 - protein, 5 aa, Unknown
  an example of localization signal peptide for nucleus
KKKRK

SEQ ID NO: 61 - protein, 12 aa, Unknown
  an example of localization signal peptide for nucleolar region
RKKRRQRRRAHQ

SEQ ID NO: 62 - protein, 16 aa, Unknown
  an example of localization signal peptide for nucleolar region
RQARRNRRRRWRERQR

SEQ ID NO: 63 - protein, 19 aa, Unknown
  an example of localization signal peptide for nucleolar region
MPLTRRRPAASQALAPPTP

SEQ ID NO: 64 - protein, 15 aa, Unknown
  an example of localization signal peptide for Endosomal compartment
MDDQRDLISNNEQLP

SEQ ID NO: 65 - protein, 32 aa, Unknown
  an example of localization signal peptide for mitochondrial matrix
MLFNLRXXLNNAAFRHGHNFMVRNFRCGQPLX

SEQ ID NO: 66 - protein, 3 aa, Unknown
  an example of localization signal peptide for peroxisome
AKL

SEQ ID NO: 67 - protein, 6 aa, Unknown
  an example of localization signal peptide for trans Golgi network
SDYQRL

SEQ ID NO: 68 - protein, 8 aa, Unknown
  an example of localization signal peptide for plasma membrane
GCVCSSNP

SEQ ID NO: 69 - protein, 8 aa, Unknown
  an example of localization signal peptide for plasma membrane
GQTVTTPL

SEQ ID NO: 70 - protein, 8 aa, Unknown
  an example of localization signal peptide for plasma membrane
GQELSQHE

SEQ ID NO: 71 - protein, 8 aa, Unknown
  an example of localization signal peptide for plasma membrane
GNSPSYNP

SEQ ID NO: 72 - protein, 8 aa, Unknown
  an example of localization signal peptide for plasma membrane
GVSGSKGQ

SEQ ID NO: 73 - protein, 8 aa, Unknown
  an example of localization signal peptide for plasma membrane
GQTITTPL

SEQ ID NO: 74 - protein, 8 aa, Unknown
  an example of localization signal peptide for plasma membrane
GQTLTTPL

SEQ ID NO: 75 - protein, 8 aa, Unknown
  an example of localization signal peptide for plasma membrane
GQIFSRSA

SEQ ID NO: 76 - protein, 8 aa, Unknown
  an example of localization signal peptide for plasma membrane
GQIHGLSP

SEQ ID NO: 77 - protein, 8 aa, Unknown
  an example of localization signal peptide for plasma membrane
GARASVLS

SEQ ID NO: 78 - protein, 8 aa, Unknown
  an example of localization signal peptide for plasma membrane
GCTLSAEE